SEQUENCE LISTING (195 sequences)

SEQ ID NO: 1 - protein, 249 aa, Homo sapiens
LHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 2 - protein, 5 aa, Artificial Sequence
  Synthetic Construct
DSYMS

SEQ ID NO: 3 - protein, 17 aa, Artificial Sequence
  Synthetic Construct
DMYPDNGDSSYNQKFRE

SEQ ID NO: 4 - protein, 8 aa, Artificial Sequence
  Synthetic Construct
APRWYFSV

SEQ ID NO: 5 - protein, 11 aa, Artificial Sequence
  Synthetic Construct
RASQDISNYLN

SEQ ID NO: 6 - protein, 7 aa, Artificial Sequence
  Synthetic Construct
YTSRLRS

SEQ ID NO: 7 - protein, 9 aa, Artificial Sequence
  Synthetic Construct
QQGHTLPPT

SEQ ID NO: 8 - protein, 5 aa, Artificial Sequence
  Synthetic Construct
DAYMS

SEQ ID NO: 9 - protein, 5 aa, Artificial Sequence
  Synthetic Construct
ESYMS

SEQ ID NO: 10 - protein, 17 aa, Artificial Sequence
  Synthetic Construct
DMYPDNADSSYNQKFRE

SEQ ID NO: 11 - protein, 17 aa, Artificial Sequence
  Synthetic Construct
DMYPDNADASYNQKFRE

SEQ ID NO: 12 - protein, 17 aa, Artificial Sequence
  Synthetic Construct
DMYPDNGDASYNQKFRE

SEQ ID NO: 13 - protein, 17 aa, Artificial Sequence
  Synthetic Construct
DMYPDSGDSSYNQKFRE

SEQ ID NO: 14 - protein, 17 aa, Artificial Sequence
  Synthetic Construct
DMYPDNGSSSYNQKFRE

SEQ ID NO: 15 - protein, 8 aa, Artificial Sequence
  Synthetic Construct
APRWYFSA

SEQ ID NO: 16 - protein, 8 aa, Artificial Sequence
  Synthetic Construct
APRWYASV

SEQ ID NO: 17 - protein, 8 aa, Artificial Sequence
  Synthetic Construct
APRWAFSV

SEQ ID NO: 18 - protein, 8 aa, Artificial Sequence
  Synthetic Construct
APAWYFSV

SEQ ID NO: 19 - protein, 8 aa, Artificial Sequence
  Synthetic Construct
APRWYFAV

SEQ ID NO: 20 - protein, 8 aa, Artificial Sequence
  Synthetic Construct
APRAYFSV

SEQ ID NO: 21 - protein, 8 aa, Artificial Sequence
  Synthetic Construct
AARWYFSV

SEQ ID NO: 22 - protein, 9 aa, Artificial Sequence
  Synthetic Construct
QQGHTLPAT

SEQ ID NO: 23 - protein, 9 aa, Artificial Sequence
  Synthetic Construct
QQGHTAPPT

SEQ ID NO: 24 - protein, 9 aa, Artificial Sequence
  Synthetic Construct
QQGATLPPT

SEQ ID NO: 25 - protein, 9 aa, Artificial Sequence
  Synthetic Construct
QQGHALPPT

SEQ ID NO: 26 - protein, 9 aa, Artificial Sequence
  Synthetic Construct
QQAHTLPPT

SEQ ID NO: 27 - protein, 9 aa, Artificial Sequence
  Synthetic Construct
QQGHTLAPT

SEQ ID NO: 28 - protein, 9 aa, Artificial Sequence
  Synthetic Construct
QAGHTLPPT

SEQ ID NO: 29 - protein, 5 aa, Artificial Sequence
  Synthetic Construct
NYLIE

SEQ ID NO: 30 - protein, 17 aa, Artificial Sequence
  Synthetic Construct
VINPGSGDTYYSEKFKG

SEQ ID NO: 31 - protein, 17 aa, Artificial Sequence
  Synthetic Construct
VINPGSGDAYYSEKFKG

SEQ ID NO: 32 - protein, 17 aa, Artificial Sequence
  Synthetic Construct
VINPGSGDQYYSEKFKG

SEQ ID NO: 33 - protein, 5 aa, Artificial Sequence
  Synthetic Construct
DRLDY

SEQ ID NO: 34 - protein, 5 aa, Artificial Sequence
  Synthetic Construct
ARLDY

SEQ ID NO: 35 - protein, 5 aa, Artificial Sequence
  Synthetic Construct
DALDY

SEQ ID NO: 36 - protein, 5 aa, Artificial Sequence
  Synthetic Construct
DRADY

SEQ ID NO: 37 - protein, 11 aa, Artificial Sequence
  Synthetic Construct
HASQDISSYIV

SEQ ID NO: 38 - protein, 7 aa, Artificial Sequence
  Synthetic Construct
HGTNLED

SEQ ID NO: 39 - protein, 7 aa, Artificial Sequence
  Synthetic Construct
HGTNLES

SEQ ID NO: 40 - protein, 7 aa, Artificial Sequence
  Synthetic Construct
HGTNLEE

SEQ ID NO: 41 - protein, 7 aa, Artificial Sequence
  Synthetic Construct
HGTNLEQ

SEQ ID NO: 42 - protein, 9 aa, Artificial Sequence
  Synthetic Construct
VHYAQFPYT

SEQ ID NO: 43 - protein, 9 aa, Artificial Sequence
  Synthetic Construct
AHYAQFPYT

SEQ ID NO: 44 - protein, 9 aa, Artificial Sequence
  Synthetic Construct
VAYAQFPYT

SEQ ID NO: 45 - protein, 9 aa, Artificial Sequence
  Synthetic Construct
VHAAQFPYT

SEQ ID NO: 46 - protein, 9 aa, Artificial Sequence
  Synthetic Construct
VHYAAFPYT

SEQ ID NO: 47 - protein, 9 aa, Artificial Sequence
  Synthetic Construct
VHYAQAPYT

SEQ ID NO: 48 - protein, 9 aa, Artificial Sequence
  Synthetic Construct
VHYAQFAYT

SEQ ID NO: 49 - protein, 9 aa, Artificial Sequence
  Synthetic Construct
VHYAQFPAT

SEQ ID NO: 50 - protein, 5 aa, Artificial Sequence
  Synthetic Construct
DYGVL

SEQ ID NO: 51 - protein, 16 aa, Artificial Sequence
  Synthetic Construct
MIWSGGTTDYNAAFIS

SEQ ID NO: 52 - protein, 5 aa, Artificial Sequence
  Synthetic Construct
EEMDY

SEQ ID NO: 53 - protein, 11 aa, Artificial Sequence
  Synthetic Construct
RASQDISNFLN

SEQ ID NO: 54 - protein, 7 aa, Artificial Sequence
  Synthetic Construct
YTSRLHS

SEQ ID NO: 55 - protein, 9 aa, Artificial Sequence
  Synthetic Construct
QQGNTLPWT

SEQ ID NO: 56 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 57 - protein, 107 aa, Artificial Sequence
  Synthetic Construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPPTFGQGTKVEIK

SEQ ID NO: 58 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITVDTSTSTAYLELSSLRSEDTAVYYCVLAPRWYFSVWGQGTLVTVSS

SEQ ID NO: 59 - protein, 107 aa, Artificial Sequence; identical to SEQ ID NO: 57
  Synthetic Construct

SEQ ID NO: 60 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTLTVDTSTSTAYLELSSLRSEDTAVYYCVLAPRWYFSVWGQGTLVTVSS

SEQ ID NO: 61 - protein, 107 aa, Artificial Sequence; identical to SEQ ID NO: 57
  Synthetic Construct

SEQ ID NO: 62 - protein, 117 aa, Artificial Sequence; identical to SEQ ID NO: 58
  Synthetic Construct

SEQ ID NO: 63 - protein, 107 aa, Artificial Sequence
  Synthetic Construct
DIQMTQSPSSLSASVGDRVTITCRASQDISNYLNWYQQKPGKTVKLLIYYTSRLRSGVPSRFSGSGSGTDFTLTISSLQPEDFATYYCQQGHTLPPTFGQGTKVEIK

SEQ ID NO: 64 - protein, 117 aa, Artificial Sequence; identical to SEQ ID NO: 58
  Synthetic Construct

SEQ ID NO: 65 - protein, 107 aa, Artificial Sequence; identical to SEQ ID NO: 63
  Synthetic Construct

SEQ ID NO: 66 - protein, 117 aa, Artificial Sequence; identical to SEQ ID NO: 58
  Synthetic Construct

SEQ ID NO: 67 - protein, 107 aa, Artificial Sequence
  Synthetic Construct
DIQMTQSPSSLSASVGDRVTITCRASQDISNYLNWYQQKPGKTVKLLIYYTSRLRSGVPSRFSGSGSGKDYTLTISSLQPEDFATYFCQQGHTLPPTFGQGTKVEIK

SEQ ID NO: 68 - protein, 117 aa, Artificial Sequence; identical to SEQ ID NO: 58
  Synthetic Construct

SEQ ID NO: 69 - protein, 107 aa, Artificial Sequence; identical to SEQ ID NO: 67
  Synthetic Construct

SEQ ID NO: 70 - protein, 117 aa, Artificial Sequence
  Synthetic Construct
EVQLVQSGAEVKKPGASVKVSCKASGYTFTDAYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 71 - protein, 107 aa, Artificial Sequence; identical to SEQ ID NO: 57
  Synthetic Construct

SEQ ID NO: 72 - protein, 117 aa, Artificial Sequence
  Synthetic Construct
EVQLVQSGAEVKKPGASVKVSCKASGYTFTESYMSWVRQAPGQGLEWIGDMYPDNGDSSYNQKFRERVTITRDTSTSTAYLELSSLRSEDTAVYYCVLAPRWYFSVWGQGTLVTVSS

SEQ ID NO: 73 - protein, 107 aa, Artificial Sequence; identical to SEQ ID NO: 57
  Synthetic Construct

SEQ ID NO: 74 - protein, 117 aa, Artificial Sequence
  Synthetic Construct
EVQLVQSGAEVKKPGASVKVSCKASGYTFTDSYMSWVRQAPGQGLEWIGDMYPDNADSSYNQKFRERVTITRDTSTSTAYLELSSLRSEDTAVYYCVLAPRWYFSVWGQGTLVTVSS

SEQ ID NO: 75 - protein, 107 aa, Artificial Sequence; identical to SEQ ID NO: 57
  Synthetic Construct

SEQ ID NO: 76 - protein, 117 aa, Artificial Sequence
  Synthetic Construct
EVQLVQSGAEVKKPGASVKVSCKASGYTFTDSYMSWVRQAPGQGLEWIGDMYPDNADASYNQKFRERVTITRDTSTSTAYLELSSLRSEDTAVYYCVLAPRWYFSVWGQGTLVTVSS

SEQ ID NO: 77 - protein, 107 aa, Artificial Sequence; identical to SEQ ID NO: 57
  Synthetic Construct

SEQ ID NO: 78 - protein, 117 aa, Artificial Sequence
  Synthetic Construct
EVQLVQSGAEVKKPGASVKVSCKASGYTFTDSYMSWVRQAPGQGLEWIGDMYPDNGDASYNQKFRERVTITRDTSTSTAYLELSSLRSEDTAVYYCVLAPRWYFSVWGQGTLVTVSS

SEQ ID NO: 79 - protein, 107 aa, Artificial Sequence; identical to SEQ ID NO: 57
  Synthetic Construct

SEQ ID NO: 80 - protein, 117 aa, Artificial Sequence
  Synthetic Construct
EVQLVQSGAEVKKPGASVKVSCKASGYTFTDSYMSWVRQAPGQGLEWIGDMYPDSGDSSYNQKFRERVTITRDTSTSTAYLELSSLRSEDTAVYYCVLAPRWYFSVWGQGTLVTVSS

SEQ ID NO: 81 - protein, 107 aa, Artificial Sequence; identical to SEQ ID NO: 57
  Synthetic Construct

SEQ ID NO: 82 - protein, 117 aa, Artificial Sequence
  Synthetic Construct
EVQLVQSGAEVKKPGASVKVSCKASGYTFTDSYMSWVRQAPGQGLEWIGDMYPDNGSSSYNQKFRERVTITRDTSTSTAYLELSSLRSEDTAVYYCVLAPRWYFSVWGQGTLVTVSS

SEQ ID NO: 83 - protein, 107 aa, Artificial Sequence; identical to SEQ ID NO: 57
  Synthetic Construct

SEQ ID NO: 84 - protein, 117 aa, Artificial Sequence
  Synthetic Construct
EVQLVQSGAEVKKPGASVKVSCKASGYTFTDAYMSWVRQAPGQGLEWIGDMYPDNADASYNQKFRERVTITRDTSTSTAYLELSSLRSEDTAVYYCVLAPRWYFSVWGQGTLVTVSS

SEQ ID NO: 85 - protein, 107 aa, Artificial Sequence; identical to SEQ ID NO: 57
  Synthetic Construct

SEQ ID NO: 86 - protein, 117 aa, Artificial Sequence; identical to SEQ ID NO: 56
  Synthetic Construct

SEQ ID NO: 87 - protein, 107 aa, Artificial Sequence
  Synthetic Construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLPATFGQGTKVEIK

SEQ ID NO: 88 - protein, 117 aa, Artificial Sequence; identical to SEQ ID NO: 56
  Synthetic Construct

SEQ ID NO: 89 - protein, 107 aa, Artificial Sequence
  Synthetic Construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTAPPTFGQGTKVEIK

SEQ ID NO: 90 - protein, 117 aa, Artificial Sequence; identical to SEQ ID NO: 56
  Synthetic Construct

SEQ ID NO: 91 - protein, 107 aa, Artificial Sequence
  Synthetic Construct
DIQMTQSPSSLSASVGDRVTITCRASQDISNYLNWYQQKPGKAPKLLIYYTSRLRSGVPSRFSGSGSGTDFTLTISSLQPEDFATYYCQQGATLPPTFGQGTKVEIK

SEQ ID NO: 92 - protein, 117 aa, Artificial Sequence; identical to SEQ ID NO: 56
  Synthetic Construct

SEQ ID NO: 93 - protein, 107 aa, Artificial Sequence
  Synthetic Construct
DIQMTQSPSSLSASVGDRVTITCRASQDISNYLNWYQQKPGKAPKLLIYYTSRLRSGVPSRFSGSGSGTDFTLTISSLQPEDFATYYCQQGHALPPTFGQGTKVEIK

SEQ ID NO: 94 - protein, 117 aa, Artificial Sequence; identical to SEQ ID NO: 56
  Synthetic Construct

SEQ ID NO: 95 - protein, 107 aa, Artificial Sequence
  Synthetic Construct
DIQMTQSPSSLSASVGDRVTITCRASQDISNYLNWYQQKPGKAPKLLIYYTSRLRSGVPSRFSGSGSGTDFTLTISSLQPEDFATYYCQQAHTLPPTFGQGTKVEIK

SEQ ID NO: 96 - protein, 117 aa, Artificial Sequence; identical to SEQ ID NO: 56
  Synthetic Construct

SEQ ID NO: 97 - protein, 107 aa, Artificial Sequence
  Synthetic Construct
DIQMTQSPSSLSASVGDRVTITCRASQDISNYLNWYQQKPGKAPKLLIYYTSRLRSGVPSRFSGSGSGTDFTLTISSLQPEDFATYYCQQGHTLAPTFGQGTKVEIK

SEQ ID NO: 98 - protein, 117 aa, Artificial Sequence; identical to SEQ ID NO: 56
  Synthetic Construct

SEQ ID NO: 99 - protein, 107 aa, Artificial Sequence
  Synthetic Construct
DIQMTQSPSSLSASVGDRVTITCRASQDISNYLNWYQQKPGKAPKLLIYYTSRLRSGVPSRFSGSGSGTDFTLTISSLQPEDFATYYCQAGHTLPPTFGQGTKVEIK

SEQ ID NO: 100 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFSAWGQGTLVTVSS

SEQ ID NO: 101 - protein, 107 aa, Artificial Sequence; identical to SEQ ID NO: 57
  Synthetic Construct

SEQ ID NO: 102 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYASVWGQGTLVTVSS

SEQ ID NO: 103 - protein, 107 aa, Artificial Sequence; identical to SEQ ID NO: 57
  Synthetic Construct

SEQ ID NO: 104 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWAFSVWGQGTLVTVSS

SEQ ID NO: 105 - protein, 107 aa, Artificial Sequence; identical to SEQ ID NO: 57
  Synthetic Construct

SEQ ID NO: 106 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPAWYFSVWGQGTLVTVSS

SEQ ID NO: 107 - protein, 107 aa, Artificial Sequence; identical to SEQ ID NO: 57
  Synthetic Construct

SEQ ID NO: 108 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRWYFAVWGQGTLVTVSS

SEQ ID NO: 109 - protein, 107 aa, Artificial Sequence; identical to SEQ ID NO: 57
  Synthetic Construct

SEQ ID NO: 110 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAPRAYFSVWGQGTLVTVSS

SEQ ID NO: 111 - protein, 107 aa, Artificial Sequence; identical to SEQ ID NO: 57
  Synthetic Construct

SEQ ID NO: 112 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVLAARWYFSVWGQGTLVTVSS

SEQ ID NO: 113 - protein, 107 aa, Artificial Sequence; identical to SEQ ID NO: 57
  Synthetic Construct

SEQ ID NO: 114 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCALAPRWYFSVWGQGTLVTVSS

SEQ ID NO: 115 - protein, 107 aa, Artificial Sequence; identical to SEQ ID NO: 57
  Synthetic Construct

SEQ ID NO: 116 - protein, 117 aa, Artificial Sequence
  Synthetic Construct
EVQLVQSGAEVKKPGASVKVSCKASGYTFTDSYMSWVRQAPGQGLEWIGDMYPDNGDSSYNQKFRERVTITRDTSTSTAYLELSSLRSEDTAVYYCVAAPRWYFSVWGQGTLVTVSS

SEQ ID NO: 117 - protein, 107 aa, Artificial Sequence; identical to SEQ ID NO: 57
  Synthetic Construct

SEQ ID NO: 118 - protein, 114 aa, Artificial Sequence
  Synthetic Construct
EVQLVQSGAEVKKPGASVKVSCKASGYAFTNYLIEWVRQAPGQGLEWIGVINPGSGDTYYSEKFKGRVTITRDTSTSTAYLELSSLRSEDTAVYYCARDRLDYWGQGTLVTVSS

SEQ ID NO: 119 - protein, 107 aa, Artificial Sequence
  Synthetic Construct
DIQMTQSPSSLSASVGDRVTITCHASQDISSYIVWYQQKPGKAPKLLIYHGTNLEDGVPSRFSGSGSGTDFTLTISSLQPEDFATYYCVHYAQFPYTFGQGTKVEIK

SEQ ID NO: 120 - protein, 114 aa, Artificial Sequence
  Synthetic Construct
EVQLVQSGAEVKKPGASVKVSCKASGYAFTNYLIEWVRQAPGQGLEWIGVINPGSGDTYYSEKFKGRVTITADTSTSTAYLELSSLRSEDTAVYYCARDRLDYWGQGTLVTVSS

SEQ ID NO: 121 - protein, 107 aa, Artificial Sequence; identical to SEQ ID NO: 119
  Synthetic Construct

SEQ ID NO: 122 - protein, 114 aa, Artificial Sequence
  Synthetic Construct
EVQLVQSGAEVKKPGASVKVSCKASGYAFTNYLIEWVRQAPGQGLEWIGVINPGSGDTYYSEKFKGRVTLTADTSTSTAYLELSSLRSEDTAVYYCARDRLDYWGQGTLVTVSS

SEQ ID NO: 123 - protein, 107 aa, Artificial Sequence; identical to SEQ ID NO: 119
  Synthetic Construct

SEQ ID NO: 124 - protein, 114 aa, Artificial Sequence; identical to SEQ ID NO: 120
  Synthetic Construct

SEQ ID NO: 125 - protein, 107 aa, Artificial Sequence
  Synthetic Construct
DIQMTQSPSSLSASVGDRVTITCHASQDISSYIVWYQQKPGKSFKGLIYHGTNLEDGVPSRFSGSGSGTDFTLTISSLQPEDFATYYCVHYAQFPYTFGQGTKVEIK

SEQ ID NO: 126 - protein, 114 aa, Artificial Sequence; identical to SEQ ID NO: 122
  Synthetic Construct

SEQ ID NO: 127 - protein, 107 aa, Artificial Sequence; identical to SEQ ID NO: 125
  Synthetic Construct

SEQ ID NO: 128 - protein, 114 aa, Artificial Sequence; identical to SEQ ID NO: 122
  Synthetic Construct

SEQ ID NO: 129 - protein, 107 aa, Artificial Sequence
  Synthetic Construct
DIQMTQSPSSLSASVGDRVTITCHASQDISSYIVWYQQKPGKSFKGLIYHGTNLESGVPSRFSGSGSGTDFTLTISSLQPEDFATYYCVHYAQFPYTFGQGTKVEIK

SEQ ID NO: 130 - protein, 114 aa, Artificial Sequence; identical to SEQ ID NO: 122
  Synthetic Construct

SEQ ID NO: 131 - protein, 107 aa, Artificial Sequence
  Synthetic Construct
DIQMTQSPSSLSASVGDRVTITCHASQDISSYIVWYQQKPGKSFKGLIYHGTNLEEGVPSRFSGSGSGTDFTLTISSLQPEDFATYYCVHYAQFPYTFGQGTKVEIK

SEQ ID NO: 132 - protein, 114 aa, Artificial Sequence; identical to SEQ ID NO: 122
  Synthetic Construct

SEQ ID NO: 133 - protein, 107 aa, Artificial Sequence
  Synthetic Construct
DIQMTQSPSSLSASVGDRVTITCHASQDISSYIVWYQQKPGKSFKGLIYHGTNLEQGVPSRFSGSGSGTDFTLTISSLQPEDFATYYCVHYAQFPYTFGQGTKVEIK

SEQ ID NO: 134 - protein, 114 aa, Artificial Sequence; identical to SEQ ID NO: 120
  Synthetic Construct

SEQ ID NO: 135 - protein, 107 aa, Artificial Sequence
  Synthetic Construct
DIQMTQSPSSLSASVGDRVTITCHASQDISSYIVWYQQKPGKSFKGLIYHGTNLEDGVPSRFSGSGSGADYTLTISSLQPEDFATYYCVHYAQFPYTFGQGTKVEIK

SEQ ID NO: 136 - protein, 114 aa, Artificial Sequence; identical to SEQ ID NO: 122
  Synthetic Construct

SEQ ID NO: 137 - protein, 107 aa, Artificial Sequence; identical to SEQ ID NO: 135
  Synthetic Construct

SEQ ID NO: 138 - protein, 114 aa, Artificial Sequence
  Synthetic Construct
EVQLVQSGAEVKKPGASVKVSCKASGYAFTNYLIEWVRQAPGQGLEWIGVINPGSGDTYYSEKFKGRVTLTRDTSTSTAYLELSSLRSEDTAVYYCARDRLDYWGQGTLVTVSS

SEQ ID NO: 139 - protein, 107 aa, Artificial Sequence; identical to SEQ ID NO: 125
  Synthetic Construct

SEQ ID NO: 140 - protein, 114 aa, Artificial Sequence; identical to SEQ ID NO: 138
  Synthetic Construct

SEQ ID NO: 141 - protein, 107 aa, Artificial Sequence
  Synthetic Construct
DIQMTQSPSSLSASVGDRVTITCHASQDISSYIVWYQQKPGKSPKLLIYHGTNLEDGVPSRFSGSGSGTDFTLTISSLQPEDFATYYCVHYAQFPYTFGQGTKVEIK

SEQ ID NO: 142 - protein, 114 aa, Artificial Sequence; identical to SEQ ID NO: 138
  Synthetic Construct

SEQ ID NO: 143 - protein, 107 aa, Artificial Sequence
  Synthetic Construct
DIQMTQSPSSLSASVGDRVTITCHASQDISSYIVWYQQKPGKAFKLLIYHGTNLEDGVPSRFSGSGSGTDFTLTISSLQPEDFATYYCVHYAQFPYTFGQGTKVEIK

SEQ ID NO: 144 - protein, 114 aa, Artificial Sequence; identical to SEQ ID NO: 138
  Synthetic Construct

SEQ ID NO: 145 - protein, 107 aa, Artificial Sequence
  Synthetic Construct
DIQMTQSPSSLSASVGDRVTITCHASQDISSYIVWYQQKPGKAPKGLIYHGTNLEDGVPSRFSGSGSGTDFTLTISSLQPEDFATYYCVHYAQFPYTFGQGTKVEIK

SEQ ID NO: 146 - protein, 114 aa, Artificial Sequence; identical to SEQ ID NO: 122
  Synthetic Construct

SEQ ID NO: 147 - protein, 107 aa, Artificial Sequence
  Synthetic Construct
DIQMTQSPSSLSASVGDRVTITCHASQDISSYIVWYQQKPGKSFKGLIYHGTNLEDGVPSRFSGSGSGTDFTLTISSLQPEDFATYYCAHYAQFPYTFGQGTKVEIK

SEQ ID NO: 148 - protein, 114 aa, Artificial Sequence; identical to SEQ ID NO: 122
  Synthetic Construct

SEQ ID NO: 149 - protein, 107 aa, Artificial Sequence
  Synthetic Construct
DIQMTQSPSSLSASVGDRVTITCHASQDISSYIVWYQQKPGKSFKGLIYHGTNLEDGVPSRFSGSGSGTDFTLTISSLQPEDFATYYCVAYAQFPYTFGQGTKVEIK

SEQ ID NO: 150 - protein, 114 aa, Artificial Sequence; identical to SEQ ID NO: 122
  Synthetic Construct

SEQ ID NO: 151 - protein, 107 aa, Artificial Sequence
  Synthetic Construct
DIQMTQSPSSLSASVGDRVTITCHASQDISSYIVWYQQKPGKSFKGLIYHGTNLEDGVPSRFSGSGSGTDFTLTISSLQPEDFATYYCVHAAQFPYTFGQGTKVEIK

SEQ ID NO: 152 - protein, 114 aa, Artificial Sequence; identical to SEQ ID NO: 122
  Synthetic Construct

SEQ ID NO: 153 - protein, 107 aa, Artificial Sequence
  Synthetic Construct
DIQMTQSPSSLSASVGDRVTITCHASQDISSYIVWYQQKPGKSFKGLIYHGTNLEDGVPSRFSGSGSGTDFTLTISSLQPEDFATYYCVHYAAFPYTFGQGTKVEIK

SEQ ID NO: 154 - protein, 114 aa, Artificial Sequence; identical to SEQ ID NO: 122
  Synthetic Construct

SEQ ID NO: 155 - protein, 107 aa, Artificial Sequence
  Synthetic Construct
DIQMTQSPSSLSASVGDRVTITCHASQDISSYIVWYQQKPGKSFKGLIYHGTNLEDGVPSRFSGSGSGTDFTLTISSLQPEDFATYYCVHYAQAPYTFGQGTKVEIK

SEQ ID NO: 156 - protein, 114 aa, Artificial Sequence; identical to SEQ ID NO: 122
  Synthetic Construct

SEQ ID NO: 157 - protein, 107 aa, Artificial Sequence
  Synthetic Construct
DIQMTQSPSSLSASVGDRVTITCHASQDISSYIVWYQQKPGKSFKGLIYHGTNLEDGVPSRFSGSGSGTDFTLTISSLQPEDFATYYCVHYAQFAYTFGQGTKVEIK

SEQ ID NO: 158 - protein, 114 aa, Artificial Sequence; identical to SEQ ID NO: 122
  Synthetic Construct

SEQ ID NO: 159 - protein, 107 aa, Artificial Sequence
  Synthetic Construct
DIQMTQSPSSLSASVGDRVTITCHASQDISSYIVWYQQKPGKSFKGLIYHGTNLEDGVPSRFSGSGSGTDFTLTISSLQPEDFATYYCVHYAQFPATFGQGTKVEIK

SEQ ID NO: 160 - protein, 114 aa, Artificial Sequence
  Synthetic Construct
EVQLVQSGAEVKKPGASVKVSCKASGYAFTNYLIEWVRQAPGQGLEWIGVINPGSGDTYYSEKFKGRVTLTADTSTSTAYLELSSLRSEDTAVYYCARARLDYWGQGTLVTVSS

SEQ ID NO: 161 - protein, 107 aa, Artificial Sequence; identical to SEQ ID NO: 125
  Synthetic Construct

SEQ ID NO: 162 - protein, 114 aa, Artificial Sequence
  Synthetic Construct
EVQLVQSGAEVKKPGASVKVSCKASGYAFTNYLIEWVRQAPGQGLEWIGVINPGSGDTYYSEKFKGRVTLTADTSTSTAYLELSSLRSEDTAVYYCARDALDYWGQGTLVTVSS

SEQ ID NO: 163 - protein, 107 aa, Artificial Sequence; identical to SEQ ID NO: 125
  Synthetic Construct

SEQ ID NO: 164 - protein, 114 aa, Artificial Sequence
  Synthetic Construct
EVQLVQSGAEVKKPGASVKVSCKASGYAFTNYLIEWVRQAPGQGLEWIGVINPGSGDTYYSEKFKGRVTLTADTSTSTAYLELSSLRSEDTAVYYCARDRADYWGQGTLVTVSS

SEQ ID NO: 165 - protein, 107 aa, Artificial Sequence; identical to SEQ ID NO: 125
  Synthetic Construct

SEQ ID NO: 166 - protein, 113 aa, Artificial Sequence
  Synthetic Construct
EVQLVESGPGLVKPSETLSLTCTVSGFSLTDYGVLWIRQPPGKGLEWIGMIWSGGTTDYNAAFISRVTISVDTSKNQFSLKLSSVTAADTAVYYCVREEMDYWGQGTLVTVSS

SEQ ID NO: 167 - protein, 107 aa, Artificial Sequence
  Synthetic Construct
DIQMTQSPSSLSASVGDRVTITCRASQDISNFLNWYQQKPGKAPKLLIYYTSRLHSGVPSRFSGSGSGTDFTLTISSLQPEDFATYYCQQGNTLPWTFGQGTKVEIK

SEQ ID NO: 168 - protein, 113 aa, Artificial Sequence
  Synthetic Construct
EVQLVESGPGLVKPSETLSLTCTVSGFSLTDYGVLWIRQPPGKGLEWIGMIWSGGTTDYNAAFISRVTISKDTSKNQVSLKLSSVTAADTAVYYCVREEMDYWGQGTLVTVSS

SEQ ID NO: 169 - protein, 107 aa, Artificial Sequence; identical to SEQ ID NO: 167
  Synthetic Construct

SEQ ID NO: 170 - protein, 113 aa, Artificial Sequence
  Synthetic Construct
EVQLVESGPGLVKPSETLSLTCTVSGFSLTDYGVLWVRQPPGKGLEWLGMIWSGGTTDYNAAFISRLTISKDTSKNQVSLKLSSVTAADTAVYYCVREEMDYWGQGTLVTVSS

SEQ ID NO: 171 - protein, 107 aa, Artificial Sequence; identical to SEQ ID NO: 167
  Synthetic Construct

SEQ ID NO: 172 - protein, 5 aa, Artificial Sequence
  Synthetic Construct
  VARIANT 1: Xaa = D or E
  VARIANT 2: Xaa = S or A
XXYMS

SEQ ID NO: 173 - protein, 17 aa, Artificial Sequence
  Synthetic Construct
  VARIANT 6: Xaa = N or S
  VARIANT 7: Xaa = A or G
  VARIANT 8: Xaa = D or S
  VARIANT 9: Xaa = A or S
DMYPDXXXXSYNQKFRE

SEQ ID NO: 174 - protein, 8 aa, Artificial Sequence
  Synthetic Construct
  VARIANT 5: Xaa = Y or A
  VARIANT 6: Xaa = A or F
  VARIANT 7: Xaa = S or A
  VARIANT 8: Xaa = A or V
APRWXXXX

SEQ ID NO: 175 - protein, 9 aa, Artificial Sequence
  Synthetic Construct
  VARIANT 2: Xaa = A or Q
  VARIANT 3: Xaa = A or G
  VARIANT 4: Xaa = A or H
  VARIANT 5: Xaa = A or T
  VARIANT 6: Xaa = A or L
  VARIANT 7: Xaa = A or P
  VARIANT 8: Xaa = A or P
QXXXXXXXT

SEQ ID NO: 176 - protein, 17 aa, Artificial Sequence
  Synthetic Construct
  VARIANT 9: Xaa = T, A or Q
VINPGSGDXYYSEKFKG

SEQ ID NO: 177 - protein, 7 aa, Artificial Sequence
  Synthetic Construct
  VARIANT 7: Xaa = S, E, or Q
HGTNLEX

SEQ ID NO: 178 - protein, 9 aa, Artificial Sequence
  Synthetic Construct
  VARIANT 1: Xaa = V or A
  VARIANT 2: Xaa = H or A
  VARIANT 9: Xaa = Y or A
XXYAQFPYX

SEQ ID NO: 179 - protein, 107 aa, Artificial Sequence
  Synthetic Construct
DIQMTQTTSSLSASLGDRVTISCRASQDISNYLNWYQQKPDGTVKLLIYYTSRLRSGVPSRFSGSGSGKDYFLTISNLEQEDVAAYFCQQGHTLPPTFGGGTKLEIK

SEQ ID NO: 180 - protein, 117 aa, Artificial Sequence
  Synthetic Construct
EVQLQQSGPELVKPGASVKISCKASGYTFTDSYMSWVKQSHGKTLEWIGDMYPDNGDSSYNQKFREKVTLTVDKSSTTAYMEFRSLTSEDSAVYYCVLAPRWYFSVWGTGTTVTVSS

SEQ ID NO: 181 - protein, 107 aa, Artificial Sequence
  Synthetic Construct
DILMTQSPSSMSVSLGDTVSITCHASQDISSYIVWLQQKPGKSFRGLIYHGTNLEDGIPSRFSGSGSGADYSLTISSLESEDFADYYCVHYAQFPYTFGGGTKLEIK

SEQ ID NO: 182 - protein, 114 aa, Artificial Sequence
  Synthetic Construct
QVQLQQSGAELVRPGTSVKVSCKASGYAFTNYLIEWVKQRPGQGLEWIGVINPGSGDTYYSEKFKGKVTLTADKSSSTAYMQLSSLTSEDSAVYFCARDRLDYWGQGTTLTVSS

SEQ ID NO: 183 - protein, 117 aa, Artificial Sequence; identical to SEQ ID NO: 60
  Synthetic Construct

SEQ ID NO: 184 - protein, 117 aa, Artificial Sequence; identical to SEQ ID NO: 60
  Synthetic Construct

SEQ ID NO: 185 - protein, 25 aa, Artificial Sequence [sequence omitted]

SEQ ID NO: 186 - protein, 13 aa, Artificial Sequence
  Synthetic Construct
WVRQAPGKGLEWV

SEQ ID NO: 187 - protein, 30 aa, Artificial Sequence
  Synthetic Construct
RFTISRDNSKNTLYLQMNSLRAEDTAVYYC

SEQ ID NO: 188 - protein, 11 aa, Artificial Sequence
  Synthetic Construct
WGQGTLVTVSS

SEQ ID NO: 189 - protein, 23 aa, Artificial Sequence
  Synthetic Construct
DIQMTQSPSSLSASVGDRVTITC

SEQ ID NO: 190 - protein, 15 aa, Artificial Sequence
  Synthetic Construct
WYQQKPGKAPKLLIY

SEQ ID NO: 191 - protein, 32 aa, Artificial Sequence
  Synthetic Construct
GVPSRFSGSGSGTDFTLTISSLQPEDFATYYC

SEQ ID NO: 192 - protein, 10 aa, Artificial Sequence
  Synthetic Construct
FGQGTKVEIK

SEQ ID NO: 193 - protein, 17 aa, Artificial Sequence
  Synthetic Construct
DMYPDAAAASYNQKFRE

SEQ ID NO: 194 - protein, 8 aa, Artificial Sequence
  Synthetic Construct
APRWAAAA

SEQ ID NO: 195 - protein, 9 aa, Artificial Sequence
  Synthetic Construct
QAAAAAAAT

(19) United States
(12) Patent Application Publication
(10) Pub. No.: US 2018/0346586 A1
(43) Pub. Date: Dec. 6, 2018

(54) METHODS OF TREATING CANCER USING ANTI-OX40 ANTIBODIES

(71) Applicant: Genentech, Inc., South San Francisco, CA (US)

(72) Inventors: Ina P. RHEE, Burlingame, CA (US); Jeong KIM, San Francisco, CA (US); Mahrukh HUSENI, Union City, CA (US); Eric STEFANICH, South San Francisco, CA (US); Sid SUKUMARAN, South San Francisco, CA (US); Chi-Chung LI, South San Francisco, CA (US)

(73) Assignee: Genentech, Inc., South San Francisco, CA (US)

(21) Appl. No.: 15/977,671

(22) Filed: May 11, 2018

Related U.S. Application Data
(63) Continuation of application No. 15/176,100, filed on Jun. 7, 2016, now abandoned.
(60) Provisional application No. 62/321,686, filed on Apr. 12, 2016.
(60) Provisional application No. 62/308,745, filed on Mar. 15, 2016.
(60) Provisional application No. 62/173,339, filed on Jun. 9, 2015.
(60) Provisional application No. 62/172,802, filed on Jun. 8, 2015.

(51) Int. Cl. C07K 16/28; C12Q 1/6886; A61K 9/00

(52) U.S. Cl. CPC C07K 16/2878; C12Q 2600/158; C12Q 2600/112; C12Q 1/6886; A61K 2039/545; A61K 9/0019; C07K 2317/56

(57) ABSTRACT

The invention provides methods of treating or delaying progression of cancer in an individual comprising administering to the individual an anti-human OX40 agonist antibody. In some embodiments, the antibody is administered in a dose selected from about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg.

### CROSS REFERENCE TO RELATED APPLICATIONS

This application claims the priority benefit of U.S. Provisional Application Ser. No. 62/172,802, filed Jun. 8, 2015; 62/173,339, filed Jun. 9, 2015; 62/308,745, filed Mar. 15, 2016; and 62/321,686, filed Apr. 12, 2016; each of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 146392033700SEQLIST.txt, date recorded: May 31, 2016, size: 141 KB.

### FIELD OF THE INVENTION

The present invention relates to methods of treating cancer using anti-OX40 antibodies.

### BACKGROUND

OX40 (also known as CD34, TNFRSF4 and ACT35) is a member of the tumor necrosis factor receptor superfamily. OX40 is not constitutively expressed on naïve T cells, but is induced after engagement of the T cell receptor (TCR). The ligand for OX40, OX40L, is predominantly expressed on antigen presenting cells. OX40 is highly expressed by activated CD4+ T cells, activated CD8+ T cells, memory T cells, and regulatory T cells. OX40 signaling can provide costimulatory signals to CD4 and CD8 T cells, leading to enhanced cell proliferation, survival, effector function and migration. OX40 signaling also enhances memory T cell development and function.

Regulatory T cells (Treg) cells are highly enriched in tumors and tumor draining lymph nodes derived from multiple cancer indications, including melanoma, NSCLC, renal, ovarian, colon, pancreatic, hepatocellular, and breast cancer. In a subset of these indications, increased intratumoral T reg cell densities are associated with poor patient prognosis, suggesting that these cells play an important role in suppressing antitumor immunity. OX40 positive tumor infiltrating lymphocytes have been described.

It is clear that there continues to be a need for agents that have clinical attributes that are optimal for development as therapeutic agents. The invention described herein meets these needs and provides other benefits.

All references cited herein, including patent applications and publications, are incorporated by reference in their entirety.

### SUMMARY

In one aspect, provided herein is method of treating or delaying progression of cancer in an individual comprising administering to the individual a dose selected from the group consisting of about 0.2 mg, about 0.8 mg, about 3.2 mg, about 12 mg, about 40 mg, about 130 mg, about 400 mg, and about 1200 mg of an anti-human OX40 agonist antibody, wherein the antibody comprises (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7, and wherein the individual is a human.

In another aspect, provided herein is a method of treating or delaying progression of cancer in an individual comprising administering to the individual an anti-human OX40 agonist antibody at a dose selected from the group consisting of about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg per administration, wherein the antibody comprises (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7, and wherein the individual is a human.

In some embodiments, the methods further comprise repeating the administration of the anti-human OX40 agonist antibody at one or more additional doses, wherein each dose of the one or more additional doses is selected from the group consisting of about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg per administration and is administered at an interval of about 2 weeks or about 14 days between each administration. In some embodiments, the methods further comprise repeating the administration of the anti-human OX40 agonist antibody at one or more additional doses, wherein each dose of the one or more additional doses is selected from the group consisting of about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg per administration and is administered at an interval of about 3 weeks or about 21 days between each administration.

In another aspect, provided herein is a kit for treating or delaying progression of cancer in an individual, comprising: (a) a container comprising an anti-human OX40 agonist antibody formulated for administration at a dose selected from the group consisting of about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg, wherein the anti-human OX40 agonist antibody comprises: an HVR-H1 comprising the amino acid sequence of SEQ ID NO:2; an HVR-H2 comprising the amino acid sequence of SEQ ID NO:3; an HVR-H3 comprising the amino acid sequence of SEQ ID NO:4; an HVR-L1 comprising the amino acid sequence of SEQ ID NO:5; an HVR-L2 comprising the amino acid sequence of SEQ ID NO:6; and an HVR-L3 comprising an amino acid sequence selected from SEQ ID NO:7; and (b) a package insert with instructions for treating or delaying progression of cancer in an individual, wherein the individual is a human. In some embodiments, the instructions are for treating or delaying progression of cancer in an individual using any of the methods described herein.

In some embodiments, the dose is administered intravenously. In some embodiments, the dose is about 300 mg.

In some embodiments, the methods further comprise: (a) after administering the antibody, monitoring the individual for an adverse event and/or efficacy of treatment; and (b) if the individual does not exhibit an adverse event, and/or if the treatment exhibits efficacy, administering to the individual a second dose of the anti-human OX40 agonist antibody, wherein the second dose is selected from the group consisting of about 0.2 mg, about 0.8 mg, about 3.2 mg, about 12 mg, about 40 mg, about 130 mg, about 400 mg, and about 1200 mg of the anti-human OX40 agonist antibody. In some embodiments, the methods further comprise administering to the individual a second dose of the anti-human OX40 agonist antibody, the second dose not being provided until from about 2 weeks to about 4 weeks after the first dose, wherein the second dose is selected from the group consisting of about 0.2 mg, about 0.8 mg, about 3.2 mg, about 12 mg, about 40 mg, about 130 mg, about 400 mg, and about 1200 mg of the anti-human OX40 agonist antibody. In some embodiments, the second dose is not provided until about 3 weeks after the first dose. In some embodiments, the second dose is not provided until about 21 days after the first dose. In some embodiments, the second dose of the anti-human OX40 agonist antibody is greater than the first dose of the anti-human OX40 agonist antibody. In some embodiments, the first dose and the second dose are administered intravenously.

In another aspect, provided herein is a method of treating or delaying progression of cancer in an individual comprising administering to the individual an anti-human OX40 agonist antibody at a dose selected from the group consisting of about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg, wherein the anti-human OX40 agonist antibody comprises (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7; wherein the individual is a human. In some embodiments, the methods further comprise repeating the administration of the anti-human OX40 agonist antibody at an interval of about 3 weeks or about 21 days between each administration.

In another aspect, provided herein is a kit for treating or delaying progression of cancer in an individual, comprising: (a) a container comprising an anti-human OX40 agonist antibody formulated for administration at an interval of about 3 weeks or about 21 days between each administration at a dose selected from the group consisting of about 0.8 mg, about 3.2 mg, about 12 mg, about 40 mg, about 80 mg, about 130 mg, about 160 mg, about 300 mg, about 320 mg, about 400 mg, about 600 mg, and about 1200 mg per administration, wherein the anti-human OX40 agonist antibody comprises: an HVR-H1 comprising the amino acid sequence of SEQ ID NO:2; an HVR-H2 comprising the amino acid sequence of SEQ ID NO:3; an HVR-H3 comprising the amino acid sequence of SEQ ID NO:4; an HVR-L1 comprising the amino acid sequence of SEQ ID NO:5; an HVR-L2 comprising the amino acid sequence of SEQ ID NO:6; and an HVR-L3 comprising an amino acid sequence selected from SEQ ID NO:7; and (b) a package insert with instructions for treating or delaying progression of cancer in an individual, wherein the individual is a human.

In another aspect, provided herein is a method of treating or delaying progression of cancer in an individual comprising administering to the individual an anti-human OX40 agonist antibody at a dose selected from the group consisting of about 0.5 mg, about 2 mg, about 8 mg, about 27 mg, about 53 mg, about 87 mg, about 107 mg, about 200 mg, about 213 mg, about 267 mg, about 400 mg, and about 800 mg, wherein the anti-human OX40 agonist antibody comprises (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7; wherein the individual is a human. In some embodiments, the methods further comprise repeating the administration of the anti-human OX40 agonist antibody at an interval of about 2 weeks or about 14 days between each administration.

In another aspect, provided herein is a kit for treating or delaying progression of cancer in an individual, comprising: (a) a container comprising an anti-human OX40 agonist antibody formulated for administration at an interval of about 2 weeks or about 14 days between each administration at a dose selected from the group consisting of about 0.5 mg, about 2 mg, about 8 mg, about 27 mg, about 53 mg, about 87 mg, about 107 mg, about 200 mg, about 213 mg, about 267 mg, about 400 mg, and about 800 mg per administration, wherein the anti-human OX40 agonist antibody comprises: an HVR-H1 comprising the amino acid sequence of SEQ ID NO:2; an HVR-H2 comprising the amino acid sequence of SEQ ID NO:3; an HVR-H3 comprising the amino acid sequence of SEQ ID NO:4; an HVR-L1 comprising the amino acid sequence of SEQ ID NO:5; an HVR-L2 comprising the amino acid sequence of SEQ ID NO:6; and an HVR-L3 comprising an amino acid sequence selected from SEQ ID NO:7; and (b) a package insert with instructions for treating or delaying progression of cancer in an individual, wherein the individual is a human.

In some embodiments, 1-10 additional doses of the anti-human OX40 agonist antibody are administered.

In some embodiments, each dose of the anti-human OX40 agonist antibody administered to the individual is the same. In some embodiments, each dose of the anti-human OX40 agonist antibody administered to the individual is not the same. In some embodiments, each dose of the anti-human OX40 agonist antibody is administered intravenously. In some embodiments, a first dose of the anti-human OX40 agonist antibody is administered to the individual at a first rate, wherein, after the administration of the first dose, one or more additional doses of the anti-human OX40 agonist antibody are administered to the individual at one or more subsequent rates, and wherein the first rate is slower than the one or more subsequent rates.

In some embodiments, the anti-human OX40 agonist antibody is a human or humanized antibody. In some embodiments, the antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 56, 58, 60, 62, 64, 66, 68, 183, or 184. In some embodiments, the VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to human OX40. In some embodiments, a total of 1 to 10 amino acids have been substituted, inserted and/or deleted in SEQ ID NO:56. In some embodiments, the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 57, 59, 61, 63, 65, 67, or 69. In some embodiments, the VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to human OX40. In some embodiments, a total of 1 to 10 amino acids have been substituted, inserted and/or deleted in SEQ ID NO: 57. In some embodiments, the antibody comprises a VH sequence of SEQ ID NO: 56. In some embodiments, the antibody comprises a VL sequence of SEQ ID NO: 57. In some embodiments, the antibody comprises a VH sequence of SEQ ID NO:56 and a VL sequence of SEQ ID NO: 57. In some embodiments, the antibody is a full length human IgG1 antibody. In some embodiments, the antibody is MOXR0916.

In some embodiments, the antibody is formulated in a pharmaceutical formulation comprising (a) the antibody at a concentration between about 10 mg/mL and about 100 mg/mL, (b) a polysorbate, wherein the polysorbate concentration is about 0.02% to about 0.06%; (c) a histidine buffer at pH 5.0 to 6.0; and (d) a saccharide, wherein the saccharide concentration is about 120 mM to about 320 mM.

In some embodiments, the treatment results in a sustained response in the individual after cessation of the treatment. In some embodiments, the treatment results in a complete response (CR) or partial response (PR) in the individual.

In some embodiments, the individual is immunotherapy-naïve. In some embodiments, the individual has a cancer selected from the group consisting of melanoma, triple-negative breast cancer, ovarian cancer, renal cell cancer, bladder cancer, non-small cell lung cancer, gastric cancer, and colorectal cancer. In some embodiments, the individual has melanoma, the melanoma has a BRAF V600 mutation, and, prior to the administration of the anti-human OX40 agonist antibody, the individual has been treated with a B-Raf and/or mitogen-activated protein kinase kinase (MEK) kinase inhibitor and exhibited disease progression or intolerance to the B-Raf and/or mitogen-activated protein kinase kinase (MEK) kinase inhibitor treatment. In some embodiments, the individual has non-small cell lung cancer, the non-small cell lung cancer has a sensitizing epidermal growth factor receptor (EGFR) mutation, and, prior to the administration of the anti-human OX40 agonist antibody, the individual has been treated with an EGFR tyrosine kinase inhibitor and exhibited disease progression or intolerance to the EGFR tyrosine kinase inhibitor treatment. In some embodiments, the individual has non-small cell lung cancer, the non-small cell lung cancer has an anaplastic lymphoma kinase (ALK) rearrangement, and, prior to the administration of the anti-human OX40 agonist antibody, the individual has been treated with an ALK tyrosine kinase inhibitor and exhibited disease progression or intolerance to the ALK tyrosine kinase inhibitor treatment. In some embodiments, the individual has renal cell cancer, and the renal cell cancer is refractory to a prior therapy. In some embodiments, the prior therapy comprises treatment with a VEGF inhibitor, an mTOR inhibitor, or both.

In another aspect, provided herein is a use of an anti-human OX40 agonist antibody in the manufacture of a medicament for treating or delaying progression of cancer in an individual, wherein the medicament comprises an anti-human OX40 agonist antibody formulated at a dose selected from the group consisting of about 0.8 mg, about 3.2 mg, about 12 mg, about 40 mg, about 80 mg, about 130 mg, about 160 mg, about 300 mg, about 320 mg, about 400 mg, about 600 mg, and about 1200 mg per administration, wherein the anti-human OX40 agonist antibody comprises (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7. In some embodiments, the individual is a human.

In another aspect, provided herein is a use of an anti-human OX40 agonist antibody in the manufacture of a medicament for treating or delaying progression of cancer in an individual, wherein the first medicament comprises an anti-human OX40 agonist antibody formulated for administration at an interval of about 3 weeks or about 21 days between each administration at a dose selected from the group consisting of about 0.8 mg, about 3.2 mg, about 12 mg, about 40 mg, about 80 mg, about 130 mg, about 160 mg, about 300 mg, about 320 mg, about 400 mg, about 600 mg, and about 1200 mg per administration, wherein the anti-human OX40 agonist antibody comprises (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7. In some embodiments, the individual is a human.

In another aspect, provided herein is a use of an anti-human OX40 agonist antibody in the manufacture of a medicament for treating or delaying progression of cancer in an individual in conjunction with a second medicament, wherein the medicament comprises an anti-human OX40 agonist antibody formulated for administration at an interval of about 2 weeks or about 14 days between each administration at a dose selected from the group consisting of about 0.5 mg, about 2 mg, about 8 mg, about 27 mg, about 53 mg, about 87 mg, about 107 mg, about 200 mg, about 213 mg, about 267 mg, about 400 mg, and about 800 mg per administration, wherein the anti-human OX40 agonist antibody comprises (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7. In some embodiments, the individual is a human.

In some embodiments, provided herein is a method of treating or delaying progression of cancer in an individual comprising administering to the individual MOXR0916 at a dose of about 300 mg, wherein the cancer is selected from the group consisting of melanoma, triple-negative breast cancer, ovarian cancer, renal cell cancer, bladder cancer, non-small cell lung cancer, gastric cancer, and colorectal cancer. In some embodiments, the method further comprises repeating the administration of MOXR0916 at a dose of about 300 mg per administration, and the administration is repeated at an interval of about 3 weeks or about 21 days between administrations. In some embodiments, the cancer is RCC. In some embodiments, the cancer is RCC, and the cancer is refractory to a treatment comprising a VEGF inhibitor and/or an mTOR inhibitor. In some embodiments, MOXR0916 is administered intravenously.

In some embodiments, provided herein is a method of treating or delaying progression of cancer in an individual comprising administering to the individual MOXR0916 at a dose of about 160 mg, wherein the cancer is selected from the group consisting of melanoma, triple-negative breast cancer, ovarian cancer, renal cell cancer, bladder cancer, non-small cell lung cancer, gastric cancer, and colorectal cancer. In some embodiments, the method further comprises repeating the administration of MOXR0916 at a dose of 160 mg per administration, and the administration is repeated at an interval of about 3 weeks or about 21 days between administrations. In some embodiments, the cancer is RCC. In some embodiments, the cancer is RCC, and the cancer is refractory to a treatment comprising a VEGF inhibitor and/or an mTOR inhibitor. In some embodiments, MOXR0916 is administered intravenously.

In some embodiments, provided herein is a method of treating or delaying progression of cancer in an individual comprising administering to the individual MOXR0916 at a dose of about 320 mg, wherein the cancer is selected from the group consisting of melanoma, triple-negative breast cancer, ovarian cancer, renal cell cancer, bladder cancer, non-small cell lung cancer, gastric cancer, and colorectal cancer. In some embodiments, the method further comprises repeating the administration of MOXR0916 at a dose of about 320 mg per administration, and the administration is repeated at an interval of about 3 weeks or about 21 days between administrations. In some embodiments, the cancer is RCC. In some embodiments, the cancer is RCC, and the cancer is refractory to a treatment comprising a VEGF inhibitor and/or an mTOR inhibitor. In some embodiments, MOXR0916 is administered intravenously.

In some embodiments, provided herein is a method of treating or delaying progression of cancer in an individual comprising administering to the individual MOXR0916 at a dose of about 400 mg, wherein the cancer is selected from the group consisting of melanoma, triple-negative breast cancer, ovarian cancer, renal cell cancer, bladder cancer, non-small cell lung cancer, gastric cancer, and colorectal cancer. In some embodiments, the method further comprises repeating the administration of MOXR0916 at a dose of about 400 mg per administration, and the administration is repeated at an interval of about 3 weeks or about 21 days between administrations. In some embodiments, the cancer is RCC. In some embodiments, the cancer is RCC, and the cancer is refractory to a treatment comprising a VEGF inhibitor and/or an mTOR inhibitor. In some embodiments, MOXR0916 is administered intravenously.

In some embodiments of any of the above embodiments, the methods may further comprise, after administering to the individual the anti-human OX40 agonist antibody, monitoring the responsiveness of the individual to said treatment by: (a) measuring an expression level of one or more marker genes in a sample obtained from the cancer of the individual, wherein the one or more marker genes are selected from the group consisting of CCR5, CD274, IL-7, TNFRSF14, TGFB1, CD40, CD4, PRF1, TNFSF4, CD86, CXCL9, CD3E, LAG3, PDCD1, CCL28, GZMB, IFNg, and IL-2RA; and (b) optionally, classifying the individual as responsive or non-responsive to treatment with the anti-human OX40 agonist antibody based on the expression level of the one or more marker genes in the sample, as compared with a reference, wherein an increased expression level of the one or more marker genes as compared with the reference indicates a responsive individual. In some embodiments of any of the above embodiments, the methods may further comprise, after administering to the individual the anti-human OX40 agonist antibody, monitoring the responsiveness of the individual to said treatment by: (a) measuring an expression level of one or more marker genes in a sample obtained from the cancer of the individual, wherein the one or more marker genes are selected from the group consisting of CD8b, EOMES, GZMA, GZMB, IFNg, and PRF1; and (b) optionally, classifying the individual as responsive or non-responsive to treatment with the anti-human OX40 agonist antibody based on the expression level of the one or more marker genes in the sample, as compared with a reference, wherein an increased expression level of the one or more marker genes as compared with the reference indicates a responsive individual. In some embodiments of any of the above embodiments, the methods may further comprise, after administering to the individual the anti-human OX40 agonist antibody, monitoring the responsiveness of the individual to said treatment by: (a) measuring an expression level of one or more marker genes in a sample obtained from the cancer of the individual, wherein the one or more marker genes are selected from the group consisting of CCL22, IL-2, RORC, IL-8, CTLA4, and FOXP3; and (b) optionally, classifying the individual as responsive or non-responsive to treatment with the anti-human OX40 agonist antibody based on the expression level of the one or more marker genes in the sample, as compared with a reference, wherein a decreased expression level of the one or more marker genes as compared with the reference indicates a responsive individual.

In another aspect, provided herein is a method for determining whether a cancer patient responds to a treatment with an anti-human OX40 agonist antibody, comprising measuring an expression level of one or more marker genes in a sample obtained from the cancer of the individual, wherein the one or more marker genes are selected from the group consisting of CCR5, CD274, IL-7, TNFRSF14, TGFB1, CD40, CD4, PRF1, TNFSF4, CD86, CXCL9, CD3E, LAG3, PDCD1, CCL28, GZMB, IFNg, and IL-2RA, wherein the expression level of the one or more marker genes is compared with a reference, and wherein an increased expression level of the one or more marker genes as compared with the reference indicates that the cancer patient responds to said treatment. In another aspect, provided herein is a method for determining whether a cancer patient responds to a treatment with an anti-human OX40 agonist antibody, comprising measuring an expression level of one or more marker genes in a sample obtained from the cancer of the individual, wherein the one or more marker genes are selected from the group consisting of CD8b, EOMES, GZMA, GZMB, IFNg, and PRF1, wherein the expression level of the one or more marker genes is compared with a reference, and wherein an increased expression level of the one or more marker genes as compared with the reference indicates that the cancer patient responds to said treatment. In another aspect, provided herein is a method for determining whether a cancer patient responds to a treatment with an anti-human OX40 agonist antibody, comprising measuring an expression level of one or more marker genes in a sample obtained from the cancer of the individual, wherein the one or more marker genes are selected from the group consisting of CCL22, IL-2, RORC, IL-8, CTLA4, and FOXP3, wherein the expression level of the one or more marker genes is compared with a reference, and wherein a decreased expression level of the one or more marker genes as compared with the reference indicates the cancer patient responds to said treatment.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE FIGURES

FIG. 1 provides a diagram of the study design and proposed cohorts.

FIG. 2 provides a pharmacokinetic (PK) plot of the mean serum concentration of MOXR0916 as a function of time from first dose for different dose groups.

FIGS. 3A-3G provide plots of OX40 receptor occupancy (%) on CD4+ T-cells at MOXR0916 doses of 0.2 mg (FIG. 3A), 3.2 mg (FIG. 3B), 12 mg (FIG. 3C), 40 mg (FIG. 3D), 80 mg (FIG. 3E), 160 mg (FIG. 3F), and 300 mg (FIG. 3G).

FIG. 4 shows expression of the effector T cell (Teff) gene signature in some tumor biopsies before (“predose”) and after (“postdose”) treatment with MOXR0916. The type of tumor and dose of MOXR0916 administered are indicated. The Teff gene signature represents an activated T effector phenotype and includes the mean expression levels of CD8b, EOMES, granzyme A, granzyme B, interferon gamma, and perforin.

FIG. 5 shows tumor immune modulation in a biopsy of an RCC tumor from a patient treated with MOXR0916 at a dose of 3.2 mg. Tumor gene expression is reported as postdose fold change, relative to predose levels.

### DETAILED DESCRIPTION OF EMBODIMENTS OF THE INVENTION

### I. Definitions

The term “dysfunction” in the context of immune dysfunction, refers to a state of reduced immune responsiveness to antigenic stimulation.

The term “dysfunctional”, as used herein, also includes refractory or unresponsive to antigen recognition, specifically, impaired capacity to translate antigen recognition into downstream T-cell effector functions, such as proliferation, cytokine production (e.g., gamma interferon) and/or target cell killing.

“Enhancing T cell function” means to induce, cause or stimulate an effector or memory T cell to have a renewed, sustained or amplified biological function. Examples of enhancing T-cell function include: increased secretion of γ-interferon from CD8⁺ effector T cells, increased secretion of γ-interferon from CD4+ memory and/or effector T-cells, increased proliferation of CD4+ effector and/or memory T cells, increased proliferation of CD8+ effector T-cells, increased antigen responsiveness (e.g., clearance), relative to such levels before the intervention. In one embodiment, the level of enhancement is at least 50%, alternatively 60%, 70%, 80%, 90%, 100%, 120%, 150%, 200%. The manner of measuring this enhancement is known to one of ordinary skill in the art.

“Tumor immunity” refers to the process in which tumors evade immune recognition and clearance. Thus, as a therapeutic concept, tumor immunity is “treated” when such evasion is attenuated, and the tumors are recognized and attacked by the immune system. Examples of tumor recognition include tumor binding, tumor shrinkage and tumor clearance.

“Sustained response” refers to the sustained effect on reducing tumor growth after cessation of a treatment. For example, the tumor size may remain to be the same or smaller as compared to the size at the beginning of the administration phase. In some embodiments, the sustained response has a duration at least the same as the treatment duration, at least 1.5×, 2.0×, 2.5×, or 3.0× length of the treatment duration.

“Immunogenicity” refers to the ability of a particular substance to provoke an immune response. Tumors are immunogenic and enhancing tumor immunogenicity aids in the clearance of the tumor cells by the immune response.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “agonist antibody,” as used herein, is an antibody which activates a biological activity of the antigen it binds.

“Antibody-dependent cell-mediated cytotoxicity” or “ADCC” refers to a form of cytotoxicity in which secreted immunoglobulin bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIII only, whereas monocytes express FcγRT, FcγRII, and FcγRIII. FcR expression on hematopoietic cells is summarized in Table 3 on page 464 of Ravetch and Kinet, Annu. Rev. Immunol 9:457-92 (1991). To assess ADCC activity of a molecule of interest, an in vitro ADCC assay, such as that described in U.S. Pat. No. 5,500,362 or 5,821,337 or U.S. Pat. No. 6,737,056 (Presta), may be performed. Useful effector cells for such assays include PBMC and NK cells. Alternatively, or additionally, ADCC activity of the molecule of interest may be assessed in vivo, e.g., in an animal model such as that disclosed in Clynes et al. PNAS (USA) 95:652-656 (1998).

The terms “anti-OX40 antibody” and “an antibody that binds to OX40” refer to an antibody that is capable of binding OX40 with sufficient affinity such that the antibody is useful as a diagnostic and/or therapeutic agent in targeting OX40. In one embodiment, the extent of binding of an anti-OX40 antibody to an unrelated, non-OX40 protein is less than about 10% of the binding of the antibody to OX40 as measured, e.g., by a radioimmunoassay (RIA). In certain embodiments, an antibody that binds to OX40 has a dissociation constant (Kd) of ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³ M). In certain embodiments, an anti-OX40 antibody binds to an epitope of OX40 that is conserved among OX40 from different species.

As use herein, the term “binds”, “specifically binds to” or is “specific for” refers to measurable and reproducible interactions such as binding between a target and an antibody, which is determinative of the presence of the target in the presence of a heterogeneous population of molecules including biological molecules. For example, an antibody that binds to or specifically binds to a target (which can be an epitope) is an antibody that binds this target with greater affinity, avidity, more readily, and/or with greater duration than it binds to other targets. In one embodiment, the extent of binding of an antibody to an unrelated target is less than about 10% of the binding of the antibody to the target as measured, e.g., by a radioimmunoassay (RIA). In certain embodiments, an antibody that specifically binds to a target has a dissociation constant (Kd) of ≤≤100 nM, ≤10 nM, ≤1 nM, or ≤0.1 nM. In certain embodiments, an antibody specifically binds to an epitope on a protein that is conserved among the protein from different species. In another embodiment, specific binding can include, but does not require exclusive binding.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “binding domain” refers to the region of a polypeptide that binds to another molecule. In the case of an FcR, the binding domain can comprise a portion of a polypeptide chain thereof (e.g. the alpha chain thereof) which is responsible for binding an Fc region. One useful binding domain is the extracellular domain of an FcR alpha chain.

A polypeptide with a variant IgG Fc with “altered” FcR, ADCC or phagocytosis activity is one which has either enhanced or diminished FcR binding activity (e.g, FcγR) and/or ADCC activity and/or phagocytosis activity compared to a parent polypeptide or to a polypeptide comprising a native sequence Fc region.

The term “OX40,” as used herein, refers to any native OX40 from any vertebrate source, including mammals such as primates (e.g. humans) and rodents (e.g., mice and rats), unless otherwise indicated. The term encompasses “full-length,” unprocessed OX40 as well as any form of OX40 that results from processing in the cell. The term also encompasses naturally occurring variants of OX40, e.g., splice variants or allelic variants. The amino acid sequence of an exemplary human OX40 is shown in SEQ ID NO:1.

“OX40 activation” refers to activation, of the OX40 receptor. Generally, OX40 activation results in signal transduction.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia or lymphoid malignancies. More particular examples of such cancers include, but not limited to, squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer and gastrointestinal stromal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, superficial spreading melanoma, lentigo maligna melanoma, acral lentiginous melanomas, nodular melanomas, multiple myeloma and B-cell lymphoma; chronic lymphocytic leukemia (CLL); acute lymphoblastic leukemia (ALL); hairy cell leukemia; chronic myeloblastic leukemia; and post-transplant lymphoproliferative disorder (PTLD), as well as abnormal vascular proliferation associated with phakomatoses, edema (such as that associated with brain tumors), Meigs' syndrome, brain, as well as head and neck cancer, and associated metastases. In certain embodiments, cancers that are amenable to treatment by the antibodies of the invention include breast cancer, colorectal cancer, rectal cancer, non-small cell lung cancer, glioblastoma, non-Hodgkins lymphoma (NHL), renal cell cancer, prostate cancer, liver cancer, pancreatic cancer, soft-tissue sarcoma, kaposi's sarcoma, carcinoid carcinoma, head and neck cancer, ovarian cancer, mesothelioma, and multiple myeloma. In some embodiments, the cancer is selected from: non-small cell lung cancer, glioblastoma, neuroblastoma, melanoma, breast carcinoma (e.g. triple-negative breast cancer), gastric cancer, colorectal cancer (CRC), and hepatocellular carcinoma. Yet, in some embodiments, the cancer is selected from: non-small cell lung cancer, colorectal cancer, breast carcinoma (e.g. triple-negative breast cancer), melanoma, ovarian cancer, renal cell cancer, and bladder cancer, including metastatic forms of those cancers. In some embodiments, the cancer is a locally advanced or metastatic solid tumor, e.g., of any of the solid cancers described above.

The terms “cell proliferative disorder” and “proliferative disorder” refer to disorders that are associated with some degree of abnormal cell proliferation. In one embodiment, the cell proliferative disorder is cancer.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

“Complement dependent cytotoxicity” or “CDC” refers to the lysis of a target cell in the presence of complement. Activation of the classical complement pathway is initiated by the binding of the first component of the complement system (Clq) to antibodies (of the appropriate subclass), which are bound to their cognate antigen. To assess complement activation, a CDC assay, e.g., as described in Gazzano-Santoro et al., J. Immunol. Methods 202:163 (1996), may be performed. Polypeptide variants with altered Fc region amino acid sequences (polypeptides with a variant Fc region) and increased or decreased Clq binding capability are described, e.g., in U.S. Pat. No. 6,194,551 B1 and WO 1999/51642. See also, e.g., Idusogie et al. J. Immunol. 164: 4178-4184 (2000).

The term “cytostatic agent” refers to a compound or composition which arrests growth of a cell either in vitro or in vivo. Thus, a cytostatic agent may be one which significantly reduces the percentage of cells in S phase. Further examples of cytostatic agents include agents that block cell cycle progression by inducing G0/G1 arrest or M-phase arrest. The humanized anti-Her2 antibody trastuzumab (HERCEPTIN®) is an example of a cytostatic agent that induces GO/G1 arrest. Classical M-phase blockers include the vincas (vincristine and vinblastine), taxanes, and topoisomerase II inhibitors such as doxorubicin, epirubicin, daunorubicin, etoposide, and bleomycin. Certain agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in Mendelsohn and Israel, eds., The Molecular Basis of Cancer, Chapter 1, entitled “Cell cycle regulation, oncogenes, and antineoplastic drugs” by Murakami et al. (W.B. Saunders, Philadelphia, 1995), e.g., p. 13. The taxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). Paclitaxel and docetaxel promote the assembly of microtubules from tubulin dimers and stabilize microtubules by preventing depolymerization, which results in the inhibition of mitosis in cells.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

A “depleting anti-OX40 antibody,” is an anti-OX40 antibody that kills or depletes OX40-expressing cells. Depletion of OX40 expressing cells can be achieved by various mechanisms, such as antibody-dependent cell-mediated cytotoxicity and/or phagocytosis. Depletion of OX40-expressing cells may be assayed in vitro, and exemplary methods for in vitro ADCC and phagocytosis assays are provided herein. In some embodiments, the OX40-expressing cell is a human CD4+ effector T cell. In some embodiments, the OX40-expressing cell is a transgenic BT474 cell that expresses human OX40.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

“Fc receptor” or “FcR” describes a receptor that binds to the Fc region of an antibody. In some embodiments, an FcR is a native human FcR. In some embodiments, an FcR is one which binds an IgG antibody (a gamma receptor) and includes receptors of the FcγRI, FcγRII, and FcγRIII subclasses, including allelic variants and alternatively spliced forms of those receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see, e.g., Daeron, Annu. Rev. Immunol. 15:203-234 (1997)). FcRs are reviewed, for example, in Ravetch and Kinet, Annu. Rev. Immunol 9:457-92 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term “Fc receptor” or “FcR” also includes the neonatal receptor, FcRn, which is responsible for the transfer of maternal IgGs to the fetus (Guyer et al., J. Immunol. 117:587 (1976) and Kim et al., J. Immunol. 24:249 (1994)) and regulation of homeostasis of immunoglobulins. Methods of measuring binding to FcRn are known (see, e.g., Ghetie and Ward., Immunol. Today 18(12):592-598 (1997); Ghetie et al., Nature Biotechnology, 15(7):637-640 (1997); Hinton et al., J. Biol. Chem. 279(8):6213-6216 (2004); WO 2004/92219 (Hinton et al.). Binding to human FcRn in vivo and serum half life of human FcRn high affinity binding polypeptides can be assayed, e.g., in transgenic mice or transfected human cell lines expressing human FcRn, or in primates to which the polypeptides with a variant Fc region are administered. WO 2000/42072 (Presta) describes antibody variants with improved or diminished binding to FcRs. See also, e.g., Shields et al. J. Biol. Chem. 9(2):6591-6604 (2001).

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

A “functional Fc region” possesses an “effector function” of a native sequence Fc region. Exemplary “effector functions” include C1q binding; CDC; Fc receptor binding; ADCC; phagocytosis; down regulation of cell surface receptors (e.g. B cell receptor; BCR), etc. Such effector functions generally require the Fc region to be combined with a binding domain (e.g., an antibody variable domain) and can be assessed using various assays as disclosed, for example, in definitions herein.

“Human effector cells” refer to leukocytes that express one or more FcRs and perform effector functions. In certain embodiments, the cells express at least FcγRIII and perform ADCC effector function(s). Examples of human leukocytes which mediate ADCC include peripheral blood mononuclear cells (PBMC), natural killer (NK) cells, monocytes, cytotoxic T cells, and neutrophils. The effector cells may be isolated from a native source, e.g., from blood.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));
(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));
(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and
(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human.

“Promoting cell growth or proliferation” means increasing a cell's growth or proliferation by at least 10%, 20%, 30%, 40%, 50%, 60%, 70%, 80%, 90%, 95%, or 100%.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding an anti-OX40 antibody” refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical formulation.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain. A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “tumor” refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

A “VH subgroup III consensus framework” comprises the consensus sequence obtained from the amino acid sequences in variable heavy subgroup III of Kabat et al. In one embodiment, the VH subgroup III consensus framework amino acid sequence comprises at least a portion or all of each of the following sequences: EVQLVESGGGLVQPGGSLRLSCAAS (SEQ ID NO:185)-H1-WVRQAPGKGLEWV (SEQ ID NO:186)-H2-RFTISRDNSKNTLYLQMNSLRAEDTAVYYC (SEQ ID NO:187)-H3-WGQGTLVTVSS (SEQ ID NO:188).

A “VL subgroup I consensus framework” comprises the consensus sequence obtained from the amino acid sequences in variable light kappa subgroup I of Kabat et al. In one embodiment, the VH subgroup I consensus framework amino acid sequence comprises at least a portion or all of each of the following sequences: DIQMTQSPSSLSASVGDRVTITC (SEQ ID NO:189)-L1-WYQQKPGKAPKLLIY (SEQ ID NO:190)-L2-GVPSRFSGSGSGTDFTLTISSLQPEDFATYYC (SEQ ID NO:191)-L3-FGQGTKVEIK (SEQ ID NO:192).

The term “cytotoxic agent” as used herein refers to a substance that inhibits or prevents a cellular function and/or causes cell death or destruction. Cytotoxic agents include, but are not limited to, radioactive isotopes (e.g., At211, I131, I125, Y90, Re186, Re188, Sm153, Bi212, P32, Pb212 and radioactive isotopes of Lu); chemotherapeutic agents; growth inhibitory agents; enzymes and fragments thereof such as nucleolytic enzymes; and toxins such as small molecule toxins or enzymatically active toxins of bacterial, fungal, plant or animal origin, including fragments and/or variants thereof. Exemplary cytotoxic agents can be selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A; inhibitors of fatty acid biosynthesis; cell cycle signalling inhibitors; HDAC inhibitors, proteasome inhibitors; and inhibitors of cancer metabolism.

In one embodiment the cytotoxic agent is selected from anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, inhibitors of LDH-A, inhibitors of fatty acid biosynthesis, cell cycle signalling inhibitors, HDAC inhibitors, proteasome inhibitors, and inhibitors of cancer metabolism. In one embodiment the cytotoxic agent is a taxane. In one embodiment the taxane is paclitaxel or docetaxel. In one embodiment the cytotoxic agent is a platinum agent. In one embodiment the cytotoxic agent is an antagonist of EGFR. In one embodiment the antagonist of EGFR is N-(3-ethynylphenyl)-6,7-bis(2-methoxyethoxy)quinazolin-4-amine (e.g., erlotinib). In one embodiment the cytotoxic agent is a RAF inhibitor. In one embodiment, the RAF inhibitor is a BRAF and/or CRAF inhibitor. In one embodiment the RAF inhibitor is vemurafenib. In one embodiment the cytotoxic agent is a PI3K inhibitor.

“Chemotherapeutic agent” includes chemical compounds useful in the treatment of cancer. Examples of chemotherapeutic agents include erlotinib (TARCEVA®, Genentech/OSI Pharm.), bortezomib (VELCADE®, Millennium Pharm.), disulfiram, epigallocatechin gallate, salinosporamide A, carfilzomib, 17-AAG (geldanamycin), radicicol, lactate dehydrogenase A (LDH-A), fulvestrant (FASLODEX®, AstraZeneca), sunitib (SUTENT®, Pfizer/Sugen), letrozole (FEMARA®, Novartis), imatinib mesylate (GLEEVEC®, Novartis), finasunate (VATALANIB®, Novartis), oxaliplatin (ELOXATIN®, Sanofi), 5-FU (5-fluorouracil), leucovorin, Rapamycin (Sirolimus, RAPAMUNEO, Wyeth), Lapatinib (TYKERB®, GSK572016, Glaxo Smith Kline), Lonafamib (SCH 66336), sorafenib (NEXAVAR®, Bayer Labs), gefitinib (IRESSA®, AstraZeneca), AG1478, alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including topotecan and irinotecan); bryostatin; callystatin; CC-1065 (including its adozelesin, carzelesin and bizelesin synthetic analogs); cryptophycins (particularly cryptophycin 1 and cryptophycin 8); adrenocorticosteroids (including prednisone and prednisolone); cyproterone acetate; 5α-reductases including finasteride and dutasteride); vorinostat, romidepsin, panobinostat, valproic acid, mocetinostat dolastatin; aldesleukin, talc duocarmycin (including the synthetic analogs, KW-2189 and CB1-TM1); eleutherobin; pancratistatin; a sarcodictyin; spongistatin; nitrogen mustards such as chlorambucil, chlomaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g., calicheamicin, especially calicheamicin γ1I and calicheamicin ω1I (Angew Chem. Intl. Ed. Engl. 1994 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® (doxorubicin), morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidamnol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL (paclitaxel; Bristol-Myers Squibb Oncology, Princeton, N.J.), ABRAXANE® (Cremophor-free), albumin-engineered nanoparticle formulations of paclitaxel (American Pharmaceutical Partners, Schaumberg, Ill.), and TAXOTERE® (docetaxel, doxetaxel; Sanofi-Aventis); chloranmbucil; GEMZAR® (gemcitabine); 6-thioguanine; mercaptopurine; methotrexate; platinum analogs such as cisplatin and carboplatin; vinblastine; etoposide (VP-16); ifosfamide; mitoxantrone; vincristine; NAVELBINE® (vinorelbine); novantrone; teniposide; edatrexate; daunomycin; aminopterin; capecitabine (XELODA®); ibandronate; CPT-11; topoisomerase inhibitor RFS 2000; difluoromethylornithine (DMFO); retinoids such as retinoic acid; and pharmaceutically acceptable salts, acids and derivatives of any of the above.

Chemotherapeutic agent also includes (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, iodoxyfene, 4-hydroxytamoxifen, trioxifene, keoxifene, LY117018, onapristone, and FARESTON® (toremifene citrate); (ii) aromatase inhibitors that inhibit the enzyme aromatase, which regulates estrogen production in the adrenal glands, such as, for example, 4(5)-imidazoles, aminoglutethimide, MEGASE® (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide and goserelin; buserelin, tripterelin, medroxyprogesterone acetate, diethylstilbestrol, premarin, fluoxymesterone, all transretionic acid, fenretinide, as well as troxacitabine (a 1,3-dioxolane nucleoside cytosine analog); (iv) protein kinase inhibitors; (v) lipid kinase inhibitors; (vi) antisense oligonucleotides, particularly those which inhibit expression of genes in signaling pathways implicated in aberrant cell proliferation, such as, for example, PKC-alpha, Ralf and H-Ras; (vii) ribozymes such as VEGF expression inhibitors (e.g., ANGIOZYME®) and HER2 expression inhibitors; (viii) vaccines such as gene therapy vaccines, for example, ALLOVECTIN®, LEUVECTIN®, and VAXID®; PROLEUKIN®, rIL-2; a topoisomerase 1 inhibitor such as LURTOTECAN®; ABARELIX® rmRH; and (ix) pharmaceutically acceptable salts, acids and derivatives of any of the above.

Chemotherapeutic agent also includes antibodies such as alemtuzumab (Campath), bevacizumab (AVASTIN®, Genentech); cetuximab (ERBITUX®, Imclone); panitumumab (VECTIBIX®, Amgen), rituximab (RITUXAN®, Genentech/Biogen Idec), pertuzumab (OMNITARG®, 2C4, Genentech), trastuzumab (HERCEPTIN®, Genentech), tositumomab (Bexxar, Corixia), and the antibody drug conjugate, gemtuzumab ozogamicin (MYLOTARG®, Wyeth). Additional humanized monoclonal antibodies with therapeutic potential as agents in combination with the compounds of the invention include: apolizumab, aselizumab, atlizumab, bapineuzumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, ocrelizumab, omalizumab, palivizumab, pascolizumab, pecfusituzumab, pectuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, tucotuzumab celmoleukin, tucusituzumab, umavizumab, urtoxazumab, ustekinumab, visilizumab, and the anti-interleukin-12 (ABT-874/J695, Wyeth Research and Abbott Laboratories) which is a recombinant exclusively human-sequence, full-length IgG1λ antibody genetically modified to recognize interleukin-12 p40 protein.

Chemotherapeutic agent also includes “EGFR inhibitors,” which refers to compounds that bind to or otherwise interact directly with EGFR and prevent or reduce its signaling activity, and is alternatively referred to as an “EGFR antagonist.” Examples of such agents include antibodies and small molecules that bind to EGFR. Examples of antibodies which bind to EGFR include MAb 579 (ATCC CRL HB 8506), MAb 455 (ATCC CRL HB8507), MAb 225 (ATCC CRL 8508), MAb 528 (ATCC CRL 8509) (see, U.S. Pat. No. 4,943,533, Mendelsohn et al.) and variants thereof, such as chimerized 225 (C225 or Cetuximab; ERBUTIX®) and reshaped human 225 (H225) (see, WO 96/40210, Imclone Systems Inc.); IMC-11F8, a fully human, EGFR-targeted antibody (Imclone); antibodies that bind type II mutant EGFR (U.S. Pat. No. 5,212,290); humanized and chimeric antibodies that bind EGFR as described in U.S. Pat. No. 5,891,996; and human antibodies that bind EGFR, such as ABX-EGF or Panitumumab (see WO98/50433, Abgenix/Amgen); EMD 55900 (Stragliotto et al. Eur. J. Cancer 32A:636-640 (1996)); EMD7200 (matuzumab) a humanized EGFR antibody directed against EGFR that competes with both EGF and TGF-alpha for EGFR binding (EMD/Merck); human EGFR antibody, HuMax-EGFR (GenMab); fully human antibodies known as E1.1, E2.4, E2.5, E6.2, E6.4, E2.11, E6. 3 and E7.6. 3 and described in U.S. Pat. No. 6,235,883; MDX-447 (Medarex Inc); and mAb 806 or humanized mAb 806 (Johns et al., J. Biol. Chem. 279(29):30375-30384 (2004)). The anti-EGFR antibody may be conjugated with a cytotoxic agent, thus generating an immunoconjugate (see, e.g., EP659,439A2, Merck Patent GmbH). EGFR antagonists include small molecules such as compounds described in U.S. Pat. Nos. 5,616,582, 5,457,105, 5,475,001, 5,654,307, 5,679,683, 6,084,095, 6,265,410, 6,455,534, 6,521,620, 6,596,726, 6,713,484, 5,770,599, 6,140,332, 5,866,572, 6,399,602, 6,344,459, 6,602,863, 6,391,874, 6,344,455, 5,760,041, 6,002,008, and 5,747,498, as well as the following PCT publications: WO98/14451, WO98/50038, WO99/09016, and WO99/24037. Particular small molecule EGFR antagonists include OSI-774 (CP-358774, erlotinib, TARCEVA® Genentech/OSI Pharmaceuticals); PD 183805 (CI 1033, 2-propenamide, N-[4-[(3-chloro-4-fluorophenyl)amino]-7-[3-(4-morpholinyl)propoxy]-6-quinazolinyl]-, dihydrochloride, Pfizer Inc.); ZD1839, gefitinib (IRESSA®) 4-(3′-Chloro-4′-fluoroanilino)-7-methoxy-6-(3-morpholinopropoxy)quinazoline, AstraZeneca); ZM 105180 ((6-amino-4-(3-methylphenyl-amino)-quinazoline, Zeneca); BIBX-1382 (N8-(3-chloro-4-fluoro-phenyl)-N2-(1-methyl-piperidin-4-yl)-pyrimido[5,4-d]pyrimidine-2,8-diamine, Boehringer Ingelheim); PKI-166 ((R)-4-[4-[(1-phenylethyl)amino]-1H-pyrrolo[2,3-d]pyrimidin-6-yl]-phenol); (R)-6-(4-hydroxyphenyl)-4-[(1-phenylethyl)amino]-7H-pyrrolo[2,3-d]pyrimidine); CL-387785 (N-[4-[(3-bromophenyl)amino]-6-quinazolinyl]-2-butynamide); EKB-569 (N-[4-[(3-chloro-4-fluorophenyl)amino]-3-cyano-7-ethoxy-6-quinolinyl]-4-(dimethylamino)-2-butenamide) (Wyeth); AG1478 (Pfizer); AG1571 (SU 5271; Pfizer); dual EGFR/HER2 tyrosine kinase inhibitors such as lapatinib (TYKERB®, GSK572016 or N-[3-chloro-4-[(3 fluorophenyl)methoxy]phenyl]-6[5[[[2methylsulfonyl)ethyl]amino]methyl]-2-furanyl]-4-quinazolinamine).

Chemotherapeutic agents also include “tyrosine kinase inhibitors” including the EGFR-targeted drugs noted in the preceding paragraph; small molecule HER2 tyrosine kinase inhibitor such as TAK165 available from Takeda; CP-724,714, an oral selective inhibitor of the ErbB2 receptor tyrosine kinase (Pfizer and OSI); dual-HER inhibitors such as EKB-569 (available from Wyeth) which preferentially binds EGFR but inhibits both HER2 and EGFR-overexpressing cells; lapatinib (GSK572016; available from Glaxo-SmithKline), an oral HER2 and EGFR tyrosine kinase inhibitor; PKI-166 (available from Novartis); pan-HER inhibitors such as canertinib (CI-1033; Pharmacia); Raf-1 inhibitors such as antisense agent ISIS-5132 available from ISIS Pharmaceuticals which inhibit Raf-1 signaling; non-HER targeted TK inhibitors such as imatinib mesylate (GLEEVEC®, available from Glaxo SmithKline); multi-targeted tyrosine kinase inhibitors such as sunitinib (SUTENT®, available from Pfizer); VEGF receptor tyrosine kinase inhibitors such as vatalanib (PTK787/ZK222584, available from Novartis/Schering AG); MAPK extracellular regulated kinase I inhibitor CI-1040 (available from Pharmacia); quinazolines, such as PD 153035,4-(3-chloroanilino) quinazoline; pyridopyrimidines; pyrimidopyrimidines; pyrrolopyrimidines, such as CGP 59326, CGP 60261 and CGP 62706; pyrazolopyrimidines, 4-(phenylamino)-7H-pyrrolo[2,3-d] pyrimidines; curcumin (diferuloyl methane, 4,5-bis (4-fluoroanilino)phthalimide); tyrphostines containing nitrothiophene moieties; PD-0183805 (Warner-Lamber); antisense molecules (e.g. those that bind to HER-encoding nucleic acid); quinoxalines (U.S. Pat. No. 5,804,396); tryphostins (U.S. Pat. No. 5,804,396); ZD6474 (Astra Zeneca); PTK-787 (Novartis/Schering AG); pan-HER inhibitors such as CI-1033 (Pfizer); Affinitac (ISIS 3521; Isis/Lilly); imatinib mesylate (GLEEVEC®); PKI 166 (Novartis); GW2016 (Glaxo SmithKline); CI-1033 (Pfizer); EKB-569 (Wyeth); Semaxinib (Pfizer); ZD6474 (AstraZeneca); PTK-787 (Novartis/Schering AG); INC-1C11 (Imclone), rapamycin (sirolimus, RAPAMUNE®); or as described in any of the following patent publications: U.S. Pat. No. 5,804,396; WO 1999/09016 (American Cyanamid); WO 1998/43960 (American Cyanamid); WO 1997/38983 (Warner Lambert); WO 1999/06378 (Warner Lambert); WO 1999/06396 (Warner Lambert); WO 1996/30347 (Pfizer, Inc); WO 1996/33978 (Zeneca); WO 1996/3397 (Zeneca) and WO 1996/33980 (Zeneca).

Chemotherapeutic agents also include dexamethasone, interferons, colchicine, metoprine, cyclosporine, amphotericin, metronidazole, alemtuzumab, alitretinoin, allopurinol, amifostine, arsenic trioxide, asparaginase, BCG live, bevacuzimab, bexarotene, cladribine, clofarabine, darbepoetin alfa, denileukin, dexrazoxane, epoetin alfa, elotinib, filgrastim, histrelin acetate, ibritumomab, interferon alfa-2a, interferon alfa-2b, lenalidomide, levamisole, mesna, methoxsalen, nandrolone, nelarabine, nofetumomab, oprelvekin, palifermin, pamidronate, pegademase, pegaspargase, pegfilgrastim, pemetrexed disodium, plicamycin, porfimer sodium, quinacrine, rasburicase, sargramostim, temozolomide, VM-26, 6-TG, toremifene, tretinoin, ATRA, valrubicin, zoledronate, and zoledronic acid, and pharmaceutically acceptable salts thereof.

Chemotherapeutic agents also include hydrocortisone, hydrocortisone acetate, cortisone acetate, tixocortol pivalate, triamcinolone acetonide, triamcinolone alcohol, mometasone, amcinonide, budesonide, desonide, fluocinonide, fluocinolone acetonide, betamethasone, betamethasone sodium phosphate, dexamethasone, dexamethasone sodium phosphate, fluocortolone, hydrocortisone-17-butyrate, hydrocortisone-17-valerate, aclometasone dipropionate, betamethasone valerate, betamethasone dipropionate, prednicarbate, clobetasone-17-butyrate, clobetasol-17-propionate, fluocortolone caproate, fluocortolone pivalate and fluprednidene acetate; immune selective anti-inflammatory peptides (ImSAIDs) such as phenylalanine-glutamine-glycine (FEG) and its D-isomeric form (feG) (IMULAN BioTherapeutics, LLC); anti-rheumatic drugs such as azathioprine, ciclosporin (cyclosporine A), D-penicillamine, gold salts, hydroxychloroquine, leflunomideminocycline, sulfasalazine, tumor necrosis factor alpha (TNFa) blockers such as etanercept (Enbrel), infliximab (Remicade), adalimumab (Humira), certolizumab pegol (Cimzia), golimumab (Simponi), Interleukin 1 (IL-1) blockers such as anakinra (Kineret), T cell costimulation blockers such as abatacept (Orencia), Interleukin 6 (IL-6) blockers such as tocilizumab (ACTEMERA®); Interleukin 13 (IL-13) blockers such as lebrikizumab; Interferon alpha (IFN) blockers such as Rontalizumab; Beta 7 integrin blockers such as rhuMAb Beta7; IgE pathway blockers such as Anti-M1 prime; Secreted homotrimeric LTa3 and membrane bound heterotrimer LTa1/β2 blockers such as Anti-lymphotoxin alpha (LTa); radioactive isotopes (e.g., At211, 1131, 1125, Y90, Re186, Re188, Sm153, Bi212, P32, Pb212 and radioactive isotopes of Lu); miscellaneous investigational agents such as thioplatin, PS-341, phenylbutyrate, ET-18-OCH3, or farnesyl transferase inhibitors (L-739749, L-744832); polyphenols such as quercetin, resveratrol, piceatannol, epigallocatechine gallate, theaflavins, flavanols, procyanidins, betulinic acid and derivatives thereof; autophagy inhibitors such as chloroquine; delta-9-tetrahydrocannabinol (dronabinol, MARINOL®); beta-lapachone; lapachol; colchicines; betulinic acid; acetylcamptothecin, scopolectin, and 9-aminocamptothecin); podophyllotoxin; tegafur (UFTORAL®); bexarotene (TARGRETIN®); bisphosphonates such as clodronate (for example, BONEFOS® or OSTAC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); and epidermal growth factor receptor (EGF-R); vaccines such as THERATOPE® vaccine; perifosine, COX-2 inhibitor (e.g. celecoxib or etoricoxib), proteosome inhibitor (e.g. PS341); CCI-779; tipifarnib (R11577); orafenib, ABT510; Bcl-2 inhibitor such as oblimersen sodium (GENASENSE®); pixantrone; farnesyltransferase inhibitors such as lonafarnib (SCH 6636, SARASAR™); and pharmaceutically acceptable salts, acids or derivatives of any of the above; as well as combinations of two or more of the above such as CHOP, an abbreviation for a combined therapy of cyclophosphamide, doxorubicin, vincristine, and prednisolone; and FOLFOX, an abbreviation for a treatment regimen with oxaliplatin (ELOXATIN™) combined with 5-FU and leucovorin.

Chemotherapeutic agents also include non-steroidal anti-inflammatory drugs with analgesic, antipyretic and anti-inflammatory effects. NSAIDs include non-selective inhibitors of the enzyme cyclooxygenase. Specific examples of NSAIDs include aspirin, propionic acid derivatives such as ibuprofen, fenoprofen, ketoprofen, flurbiprofen, oxaprozin and naproxen, acetic acid derivatives such as indomethacin, sulindac, etodolac, diclofenac, enolic acid derivatives such as piroxicam, meloxicam, tenoxicam, droxicam, lornoxicam and isoxicam, fenamic acid derivatives such as mefenamic acid, meclofenamic acid, flufenamic acid, tolfenamic acid, and COX-2 inhibitors such as celecoxib, etoricoxib, lumiracoxib, parecoxib, rofecoxib, rofecoxib, and valdecoxib. NSAIDs can be indicated for the symptomatic relief of conditions such as rheumatoid arthritis, osteoarthritis, inflammatory arthropathies, ankylosing spondylitis, psoriatic arthritis, Reiter's syndrome, acute gout, dysmenorrhoea, metastatic bone pain, headache and migraine, postoperative pain, mild-to-moderate pain due to inflammation and tissue injury, pyrexia, ileus, and renal colic.

The term “cytokine” is a generic term for proteins released by one cell population that act on another cell as intercellular mediators. Examples of such cytokines are lymphokines, monokines; interleukins (ILs) such as IL-1, IL-1a, IL-2, IL-3, IL-4, IL-5, IL-6, IL-7, IL-8, IL-9, IL-11, IL-12, IL-15; a tumor necrosis factor such as TNF-α or TNF-β; and other polypeptide factors including LIF and kit ligand (KL) and gamma interferon. As used herein, the term cytokine includes proteins from natural sources or from recombinant cell culture and biologically active equivalents of the native-sequence cytokines, including synthetically produced small-molecule entities and pharmaceutically acceptable derivatives and salts thereof.

The term “phagocytosis” means the internalization of cells or particulate matter by cells. In some embodiments, the phagocytic cells or phagocytes are macrophages or neutrophils. In some embodiments, the cells are cells that express human OX40. Methods for assaying phagocytosis are known in the art and include use of microscopy to detect the presence of cells internalized within another cells. In other embodiments, phagocytosis is detected using FACS, e.g., by detecting presence of a detectably labeled cell within another cell (which may be detectably labeled, e.g., with a different label than the first cell).

The phrase “does not possess substantial activity” or “substantially no activity” with respect to an antibody, as used herein, means the antibody does not exhibit an activity that is above background level (in some embodiments, that is above background level that is statistically significant). The phrase “little to no activity” with respect to an antibody, as used herein, means the antibody does not display a biologically meaningful amount of a function. The function can be measured or detected according to any assay or technique known in the art, including, e.g., those described herein. In some embodiments, antibody function is stimulation of effector T cell proliferation and/or cytokine secretion.

The term “biomarker” or “marker” as used herein refers generally to a molecule, including a gene, mRNA, protein, carbohydrate structure, or glycolipid, the expression of which in or on a tissue or cell or secreted can be detected by known methods (or methods disclosed herein) and is predictive or can be used to predict (or aid prediction) for a cell, tissue, or patient's responsiveness to treatment regimes.

By “patient sample” is meant a collection of cells or fluids obtained from a cancer patient. The source of the tissue or cell sample may be solid tissue as from a fresh, frozen and/or preserved organ or tissue sample or biopsy or aspirate; blood or any blood constituents; bodily fluids such as cerebrospinal fluid, amniotic fluid, peritoneal fluid, or interstitial fluid; cells from any time in gestation or development of the subject. The tissue sample may contain compounds which are not naturally intermixed with the tissue in nature such as preservatives, anticoagulants, buffers, fixatives, nutrients, antibiotics, or the like. Examples of tumor samples herein include, but are not limited to, tumor biopsy, fine needle aspirate, bronchiolar lavage, pleural fluid, sputum, urine, a surgical specimen, circulating tumor cells, serum, plasma, circulating plasma proteins, ascitic fluid, primary cell cultures or cell lines derived from tumors or exhibiting tumor-like properties, as well as preserved tumor samples, such as formalin-fixed, paraffin-embedded tumor samples or frozen tumor samples.

The phrase “based on expression of” when used herein means that information about expression level or presence or absence of expression (e.g., presence or absence or prevalence of (e.g., percentage of cells displaying) of the one or more biomarkers herein (e.g., presence or absence of or amount or prevelance of FcR-expressing cells, or e.g., presence or absence or amount or prevelance of human effector cells) is used to inform a treatment decision, information provided on a package insert, or marketing/promotional guidance etc.

A cancer or biological sample which “has human effector cells” is one which, in a diagnostic test, has human effector cells present in the sample (e.g., infiltrating human effector cells).

A cancer or biological sample which “has FcR-expressing cells” is one which, in a diagnostic test, has FcR-expressing present in the sample (e.g., infiltrating FcR-expressing cells). In some embodiments, FcR is FcγR. In some embodiments, FcR is an activating FcγR.

The phrase “recommending a treatment” as used herein refers to using the information or data generated relating to the level or presence of c-met in a sample of a patient to identify the patient as suitably treated or not suitably treated with a therapy. In some embodiments the therapy may comprise c-met antibody (e.g., onartuzumab). In some embodiments, the therapy may comprise VEGF antagonist (e.g., bevacizumab). In some embodiments, the therapy may comprise anti-human OX40 agonist antibody. The information or data may be in any form, written, oral or electronic. In some embodiments, using the information or data generated includes communicating, presenting, reporting, storing, sending, transferring, supplying, transmitting, delivering, dispensing, or combinations thereof. In some embodiments, communicating, presenting, reporting, storing, sending, transferring, supplying, transmitting, delivering, dispensing, or combinations thereof are performed by a computing device, analyzer unit or combination thereof. In some further embodiments, communicating, presenting, reporting, storing, sending, transferring, supplying, transmitting, dispensing, or combinations thereof are performed by an individual (e.g., a laboratory or medical professional). In some embodiments, the information or data includes a comparison of the amount or prevelance of FcR expressing cells to a reference level. In some embodiments, the information or data includes a comparison of the amount or prevelance of human effector cells to a reference level. In some embodiments, the information or data includes an indication that human effector cells or FcR-expressing cells are present or absent in the sample. In some embodiments, the information or data includes an indication that FcR-expressing cells and/or human effector cells are present in a particular percentage of cells (e.g., high prevelance). In some embodiments, the information or data includes an indication that the patient is suitably treated or not suitably treated with a therapy comprising anti-human OX40 agonist antibody.

In reference to a patient, “immunotherapy-naïve” is meant a patient who has not received prior treatment with an immunotherapeutic agent. In some embodiments, immunotherapeutic agents may refer to costimulatory agonists and/or immune checkpoint blockade therapies. Costimulatory agonists may target, for example and without limitation, OX40, CD40, CD226, CD28, OX40, GITR, CD137, CD27, HVEM, or CD127. In some embodiments, the costimulatory agonist may be an OX40, CD137, CD27, GITR, or CD40 agonist. Immune checkpoint blockade therapies may include, for example and without limitation, an antagonist directed against an inhibitory co-stimulatory molecule. Inhibitory co-stimulatory molecules may include without limitation CTLA-4, PD-1, PD-L1, PD-L2, TIM-3, BTLA, VISTA, LAG-3, B7-H3, B7-H4, IDO, TIGIT, MICA/B, or arginase. In some embodiments, the immune checkpoint blockade therapy may be a CTLA4 (also known as CD152) antagonist or a PD-1 axis binding antagonist.

The term “PD-1 axis binding antagonist” is a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist.

The term “PD-1 binding antagonist” is a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1 106 described herein. In another specific aspect, a PD-1 binding antagonist is Merck 3745 described herein. In another specific aspect, a PD-1 binding antagonist is CT-011 described herein.

The term “PD-L1 binding antagonist” is a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is YW243.55.S70 described herein. In another specific aspect, an anti-PD-L1 antibody is MDX-1 105 described herein. In still another specific aspect, an anti-PD-L1 antibody is MPDL3280A described herein.

The term “PD-L2 binding antagonist” is a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

### II. Compositions and Methods

In one aspect, the invention is based, in part, on identification of a variety of OX40 binding agents. In certain embodiments, antibodies (e.g., agonist antibodies) that bind to human OX40 are provided. Antibodies of the invention are useful, e.g., for the diagnosis or treatment of cancer and other disorders associated with OX40 expression and/or activity.

### A. Exemplary Anti-OX40 Antibodies

In one aspect, the invention provides isolated antibodies that bind to human OX40.

In some embodiments, the anti-human OX40 agonist antibody binds human OX40 with an affinity of less than or equal to about 0.45 nM. In some embodiments, the anti-human OX40 antibody binds human OX40 with an affinity of less than or equal to about 0.4 nM. In some embodiments, the anti-human OX40 antibody binds human OX40 with an affinity of less than or equal to about 0.5 nM. In some embodiments, the binding affinity is determined using radioimmunoassay.

In some embodiments, the anti-human OX40 agonist antibody binds human OX40 and cynomolgus OX40. In some embodiments, binding is determined using a FACS assay. In some embodiments, binding to human OX40 has an EC50 of about 0.2 ug/ml. In some embodiments, binding to human OX40 has an EC50 of about 0.3 ug/ml or lower. In some embodiments, binding to cynomolgus OX40 has an EC50 of about 1.5 ug/ml. In some embodiments, binding to cynomolgus OX40 has an EC50 of about 1.4 ug/ml.

In some embodiments, the anti-human OX40 agonist antibody does not bind to rat OX40 or mouse OX40.

In some embodiments, the anti-human OX40 agonist antibody is a depleting anti-human OX40 antibody (e.g., depletes cells that express human OX40). In some embodiments, the human OX40 expressing cells are CD4+ effector T cells. In some embodiments, the human OX40 expressing cells are Treg cells. In some embodiments, depleting is by ADCC and/or phagocytosis. In some embodiments, the antibody mediates ADCC by binding FcγR expressed by a human effector cell and activating the human effector cell function. In some embodiments, the antibody mediates phagocytosis by binding FcγR expressed by a human effector cell and activating the human effector cell function. Exemplary human effector cells include, e.g., macrophage, natural killer (NK) cells, monocytes, neutrophils. In some embodiments, the human effector cell is macrophage. In some embodiments, the human effector cell is NK cells. In some embodiments, depletion is not by apoptosis.

In some embodiments, the anti-human OX40 agonist antibody has a functional Fc region. In some embodiments, effector function of a functional Fc region is ADCC. In some embodiments, effector function of a functional Fc region is phagocytosis. In some embodiments, effector function of a functional Fc region is ADCC and phagocytosis. In some embodiments, the Fc region is human IgG1. In some embodiments, the Fc region is human IgG4.

In some embodiments, the anti-human OX40 agonist antibody does not induce apoptosis in OX40-expressing cells (e.g., Treg). In some embodiments, apoptosis is assayed using an antibody concentration of 30 ug/ml, e.g., by determining whether apoptosis has occurred using annexin V and proprodium iodide stained Treg.

In some embodiments, the anti-human OX40 agonist antibody enhances CD4+ effector T cell function, for example, by increasing CD4+ effector T cell proliferation and/or increasing gamma interferon production by the CD4+ effector T cell (for example, as compared to proliferation and/or cytokine production prior to treatment with anti-human OX40 agonist antibody). In some embodiments, the cytokine is gamma interferon. In some embodiments, the anti-human OX40 agonist antibody increases number of intratumoral (infiltrating) CD4+ effector T cells (e.g., total number of CD4+ effector T cells, or e.g., percentage of CD4+ cells in CD45+ cells), e.g., as compared to number of intratumoral (infiltrating) CD4+ T cells prior to treatment with anti-human OX40 agonist antibody. In some embodiments, the anti-human OX40 agonist antibody increases number of intratumoral (infiltrating) CD4+ effector T cells that express gamma interferon (e.g., total gamma interferon expressing CD4+ cells, or e.g., percentage of gamma interferon expressing CD4+ cells in total CD4+ cells), e.g., as compared to number of intratumoral (infiltrating) CD4+ T cells that express gamma interferon prior to treatment with anti-human OX40 agonist antibody.

In some embodiments, the anti-human OX40 agonist antibody increases number of intratumoral (infiltrating) CD8+ effector T cells (e.g., total number of CD8+ effector T cells, or e.g., percentage of CD8+ in CD45+ cells), e.g., as compared to number of intratumoral (infiltrating) CD8+T effector cells prior to treatment with anti-human OX40 agonist antibody. In some embodiments, the anti-human OX40 agonist antibody increases number of intratumoral (infiltrating) CD8+ effector T cells that express gamma interferon (e.g., percentage of CD8+ cells that express gamma interferon in total CD8+ cells), e.g., compared to number of intratumoral (infiltrating) CD8+ T cells that express gamma interferon prior to treatment with anti-human OX40 agonist antibody.

In some embodiments, the anti-human OX40 agonist antibody enhances memory T cell function, for example by increasing memory T cell proliferation and/or increasing cytokine production by the memory cell. In some embodiments, the cytokine is gamma interferon.

In some embodiments, the anti-human OX40 agonist antibody inhibits Treg function, for example, by decreasing Treg suppression of effector T cell function (e.g., effector T cell proliferation and/or effector T cell cytokine secretion). In some embodiments, the effector T cell is a CD4+ effector T cell. In some embodiments, the anti-human OX40 agonist antibody reduces the number of intratumoral (infiltrating) Treg (e.g., total number of Treg or e.g., percentage of Fox3p+ cells in CD4+ cells).

In some embodiments, the anti-human OX40 agonist antibody is engineered to increase effector function (e.g., compared to effector function in a wild-type IgG1). In some embodiments, the antibody has increased binding to a Fcγ receptor. In some embodiments, the antibody lacks fucose attached (directly or indirectly) to the Fc region. For example, the amount of fucose in such antibody may be from 1% to 80%, from 1% to 65%, from 5% to 65% or from 20% to 40%. In some embodiments, the Fc region comprises bisected oligosaccharides, e.g., in which a biantennary oligosaccharide attached to the Fc region of the antibody is bisected by GlcNAc. In some embodiments, the antibody comprises an Fc region with one or more amino acid substitutions which improve ADCC, e.g., substitutions at positions 298, 333, and/or 334 of the Fc region (EU numbering of residues).

In some embodiments, the anti-human OX40 agonist antibody increases OX40 signal transduction in a target cell that expresses OX40. In some embodiments, OX40 signal transduction is detected by monitoring NFkB downstream signaling.

In some embodiments, the anti-human OX40 agonist antibody is stable after treatment at 40C for two weeks.

In some embodiments, the anti-human OX40 agonist antibody binds human effector cells, e.g., binds FcγR (e.g., an activating FcγR) expressed by human effector cells. In some embodiments, the human effector cell performs (is capable of performing) ADCC effector function. In some embodiments, the human effector cell performs (is capable of performing) phagocytosis effector function.

In some embodiments, the anti-human OX40 agonist antibody comprising a variant IgG1 Fc polypeptide comprising a mutation that eliminates binding to human effector cells (e.g., a DANA mutation) has diminished activity (e.g., CD4+ effector T cell function, e.g., proliferation), relative to anti-human OX40 agonist antibody comprising native sequence IgG1 Fc portion. In some embodiment, the anti-human OX40 agonist antibody comprising a variant IgG1 Fc polypeptide comprising a mutation that eliminates binding to human effector cells (e.g., a DANA mutation) does not possess substantial activity (e.g., CD4+ effector T cell function, e.g., proliferation).

In some embodiments, antibody cross-linking is required for anti-human OX40 agonist antibody function. In some embodiments, function is stimulation of CD4+ effector T cell proliferation. In some embodiments, antibody cross-linking is determined by providing anti-human OX40 agonist antibody adhered on a solid surface (e.g., a cell culture plate). In some embodiments, antibody cross-linking is determined by introducing a mutation in the antibody's IgG1 Fc portion (e.g., a DANA mutation) and testing function of the mutant antibody.

In some embodiments, the anti-human OX40 agonist antibody competes for binding to human OX40 with OX40L. In some embodiments, addition of OX40L does not enhance anti-human OX40 antibody function in an in vitro assay.

According to another embodiment, the anti-human OX40 agonist antibodies include any one, any combination, or all of the following properties: (1) binds human OX40 with an affinity of less than or equal to about 0.45 nM, in some embodiments, binds human OX40 with an affinity of less than or equal to about 0.4 nM, in some embodiments, binds human OX40 with an affinity of less than or equal to about 0.5 nM, in some embodiments, the binding affinity is determined using radioimmunoassay; (2) binds human OX40 and cynomolgus OX40, in some embodiments, binding is determined using a FACS assay, (3) binds human OX40 with an EC50 of about 0.2 ug/ml, in some embodiments, binds to human OX40 has an EC50 of about 0.3 ug/ml or lower, in some embodiments, binds to cynomolgus OX40 with an EC50 of about 1.5 ug/ml, in some embodiments, binds to cynomolgus OX40 has an EC50 of about 1.4 ug/ml, (4) does not substantially bind to rat OX40 or mouse OX40, (6) is a depleting anti-human OX40 antibody (e.g., depletes cells that express human OX40), in some embodiments, the cells are CD4+ effector T cells and/or Treg cells, (7) enhances CD4+ effector T cell function, for example, by increasing CD4+ effector T cell proliferation and/or increasing gamma interferon production by the CD4+ effector T cell (for example, as compared to proliferation and/or cytokine production prior to treatment with anti-human OX40 agonist antibody), (8) enhances memory T cell function, for example by increasing memory T cell proliferation and/or increasing cytokine production by the memory cell, (9) inhibits Treg function, for example, by decreasing Treg suppression of effector T cell function (e.g., effector T cell proliferation and/or effector T cell cytokine secretion). In some embodiments, the effector T cell is a CD4+ effector T cell, (10) increases OX40 signal transduction in a target cell that expresses OX40 (in some embodiments, OX40 signal transduction is detected by monitoring NFkB downstream signaling), (11) is stable after treatment at 40 C for two weeks, (12) binds human effector cells, e.g., binds FcγR expressed by human effector cells, (13) anti-human OX40 agonist antibody comprising a variant IgG1 Fc polypeptide comprising a mutation that eliminates binding to human effector cells (e.g., N297G) has diminished activity (e.g., CD4+ effector T cell function, e.g., proliferation), relative to anti-human OX40 agonist antibody comprising native sequence IgG1 Fc portion, in some embodiment, the anti-human OX40 agonist antibody comprising a variant IgG1 Fc polypeptide comprising a mutation that eliminates binding to human effector cells (e.g., N297G) does not possess substantial activity (e.g., CD4+ effector T cell function, e.g., proliferation), (14) antibody cross-linking (e.g., by Fc receptor binding) is required for anti-human OX40 agonist antibody function.

In one aspect, the invention provides an anti-human OX40 agonist antibody comprising at least one, two, three, four, five, or six HVRs selected from (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising the amino acid sequence of SEQ ID NO:7.

In one aspect, the invention provides an anti-human OX40 agonist antibody comprising at least one, at least two, or all three VH HVR sequences selected from (a) HVR-H1 comprising the amino acid sequence of SEQ ID NO:2; (b) HVR-H2 comprising the amino acid sequence of SEQ ID NO:3; and (c) HVR-H3 comprising the amino acid sequence of SEQ ID NO:4. In one embodiment, the antibody comprises HVR-H3 comprising the amino acid sequence of SEQ ID NO:4. In another embodiment, the antibody comprises HVR-H3 comprising the amino acid sequence of SEQ ID NO:4 and HVR-L3 comprising the amino acid sequence of SEQ ID NO:7. In a further embodiment, the antibody comprises HVR-H3 comprising the amino acid sequence of SEQ ID NO:4, HVR-L3 comprising the amino acid sequence of SEQ ID NO:7, and HVR-H2 comprising the amino acid sequence of SEQ ID NO:3. In a further embodiment, the antibody comprises (a) HVR-H1 comprising the amino acid sequence of SEQ ID NO:2; (b) HVR-H2 comprising the amino acid sequence of SEQ ID NO:3; and (c) HVR-H3 comprising the amino acid sequence of SEQ ID NO:4.

In another aspect, the invention provides an anti-human OX40 agonist antibody comprising at least one, at least two, or all three VL HVR sequences selected from (a) HVR-L1 comprising the amino acid sequence of SEQ ID NO:5; (b) HVR-L2 comprising the amino acid sequence of SEQ ID NO:6; and (c) HVR-L3 comprising the amino acid sequence of SEQ ID NO:7. In one embodiment, the antibody comprises (a) HVR-L1 comprising the amino acid sequence of SEQ ID NO:5; (b) HVR-L2 comprising the amino acid sequence of SEQ ID NO:6; and (c) HVR-L3 comprising the amino acid sequence of SEQ ID NO:7.

In another aspect, an anti-human OX40 agonist antibody of the invention comprises (a) a VH domain comprising at least one, at least two, or all three VH HVR sequences selected from (i) HVR-H1 comprising the amino acid sequence of SEQ ID NO:2, (ii) HVR-H2 comprising the amino acid sequence of SEQ ID NO:3, and (iii) HVR-H3 comprising an amino acid sequence selected from SEQ ID NO:4; and (b) a VL domain comprising at least one, at least two, or all three VL HVR sequences selected from (i) HVR-L1 comprising the amino acid sequence of SEQ ID NO:5, (ii) HVR-L2 comprising the amino acid sequence of SEQ ID NO:6, and (c) HVR-L3 comprising the amino acid sequence of SEQ ID NO:7.

In another aspect, the invention provides an anti-human OX40 agonist antibody comprising (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7.

In one aspect, the invention provides an anti-human OX40 agonist antibody comprising at least one, two, three, four, five, or six HVRs selected from (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising the amino acid sequence of SEQ ID NO:26.

In another embodiment, the antibody comprises HVR-H3 comprising the amino acid sequence of SEQ ID NO:4 and HVR-L3 comprising the amino acid sequence of SEQ ID NO:26. In a further embodiment, the antibody comprises HVR-H3 comprising the amino acid sequence of SEQ ID NO:4, HVR-L3 comprising the amino acid sequence of SEQ ID NO:26, and HVR-H2 comprising the amino acid sequence of SEQ ID NO:3.

In another aspect, an antibody of the invention comprises (a) a VH domain comprising at least one, at least two, or all three VH HVR sequences selected from (i) HVR-H1 comprising the amino acid sequence of SEQ ID NO:2, (ii) HVR-H2 comprising the amino acid sequence of SEQ ID NO:3, and (iii) HVR-H3 comprising an amino acid sequence selected from SEQ ID NO:4; and (b) a VL domain comprising at least one, at least two, or all three VL HVR sequences selected from (i) HVR-L1 comprising the amino acid sequence of SEQ ID NO:5, (ii) HVR-L2 comprising the amino acid sequence of SEQ ID NO:6, and (c) HVR-L3 comprising the amino acid sequence of SEQ ID NO:26.

In another aspect, the invention provides an antibody comprising (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:26.

In one aspect, the invention provides an anti-human OX40 agonist antibody comprising at least one, two, three, four, five, or six HVRs selected from (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising the amino acid sequence of SEQ ID NO:27.

In another embodiment, the antibody comprises HVR-H3 comprising the amino acid sequence of SEQ ID NO:4 and HVR-L3 comprising the amino acid sequence of SEQ ID NO:27. In a further embodiment, the antibody comprises HVR-H3 comprising the amino acid sequence of SEQ ID NO:4, HVR-L3 comprising the amino acid sequence of SEQ ID NO:27, and HVR-H2 comprising the amino acid sequence of SEQ ID NO:3.

In another aspect, an antibody of the invention comprises (a) a VH domain comprising at least one, at least two, or all three VH HVR sequences selected from (i) HVR-H1 comprising the amino acid sequence of SEQ ID NO:2, (ii) HVR-H2 comprising the amino acid sequence of SEQ ID NO:3, and (iii) HVR-H3 comprising an amino acid sequence selected from SEQ ID NO:4; and (b) a VL domain comprising at least one, at least two, or all three VL HVR sequences selected from (i) HVR-L1 comprising the amino acid sequence of SEQ ID NO:5, (ii) HVR-L2 comprising the amino acid sequence of SEQ ID NO:6, and (c) HVR-L3 comprising the amino acid sequence of SEQ ID NO:27.

In another aspect, the invention provides an antibody comprising (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:27.

In one aspect, the invention provides an anti-human OX40 agonist antibody comprising at least one, two, three, four, five, or six HVRs selected from (a) HVR-H1 comprising the amino acid sequence of SEQ ID NO:2, 8 or 9; (b) HVR-H2 comprising the amino acid sequence of SEQ ID NO:3, 10, 11, 12, 13 or 14; (c) HVR-H3 comprising the amino acid sequence of SEQ ID NO:4, 15, or 19; (d) HVR-L1 comprising the amino acid sequence of SEQ ID NO:5; (e) HVR-L2 comprising the amino acid sequence of SEQ ID NO:6; and (f) HVR-L3 comprising the amino acid sequence of SEQ ID NO:7, 22, 23, 24, 25, 26, 27, or 28.

In one aspect, the invention provides an antibody comprising at least one, at least two, or all three VH HVR sequences selected from (a) HVR-H1 comprising the amino acid sequence of SEQ ID NO: 2, 8 or 9; (b) HVR-H2 comprising the amino acid sequence of SEQ ID NO: 3, 10, 11, 12, 13 or 14; and (c) HVR-H3 comprising the amino acid sequence of SEQ ID NO: 4, 15, or 19. In one embodiment, the antibody comprises HVR-H3 comprising the amino acid sequence of SEQ ID NO: 4, 15, or 19. In another embodiment, the antibody comprises HVR-H3 comprising the amino acid sequence of SEQ ID NO:4, 15, or 19 and HVR-L3 comprising the amino acid sequence of SEQ ID NO: 7, 22, 23, 24, 25, 26, 27, or 28. In a further embodiment, the antibody comprises HVR-H3 comprising the amino acid sequence of SEQ ID NO: 4, 15, or 19, HVR-L3 comprising the amino acid sequence of SEQ ID NO: 7, 22, 23, 24, 25, 26, 27, or 28, and HVR-H2 comprising the amino acid sequence of SEQ ID NO: 3, 10, 11, 12, 13 or 14. In a further embodiment, the antibody comprises (a) HVR-H1 comprising the amino acid sequence of SEQ ID NO: 2, 8 or 9; (b) HVR-H2 comprising the amino acid sequence of SEQ ID NO: 3, 10, 11, 12, 13 or 14; and (c) HVR-H3 comprising the amino acid sequence of SEQ ID NO: 4, 15, or 19.

In another aspect, the invention provides an antibody comprising at least one, at least two, or all three VL HVR sequences selected from (a) HVR-L1 comprising the amino acid sequence of SEQ ID NO: 5; (b) HVR-L2 comprising the amino acid sequence of SEQ ID NO:6; and (c) HVR-L3 comprising the amino acid sequence of SEQ ID NO: 7, 22, 23, 24, 25, 26, 27, or 28. In one embodiment, the antibody comprises (a) HVR-L1 comprising the amino acid sequence of SEQ ID NO:5; (b) HVR-L2 comprising the amino acid sequence of SEQ ID NO:6; and (c) HVR-L3 comprising the amino acid sequence of SEQ ID NO: 7, 22, 23, 24, 25, 26, 27, or 28.

In another aspect, an antibody of the invention comprises (a) a VH domain comprising at least one, at least two, or all three VH HVR sequences selected from (i) HVR-H1 comprising the amino acid sequence of SEQ ID NO: 2, 8 or 9, (ii) HVR-H2 comprising the amino acid sequence of SEQ ID NO: 3, 10, 11, 12, 13 or 14, and (iii) HVR-H3 comprising an amino acid sequence selected from SEQ ID NO: 4, 15, or 19; and (b) a VL domain comprising at least one, at least two, or all three VL HVR sequences selected from (i) HVR-L1 comprising the amino acid sequence of SEQ ID NO:5, (ii) HVR-L2 comprising the amino acid sequence of SEQ ID NO:6, and (c) HVR-L3 comprising the amino acid sequence of SEQ ID NO: 7, 22, 23, 24, 25, 26, 27, or 28.

In another aspect, the invention provides an antibody comprising (a) HVR-H1 comprising the amino acid sequence of SEQ ID NO: 2, 8 or 9; (b) HVR-H2 comprising the amino acid sequence of SEQ ID NO: 3, 10, 11, 12, 13 or 14; (c) HVR-H3 comprising the amino acid sequence of SEQ ID NO: 4, 15, or 19; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO: 7, 22, 23, 24, 25, 26, 27, or 28.

In one aspect, the invention provides an anti-human OX40 agonist antibody comprising at least one, two, three, four, five, or six HVRs selected from (a) HVR-H1 comprising the amino acid sequence of SEQ ID NO:172; (b) HVR-H2 comprising the amino acid sequence of SEQ ID NO:173; (c) HVR-H3 comprising the amino acid sequence of SEQ ID NO:174; (d) HVR-L1 comprising the amino acid sequence of SEQ ID NO:5; (e) HVR-L2 comprising the amino acid sequence of SEQ ID NO:6; and (f) HVR-L3 comprising the amino acid sequence of SEQ ID NO:175. In some embodiment, HVR-H2 is not DMYPDAAAASYNQKFRE (SEQ ID NO:193),In some embodiments, HVR-H3 is not APRWAAAA (SEQ ID NO:194). In some embodiments, HVR-L3 is not QAAAAAAAT (SEQ ID NO:195).

In one aspect, the invention provides an antibody comprising at least one, at least two, or all three VH HVR sequences selected from (a) HVR-H1 comprising the amino acid sequence of SEQ ID NO:172; (b) HVR-H2 comprising the amino acid sequence of SEQ ID NO:173; and (c) HVR-H3 comprising the amino acid sequence of SEQ ID NO:174. In one embodiment, the antibody comprises HVR-H3 comprising the amino acid sequence of SEQ ID NO:174. In another embodiment, the antibody comprises HVR-H3 comprising the amino acid sequence of SEQ ID NO:174 and HVR-L3 comprising the amino acid sequence of SEQ ID NO:175. In a further embodiment, the antibody comprises HVR-H3 comprising the amino acid sequence of SEQ ID NO:174, HVR-L3 comprising the amino acid sequence of SEQ ID NO:175, and HVR-H2 comprising the amino acid sequence of SEQ ID NO:173. In a further embodiment, the antibody comprises (a) HVR-H1 comprising the amino acid sequence of SEQ ID NO:172; (b) HVR-H2 comprising the amino acid sequence of SEQ ID NO:173; and (c) HVR-H3 comprising the amino acid sequence of SEQ ID NO:174. In some embodiment, HVR-H2 is not DMYPDAAAASYNQKFRE (SEQ ID NO:193),In some embodiments, HVR-H3 is not APRWAAAA (SEQ ID NO:194). In some embodiments, HVR-L3 is not QAAAAAAAT (SEQ ID NO:195).

In another aspect, the invention provides an antibody comprising (a) HVR-L1 comprising the amino acid sequence of SEQ ID NO:5; (b) HVR-L2 comprising the amino acid sequence of SEQ ID NO:6; and (c) HVR-L3 comprising the amino acid sequence of SEQ ID NO:175. In some embodiments, HVR-L3 is not QAAAAAAAT (SEQ ID NO:195).

In another aspect, an antibody of the invention comprises (a) a VH domain comprising at least one, at least two, or all three VH HVR sequences selected from (i) HVR-H1 comprising the amino acid sequence of SEQ ID NO:172, (ii) HVR-H2 comprising the amino acid sequence of SEQ ID NO:173, and (iii) HVR-H3 comprising an amino acid sequence selected from SEQ ID NO:174; and (b) a VL domain comprising at least one, at least two, or all three VL HVR sequences selected from (i) HVR-L1 comprising the amino acid sequence of SEQ ID NO:5, (ii) HVR-L2 comprising the amino acid sequence of SEQ ID NO:6, and (c) HVR-L3 comprising the amino acid sequence of SEQ ID NO:175.

In another aspect, the invention provides an antibody comprising (a) HVR-H1 comprising the amino acid sequence of SEQ ID NO:172; (b) HVR-H2 comprising the amino acid sequence of SEQ ID NO:173; (c) HVR-H3 comprising the amino acid sequence of SEQ ID NO:174; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:175. In some embodiment, HVR-H2 is not DMYPDAAAASYNQKFRE (SEQ ID NO:193),In some embodiments, HVR-H3 is not APRWAAAA (SEQ ID NO:194). In some embodiments, HVR-L3 is not QAAAAAAAT (SEQ ID NO:195).

All possible combinations of the above substitutions are encompassed by the consensus sequences of SEQ ID NO:172, 173, 174 and 175.

In one aspect, the invention provides an anti-human OX40 agonist antibody comprising at least one, two, three, four, five, or six HVRs selected from (a) HVR-H1 comprising the amino acid sequence of SEQ ID NO:29; (b) HVR-H2 comprising the amino acid sequence of SEQ ID NO:30; (c) HVR-H3 comprising the amino acid sequence of SEQ ID NO:33; (d) HVR-L1 comprising the amino acid sequence of SEQ ID NO:37; (e) HVR-L2 comprising the amino acid sequence of SEQ ID NO:39; and (f) HVR-L3 comprising the amino acid sequence of SEQ ID NO:42.

In one aspect, the invention provides an antibody comprising at least one, at least two, or all three VH HVR sequences selected from (a) HVR-H1 comprising the amino acid sequence of SEQ ID NO:29; (b) HVR-H2 comprising the amino acid sequence of SEQ ID NO:30; and (c) HVR-H3 comprising the amino acid sequence of SEQ ID NO:33. In one embodiment, the antibody comprises HVR-H3 comprising the amino acid sequence of SEQ ID NO:33. In another embodiment, the antibody comprises HVR-H3 comprising the amino acid sequence of SEQ ID NO:33 and HVR-L3 comprising the amino acid sequence of SEQ ID NO:42. In a further embodiment, the antibody comprises HVR-H3 comprising the amino acid sequence of SEQ ID NO:33, HVR-L3 comprising the amino acid sequence of SEQ ID NO:42, and HVR-H2 comprising the amino acid sequence of SEQ ID NO:30. In a further embodiment, the antibody comprises (a) HVR-H1 comprising the amino acid sequence of SEQ ID NO:29; (b) HVR-H2 comprising the amino acid sequence of SEQ ID NO:30; and (c) HVR-H3 comprising the amino acid sequence of SEQ ID NO:33.

In another aspect, the invention provides an antibody comprising at least one, at least two, or all three VL HVR sequences selected from (a) HVR-L1 comprising the amino acid sequence of SEQ ID NO:37; (b) HVR-L2 comprising the amino acid sequence of SEQ ID NO:39; and (c) HVR-L3 comprising the amino acid sequence of SEQ ID NO:42. In one embodiment, the antibody comprises (a) HVR-L1 comprising the amino acid sequence of SEQ ID NO:37; (b) HVR-L2 comprising the amino acid sequence of SEQ ID NO:39; and (c) HVR-L3 comprising the amino acid sequence of SEQ ID NO:42.

In another aspect, an antibody of the invention comprises (a) a VH domain comprising at least one, at least two, or all three VH HVR sequences selected from (i) HVR-H1 comprising the amino acid sequence of SEQ ID NO:29, (ii) HVR-H2 comprising the amino acid sequence of SEQ ID NO:30, and (iii) HVR-H3 comprising an amino acid sequence selected from SEQ ID NO:33; and (b) a VL domain comprising at least one, at least two, or all three VL HVR sequences selected from (i) HVR-L1 comprising the amino acid sequence of SEQ ID NO:37, (ii) HVR-L2 comprising the amino acid sequence of SEQ ID NO:39, and (c) HVR-L3 comprising the amino acid sequence of SEQ ID NO:42.

In another aspect, the invention provides an antibody comprising (a) HVR-H1 comprising the amino acid sequence of SEQ ID NO:29; (b) HVR-H2 comprising the amino acid sequence of SEQ ID NO:30; (c) HVR-H3 comprising the amino acid sequence of SEQ ID NO:33; (d) HVR-L1 comprising the amino acid sequence of SEQ ID NO:37; (e) HVR-L2 comprising the amino acid sequence of SEQ ID NO:39; and (f) HVR-L3 comprising an amino acid sequence selected from SEQ ID NO:42.

In one aspect, the invention provides an anti-human OX40 agonist antibody comprising at least one, two, three, four, five, or six HVRs selected from (a) HVR-H1 comprising the amino acid sequence of SEQ ID NO:29; (b) HVR-H2 comprising the amino acid sequence of SEQ ID NO:30; (c) HVR-H3 comprising the amino acid sequence of SEQ ID NO:33; (d) HVR-L1 comprising the amino acid sequence of SEQ ID NO:37; (e) HVR-L2 comprising the amino acid sequence of SEQ ID NO:40; and (f) HVR-L3 comprising the amino acid sequence of SEQ ID NO:42.

In another aspect, the invention provides an antibody comprising at least one, at least two, or all three VL HVR sequences selected from (a) HVR-L1 comprising the amino acid sequence of SEQ ID NO:37; (b) HVR-L2 comprising the amino acid sequence of SEQ ID NO:40; and (c) HVR-L3 comprising the amino acid sequence of SEQ ID NO:42. In one embodiment, the antibody comprises (a) HVR-L1 comprising the amino acid sequence of SEQ ID NO:37; (b) HVR-L2 comprising the amino acid sequence of SEQ ID NO:40; and (c) HVR-L3 comprising the amino acid sequence of SEQ ID NO:42.

In another aspect, an antibody of the invention comprises (a) a VH domain comprising at least one, at least two, or all three VH HVR sequences selected from (i) HVR-H1 comprising the amino acid sequence of SEQ ID NO:29, (ii) HVR-H2 comprising the amino acid sequence of SEQ ID NO:30, and (iii) HVR-H3 comprising an amino acid sequence selected from SEQ ID NO:33; and (b) a VL domain comprising at least one, at least two, or all three VL HVR sequences selected from (i) HVR-L1 comprising the amino acid sequence of SEQ ID NO:37, (ii) HVR-L2 comprising the amino acid sequence of SEQ ID NO:40, and (c) HVR-L3 comprising the amino acid sequence of SEQ ID NO:42.

In another aspect, the invention provides an antibody comprising (a) HVR-H1 comprising the amino acid sequence of SEQ ID NO:29; (b) HVR-H2 comprising the amino acid sequence of SEQ ID NO:30; (c) HVR-H3 comprising the amino acid sequence of SEQ ID NO:33; (d) HVR-L1 comprising the amino acid sequence of SEQ ID NO:37; (e) HVR-L2 comprising the amino acid sequence of SEQ ID NO:40; and (f) HVR-L3 comprising an amino acid sequence selected from SEQ ID NO:42.

In one aspect, the invention provides an anti-human OX40 agonist antibody comprising at least one, two, three, four, five, or six HVRs selected from (a) HVR-H1 comprising the amino acid sequence of SEQ ID NO:29; (b) HVR-H2 comprising the amino acid sequence of SEQ ID NO:30, 31, or 32; (c) HVR-H3 comprising the amino acid sequence of SEQ ID NO:33; (d) HVR-L1 comprising the amino acid sequence of SEQ ID NO:37; (e) HVR-L2 comprising the amino acid sequence of SEQ ID NO:39, 40 or 41; and (f) HVR-L3 comprising the amino acid sequence of SEQ ID NO:42, 43, or 44.

In one aspect, the invention provides an antibody comprising at least one, at least two, or all three VH HVR sequences selected from (a) HVR-H1 comprising the amino acid sequence of SEQ ID NO:29; (b) HVR-H2 comprising the amino acid sequence of SEQ ID NO: 30, 31, or 32; and (c) HVR-H3 comprising the amino acid sequence of SEQ ID NO:33. In another embodiment, the antibody comprises HVR-H3 comprising the amino acid sequence of SEQ ID NO:33 and HVR-L3 comprising the amino acid sequence of SEQ ID NO: 42, 43, or 44. In a further embodiment, the antibody comprises HVR-H3 comprising the amino acid sequence of SEQ ID NO:33, HVR-L3 comprising the amino acid sequence of SEQ ID NO: 42, 43, or 44, and HVR-H2 comprising the amino acid sequence of SEQ ID NO: 39, 40 or 41. In a further embodiment, the antibody comprises (a) HVR-H1 comprising the amino acid sequence of SEQ ID NO:29; (b) HVR-H2 comprising the amino acid sequence of SEQ ID NO:30, 31, or 32; and (c) HVR-H3 comprising the amino acid sequence of SEQ ID NO:33.

In another aspect, the invention provides an antibody comprising at least one, at least two, or all three VL HVR sequences selected from (a) HVR-L1 comprising the amino acid sequence of SEQ ID NO:37; (b) HVR-L2 comprising the amino acid sequence of SEQ ID NO: 39, 40 or 41; and (c) HVR-L3 comprising the amino acid sequence of SEQ ID NO: 42, 43, or 44. In one embodiment, the antibody comprises (a) HVR-L1 comprising the amino acid sequence of SEQ ID NO:37; (b) HVR-L2 comprising the amino acid sequence of SEQ ID NO: 39, 40 or 41; and (c) HVR-L3 comprising the amino acid sequence of SEQ ID NO: 42, 43, or 44.

In another aspect, an antibody of the invention comprises (a) a VH domain comprising at least one, at least two, or all three VH HVR sequences selected from (i) HVR-H1 comprising the amino acid sequence of SEQ ID NO:29, (ii) HVR-H2 comprising the amino acid sequence of SEQ ID NO: 30, 31, or 32, and (iii) HVR-H3 comprising an amino acid sequence selected from SEQ ID NO:33; and (b) a VL domain comprising at least one, at least two, or all three VL HVR sequences selected from (i) HVR-L1 comprising the amino acid sequence of SEQ ID NO:37, (ii) HVR-L2 comprising the amino acid sequence of SEQ ID NO: 39, 40 or 41, and (c) HVR-L3 comprising the amino acid sequence of SEQ ID NO: 42, 43, or 44.

In another aspect, the invention provides an antibody comprising (a) HVR-H1 comprising the amino acid sequence of SEQ ID NO:29; (b) HVR-H2 comprising the amino acid sequence of SEQ ID NO: 30, 31, or 32; (c) HVR-H3 comprising the amino acid sequence of SEQ ID NO:33; (d) HVR-L1 comprising the amino acid sequence of SEQ ID NO:37; (e) HVR-L2 comprising the amino acid sequence of SEQ ID NO: 39, 40 or 41; and (f) HVR-L3 comprising an amino acid sequence selected from SEQ ID NO: 42, 43, or 44.

In one aspect, the invention provides an anti-human OX40 agonist antibody comprising at least one, two, three, four, five, or six HVRs selected from (a) HVR-H1 comprising the amino acid sequence of SEQ ID NO:29; (b) HVR-H2 comprising the amino acid sequence of SEQ ID NO:175; (c) HVR-H3 comprising the amino acid sequence of SEQ ID NO:33; (d) HVR-L1 comprising the amino acid sequence of SEQ ID NO:37; (e) HVR-L2 comprising the amino acid sequence of SEQ ID NO:177; and (f) HVR-L3 comprising the amino acid sequence of SEQ ID NO:178.

In one aspect, the invention provides an antibody comprising at least one, at least two, or all three VH HVR sequences selected from (a) HVR-H1 comprising the amino acid sequence of SEQ ID NO:29; (b) HVR-H2 comprising the amino acid sequence of SEQ ID NO:175; and (c) HVR-H3 comprising the amino acid sequence of SEQ ID NO:33. In another embodiment, the antibody comprises HVR-H3 comprising the amino acid sequence of SEQ ID NO:33 and HVR-L3 comprising the amino acid sequence of SEQ ID NO:177. In a further embodiment, the antibody comprises HVR-H3 comprising the amino acid sequence of SEQ ID NO:33, HVR-L3 comprising the amino acid sequence of SEQ ID NO:178, and HVR-H2 comprising the amino acid sequence of SEQ ID NO:176. In a further embodiment, the antibody comprises (a) HVR-H1 comprising the amino acid sequence of SEQ ID NO:29; (b) HVR-H2 comprising the amino acid sequence of SEQ ID NO:176; and (c) HVR-H3 comprising the amino acid sequence of SEQ ID NO:33.

In another aspect, the invention provides an antibody comprising at least one, at least two, or all three VL HVR sequences selected from (a) HVR-L1 comprising the amino acid sequence of SEQ ID NO:37; (b) HVR-L2 comprising the amino acid sequence of SEQ ID NO:177; and (c) HVR-L3 comprising the amino acid sequence of SEQ ID NO:177. In one embodiment, the antibody comprises (a) HVR-L1 comprising the amino acid sequence of SEQ ID NO:37; (b) HVR-L2 comprising the amino acid sequence of SEQ ID NO:177; and (c) HVR-L3 comprising the amino acid sequence of SEQ ID NO:178.

In another aspect, an antibody of the invention comprises (a) a VH domain comprising at least one, at least two, or all three VH HVR sequences selected from (i) HVR-H1 comprising the amino acid sequence of SEQ ID NO:29, (ii) HVR-H2 comprising the amino acid sequence of SEQ ID NO:176, and (iii) HVR-H3 comprising an amino acid sequence selected from SEQ ID NO:33; and (b) a VL domain comprising at least one, at least two, or all three VL HVR sequences selected from (i) HVR-L1 comprising the amino acid sequence of SEQ ID NO:37, (ii) HVR-L2 comprising the amino acid sequence of SEQ ID NO:177, and (c) HVR-L3 comprising the amino acid sequence of SEQ ID NO:178.

In another aspect, the invention provides an antibody comprising (a) HVR-H1 comprising the amino acid sequence of SEQ ID NO:29; (b) HVR-H2 comprising the amino acid sequence of SEQ ID NO:176; (c) HVR-H3 comprising the amino acid sequence of SEQ ID NO:33; (d) HVR-L1 comprising the amino acid sequence of SEQ ID NO:37; (e) HVR-L2 comprising the amino acid sequence of SEQ ID NO:177; and (f) HVR-L3 comprising an amino acid sequence selected from SEQ ID NO:178.

In any of the above embodiments, an anti-OX40 agonist antibody is humanized.

In another aspect, an anti-human OX40 agonist antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:56, 58, 60, 62, 64, 66, 68, 70, 72, 74, 76, 78, 80, 82, 84, 86, 88, 90, 92, 94, 96, 98, 100, 108, 114, 116, 183, or 184. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO:56, 58, 60, 62, 64, 66, 68, 70, 72, 74, 76, 78, 80, 82, 84, 86, 88, 90, 92, 94, 96, 98, 100, 108, 114, 116, 183, or 184. In certain embodiments, substitutions, insertions, or deletions occur in regions outside the HVRs (i.e., in the FRs). Optionally, the anti-human OX40 agonist antibody comprises the VH sequence in SEQ ID NO: SEQ ID NO:56, 58, 60, 62, 64, 66, 68, 70, 72, 74, 76, 78, 80, 82, 84, 86, 88, 90, 92, 94, 96, 98, 100, 108, 114, 116, 183, or 184, including post-translational modifications of that sequence. In a particular embodiment, the VH comprises one, two or three HVRs selected from: (a) HVR-H1 comprising the amino acid sequence of SEQ ID NO:2, (b) HVR-H2 comprising the amino acid sequence of SEQ ID NO:3, and (c) HVR-H3 comprising the amino acid sequence of SEQ ID NO:4.

In another aspect, an anti-human OX40 agonist antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:57, 59, 61, 63, 65, 67, 69, 71, 73, 75, 77, 79, 81, 83, 85, 87, 89, 91, 93, 95, 97, 99, 101, 109, 115 or 117. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO: 57, 59, 61, 63, 65, 67, 69, 71, 73, 75, 77, 79, 81, 83, 85, 87, 89, 91, 93, 95, 97, 99, 101, 109, 115 or 117. In certain embodiments, the substitutions, insertions, or deletions occur in regions outside the HVRs (i.e., in the FRs). Optionally, the anti-human OX40 agonist antibody comprises the VL sequence in SEQ ID NO: 57, 59, 61, 63, 65, 67, 69, 71, 73, 75, 77, 79, 81, 83, 85, 87, 89, 91, 93, 95, 97, 99, 101, 109, 115 or 117, including post-translational modifications of that sequence. In a particular embodiment, the VL comprises one, two or three HVRs selected from (a) HVR-L1 comprising the amino acid sequence of SEQ ID NO:5; (b) HVR-L2 comprising the amino acid sequence of SEQ ID NO:6; and (c) HVR-L3 comprising the amino acid sequence of SEQ ID NO:7.

In another aspect, an anti-human OX40 agonist antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:56. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO:56. In certain embodiments, substitutions, insertions, or deletions occur in regions outside the HVRs (i.e., in the FRs). Optionally, the anti-human OX40 agonist antibody comprises the VH sequence in SEQ ID NO:56, including post-translational modifications of that sequence. In a particular embodiment, the VH comprises one, two or three HVRs selected from: (a) HVR-H1 comprising the amino acid sequence of SEQ ID NO:2, (b) HVR-H2 comprising the amino acid sequence of SEQ ID NO:3, and (c) HVR-H3 comprising the amino acid sequence of SEQ ID NO:4.

In another aspect, an anti-human OX40 agonist antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:57. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO: 57. In certain embodiments, the substitutions, insertions, or deletions occur in regions outside the HVRs (i.e., in the FRs). Optionally, the anti-human OX40 agonist antibody comprises the VL sequence in SEQ ID NO: 57, including post-translational modifications of that sequence. In a particular embodiment, the VL comprises one, two or three HVRs selected from (a) HVR-L1 comprising the amino acid sequence of SEQ ID NO:5; (b) HVR-L2 comprising the amino acid sequence of SEQ ID NO:6; and (c) HVR-L3 comprising the amino acid sequence of SEQ ID NO:7.

In another aspect, an anti-human OX40 agonist antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:180. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO:180. In certain embodiments, substitutions, insertions, or deletions occur in regions outside the HVRs (i.e., in the FRs). Optionally, the anti-human OX40 agonist antibody comprises the VH sequence in SEQ ID NO:180, including post-translational modifications of that sequence. In a particular embodiment, the VH comprises one, two or three HVRs selected from: (a) HVR-H1 comprising the amino acid sequence of SEQ ID NO:2, (b) HVR-H2 comprising the amino acid sequence of SEQ ID NO:3, and (c) HVR-H3 comprising the amino acid sequence of SEQ ID NO:4.

In another aspect, an anti-human OX40 agonist antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:179. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO: 179. In certain embodiments, the substitutions, insertions, or deletions occur in regions outside the HVRs (i.e., in the FRs). Optionally, the anti-human OX40 agonist antibody comprises the VL sequence in SEQ ID NO: 179, including post-translational modifications of that sequence. In a particular embodiment, the VL comprises one, two or three HVRs selected from (a) HVR-L1 comprising the amino acid sequence of SEQ ID NO:5; (b) HVR-L2 comprising the amino acid sequence of SEQ ID NO:6; and (c) HVR-L3 comprising the amino acid sequence of SEQ ID NO:7.

In another aspect, an anti-human OX40 agonist antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:94. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO:94. In certain embodiments, substitutions, insertions, or deletions occur in regions outside the HVRs (i.e., in the FRs). Optionally, the anti-human OX40 agonist antibody comprises the VH sequence in SEQ ID NO:94, including post-translational modifications of that sequence. In a particular embodiment, the VH comprises one, two or three HVRs selected from: (a) HVR-H1 comprising the amino acid sequence of SEQ ID NO:2, (b) HVR-H2 comprising the amino acid sequence of SEQ ID NO:3, and (c) HVR-H3 comprising the amino acid sequence of SEQ ID NO:4.

In another aspect, an anti-human OX40 agonist antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:95. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO:95. In certain embodiments, the substitutions, insertions, or deletions occur in regions outside the HVRs (i.e., in the FRs). Optionally, the anti-human OX40 agonist antibody comprises the VL sequence in SEQ ID NO:95, including post-translational modifications of that sequence. In a particular embodiment, the VL comprises one, two or three HVRs selected from (a) HVR-L1 comprising the amino acid sequence of SEQ ID NO:5; (b) HVR-L2 comprising the amino acid sequence of SEQ ID NO:6; and (c) HVR-L3 comprising the amino acid sequence of SEQ ID NO:26.

In another aspect, an anti-human OX40 agonist antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:96. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO:96. In certain embodiments, substitutions, insertions, or deletions occur in regions outside the HVRs (i.e., in the FRs). Optionally, the anti-human OX40 agonist antibody comprises the VH sequence in SEQ ID NO:96, including post-translational modifications of that sequence. In a particular embodiment, the VH comprises one, two or three HVRs selected from: (a) HVR-H1 comprising the amino acid sequence of SEQ ID NO:2, (b) HVR-H2 comprising the amino acid sequence of SEQ ID NO:3, and (c) HVR-H3 comprising the amino acid sequence of SEQ ID NO:4.

In another aspect, an anti-human OX40 agonist antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:97. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO:97. In certain embodiments, the substitutions, insertions, or deletions occur in regions outside the HVRs (i.e., in the FRs). Optionally, the anti-human OX40 agonist antibody comprises the VL sequence in SEQ ID NO:97, including post-translational modifications of that sequence. In a particular embodiment, the VL comprises one, two or three HVRs selected from (a) HVR-L1 comprising the amino acid sequence of SEQ ID NO:5; (b) HVR-L2 comprising the amino acid sequence of SEQ ID NO:6; and (c) HVR-L3 comprising the amino acid sequence of SEQ ID NO:27.

In another aspect, an anti-human OX40 agonist antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 118, 120, 122, 124, 126, 128, 130, 132, 134, 136, 138, 140, 142, 144, 146, 148. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO: 118, 120, 122, 124, 126, 128, 130, 132, 134, 136, 138, 140, 142, 144, 146, 148. In certain embodiments, substitutions, insertions, or deletions occur in regions outside the HVRs (i.e., in the FRs). Optionally, the anti-human OX40 agonist antibody comprises the VH sequence in SEQ ID NO: SEQ ID NO: 118, 120, 122, 124, 126, 128, 130, 132, 134, 136, 138, 140, 142, 144, 146, 148, including post-translational modifications of that sequence. In a particular embodiment, the VH comprises one, two or three HVRs selected from: (a) HVR-H1 comprising the amino acid sequence of SEQ ID NO: 29, (b) HVR-H2 comprising the amino acid sequence of SEQ ID NO:30, and (c) HVR-H3 comprising the amino acid sequence of SEQ ID NO:33.

In another aspect, an anti-human OX40 agonist antibody is provided, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 119, 121, 123, 125, 127, 129, 131, 133, 135, 137, 139, 141, 143, 145, 147, 149. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO: 119, 121, 123, 125, 127, 129, 131, 133, 135, 137, 139, 141, 143, 145, 147, 149. In certain embodiments, the substitutions, insertions, or deletions occur in regions outside the HVRs (i.e., in the FRs). Optionally, the anti-human OX40 agonist antibody comprises the VL sequence in SEQ ID NO: 119, 121, 123, 125, 127, 129, 131, 133, 135, 137, 139, 141, 143, 145, 147, 149, including post-translational modifications of that sequence. In a particular embodiment, the VL comprises one, two or three HVRs selected from (a) HVR-L1 comprising the amino acid sequence of SEQ ID NO:37; (b) HVR-L2 comprising the amino acid sequence of SEQ ID NO:39; and (c) HVR-L3 comprising the amino acid sequence of SEQ ID NO:42.

In one embodiment, the antibody comprises the VH and VL sequences in SEQ ID NO:56 and SEQ ID NO:57, respectively, including post-translational modifications of those sequences. In one embodiment, the antibody comprises the VH and VL sequences in SEQ ID NO:58 and SEQ ID NO:59, respectively, including post-translational modifications of those sequences. In one embodiment, the antibody comprises the VH and VL sequences in SEQ ID NO:60 and SEQ ID NO:61, respectively, including post-translational modifications of those sequences. In one embodiment, the antibody comprises the VH and VL sequences in SEQ ID NO:62 and SEQ ID NO:63, respectively, including post-translational modifications of those sequences. In one embodiment, the antibody comprises the VH and VL sequences in SEQ ID NO:64 and SEQ ID NO:65, respectively, including post-translational modifications of those sequences. In one embodiment, the antibody comprises the VH and VL sequences in SEQ ID NO:66 and SEQ ID NO:67, respectively, including post-translational modifications of those sequences. In one embodiment, the antibody comprises the VH and VL sequences in SEQ ID NO:68 and SEQ ID NO:69, respectively, including post-translational modifications of those sequences. In one embodiment, the antibody comprises the VH and VL sequences in SEQ ID NO:70 and SEQ ID NO:71, respectively, including post-translational modifications of those sequences. In one embodiment, the antibody comprises the VH and VL sequences in SEQ ID NO:72 and SEQ ID NO:73, respectively, including post-translational modifications of those sequences. In one embodiment, the antibody comprises the VH and VL sequences in SEQ ID NO:74 and SEQ ID NO:75, respectively, including post-translational modifications of those sequences. In one embodiment, the antibody comprises the VH and VL sequences in SEQ ID NO:76 and SEQ ID NO:77, respectively, including post-translational modifications of those sequences. In one embodiment, the antibody comprises the VH and VL sequences in SEQ ID NO:78 and SEQ ID NO:79, respectively, including post-translational modifications of those sequences. In one embodiment, the antibody comprises the VH and VL sequences in SEQ ID NO:80 and SEQ ID NO:81, respectively, including post-translational modifications of those sequences. In one embodiment, the antibody comprises the VH and VL sequences in SEQ ID NO:82 and SEQ ID NO:83, respectively, including post-translational modifications of those sequences. In one embodiment, the antibody comprises the VH and VL sequences in SEQ ID NO:84 and SEQ ID NO:85, respectively, including post-translational modifications of those sequences. In one embodiment, the antibody comprises the VH and VL sequences in SEQ ID NO:86 and SEQ ID NO:87, respectively, including post-translational modifications of those sequences. In one embodiment, the antibody comprises the VH and VL sequences in SEQ ID NO:88 and SEQ ID NO:89, respectively, including post-translational modifications of those sequences. In one embodiment, the antibody comprises the VH and VL sequences in SEQ ID NO:90 and SEQ ID NO:91, respectively, including post-translational modifications of those sequences. In one embodiment, the antibody comprises the VH and VL sequences in SEQ ID NO:92 and SEQ ID NO:93, respectively, including post-translational modifications of those sequences. In one embodiment, the antibody comprises the VH and VL sequences in SEQ ID NO:94 and SEQ ID NO:95, respectively, including post-translational modifications of those sequences. In one embodiment, the antibody comprises the VH and VL sequences in SEQ ID NO:96 and SEQ ID NO:97, respectively, including post-translational modifications of those sequences. In one embodiment, the antibody comprises the VH and VL sequences in SEQ ID NO:98 and SEQ ID NO:99, respectively, including post-translational modifications of those sequences. In one embodiment, the antibody comprises the VH and VL sequences in SEQ ID NO:100 and SEQ ID NO:101, respectively, including post-translational modifications of those sequences. In one embodiment, the antibody comprises the VH and VL sequences in SEQ ID NO:108 and SEQ ID NO:109, respectively, including post-translational modifications of those sequences. In one embodiment, the antibody comprises the VH and VL sequences in SEQ ID NO:114 and SEQ ID NO:115, respectively, including post-translational modifications of those sequences. In one embodiment, the antibody comprises the VH and VL sequences in SEQ ID NO:116 and SEQ ID NO:117, respectively, including post-translational modifications of those sequences. In one embodiment, the antibody comprises the VH and VL sequences in SEQ ID NO:183 and SEQ ID NO:65, respectively, including post-translational modifications of those sequences. In one embodiment, the antibody comprises the VH and VL sequences in SEQ ID NO:184 and SEQ ID NO:69, respectively, including post-translational modifications of those sequences.

In one embodiment, the antibody comprises the VH and VL sequences in SEQ ID NO:118 and SEQ ID NO:119, respectively, including post-translational modifications of those sequences. In one embodiment, the antibody comprises the VH and VL sequences in SEQ ID NO:120 and SEQ ID NO:121, respectively, including post-translational modifications of those sequences. In one embodiment, the antibody comprises the VH and VL sequences in SEQ ID NO:122 and SEQ ID NO:123, respectively, including post-translational modifications of those sequences. In one embodiment, the antibody comprises the VH and VL sequences in SEQ ID NO:124 and SEQ ID NO:125, respectively, including post-translational modifications of those sequences. In one embodiment, the antibody comprises the VH and VL sequences in SEQ ID NO:126 and SEQ ID NO:127, respectively, including post-translational modifications of those sequences. In one embodiment, the antibody comprises the VH and VL sequences in SEQ ID NO:128 and SEQ ID NO:129, respectively, including post-translational modifications of those sequences. In one embodiment, the antibody comprises the VH and VL sequences in SEQ ID NO:130 and SEQ ID NO:131, respectively, including post-translational modifications of those sequences. In one embodiment, the antibody comprises the VH and VL sequences in SEQ ID NO:132 and SEQ ID NO:133, respectively, including post-translational modifications of those sequences. In one embodiment, the antibody comprises the VH and VL sequences in SEQ ID NO:134 and SEQ ID NO:135, respectively, including post-translational modifications of those sequences. In one embodiment, the antibody comprises the VH and VL sequences in SEQ ID NO:136 and SEQ ID NO:137, respectively, including post-translational modifications of those sequences. In one embodiment, the antibody comprises the VH and VL sequences in SEQ ID NO:138 and SEQ ID NO:139, respectively, including post-translational modifications of those sequences. In one embodiment, the antibody comprises the VH and VL sequences in SEQ ID NO:140 and SEQ ID NO:141, respectively, including post-translational modifications of those sequences. In one embodiment, the antibody comprises the VH and VL sequences in SEQ ID NO:142 and SEQ ID NO:143, respectively, including post-translational modifications of those sequences. In one embodiment, the antibody comprises the VH and VL sequences in SEQ ID NO:144 and SEQ ID NO:145, respectively, including post-translational modifications of those sequences. In one embodiment, the antibody comprises the VH and VL sequences in SEQ ID NO:146 and SEQ ID NO:147, respectively, including post-translational modifications of those sequences.

In another aspect, an anti-human OX40 agonist antibody is provided, wherein the antibody comprises a VH as in any of the embodiments provided above, and a VL as in any of the embodiments provided above.

In a further aspect, the invention provides an antibody that binds to the same epitope as an anti-human OX40 antibody provided herein. In some embodiments, the antibody is an anti-human OX40 agonist antibody.

In a further aspect of the invention, an anti-OX40 antibody according to any of the above embodiments is a monoclonal antibody, including a chimeric, humanized or human antibody. In one embodiment, an anti-OX40 antibody is an antibody fragment, e.g., a Fv, Fab, Fab′, scFv, diabody, or F(ab′)₂ fragment. In another embodiment, the antibody is a full length antibody, e.g., an intact IgG1 antibody or other antibody class or isotype as defined herein. In some embodiments, the antibody is a full length intact IgG4 antibody.

In a further aspect, an anti-OX40 antibody according to any of the above embodiments may incorporate any of the features, singly or in combination, as described in Sections 1-7 below:

1. Antibody Affinity

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵D-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CMS chips at ˜10 response units (RU). In one embodiment, carboxymethylated dextran biosensor chips (CMS, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20^{Tm}) surfactant (PBST) at 25° C. at a flow rate of approximately 25 μl/min. Association rates (kₒₙ) and dissociation rates (k_{off}) are calculated using a simple one-to-one Langmuir binding model (BIACORE ° Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 106 M-1 s-1 by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al. Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for OX40 and the other is for any other antigen. In certain embodiments, bispecific antibodies may bind to two different epitopes of OX40. Bispecific antibodies may also be used to localize cytotoxic agents to cells which express OX40. Bispecific antibodies can be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO 10: 3655 (1991)), and “knob-in-hole” engineering (see, e.g., U.S. Pat. No. 5,731,168). Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see,e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to OX40 as well as another, different antigen (see, US 2008/0069820, for example).

7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table A under the heading of “preferred substitutions.” More substantial changes are provided in Table A under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE A

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:

(1) hydrophobic: Norleucine, Met, Ala, Val, Leu, Ile;

(2) neutral hydrophilic: Cys, Ser, Thr, Asn, Gln;

(3) acidic: Asp, Glu;

(4) basic: His, Lys, Arg;

(5) residues that influence chain orientation: Gly, Pro;

(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. No. 5,648,260; U.S. Pat. No. 5,624,821; and WO 94/29351 concerning other examples of Fc region variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

e) Antibody Derivatives

In certain embodiments, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### B. Recombinant Methods and Compositions

Antibodies may be produced using recombinant methods and compositions, e.g., as described in U.S. Pat. No. 4,816,567. In one embodiment, isolated nucleic acid encoding an anti-OX40 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., YO, NSO, Sp20 cell). In one embodiment, a method of making an anti-OX40 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-OX40 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR⁻ CHO cells (Urlaub et al., Proc. Natl. Acad. Sci. USA 77:4216 (1980)); and myeloma cell lines such as YO, NSO and Sp2/0. For a review of certain mammalian host cell lines suitable for antibody production, see, e.g., Yazaki and Wu, Methods in Molecular Biology, Vol. 248 (B.K.C. Lo, ed., Humana Press, Totowa, N.J.), pp. 255-268 (2003).

### C. Assays

Anti-OX40 antibodies provided herein may be identified, screened for, or characterized for their physical/chemical properties and/or biological activities by various assays known in the art.

1. Binding Assays and Other Assays

In one aspect, an antibody of the invention is tested for its antigen binding activity, e.g., by known methods such as ELISA, Western blot, etc. OX40 binding may be determined using methods known in the art and exemplary methods are disclosed herein. In one embodiment, binding is measured using radioimmunoassay. In an exemplary radioimmunassay, OX40 antibody is iodinated, and competition reaction mixtures are prepared containing a fixed concentration of iodinated antibody and decreasing concentrations of serially diluted, unlabeled OZ X40 antibody. Cells expressing OX40 (e.g., BT474 cells stably transfected with human OX40) are added to the reaction mixture. Following an incubation, cells are washed to separate the free iodinated OX40 antibody from the OX40 antibody bound to the cells. Level of bound iodinated OX40 antibody is determined, e.g., by counting radioactivity associated with cells, and binding affinity determined using standard methods. In another embodiment, ability of OX40 antibody to bind to surface-expressed OX40 (e.g., on T cell subsets) is assessed using flow cytometry. Peripheral white blood cells are obtained (e.g., from human, cynomolgus monkey, rat or mouse) and cells are blocked with serum. Labeled OX40 antibody is added in serial dilutions, and T cells are also stained to identify T cell subsets (using methods known in the art). Following incubation of the samples and washing, the cells are sorted using flow cytometer, and data analyzed using methods well known in the art. In another embodiment, OX40 binding may be analyzed using surface plasmon resonance. An exemplary surface plasmon resonance method is exemplified in the Examples.

In another aspect, competition assays may be used to identify an antibody that competes with any of the anti-OX40 antibodies disclosed herein for binding to OX40. In certain embodiments, such a competing antibody binds to the same epitope (e.g., a linear or a conformational epitope) that is bound by any of the anti-OX40 antibodies disclosed herein. Detailed exemplary methods for mapping an epitope to which an antibody binds are provided in Morris (1996) “Epitope Mapping Protocols,” in Methods in Molecular Biology vol. 66 (Humana Press, Totowa, N.J.). A competition assay is exemplified in the Examples.

In an exemplary competition assay, immobilized OX40 is incubated in a solution comprising a first labeled antibody that binds to OX40 (e.g., mab 1A7.gr.1, mab 3C8.gr5) and a second unlabeled antibody that is being tested for its ability to compete with the first antibody for binding to OX40. The second antibody may be present in a hybridoma supernatant. As a control, immobilized OX40 is incubated in a solution comprising the first labeled antibody but not the second unlabeled antibody. After incubation under conditions permissive for binding of the first antibody to OX40, excess unbound antibody is removed, and the amount of label associated with immobilized OX40 is measured. If the amount of label associated with immobilized OX40 is substantially reduced in the test sample relative to the control sample, then that indicates that the second antibody is competing with the first antibody for binding to OX40. See Harlow and Lane (1988) Antibodies: A Laboratory Manual ch.14 (Cold Spring Harbor Laboratory, Cold Spring Harbor, N.Y.).

2. Activity Assays

In one aspect, assays are provided for identifying anti-OX40 antibodies thereof having biological activity. Biological activity may include, e.g., binding OX40 (e.g., binding human and/or cynomolgus OX40), increasing OX40-mediated signal transduction (e.g., increasing NFkB-mediated transcription), depleting cells that express human OX40 (e.g., T cells), depleting cells that express human OX40 by ADCC and/or phagocytosis, enhancing T effector cell function (e.g., CD4+ effector T cell), e.g., by increasing effector T cell proliferation and/or increasing cytokine production (e.g., gamma interferon) by effector T cells, enhancing memory T cell function (e.g., CD4+ memory T cell), e.g., by increasing memory T cell proliferation and/or increasing cytokine production by memory T cells (e.g., gamma interferon), inhibiting regulatory T cell function (e.g., by decreasing Treg suppression of effector T cell function (e.g., CD4+ effector T cell function), binding human effector cells. Antibodies having such biological activity in vivo and/or in vitro are also provided.

In certain embodiments, an antibody of the invention is tested for such biological activity.

T cell costimulation may be assayed using methods known in the art and exemplary methods are disclosed herein. For example, T cells (e.g., memory or effector T cells) may be obtained from peripheral white blood cells (e.g., isolated from human whole blood using Ficoll gradient centrifugation). Memory T cells (e.g., CD4+ memory T cells) or effector T cells (e.g. CD4+ Teff cells) may be isolated from PBMC using methods known in the art. For example, the Miltenyi CD4+ memory T cell isolation kit or Miltenyi naïve CD4+ T cell isolation kit may be used. Isolated T cells are cultured in the presence of antigen presenting cells (e.g., irradiated L cells that express CD32 and CD80), and activated by addition of anti-CD3 antibody in the presence or absence of OX40 agonist antibody. Effect of agonist OX40 antibody of T cell proliferation may be measured using methods well known in the art. For example, the CellTiter Glo kit (Promega) may be used, and results read on a Multilabel Reader (Perkin Elmer). Effect of agonist OX40 antibody on T cell function may also be determined by analysis of cytokines produced by the T cell. In one embodiment, production of interferon gamma by CD4+ T cells is determined, e.g., by measurement of interferon gamma in cell culture supernatant. Methods for measuring interferon gamma are well-known in the art.

Treg cell function may be assayed using methods known in the art and exemplary methods are disclosed herein. In one example, the ability of Treg to suppress effector T cell proliferation is assayed. T cells are isolated from human whole blood using methods known in the art (e.g., isolating memory T cells or naïve T cells). Purified CD4+ naïve T cells are labeled (e.g., with CFSE) and purified Treg cells are labeled with a different reagent. Irradiated antigen presenting cells (e.g., L cells expressing CD32 and CD80) are co-cultured with the labeled purified naïve CD4+ T cells and purified Tregs. The co-cultures are activated using anti-CD3 antibody and tested in the presence or absence of agonist OX40 antibody. Following a suitable time (e.g., 6 days of coculture), level of CD4+ naïve T cell proliferation is tracked by dye dilution in reduced label staining (e.g., reduced CFSE label staining) using FACS analysis.

OX40 signaling may be assayed using methods well known in the art and exemplary methods are disclosed herein. In one embodiment, transgenic cells are generated that express human OX40 and a reporter gene comprising the NFkB promoter fused to a reporter gene (e.g., beta luciferase). Addition of OX40 agonist antibody to the cells results in increased NFkB transcription, which is detected using an assay for the reporter gene.

Phagocytosis may be assayed, e.g., by using monocyte-derived macrophages, or U937 cells (a human histiocytic lymphoma cells line with the morphology and characteristics of mature macrophages). OX40 expressing cells are added to the monocyte-derived macrophages or U937 cells in the presence or absence of anti-OX40 agonist antibody. Following culturing of the cells for a suitable period of time, the percentage of phagocytosis is determined by examining percentage of cells that double stain for markers of 1) the macrophage or U937 cell and 2) the OX40 expressing cell, and dividing this by the total number of cells that show markers of the OX40 expressing cell (e.g., GFP). Analysis may be done by flow cytometry. In another embodiment, analysis may be done by fluorescent microscopy analysis.

ADCC may be assayed, e.g., using methods well known in the art. Exemplary methods are described in the definition section and an exemplary assay is disclosed in the Examples. In some embodiments, level of OX40 is characterized on an OX40 expressing cell that is used for testing in an ADCC assay. The cell may be stained with a detectably labeled anti-OX40 antibody (e.g., PE labeled), then level of fluorescence determined using flow cytometry, and results presented as median fluorescence intensity (MFI). In another embodiment, ADCC may be analyzed by CellTiter Glo assay kit and cell viability/cytotoxicity may be determined by chemioluminescence.

The binding affinities of various antibodies to FcγRIA, FcγRIIA, FcγRIIB, and two allotypes of FcγRIIIA (F158 and V158) may be measured in ELISA-based ligand-binding assays using the respective recombinant Fcγ receptors. Purified human Fcγ receptors are expressed as fusion proteins containing the extracellular domain of the receptor γ chain linked to a Gly/6×His/glutathione S-transferase (GST) polypeptide tag at the C-terminus. The binding affinities of antibodies to those human Fcγ receptors are assayed as follows. For the low-affinity receptors, i.e. FcγRIIA (CD32A), FcγRIIB (CD32B), and the two allotypes of FcγRIIIA (CD16), F-158 and V-158, antibodies may be tested as multimers by cross-linking with a F(ab′)2 fragment of goat anti-human kappa chain (ICN Biomedical; Irvine, Calif.) at an approximate molar ratio of 1:3 antibody:cross-linking F(ab′)₂. Plates are coated with an anti-GST antibody (Genentech) and blocked with bovine serum albumin (BSA). After washing with phosphate-buffered saline (PBS) containing 0.05% Tween-20 with an ELx405™ plate washer (Biotek Instruments; Winooski, Vt.), Fcγ receptors are added to the plate at 25 ng/well and incubated at room temperature for 1 hour. After the plates are washed, serial dilutions of test antibodies are added as multimeric complexes and the plates were incubated at room temperature for 2 hours. Following plate washing to remove unbound antibodies, the antibodies bound to the Fcγ receptor are detected with horseradish peroxidase (HRP)-conjugated F(ab′)₂ fragment of goat anti-human F(ab′)₂ (Jackson ImmunoResearch Laboratories; West Grove, Pa.) followed by the addition of substrate, tetramethylbenzidine (TMB) (Kirkegaard & Perry Laboratories; Gaithersburg, Md.). The plates are incubated at room temperature for 5-20 minutes, depending on the Fcγ receptors tested, to allow color development. The reaction is terminated with 1 M H₃PO₄ and absorbance at 450 nm was measured with a microplate reader (SpectraMax® 190, Molecular Devices; Sunnyvale, Calif.). Dose-response binding curves are generated by plotting the mean absorbance values from the duplicates of antibody dilutions against the concentrations of the antibody. Values for the effective concentration of the antibody at which 50% of the maximum response from binding to the Fcγ receptor is detected (EC₅₀) were determined after fitting the binding curve with a four-parameter equation using SoftMax Pro (Molecular Devices).

To select for antibodies which induce cell death, loss of membrane integrity as indicated by, e.g., propidium iodide (PI), trypan blue or 7AAD uptake may be assessed relative to control. A PI uptake assay can be performed in the absence of complement and immune effector cells. OX40 expressing cells are incubated with medium alone or medium containing of the appropriate monoclonal antibody at e.g., about 10 μg/ml. The cells are incubated for a time period (e.g., 1 or 3 days). Following each treatment, cells are washed and aliquoted. In some embodiments, cells are aliquoted into 35 mm strainer-capped 12×75 tubes (1 ml per tube, 3 tubes per treatment group) for removal of cell clumps. Tubes then receive PI (10 μg/ml). Samples may be analyzed using a FACSCAN™ flow cytometer and FACSCONVERT™ CellQuest software (Becton Dickinson).

Cells for use in any of the above in vitro assays include cells or cell lines that naturally express OX40 or that have been engineered to express OX40. Such cells include activated T cells, Treg cells and activated memory T cells that naturally express OX40. Such cells also include cell lines that express OX40 and cell lines that do not normally express OX40 but have been transfected with nucleic acid encoding OX40. Exemplary cell lines provided herein for use in any of the above in vitro assays include transgenic BT474 cells (a human breast cancer cell line) that express human OX40

It is understood that any of the above assays may be carried out using an immunoconjugate of the invention in place of or in addition to an anti-OX40 antibody.

It is understood that any of the above assays may be carried out using anti-OX40 antibody and an additional therapeutic agent.

### D. Immunoconjugates

The invention also provides immunoconjugates comprising an anti-OX40 antibody herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or I123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of an antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

### E. Methods and Compositions for Diagnostics and Detection

In certain embodiments, any of the anti-OX40 antibodies provided herein is useful for detecting the presence of OX40 in a biological sample. The term “detecting” as used herein encompasses quantitative or qualitative detection. In certain embodiments, a biological sample comprises a cell or tissue, such as a sample of a tumor (e.g., NSCLC or breast tumor).

In one embodiment, an anti-OX40 antibody for use in a method of diagnosis or detection is provided. In a further aspect, a method of detecting the presence of OX40 in a biological sample is provided. In certain embodiments, the method comprises contacting the biological sample with an anti-OX40 antibody as described herein under conditions permissive for binding of the anti-OX40 antibody to OX40, and detecting whether a complex is formed between the anti-OX40 antibody and OX40. Such method may be an in vitro or in vivo method. In one embodiment, an anti-OX40 antibody is used to select subjects eligible for therapy with an anti-OX40 antibody, e.g. where OX40 is a biomarker for selection of patients.

In some embodiments, the anti-OX40 antibody for use in a method of diagnosis or detection is an anti-human OX40 antibody comprising at least one, two, three, four, five, or six HVRs selected from (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising the amino acid sequence of SEQ ID NO:7. In some embodiments, the anti-OX40 antibody comprises (a) a VH domain comprising at least one, at least two, or all three VH HVR sequences selected from (i) HVR-H1 comprising the amino acid sequence of SEQ ID NO:2, (ii) HVR-H2 comprising the amino acid sequence of SEQ ID NO:3, and (iii) HVR-H3 comprising an amino acid sequence selected from SEQ ID NO:4; and (b) a VL domain comprising at least one, at least two, or all three VL HVR sequences selected from (i) HVR-L1 comprising the amino acid sequence of SEQ ID NO:5, (ii) HVR-L2 comprising the amino acid sequence of SEQ ID NO:6, and (c) HVR-L3 comprising the amino acid sequence of SEQ ID NO:7. In some embodiments, the OX40 antibody comprises (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7. In some embodiments, the antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:180. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO:180. In certain embodiments, substitutions, insertions, or deletions occur in regions outside the HVRs (i.e., in the FRs). Optionally, the anti-human OX40 agonist antibody comprises the VH sequence in SEQ ID NO:180, including post-translational modifications of that sequence. In a particular embodiment, the VH comprises one, two or three HVRs selected from: (a) HVR-H1 comprising the amino acid sequence of SEQ ID NO:2, (b) HVR-H2 comprising the amino acid sequence of SEQ ID NO:3, and (c) HVR-H3 comprising the amino acid sequence of SEQ ID NO:4. In some embodiments, the antibody comprises a light chain variable domain (VL) having at least 90%, 91%, 92%, 93%, 94%, 95%, 96%, 97%, 98%, 99%, or 100% sequence identity to the amino acid sequence of SEQ ID NO:179. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO: 179. In certain embodiments, the substitutions, insertions, or deletions occur in regions outside the HVRs (i.e., in the FRs). Optionally, the anti-human OX40 agonist antibody comprises the VL sequence in SEQ ID NO: 179, including post-translational modifications of that sequence. In a particular embodiment, the VL comprises one, two or three HVRs selected from (a) HVR-L1 comprising the amino acid sequence of SEQ ID NO:5; (b) HVR-L2 comprising the amino acid sequence of SEQ ID NO:6; and (c) HVR-L3 comprising the amino acid sequence of SEQ ID NO:7.

In some embodiments, the anti-OX40 antibody used in the method of diagnosis or detection is an anti-human OX40 antibody comprising at least one, two, three, four, five, or six HVRs selected from (a) HVR-H1 comprising the amino acid sequence of SEQ ID NO:29; (b) HVR-H2 comprising the amino acid sequence of SEQ ID NO:30; (c) HVR-H3 comprising the amino acid sequence of SEQ ID NO:31; (d) HVR-L1 comprising the amino acid sequence of SEQ ID NO:37; (e) HVR-L2 comprising the amino acid sequence of SEQ ID NO:39; and (f) HVR-L3 comprising the amino acid sequence of SEQ ID NO:42. In some embodiments, the anti-OX40 antibody comprises (a) a VH domain comprising at least one, at least two, or all three VH HVR sequences selected from (i) HVR-H1 comprising the amino acid sequence of SEQ ID NO:29, (ii) HVR-H2 comprising the amino acid sequence of SEQ ID NO:30, and (iii) HVR-H3 comprising an amino acid sequence selected from SEQ ID NO:31; and (b) a VL domain comprising at least one, at least two, or all three VL HVR sequences selected from (i) HVR-L1 comprising the amino acid sequence of SEQ ID NO:37, (ii) HVR-L2 comprising the amino acid sequence of SEQ ID NO:39, and (c) HVR-L3 comprising the amino acid sequence of SEQ ID NO:42. In some embodiments, the anti-OX40 antibody comprises (a) HVR-H1 comprising the amino acid sequence of SEQ ID NO:29; (b) HVR-H2 comprising the amino acid sequence of SEQ ID NO:30; (c) HVR-H3 comprising the amino acid sequence of SEQ ID NO:31; (d) HVR-L1 comprising the amino acid sequence of SEQ ID NO:37; (e) HVR-L2 comprising the amino acid sequence of SEQ ID NO:39; and (f) HVR-L3 comprising an amino acid sequence selected from SEQ ID NO:42. In some embodiment, the anti-OX40 antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO:182. In certain embodiments, a VH sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO:182. In certain embodiments, substitutions, insertions, or deletions occur in regions outside the HVRs (i.e., in the FRs). Optionally, the anti-human OX40 agonist antibody comprises the VH sequence in SEQ ID NO:182, including post-translational modifications of that sequence. In a particular embodiment, the VH comprises one, two or three HVRs selected from: (a) HVR-H1 comprising the amino acid sequence of SEQ ID NO:29, (b) HVR-H2 comprising the amino acid sequence of SEQ ID NO:30, and (c) HVR-H3 comprising the amino acid sequence of SEQ ID NO:31. In some embodiments, the anti-OX40 antibody comprises a light chain variable domain (VL) having at least 90%, 91%, 92%, 93%, 94%, 95%, 96%, 97%, 98%, 99%, or 100% sequence identity to the amino acid sequence of SEQ ID NO:181. In certain embodiments, a VL sequence having at least 90%, 91%, 92%, 93%, 94%, 95%, 96%, 97%, 98%, or 99% identity contains substitutions (e.g., conservative substitutions), insertions, or deletions relative to the reference sequence, but an anti-human OX40 agonist antibody comprising that sequence retains the ability to bind to OX40. In certain embodiments, a total of 1 to 10 amino acids have been substituted, inserted and/or deleted in SEQ ID NO: 181. In certain embodiments, the substitutions, insertions, or deletions occur in regions outside the HVRs (i.e., in the FRs). Optionally, the anti-human OX40 agonist antibody comprises the VL sequence in SEQ ID NO: 181, including post-translational modifications of that sequence. In a particular embodiment, the VL comprises one, two or three HVRs selected from (a) HVR-L1 comprising the amino acid sequence of SEQ ID NO:37; (b) HVR-L2 comprising the amino acid sequence of SEQ ID NO:39; and (c) HVR-L3 comprising the amino acid sequence of SEQ ID NO:42.

In some embodiments, the anti-OX40 antibody comprises a VH sequence of SEQ ID NO: 180. In some embodiments, the anti-OX40 antibody comprises a VL sequence of SEQ ID NO: 179. In some embodiments, the anti-OX40 antibody comprises a VH sequence of SEQ ID NO:180 and a VL sequence of SEQ ID NO: 179. In some embodiments, the anti-OX40 antibody comprises a VH sequence of SEQ ID NO: 182. In some embodiments, the anti-OX40 antibody comprises a VL sequence of SEQ ID NO: 181. In some embodiments, the anti-OX40 antibody comprises a VH sequence of SEQ ID NO:182 and a VL sequence of SEQ ID NO: 181.

Exemplary disorders that may be diagnosed using an antibody of the invention include cancer.

In certain embodiments, labeled anti-OX40 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

In one aspect, the invention provides diagnostic methods, e.g. for identifying a cancer patient who is likely to respond to treatment with an anti-human OX40 agonist antibody.

In some embodiments, methods are provided for identifying patients who are likely to respond to treatment with anti-human OX40 agonist antibody, the methods comprising (i) determining presence or absence or amount (e.g., number per given sample size) of cells expressing FcR in a sample of cancer from the patient, and (ii) identifying the patient as likely to respond if the sample comprises cells expressing FcR (e.g., high number of cells expressing FcR). Methods for detecting cells that express FcR are well known in the art, including, e.g., by IHC. In some embodiments, FcR is FcγR. In some embodiments, FcR is an activating FcγR. In some embodiments, the cancer is any cancer described herein. In some embodiments, the cancer is non-small cell lung cancer (NSCLC), glioblastoma, neuroblastoma, melanoma, breast carcinoma (e.g. triple-negative breast cancer), gastric cancer, colorectal cancer (CRC), or hepatocellular carcinoma. In some embodiments, the method is an in vitro method. In some embodiments, the methods further comprise (iii) recommending treatment with the anti-human OX40 agonist antibody (e.g., any of the anti-human OX40 agonist antibodies described herein). In some embodiments, the methods further comprise (iv) treating the patient with the anti-human OX40 agonist antibody.

In some embodiments, methods are provided for identifying patients who are likely to respond to treatment with anti-human OX40 agonist antibody, the methods comprising (i) determining presence or absence or amount (e.g., number per given sample size) of human effector cells (e.g., infiltrating effector cells) in a sample of cancer from the patient, and (ii) identifying the patient as likely to respond if the sample comprises effector cells (e.g., high number of effector cells). Methods for detecting infiltrating human effector cells are well known in the art, including, e.g., by IHC. In some embodiments, human effector cells are one or more of NK cells, macrophages, monocytes. In some embodiments, the effector cells express activating FcγR. In some embodiments, the method is an in vitro method. In some embodiments, the cancer is any cancer described herein. In some embodiments, the cancer is non-small cell lung cancer (NSCLC), glioblastoma, neuroblastoma, melanoma, breast carcinoma (e.g. triple-negative breast cancer), gastric cancer, colorectal cancer (CRC), or hepatocellular carcinoma. In some embodiments, the methods further comprise (iii) recommending treatment with the anti-human OX40 agonist antibody (e.g., any of the anti-human OX40 agonist antibodies described herein). In some embodiments, the methods further comprise (iv) treating the patient with the anti-human OX40 agonist antibody.

Provided are methods of providing a cancer diagnosis comprising: (i) measuring FcR expressing cells (e.g., the level or presence or absence of or prevalence (e.g., percentage of cells expressing FcR, e.g., by IHC) of FcR) in a sample from the patient; (ii) diagnosing the patient as having cancer comprising FcR biomarker (e.g., high FcR biomarker) when the sample has FcR biomarker expression. In some embodiments, the method further comprises (iii) selecting a therapy comprising (a) anti-human OX40 agonist antibody or (b) recommending a therapy comprising anti-human OX40 agonist antibody for the patient. In some embodiments, the method is an in vitro method.

Provided are methods of providing a cancer diagnosis comprising: (i) measuring human effector cells (e.g., the level or presence or absence of or prevalence (e.g., percentage of human effector cells) of human effector cells) in a sample from the patient; (ii) diagnosing the patient as having cancer comprising human effector cells (e.g., high human effector cells) when the sample has human effector cell biomarker. In some embodiments, the method further comprises (iii) selecting a therapy comprising (a) anti-human OX40 agonist antibody or (b) recommending a therapy comprising anti-human OX40 agonist antibody for the patient. In some embodiments, the method is an in vitro method.

Provided are methods of recommending a treatment to a cancer patient comprising: (i) measuring FcR expressing cells (e.g., the level or presence or absence of or prevalence (e.g., percentage of cells expressing FcR) of FcR) in a sample from the patient; (ii) recommending treatment with an anti-human OX40 agonist antibody when the sample has FcR expressing cells (in some embodiments, high FcR expressing cells). In some embodiments, the method further comprises (iii) selecting a therapy comprising anti-human OX40 agonist antibody for the patient. In some embodiments, the method is an in vitro method.

Provided are methods of recommending a treatment to a cancer patient comprising: (i) measuring human effector cells (e.g., the level or presence or absence of or prevalence (e.g., percentage of human effector cells) of human effector cells) in a sample from the patient; (ii) recommending treatment with an anti-human OX40 agonist antibody when the sample has human effector cells (in some embodiments, high human effector cells). In some embodiments, the method further comprises (iii) selecting a therapy comprising anti-human OX40 agonist antibody for the patient. In some embodiments, the method is an in vitro method.

In some embodiments of any of the inventions provided herein, the sample is obtained prior to treatment with anti-human OX40 agonist antibody. In some embodiments, the sample is obtained prior to treatment with a cancer medicament. In some embodiments, the sample is obtained after the cancer has metastasized. In some embodiments, the sample is formalin fixed and paraffin embedded (FFPE). In some embodiments, the sample is of a biopsy (e.g., a core biopsy), a surgical specimen (e.g., a specimen from a surgical resection), or a fine needle aspirate.

### F. Pharmaceutical Formulations

Pharmaceutical formulations of an anti-OX40 antibody as described herein are prepared by mixing such antibody having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

In some embodiments, a “histidine buffer” is a buffer comprising histidine ions. Examples of histidine buffers include histidine chloride, histidine acetate, histidine phosphate, histidine sulfate. The preferred histidine buffer identified in the examples herein was found to be histidine acetate. In the preferred embodiment, the histidine acetate buffer is prepared by titrating L-histidine (free base, solid) with acetic acid (liquid). In some embodiments, the histidine buffer or histidine-acetate buffer is at pH 5.0 to 6.0, in some embodiments, pH 5.3 to 5.8.

In some embodiments, a “saccharide” herein comprises the general composition (CH2O)n and derivatives thereof, including monosaccharides, disaccharides, trisaccharides, polysaccharides, sugar alcohols, reducing sugars, nonreducing sugars, etc. Examples of saccharides herein include glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc. In some embodiments, the saccharide is a nonreducing disaccharide, such as trehalose or sucrose.

In some embodiments herein, a “surfactant” refers to a surface-active agent, preferably a nonionic surfactant. Examples of surfactants herein include polysorbate (for example, polysorbate 20 and polysorbate 80); poloxamer (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.); polyethyl glycol, polypropyl glycol, and copolymers of ethylene and propylene glycol (e.g. Pluronics, PF68 etc); etc. In some embodiments, the surfactant is polysorbate 20. In some embodiments, the surfactant is polysorbate 80.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide an additional medicament (examples of which are provided herein). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules.

The formulations to be used for in vivo administration are generally sterile. Sterility may be readily accomplished, e.g., by filtration through sterile filtration membranes.

In some embodiments, provided herein are pharmaceutical formulations comprising: (a) any of the anti-human OX40 agonist antibodies described herein; (b) a histidine buffer at pH 5.0-6.0.

In some embodiments, provided herein are pharmaceutical formulations comprising: (a) any of the anti-human OX40 agonist antibodies described herein; (b) a histidine buffer at pH 5.0-6.0; (c) a saccharide; and (d) a surfactant.

In some embodiments of any of the formulations, the anti-human OX40 agonist antibody is present at a concentration between about 10 mg/mL and about 100 mg/mL (e.g. about 15 mg/mL, 18 mg/mL, 20 mg/mL, 60 mg/mL, and 75 mg/mL). In some embodiments, the anti-human OX40 agonist antibody is present at a concentration of about 20 mg/mL. In some embodiments, the anti-human OX40 agonist antibody is present at a concentration of about 50 mg/mL. In some embodiments, the anti-human OX40 agonist antibody is present at a concentration of about 60 mg/mL. In some embodiments, the anti-human OX40 agonist antibody is present at a concentration of about 70 mg/mL.

In some embodiments of any of the formulations, the saccharide is present at a concentration of about 75 mM to about 360 mM (e.g., about 100 mM, about 120 mM, about 240 mM, about 320 mM to about 360 mM). In some embodiments, the saccharide is present at a concentration of about 120 mM. In some embodiments, the saccharide is present at a concentration of about 240 mM. In some embodiments, the saccharide is present at a concentration of about 320 mM. In some embodiments, the saccharide is a disaccharide. In some embodiments, the disaccharide is trehalose. In some embodiments, the disaccharide is sucrose.

In some embodiments of any of the formulations, the histidine buffer is at a concentration of about 1 mM to about 50 mM (e.g. about 1 mM to about 25 mM). In some embodiments, the histidine buffer is at a concentration of about 10 mM. In some embodiments, the histidine buffer is at a concentration of about 20 mM. In some embodiments, the histidine buffer is at a concentration of about 30 mM. In some embodiments, the histidine buffer is histidine acetate.

In some embodiments of any of the formulations, the surfactant is polysorbate (e.g., polysorbate 20 or polysorbate 40), poloxamer (e.g. poloxamer 188); Triton; sodium dodecyl sulfate (SDS); sodium laurel sulfate; or sodium octyl glycoside.

In some embodiments of any of the formulations, the surfactant is polysorbate. In some embodiments, the polysorbate is present at a concentration of about 0.005% to about 0.1%. In some embodiments, the polysorbate is present at a concentration of about 0.005%. In some embodiments, the polysorbate is present at a concentration of about 0.02%. In some embodiments, the polysorbate is present at a concentration of about 0.04%. In some embodiments, the polysorbate is present at a concentration of about 0.06%. In some embodiments, the polysorbate is polysorbate 20. In some embodiments, the polysorbate is polysorbate 80.

In some embodiments of any of the formulations, the formulation is diluted with a diluent (e.g., 0.9% NaCl). In some embodiments, the anti-human OX40 agonist antibody is present at a concentration of about 1 mg/mL.

In particular, provided herein are pharmaceutical formulations comprising (a) any of the anti-human OX40 agonist antibodies described herein, (b) a polysorbate, wherein the polysorbate concentration is about 0.005% to about 0.1%.; and (c) a histidine buffer (e.g., a histidine buffer at a pH between 5.0 and 6.0).

In some embodiments, the pharmaceutical formulation comprises (a) any of the anti-human OX40 agonist antibodies described herein (e.g., at a concentration between about 10 mg/mL and about 100 mg/mL), (b) a polysorbate, wherein the polysorbate concentration is about 0.02% to about 0.06%; (c) a histidine buffer (e.g., a histidine buffer at pH 5.0 to 6.0); and a saccharide, wherein the saccharide concentration is about 120 mM to about 320 mM. In some embodiments, the saccharide is sucrose.

In some embodiments, the pharmaceutical formulation comprises (a) any of the anti-human OX40 agonist antibodies described herein at a concentration between about 10 mg/mL and about 100 mg/mL, (b) a polysorbate, wherein the polysorbate concentration is about 0.02% to about 0.06%, wherein the polysorbate is polysorbate 20 or polysorbate 40; (c) a histidine acetate buffer at pH 5.0 to 6.0; and a saccharide (e.g., sucrose) at a concentration of about 120 mM to about 320 mM.

In some embodiments, the pharmaceutical formulation comprises (a) any of the anti-human OX40 agonist antibodies described herein, (b) polysorbate 20, wherein the polysorbate concentration is about 0.02% to about 0.06%; (c) a histidine acetate buffer (e.g., a histidine acetate buffer at pH 5.0 to 6.0); and (d) sucrose, wherein the sucrose concentration is about 120 mM to about 320 mM.

In some embodiments, the pharmaceutical formulation comprises (a) any of the anti-human OX40 agonist antibodies described herein, (b) polysorbate 40, wherein the polysorbate concentration is about 0.02% to about 0.06%; (c) a histidine acetate buffer (e.g., a histidine acetate buffer at a pH between 5.0 and 6.0); and sucrose, wherein the sucrose concentration is about 120 mM to about 320 mM.

In some embodiments, the pharmaceutical formulation comprises (a) any of the anti-human OX40 agonist antibodies described herein, (b) polysorbate 20, wherein the polysorbate concentration is about 0.02%; (c) a histidine acetate buffer at pH 6.0; and (d) sucrose, wherein the sucrose concentration is about 320 mM.

In some embodiments, the pharmaceutical formulation comprises (a) any of the anti-human OX40 agonist antibodies described herein, (b) polysorbate 20, wherein the polysorbate concentration is about 0.02%; (c) a histidine acetate buffer at pH 5.5; and (d) sucrose, wherein the sucrose concentration is about 240 mM.

In some embodiments, the pharmaceutical formulation comprises (a) any of the anti-human OX40 agonist antibodies described herein, (b) polysorbate 20, wherein the polysorbate concentration is about 0.04%; (c) a histidine acetate buffer at pH 6.0; and (d) sucrose, wherein the sucrose concentration is about 120 mM.

In some embodiments, the pharmaceutical formulation comprises (a) any of the anti-human OX40 agonist antibodies described herein, (b) polysorbate 40, wherein the polysorbate concentration is about 0.04%; (c) a histidine acetate buffer at pH 5.0; and (d) sucrose, wherein the sucrose concentration is about 240 mM.

In some embodiments, the pharmaceutical formulation comprises (a) any of the anti-human OX40 agonist antibodies described herein, (b) polysorbate 40, wherein the polysorbate concentration is about 0.04%; (c) a histidine acetate buffer at pH 6.0; and (d) sucrose, wherein the sucrose concentration is about 120 mM.

In some embodiments, the pharmaceutical formulation is a liquid pharmaceutical formulation. In some embodiments, the pharmaceutical formulation is a stable pharmaceutical formulation. In some embodiments, the pharmaceutical formulation is a stable liquid pharmaceutical formulation.

In some embodiments of any of the pharmaceutical formulations described herein, the anti-human OX40 agonist antibody of the pharmaceutical formulation is present at a concentration between about 10 mg/mL and about 100 mg/mL. In some embodiments, the concentration of the human OX40 agonist antibody is between about any of 10 mg/mL to 50 mg/mL, 10 mg/mL to 75 mg/mL, 25 mg/mL to 75 mg/mL, 50 mg/mL to 100 mg/mL, 50 mg/mL to 75 mg/mL, and/or 75 mg/mL to 100 mg/mL. In some embodiments, the concentration of the human OX40 agonist antibody is greater than about any of 20 mg/mL, 30 mg/mL, 40 mg/mL, 50 mg/mL, 60 mg/mL, 70 mg/mL, or 100 mg/mL.

The pharmaceutical formulation preferably comprises a polysorbate. The polysorbate is generally included in an amount which reduces aggregate formation (such as that which occurs upon shaking or shipping). Examples of polysorbate include, but are not limited to, polysorbate 20 (polyoxyethylene (20) sorbitan monolaurate), polysorbate 40 (polyoxyethylene (20) sorbitan monopalmitate), polysorbate 60 (polyoxyethylene (20) sorbitan monostearate), and/or polysorbate 80 (polyoxyethylene (20) sorbitan monooleate). In some embodiments, the polysorbate is polysorbate 20 (polyoxyethylene (20) sorbitan monolaurate). In some embodiments of any of the pharmaceutical formulations described herein, the polysorbate concentration is sufficient to minimize aggregation and/or maintain stability upon long term storage and/or during administration (e.g., after dilution in an IV bag). In some embodiments, the polysorbate concentration is about 0.005% w/v, about 0.02% w/v, about 0.04% w/v and less than about 0.1% w/v. In some embodiments, the polysorbate concentration is greater than 0.01% w/v and less than about 0.1% w/v. In some embodiments, the polysorbate concentration is about any of 0.005% w/v, about 0.02% w/v, 0.03% w/v, 0.04% w/v, or 0.05% w/v. In some embodiments, the polysorbate is present at a concentration of about 0.04% w/v. In some embodiments, the polysorbate is present at a concentration of about 0.02% w/v.

The pharmaceutical formulation preferably comprises a saccharide. Saccharides include monosaccharides, disaccharides, trisaccharides, polysaccharides, sugar alcohols, reducing sugars, nonreducing sugars, etc. Further examples of saccharides include, but are not limited to, glucose, sucrose, trehalose, lactose, fructose, maltose, dextran, glycerin, dextran, erythritol, glycerol, arabitol, sylitol, sorbitol, mannitol, mellibiose, melezitose, raffinose, mannotriose, stachyose, maltose, lactulose, maltulose, glucitol, maltitol, lactitol, iso-maltulose, etc. In some embodiments, the saccharide is a disaccharide. In some embodiments, the saccharide is a nonreducing disaccharide. In some embodiments, the saccharide is trehalose.

The saccharide is generally included in an amount which reduces aggregate formation. In some embodiments of any of the pharmaceutical formulations described herein, the saccharide is present at a concentration of between about any of 50 mM to 250 mM, 75 mM to 200 mM, 75 mM to 150 mM, 100 mM to 150 mM, or 110 mM to 130 mM, or 100 mM to 320 mM, or 240 mM to 320 mM, or 240 mM to 400 mM. In some embodiments, the saccharide is present at a concentration greater than about any of 50 mM, 75 mM, 100 mM, 110 mM, or 115 mM. In some embodiments, the saccharide is present at a concentration of about any of 100 mM, 110 mM, 120 mM, 130 mM, or 140 mM. In some embodiments, the saccharide is present at a concentration of about 120 mM. In some embodiments of any of the formulations, the saccharide is present at a concentration of about 75 mM to about 360 mM (e.g., about 100 mM, about 120 mM, about 240 mM, about 320 mM to about 360 mM). In some embodiments, the saccharide is present at a concentration of about 240 mM. In some embodiments, the saccharide is present at a concentration of about 320 mM.

The pharmaceutical formulation preferably comprises a histidine buffer. Examples of histidine buffers include, but are not limited to, histidine chloride, histidine succinate, histidine acetate, histidine phosphate, histidine sulfate. In some embodiments, the histidine buffer is histidine acetate. In some embodiments of any of the pharmaceutical formulations described herein, the histidine buffer concentration is between about any of 1 mM to 50 mM, 1 mM to 35 mM, 1 mM to 25 mM, 1 mM to 20 mM, 7.5 mM to 12.5 mM, or 5 mM to 15 mM, 20 mM to 30 mM, 25 mM to 35 mM. In some embodiments, the histidine buffer concentration is about any of 5 mM, 7.5 mM, 10 mM, 12.5 mM, 15 mM, 20 mM, 25 mM, 30 mM, 35 mM or 40 mM. In some embodiments, the histidine buffer concentration is about 10 mM. In some embodiments, the histidine buffer concentration is about 20 mM. In some embodiments, the histidine buffer concentration is about 30 mM. In some embodiments, the histidine buffer concentration is about 40 mM. In some embodiments of any of the pharmaceutical formulations described herein, the histidine buffer is at a pH of between pH 5.0 and 6.0, for example, about any of pH 5.0, pH 5.1, pH 5.2, pH 5.3, pH 5.4, pH 5.5, pH 5.6, pH 5.7, pH 5.8, pH 5.9 or pH 6.0. In some embodiments, the pH is between pH 4.9 to pH 6.3.

The pharmaceutical formulation herein may also contain more than one active compound as necessary for the particular indication being treated, preferably those with complementary activities that do not adversely affect each other. Such molecules are suitably present in combination in amounts that are effective for the purpose intended.

Further, provided herein are vials and methods of filing a vial comprising a pharmaceutical formulation described herein. In some embodiments, the pharmaceutical formulation is provided inside a vial with a stopper pierceable by a syringe, preferably in aqueous form. The vial is desirably stored at about 2-8° C. as well as up to 30° C. for 24 hours until it is administered to a subject in need thereof. The vial may for example be a 15 cc vial (for example for a 200 mg dose).

The pharmaceutical formulation for administration is preferably a liquid formulation (not lyophilized) and has not been subjected to prior lyophilization. While the pharmaceutical formulation may be lyophilized, preferably it is not. In some embodiments of any of the pharmaceutical formulations, the pharmaceutical formulation, the pharmaceutical formulation is a lyophilized pharmaceutical formulation. In some embodiments, the pharmaceutical formulation is a liquid formulation. In some embodiments, the pharmaceutical formulation does not contain a tonicifying amount of a salt such as sodium chloride. In some embodiments of any of the pharmaceutical formulations, the pharmaceutical formulation is diluted.

### G. Therapeutic Methods and Compositions

Any of the anti-human OX40 antibodies provided herein may be used in therapeutic methods. For example, in certain aspects, the invention provides methods of treating or delaying progression of cancer in an individual by administering to the individual a dose of an anti-human OX40 agonist antibody of the present disclosure. In some embodiments, the dose of the antibody may be part of a pharmaceutical formulation. In some embodiments, the anti-human OX40 agonist antibody comprises (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7. In certain embodiments, the antibody is MOXR0916 (1A7.gr1 IgG1).

In some embodiments, the dose may be between about 0.1 mg and about 1500 mg of the antibody. For example, the dose of the antibody may be between about 0.1 mg and about 1500 mg, between about 0.1 mg and about 1400 mg, between about 0.1 mg and about 1200 mg, between about 0.1 mg and about 1000 mg, between about 0.1 mg and about 800 mg, between about 0.1 mg and about 600 mg, between about 0.1 mg and about 500 mg, between about 0.1 mg and about 400 mg, between about 0.1 mg and about 200 mg, between about 0.1 mg and about 150 mg, between about 0.1 mg and about 100 mg, between about 0.1 mg and about 50 mg, between about 0.1 mg and about 25 mg, between about 0.1 mg and about 15 mg, between about 0.1 mg and about 10 mg, between about 0.1 mg and about 5 mg, or between about 0.1 mg and about 1 mg. In some embodiments, the dose is less than about any of the following doses (in mg): 1500, 1400, 1200, 1000, 800, 600, 500, 400, 200, 150, 100, 50, 25, 15, 10, 5, 1, or 0.8. In some embodiments, the dose is greater than about any of the following doses (in mg): 0.2, 0.5, 0.8, 1, 5, 10, 15, 25, 50, 100, 150, 200, 400, 500, 600, 800, 1000, 1200, or 1400. That is, the dose can be any of a range of doses (in mg) having an upper limit of 1500, 1400, 1200, 1000, 800, 600, 500, 400, 200, 150, 100, 50, 25, 15, 10, 5, 1, or 0.8 and an independently selected lower limit of 0.2, 0.5, 0.8, 1, 5, 10, 15, 25, 50, 100, 150, 200, 400, 500, 600, 800, 1000, 1200, or 1400, wherein the lower limit is less than the upper limit.

In some embodiments, the anti-human OX40 agonist antibody dose is selected from about 0.2 mg, 0.8 mg, about 3.2 mg, about 12 mg, about 40 mg, about 80 mg, about 130 mg, about 160 mg, about 300 mg, about 320 mg, about 400 mg, about 600 mg, and about 1200 mg, e.g., per administration. In certain embodiments, the anti-human OX40 agonist antibody dose is about 300 mg. In certain embodiments, the anti-human OX40 agonist antibody dose is selected from 0.2 mg, 0.8 mg, 3.2 mg, 12 mg, 40 mg, 80 mg, 130 mg, 160 mg, 300 mg, 320 mg, 400 mg, 600 mg, and 1200 mg. In certain embodiments, the anti-human OX40 agonist antibody dose is 300 mg.

In some embodiments, the anti-human OX40 agonist antibody dose is selected from about 0.1 mg, 0.5 mg, about 2 mg, about 8 mg, about 27 mg, about 53 mg, about 87 mg, about 107 mg, about 200 mg, about 213 mg, about 267 mg, about 400 mg, and about 800 mg, e.g., per administration. In certain embodiments, the anti-human OX40 agonist antibody dose is selected from 0.1 mg, 0.5 mg, 2 mg, 8 mg, 27 mg, 53 mg, 87 mg, 107 mg, 200 mg, 213 mg, 267 mg, 400 mg, and 800 mg.

In some embodiments, the administration of the anti-human OX40 agonist antibody may be repeated at one or more additional doses. In some embodiments, each dose of the one or more additional doses is selected from about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg, e.g., per administration. In some embodiments, each dose of the one or more additional doses is about 300 mg.

The administration of the anti-human OX40 agonist antibody may be adjusted, e.g., based on the dosing cycle. In some embodiments, the anti-human OX40 agonist antibody dose is selected from about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg, e.g., per administration, and the anti-human OX40 agonist antibody may be administered at an interval of about 3 weeks or about 21 days between each administration. In some embodiments, the anti-human OX40 agonist antibody dose is selected from about 0.1 mg, about 0.5 mg, about 2 mg, about 8 mg, about 27 mg, about 53 mg, about 87 mg, about 107 mg, about 200 mg, about 213 mg, about 267 mg, about 400 mg, and about 800 mg, e.g., per administration, and the anti-human OX40 agonist antibody may be administered at an interval of about 2 weeks or about 14 days between each administration. In some embodiments, the dosing interval for the anti-human OX40 agonist antibody may be adjusted, e.g., to match a dosing interval or protocol of a concomitant therapeutic agent or protocol (e.g., a 2-week dosing interval for FOLFOX).

In some embodiments, 1-10 additional doses of the anti-human OX40 agonist antibody are administered, e.g., in repeated administration as described above. For example, in some embodiments, 1, 2, 3, 4, 5, 6, 7, 8, 9, or 10 additional doses of the anti-human OX40 agonist antibody may be administered.

In some embodiments, each dose of the anti-human OX40 agonist antibody administered to the individual may be the same. In other embodiments, each dose of the anti-human OX40 agonist antibody administered to the individual is not the same. Dosing may be modified as described herein, e.g., based on efficacy, toxicity, adverse events, progression, PD, PK, an effect of a second therapeutic agent, and so forth.

In some embodiments, the anti-human OX40 agonist antibody is administered intravenously. In some embodiments, the anti-human OX40 agonist antibody is administered at a different rate between different administrations. For example, as described herein, an initial administration may be performed at a slower rate (e.g., by IV infusion) than a subsequent administration, e.g., to prevent or mitigate infusion-related reactions.

In some embodiments, after administration of a first dose of the anti-human OX40 agonist antibody, one or more additional doses of the anti-human OX40 agonist antibody may be administered. In some embodiments, after administering the antibody, the individual is monitored for an adverse event (e.g., as exemplified below), progression and/or treatment efficacy. In some embodiments, if the individual does not exhibit an adverse event (e.g., as described herein), a second dose of the antibody may be administered. In some embodiments, if the treatment exhibits efficacy, a second dose of the antibody may be administered. In some embodiments, even if progression is observed, a second dose of the antibody may be administered. As described herein, and without wishing to be bound to theory, it is thought that in some cases immunotherapeutic agents such as anti-human OX40 agonist antibodies may induce an initial progression, followed by a response.

In some embodiments, the second dose is the same amount as the first dose. In other embodiments, the second dose may be greater than the first dose. It will be appreciated that the particular doses and dose ranges described above may apply to second doses as well as first doses in any combination or order.

In some embodiments, the second dose is not provided until from about 2 weeks to about 4 weeks after the first dose. In some embodiments, the second dose is not provided until about 14 days, about 21 days, or about 28 days after the first dose. In some embodiments, the second dose is not provided until about 21 days after the first dose. In certain embodiments, the second dose is provided about 21 days after the first dose. In some embodiments, the second dose is not provided until about 3 weeks after the first dose. In certain embodiments, the second dose is provided about 3 weeks after the first dose.

In some embodiments, the first dose and the second dose are administered via the same route. In certain embodiments, the first dose and the second dose are administered intravenously.

In one aspect, an anti-human OX40 agonist antibody for use as a medicament is provided. In further aspects, an anti-human OX40 agonist antibody for use in treating cancer is provided. In certain embodiments, an anti-human OX40 agonist antibody for use in a method of treatment is provided. In certain embodiments, the invention provides an anti-human OX40 agonist antibody for use in a method of treating an individual having cancer comprising administering to the individual an effective amount of the anti-human agonist OX40 antibody. In one such embodiment, the method further comprises administering to the individual an effective amount of at least one additional therapeutic agent, e.g., as described below.

In one aspect, provided is an anti-human OX40 agonist antibody for use in enhancing immune function (e.g., by upregulating cell-mediated immune responses) in an individual having cancer comprising administering to the individual an effective amount of the anti-human agonist OX40 antibody. In one aspect, provided is an anti-human OX40 agonist antibody for use in enhancing T cell function in an individual having cancer comprising administering to the individual an effective amount of the anti-human agonist OX40 antibody. In one aspect, provided are an anti-human OX40 agonist antibody for use in depleting human OX40-expressing cells (e.g., OX40 expressing T cells, e.g., OX40 expressing Treg) comprising administering to the individual an effective amount of the anti-human agonist OX40 antibody. In some embodiments, depletion is by ADCC. In some embodiments, depletion is by phagocytosis. Provided is an anti-human OX40 agonist antibody for treating an individual having tumor immunity.

In further aspects, an anti-human OX40 agonist antibody for use in treating infection (e.g., with a bacteria or virus or other pathogen) is provided. In certain embodiments, the invention provides an anti-human OX40 agonist antibody for use in a method of treating an individual having an infection comprising administering to the individual an effective amount of the anti-human agonist OX40 antibody. In some embodiments, the infection is with a virus and/or a bacteria. In some embodiments, the infection is with a pathogen.

In a further aspect, the invention provides for the use of an anti-OX40 antibody in the manufacture or preparation of a medicament. In one embodiment, the medicament is for treatment of cancer. In a further embodiment, the medicament is for use in a method of treating cancer comprising administering to an individual having cancer an effective amount of the medicament. In one such embodiment, the method further comprises administering to the individual an effective amount of at least one additional therapeutic agent, e.g., as described below.

In one aspect, the medicament is for use in enhancing immune function (e.g., by upregulating cell-mediated immune responses) in an individual having cancer comprising administering to the individual an effective amount of the medicament. In one aspect, the medicament is for use in enhancing T cell function in an individual having cancer comprising administering to the individual an effective amount of the medicament. In some embodiments, the T cell dysfunctional disorder is cancer. In one aspect, the medicament is for use in depleting human OX40-expressing cells (e.g., cell expressing high OX40, e.g., OX40 expressing T cells) comprising administering to the individual an effective amount of the medicament. In some embodiments, depletion is by ADCC. In some embodiments, depletion is by phagocytosis. In one aspect, the medicament is for treating an individual having tumor immunity.

In further aspects, the medicament is for use in treating infection (e.g., with a bacteria or virus or other pathogen) is provided. In certain embodiments, the medicament is for use in a method of treating an individual having an infection comprising administering to the individual an effective amount of the medicament. In some embodiments, the infection is with virus and/or bacteria. In some embodiments, the infection is with a pathogen.

In a further aspect, the invention provides a method for treating a cancer. In one embodiment, the method comprises administering to an individual having such cancer an effective amount of an anti-OX40 antibody. In one such embodiment, the method further comprises administering to the individual an effective amount of at least one additional therapeutic agent, as described below. An “individual” according to any of the above embodiments may be a human.

In one aspect, provided is a method for enhancing immune function (e.g., by upregulating cell-mediated immune responses) in an individual having cancer comprising administering to the individual an effective amount of the anti-human agonist OX40 antibody. In one aspect, provided is a method for enhancing T cell function in an individual having cancer comprising administering to the individual an effective amount of the anti-human agonist OX40 antibody. In one aspect, provided are a method for depleting human OX40-expressing cells (e.g., cells that express high level of OX40, e.g., OX40 expressing T cells) comprising administering to the individual an effective amount of the anti-human agonist OX40 antibody. In some embodiments, depletion is by ADCC. In some embodiments, depletion is by phagocytosis. Provided is an anti-human OX40 agonist antibody for treating an individual having tumor immunity.

In some embodiments, examples of cancer further include, but are not limited to, B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), B-cell proliferative disorders, and Meigs' syndrome. More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B-cell chronic lymphocytic leukemia and/or prolymphocytic leukemia and/or small lymphocytic lymphoma, B-cell prolymphocytic lymphoma, immunocytoma and/or lymphoplasmacytic lymphoma, lymphoplasmacytic lymphoma, marginal zone B-cell lymphoma, splenic marginal zone lymphoma, extranodal marginal zone—MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) large granular lymphocytic leukemia, mycosis fungoides and/or Sezary syndrome, skin (cutaneous) lymphomas, anaplastic large cell lymphoma, angiocentric lymphoma.

In some embodiments, examples of cancer further include, but are not limited to, B-cell proliferative disorders, which further include, but are not limited to, lymphomas (e.g., B-Cell Non-Hodgkin's lymphomas (NHL)) and lymphocytic leukemias. Such lymphomas and lymphocytic leukemias include e.g. a) follicular lymphomas, b) Small Non-Cleaved Cell Lymphomas/Burkitt's lymphoma (including endemic Burkitt's lymphoma, sporadic Burkitt's lymphoma and Non-Burkitt's lymphoma), c) marginal zone lymphomas (including extranodal marginal zone B-cell lymphoma (Mucosa-associated lymphatic tissue lymphomas, MALT), nodal marginal zone B-cell lymphoma and splenic marginal zone lymphoma), d) Mantle cell lymphoma (MCL), e) Large Cell Lymphoma (including B-cell diffuse large cell lymphoma (DLCL), Diffuse Mixed Cell Lymphoma, Immunoblastic Lymphoma, Primary Mediastinal B-Cell Lymphoma, Angiocentric Lymphoma-Pulmonary B-Cell Lymphoma), f) hairy cell leukemia, g) lymphocytic lymphoma, Waldenstrom's macroglobulinemia, h) acute lymphocytic leukemia (ALL), chronic lymphocytic leukemia (CLL)/small lymphocytic lymphoma (SLL), B cell prolymphocytic leukemia, i) plasma cell neoplasms, plasma cell myeloma, multiple myeloma, plasmacytoma, and/or j) Hodgkin's disease.

In some embodiments of any of the methods, the cancer is melanoma, triple-negative breast cancer, ovarian cancer, renal cell cancer, bladder cancer, non-small cell lung cancer, gastric cancer, or colorectal cancer (including both primary and metastatic tumors). In certain embodiments, the cancer is a renal cell carcinoma (e.g., clear cell renal cell carcinoma).

In some embodiments of any of the methods, the cancer is a B-cell proliferative disorder. In some embodiments, the B-cell proliferative disorder is lymphoma, non-Hodgkins lymphoma (NHL), aggressive NHL, relapsed aggressive NHL, relapsed indolent NHL, refractory NHL, refractory indolent NHL, chronic lymphocytic leukemia (CLL), small lymphocytic lymphoma, leukemia, hairy cell leukemia (HCL), acute lymphocytic leukemia (ALL), or mantle cell lymphoma. In some embodiments, the B-cell proliferative disorder is NHL, such as indolent NHL and/or aggressive NHL. In some embodiments, the B-cell proliferative disorder is indolent follicular lymphoma or diffuse large B-cell lymphoma. In certain embodiments, the cancer is selected from melanoma, triple-negative breast cancer, ovarian cancer, renal cell cancer, bladder cancer, non-small cell lung cancer, gastric cancer, and colorectal cancer. In some embodiments, the cancer is a locally advanced or metastatic solid tumor, e.g., of any of the solid cancers described herein.

In some embodiments, the cancer is melanoma. In certain embodiments, the melanoma is advanced or metastatic melanoma. In some embodiments, the melanoma exhibits a BRAF V600 mutation (e.g., a V600E, V600K, or V600D mutation). Melanomas with a BRAF V600 mutation have been treated with B-Raf and/or mitogen-activated protein kinase kinase (MEK) kinase inhibitors. Examples of such inhibitors include without limitation sorafenib, vemurafenib, dabrafenib (GSK2118436), RAF265, LGX818, trametinib, selumetinib, binimetinib, cobimetinib, PD-325901, CI-1040 (PD 184352), PD035901, and the like. In some embodiments, the individual has been treated with a B-Raf and/or mitogen-activated protein kinase kinase (MEK) kinase inhibitor prior to treatment with the anti-human OX40 agonist antibody. In some embodiments, the patient has exhibited disease progression or intolerance to the B-Raf and/or mitogen-activated protein kinase kinase (MEK) kinase inhibitor treatment prior to treatment with the anti-human OX40 agonist antibody.

In some embodiments, the cancer is renal cell cancer (RCC). In certain embodiments, the RCC is advanced or metastatic RCC. In some embodiments, the RCC exhibits a component of clear cell histology and/or a component of sarcomatoid histology.

In some embodiments, the cancer is triple-negative breast cancer (TNBC). In certain embodiments, the TNBC is advanced or metastatic TNBC. In some embodiments, TNBC may refer to an adenocarcinoma of the breast that is estrogen receptor negative, progesterone receptor negative, and human epidermal growth factor receptor 2 negative, e.g., as defined by the American Society of Clinical Oncology-College of American Pathologists (ASCO-CAP) guidelines. For example, <1% of tumor cell nuclei may be immunoreactive for estrogen receptor, and <1% of tumor cell nuclei may be immunoreactive for progesterone receptor (Hammond, M. E. et al. (2010) J. Clin. Oncol. 28:2784-2795) and HER2 tests demonstrate either immunohistochemistry (IHC) 1+, IHC 0 or in situ hybridization (ISH) negative (Wolff, A. C. et al. (2013) J. Clin. Oncol. 31:3997:4013).

In some embodiments, the cancer is non-small cell lung cancer (NSCLC). In certain embodiments, the NSCLC is advanced or metastatic NSCLC. In some embodiments, the NSCLC exhibits a sensitizing epidermal growth factor (EGFR) mutation. Sensitizing EGFR mutations are known to involve the EGFR kinase domain and may include without limitation mutations in exons 18-21, such as exon 19 deletions and the L858R point mutation in exon 21 (for further description and/or additional mutations, see, e.g., Lynch, T. J. et al. (2004) N Engl. I Med. 350:2129-2139; Pao, W. et al. (2004) Proc. Natl. Acad. Sci. 101:13306-13311; and Paez, J. G. et al. (2004) Science 304:1497-1500). In some embodiments, the individual has been treated with an EGFR tyrosine kinase inhibitor prior to treatment with the anti-human OX40 agonist antibody. In some embodiments, the patient has exhibited disease progression or intolerance to the EGFR tyrosine kinase inhibitor treatment prior to treatment with the anti-human OX40 agonist antibody. In some embodiments, the NSCLC exhibits an anaplastic lymphoma kinase (ALK) rearrangement. ALK rearrangements have been implicated in NSCLC, particularly in EGFR tyrosine kinase inhibitor resistance, and many ALK rearrangements are known in the art, including without limitation EML4-ALK, KIFSB-ALK, and TFG-ALK rearrangements (for further description and/or additional mutations, see, e.g., Koivunen, J. P. et al. (2008) Clin. Cancer Res. 14:4275-4283; and Soda, E. M. et al. (2007) Nature 448:561-566). In some embodiments, the individual has been treated with an ALK tyrosine kinase inhibitor prior to treatment with the anti-human OX40 agonist antibody. In some embodiments, the patient has exhibited disease progression or intolerance to the ALK tyrosine kinase inhibitor treatment prior to treatment with the anti-human OX40 agonist antibody.

In some embodiments, the cancer is urothelial bladder cancer (UBC). In certain embodiments, the UBC is advanced or metastatic UBC. In some embodiments, the UBC exhibits a transitional cell pattern and includes carcinomas of the renal pelvis, ureters, urinary bladder, and/or urethra.

In some embodiments, the cancer is colorectal cancer (CRC). In certain embodiments, the CRC is advanced or metastatic CRC. In some embodiments, the CRC is an adenocarcinoma of the colon or rectum.

In some embodiments, the cancer is ovarian cancer (OC). In certain embodiments, the OC is advanced or metastatic OC. In some embodiments, the OC is an epithelial ovarian, fallopian tube, or primary peritoneal cancer.

In some embodiments, the individual is immunotherapy-naïve. For example, the patient may not have previously been treated with an immunotherapy. Numerous immunotherapies are known in the art and described herein. Types of immunotherapy may include, without limitation, costimulatory agonists and/or immune checkpoint blockade therapies. As described herein, costimulatory agonists include without limitation agonists (e.g., agonist antibodies) that bind to CD40, CD226, CD28, OX40, GITR, CD137, CD27, HVEM, or CD127; or antagonists directed against an inhibitory co-stimulatory molecule (e.g., CTLA-4, PD-1, TIM-3, BTLA, VISTA, LAG-3, B7-H3, B7-H4, IDO, TIGIT, MICA/B, or arginase). As described herein, immune checkpoint blockade therapies may include without limitation PD-1 axis binding antagonists (e.g., a PD-1 binding antagonist, a PD-L1 binding antagonist or a PD-L2 binding antagonist) and antagonists directed against CTLA-4 (also known as CD152), e.g., a blocking antibody.

In some embodiments of any of the methods, the tumor or cancer is refractory. As used herein, the term “refractory” may refer to a tumor/cancer, or be used to describe a patient with said tumor/cancer, for which a prior therapy has been ineffective and/or intolerable. For example, for RCC, a “refractory” patient may be one for whom prior anti-cancer therapy comprising a VEGF inhibitor and/or an mTOR inhibitor has proven to be ineffective and/or intolerable. One skilled in the art will appreciate that such therapies are merely exemplary, and the methods of the present disclosure may be used to treat or delay progression of a cancer such as RCC or any of the other cancers described herein that is refractory to one or more other therapies, as the appropriateness of the benefit/risk profile of an anti-cancer therapy may in some cases be up to clinical judgement of the prescribing oncologist.

In some embodiments, provided herein is a method of treating or delaying progression of cancer in an individual comprising administering to the individual MOXR0916 at a dose of 300 mg, wherein the cancer is selected from the group consisting of melanoma, triple-negative breast cancer, ovarian cancer, renal cell cancer, bladder cancer, non-small cell lung cancer, gastric cancer, and colorectal cancer. In some embodiments, the method further comprises repeating the administration of MOXR0916 at a dose of 300 mg per administration, and the administration is repeated at an interval of about 3 weeks or about 21 days between administrations. In some embodiments, the cancer is RCC. In some embodiments, the cancer is RCC, and the cancer is refractory to a treatment comprising a VEGF inhibitor and/or an mTOR inhibitor. In some embodiments, MOXR0916 is administered intravenously.

In some embodiments, provided herein is a method of treating or delaying progression of cancer in an individual comprising administering to the individual MOXR0916 at a dose of 160 mg, wherein the cancer is selected from the group consisting of melanoma, triple-negative breast cancer, ovarian cancer, renal cell cancer, bladder cancer, non-small cell lung cancer, gastric cancer, and colorectal cancer. In some embodiments, the method further comprises repeating the administration of MOXR0916 at a dose of 160 mg per administration, and the administration is repeated at an interval of about 3 weeks or about 21 days between administrations. In some embodiments, the cancer is RCC. In some embodiments, the cancer is RCC, and the cancer is refractory to a treatment comprising a VEGF inhibitor and/or an mTOR inhibitor. In some embodiments, MOXR0916 is administered intravenously.

In some embodiments, provided herein is a method of treating or delaying progression of cancer in an individual comprising administering to the individual MOXR0916 at a dose of 320 mg, wherein the cancer is selected from the group consisting of melanoma, triple-negative breast cancer, ovarian cancer, renal cell cancer, bladder cancer, non-small cell lung cancer, gastric cancer, and colorectal cancer. In some embodiments, the method further comprises repeating the administration of MOXR0916 at a dose of 320 mg per administration, and the administration is repeated at an interval of about 3 weeks or about 21 days between administrations. In some embodiments, the cancer is RCC. In some embodiments, the cancer is RCC, and the cancer is refractory to a treatment comprising a VEGF inhibitor and/or an mTOR inhibitor. In some embodiments, MOXR0916 is administered intravenously.

In some embodiments, provided herein is a method of treating or delaying progression of cancer in an individual comprising administering to the individual MOXR0916 at a dose of 400 mg, wherein the cancer is selected from the group consisting of melanoma, triple-negative breast cancer, ovarian cancer, renal cell cancer, bladder cancer, non-small cell lung cancer, gastric cancer, and colorectal cancer. In some embodiments, the method further comprises repeating the administration of MOXR0916 at a dose of 400 mg per administration, and the administration is repeated at an interval of about 3 weeks or about 21 days between administrations. In some embodiments, the cancer is RCC. In some embodiments, the cancer is RCC, and the cancer is refractory to a treatment comprising a VEGF inhibitor and/or an mTOR inhibitor. In some embodiments, MOXR0916 is administered intravenously.

In a further aspect, the invention provides pharmaceutical formulations comprising any of the anti-OX40 antibodies provided herein, e.g., for use in any of the above therapeutic methods. In one embodiment, a pharmaceutical formulation comprises any of the anti-OX40 antibodies provided herein and a pharmaceutically acceptable carrier. In another embodiment, a pharmaceutical formulation comprises any of the anti-OX40 antibodies provided herein and at least one additional therapeutic agent, e.g., as described below.

In some embodiments of any of the methods of the invention, the anti-human OX40 agonist antibodies inhibits tumor immunity by inhibiting Treg function (e.g., inhibiting the suppressive function of Tregs), killing OX40 expressing cells (e.g., cells that express high levels of OX40), increasing effector T cell function and/or increasing memory T cell function. In some embodiments of any of the methods of the invention, the anti-human OX40 agonist antibodies treat cancer by inhibiting Treg function (e.g., inhibiting the suppressive function of Tregs), killing OX40 expressing cells (e.g., cells that express high levels of OX40), increasing effector T cell function and/or increasing memory T cell function. In some embodiments of any of the methods of the invention, the anti-human OX40 agonist antibodies enhance immune function by inhibiting Treg function (e.g., inhibiting the suppressive function of Tregs), killing OX40 expressing cells (e.g., cells that express high levels of OX40), increasing effector T cell function and/or increasing memory T cell function. In some embodiments of any of the methods of the invention, the anti-human OX40 agonist antibodies enhance T cell function by inhibiting Treg function (e.g., inhibiting the suppressive function of Tregs), killing OX40 expressing cells (e.g., cells that express high levels of OX40), increasing effector T cell function and/or increasing memory T cell function.

In some embodiments of any of the methods, the anti-human OX40 agonist antibody is a depleting anti-human agonist antibody. In some embodiments, treatment with the anti-human OX40 agonist antibody results in cell depletion (e.g., depletion of OX40-expressing cells, e.g., depletion of cells that express high levels of OX40). In some embodiments, depletion is by ADCC. In some embodiments, depletion is by phagocytosis.

In some embodiments of any of the methods, the anti-human OX40 agonist antibody inhibits Treg function, e.g., by inhibiting Treg suppression of effector and/or memory T cell function (in some embodiments, effector T cell and/or memory T cell proliferation and/or cytokine secretion), relative to Treg function prior to administration of the OX40 agonist antibody. In some embodiments of any of the methods, the anti-human OX40 agonist antibody increases effector T cell proliferation, relative to effector T cell proliferation prior to administration of the OX40 agonist antibody. In some embodiments of any of the methods, the anti-human OX40 agonist antibody increases memory T cell proliferation, relative to memory T cell proliferation prior to administration of the OX40 agonist antibody. In some embodiments of any of the methods, the anti-human OX40 agonist antibody increases effector T cell cytokine production (e.g., gamma interferon production), relative to effector T cell cytokine production prior to administration of the OX40 agonist antibody. In some embodiments of any of the methods, the anti-human OX40 agonist antibody increases memory T cell cytokine production (e.g., gamma interferon production), relative to memory T cell cytokine production prior to administration of the OX40 agonist antibody. In some embodiments of any of the methods, the anti-human OX40 agonist antibody increases CD4+ effector T cell proliferation and/or CD8+ effector T cell proliferation relative to CD4+ effector T cell proliferation and/or CD8+ effector T cell proliferation prior to administration of the OX40 agonist antibody. In some embodiments of any of the methods, the anti-human OX40 agonist antibody increases memory T cell proliferation (e.g., CD4+ memory T cell proliferation), relative to memory T cell proliferation prior to administration of the OX40 agonist antibody. In some embodiments, the CD4+ effector T cells in the individual have enhanced proliferation, cytokine secretion and/or cytolytic activity relative to proliferation, cytokine secretion and/or cytolytic activity prior to the administration of the anti-human OX40 agonist antibody.

In some embodiments of any of the methods of the invention, the number of CD4+ effector T cells is elevated relative to prior to administration of the anti-human OX40 agonist antibody. In some embodiments, CD4+ effector T cell cytokine secretion is elevated relative to prior to administration of the anti-human OX40 agonist antibody. In some embodiments of any of the methods, the CD8+ effector T cells in the individual have enhanced proliferation, cytokine secretion and/or cytolytic activity relative to prior to the administration of the anti-human OX40 agonist antibody. In some embodiments, the number of CD8+ effector T cells is elevated relative to prior to administration of the anti-human OX40 agonist antibody. In some embodiments, CD8+ effector T cell cytokine secretion is elevated relative to prior to administration of the anti-human OX40 agonist antibody.

In some embodiments of any of the methods of the invention, the anti-human OX40 agonist antibody binds human effector cells, e.g., binds FcγR expressed by human effector cells. In some embodiments, the human effector cell performs ADCC effector function. In some embodiments, the human effector cell performs phagocytosis effector function.

In some embodiments of any of the methods of the invention, the anti-human OX40 agonist antibody comprising a variant IgG1 Fc polypeptide comprising a mutation that eliminates binding to human effector cells (e.g., a DANA or N297G mutation) has diminished activity (e.g., CD4+ effector T cell function, e.g., proliferation), relative to anti-human OX40 agonist antibody comprising native sequence IgG1 Fc portion. In some embodiment, the anti-human OX40 agonist antibody comprising a variant IgG1 Fc polypeptide comprising a mutation that eliminates binding to human effector cells (e.g., a DANA or N297G mutation) does not possess substantial activity (e.g., CD4+ effector T cell function, e.g., proliferation).

In some embodiments of any of the methods of the invention, antibody cross-linking is required for anti-human OX40 agonist antibody function. In some embodiments, function is stimulation of CD4+ effector T cell proliferation. In some embodiments, antibody cross-linking is determined by providing anti-human OX40 agonist antibody adhered on a solid surface (e.g., a cell culture plate). In some embodiments, antibody cross-linking is determined by introducing a mutation in the antibody's IgG1 Fc portion (e.g., a DANA or N297S mutation) and testing function of the mutant antibody.

In some embodiments of any of the methods, the memory T cells in the individual have enhanced proliferation and/or cytokine secretion relative to prior to the administration of the anti-human OX40 agonist antibody. In some embodiments, the number of memory T cells is elevated relative to prior to administration of the anti-human OX40 agonist antibody. In some embodiments, memory T cell cytokine secretion (level) is elevated relative to prior to administration of the anti-human OX40 agonist antibody. In some embodiments of any of the methods, the Treg in the individual have decreased inhibition of effector T cell function (e.g., proliferation and/or cytokine secretion) relative to prior to the administration of the anti-human OX40 agonist antibody. In some embodiments, the number of effector T cells is elevated relative to prior to administration of the anti-human OX40 agonist antibody. In some embodiments, effector T cell cytokine secretion (level) is elevated relative to prior to administration of the anti-human OX40 agonist antibody.

In some embodiments of any of the methods of the invention, the number of intratumoral (infiltrating) CD4+ effector T cells (e.g., total number of CD4+ effector T cells, or e.g., percentage of CD4+ cells in CD45+ cells) is elevated relative to prior to administration of the anti-human OX40 agonist antibody. In some embodiments of any of the methods of the invention, number of intratumoral (infiltrating) CD4+ effector T cells that express gamma interferon (e.g., total gamma interferon expressing CD4+ cells, or e.g., percentage of gamma interferon expressing CD4+ cells in total CD4+ cells) is elevated relative to prior to administration anti-human OX40 agonist antibody.

In some embodiments of any of the methods of the invention, the number of intratumoral (infiltrating) CD8+ effector T cells (e.g., total number of CD8+ effector T cells, or e.g., percentage of CD8+ in CD45+ cells) is elevated relative to prior to administration of anti-human OX40 agonist antibody. In some embodiments of any of the methods of the invention, number of intratumoral (infiltrating) CD8+ effector T cells that express gamma interferon (e.g., percentage of CD8+ cells that express gamma interferon in total CD8+ cells) is increased relative to prior to administration of anti-human OX40 agonist antibody.

In some embodiments of any of the methods of the invention, the number of intratumoral (infiltrating) Treg (e.g., total number of Treg or e.g., percentage of Fox3p+ cells in CD4+ cells) is reduced relative to prior to administration of anti-human OX40 agonist antibody.

In some embodiments of any of the methods of the invention, administration of anti-human OX40 agonist antibody is in combination with administration of a tumor antigen. In some embodiments, the tumor antigen comprises protein. In some embodiments, the tumor antigen comprises nucleic acid. In some embodiments, the tumor antigen is a tumor cell.

In some embodiments of any of the methods of the invention, a tumor response to treatment may be evaluated. In some embodiments, RECIST criteria, such as RECIST v1.1, may be used to evaluate tumor response. These criteria are known in the art and may be used to measure a patient's response to a treatment; see, e.g., Eisenhauer, E. A. et al. (2009) Eur. J. Cancer 45:228-247. In some embodiments, RECIST response criteria may include:

(a) Complete response (CR): disappearance of all target lesions. Any pathological lymph nodes (whether target or non-target) must have reduction in short axis to <10 mm;
(b) Partial response (PR): at least a 30% decrease in the sum of diameters of target lesions, taking as reference the baseline sum of diameters;
(c) Progressive disease (PD): at least a 20% increase in the sum of diameters of target lesions, taking as reference the smallest sum on study (nadir), including baseline. In addition to the relative increase of 20%, the sum must also demonstrate an absolute increase of at least 5 mm. The appearance of one or more new lesions is also considered progression; and
(d) Stable disease (SD): neither sufficient shrinkage to qualify for PR nor sufficient increase to qualify for PD, taking as reference the smallest sum on study.

In other embodiments, modified RECIST criteria may be used to evaluate tumor response.

Modified Response Evaluation Criteria in Solid Tumors (RECIST) is derived from RECIST, Version 1.1 (v1.1) conventions (see, e.g., Eisenhauer, E. A. et al. (2009) Eur. J. Cancer 45:228-247) and immune-related response criteria (irRC; see, e.g., Wolchok et al. (2009) Clin. Can. Res. 15:7412-7420; Nishino et al. (2014) J. Immunother. Can. 2:17; and Nishino et al. (2013) Clin. Can. Res. 19:3936-3943). Without wishing to be bound to theory, it is thought that conventional response criteria may not be adequate to characterize the anti-tumor activity of immunotherapeutic agents like anti-human OX40 agonist antibodies, which can produce delayed responses that may be preceded by initial apparent radiographic progression, including the appearance of new lesions. Therefore, modified response criteria have been developed that account for the possible appearance of new lesions and allow radiological progression to be confirmed at a subsequent assessment. A summary of the changes between modified RECIST and RECIST v1.1 is provided in Table B below.

TABLE B

| | RECIST v1.1 | Modified RECIST |
|---|---|---|
| New lesions | Define progression | New measurable lesions are |
| after baseline | | added into the total tumor |
| | | burden and followed. |
| Non-target | May contribute to the | Contribute only in the |
| lesions | designation of overall | assessment of a complete |
| | progression. | response. |
| Radiographic | First instance of ≥20% | Determined only on the basis |
| progression | increase in the sum of | of measurable disease; may be |
| | diameters or unequivocal | confirmed by a consecutive |
| | progression in non- | assessment ≥4 weeks from the |
| | target disease. | date first documented. |

In some embodiments, modified RECIST response criteria may include:

(a) Complete response (CR): disappearance of all target and non-target lesions. Lymph nodes that shrink to <10 mm short axis are considered normal;
(b) Partial response (PR): at least a 30% decrease in the sum of the diameters of all target and all new measurable lesions, taking as reference the baseline sum of diameters, in the absence of CR. Note: the appearance of new measurable lesions is factored into the overall tumor burden, but does not automatically qualify as progressive disease until the sum of the diameters increases by ≥20% when compared with the sum of the diameters at nadir;
(c) Progressive disease (PD): at least a 20% increase in the sum of diameters of all target and selected new measurable lesions, taking as reference the smallest sum on study (nadir SLD; this includes the baseline sum if that is the smallest on study). In addition to the relative increase of 20%, the sum must also demonstrate an absolute increase of at least 5 mm; and
(d) Stable disease (SD): neither sufficient shrinkage to qualify for PR nor sufficient increase to qualify for PD, taking as reference the smallest sum of the diameters while on study.

The assessment of non-target lesions may be captured on the CRF at each timepoint using standard RECIST v1.1 definitions of CR, non-CR/non-PD, and PD (unequivocal progression). However, in determining the overall modified RECIST tumor response, non-target lesions contribute only to the assessment of a complete response. Non-target lesions are not considered in the overall definition of PR, SC, or PD per modified RECIST.

In some embodiments, new lesions alone do not qualify as progressive disease. However, their contribution to total tumor burden may be included in the sum of the diameters, which may be used to determine the overall modified RECIST tumor response.

In some embodiments, responsiveness to treatment may refer to any one or more of: extending survival (including overall survival and progression free survival); resulting in an objective response (including a complete response or a partial response); or improving signs or symptoms of cancer. In some embodiments, responsiveness may refer to improvement of one or more factors according to the published set of RECIST guidelines for determining the status of a tumor in a cancer patient, i.e., responding, stabilizing, or progressing. For a more detailed discussion of these guidelines, see Eisenhauer et al., Eur J Cancer 2009; 45: 228-47; Topalian et al., N Engl J Med 2012; 366:2443-54; Wolchok et al., Clin Can Res 2009; 15:7412-20; and Therasse, P., et al. J. Natl. Cancer Inst. 92:205-16 (2000). A responsive subject may refer to a subject whose cancer(s) show improvement, e.g., according to one or more factors based on RECIST criteria. A non-responsive subject may refer to a subject whose cancer(s) do not show improvement, e.g., according to one or more factors based on RECIST criteria.

Conventional response criteria may not be adequate to characterize the anti-tumor activity of immunotherapeutic agents, which can produce delayed responses that may be preceded by initial apparent radiological progression, including the appearance of new lesions. Therefore, modified response criteria have been developed that account for the possible appearance of new lesions and allow radiological progression to be confirmed at a subsequent assessment. Accordingly, in some embodiments, responsiveness may refer to improvement of one of more factors according to immune-related response criteria2 (irRC). See, e.g., Wolchok et al., Clin Can Res 2009; 15:7412-20. In some embodiments, new lesions are added into the defined tumor burden and followed, e.g., for radiological progression at a subsequent assessment. In some embodiments, presence of non-target lesions are included in assessment of complete response and not included in assessment of radiological progression. In some embodiments, radiological progression may be determined only on the basis of measurable disease and/or may be confirmed by a consecutive assessment ≥4 weeks from the date first documented.

In some embodiments, responsiveness may include immune activation. In some embodiments, responsiveness may include treatment efficacy. In some embodiments, responsiveness may include immune activation and treatment efficacy.

In some embodiments of any of the methods of the invention, the cancer displays human effector cells (e.g., is infiltrated by human effector cells). Methods for detecting human effector cells are well known in the art, including, e.g., by IHC. In some embodiments, the cancer display high levels of human effector cells. In some embodiments, human effector cells are one or more of NK cells, macrophages, monocytes. In some embodiments, the cancer is any cancer described herein. In some embodiments, the cancer is non-small cell lung cancer (NSCLC), glioblastoma, neuroblastoma, melanoma, breast carcinoma (e.g. triple-negative breast cancer), gastric cancer, colorectal cancer (CRC), or hepatocellular carcinoma.

In some embodiments of any of the methods of the invention, the cancer displays cells expressing FcR (e.g., is infiltrated by cells expressing FcR). Methods for detecting FcR are well known in the art, including, e.g., by IHC. In some embodiments, the cancer display high levels of cells expressing FcR. In some embodiments, FcR is FcγR. In some embodiments, FcR is activating FcγR. In some embodiments, the cancer is non-small cell lung cancer (NSCLC), glioblastoma, neuroblastoma, melanoma, breast carcinoma (e.g. triple-negative breast cancer), gastric cancer, colorectal cancer (CRC), or hepatocellular carcinoma.

In some embodiments, any of the methods of the invention may further comprise monitoring the responsiveness of the individual to treatment, e.g., with an anti-human OX40 agonist antibody as described herein. In some embodiments, monitoring the responsiveness of an individual to treatment may include measuring the expression level of one or more marker genes in a sample (e.g., a tumor sample) obtained from the individual after treatment. In some embodiments, the individual may be classified as responsive or non-responsive to treatment based on the expression level of one or more marker genes in a sample (e.g., a tumor sample) obtained from the individual, e.g., as compared with a reference. In some embodiments, the one or more marker genes may be selected from CCR5, CD274 (also known as PD-L1), IL-7, TNFRSF14, TGFB1, CD40, CD4, PRF1, TNFSF4, CD86, CXCL9, CD3E, LAG3, PDCD1, CCL28, GZMB, IFNg, and IL-2RA, and an increased expression level (e.g., as compared with a reference) may indicate responsiveness to treatment. In certain embodiments, increased expression of PD-L1 (e.g., as compared with a reference) may indicate responsiveness to treatment. In some embodiments, the one or more marker genes may be selected from CD8b, EOMES, GZMA, GZMB, IFNg, and PRF1, and an increased expression level (e.g., as compared with a reference) may indicate responsiveness to treatment. Without wishing to be bound to theory, it is thought that increased expression of CCR5, CD274 (also known as PD-L1), IL-7, TNFRSF14, TGFB1, CD40, CD4, PRF1, TNFSF4, CD86, CXCL9, CD3E, LAG3, PDCD1, CCL28, GZMB, IFNg, IL-2RA, GZMA, CD8b, and/or EOMES may be associated with increased Teff activity. In other embodiments, the one or more marker genes may be selected from CCL22, IL-2, RORC, IL-8, CTLA4, and FOXP3, and a decreased expression level (e.g., as compared with a reference) may indicate responsiveness to treatment. Without wishing to be bound to theory, it is thought that decreased expression of CCL22, IL-2, RORC, IL-8, CTLA4, and/or FOXP3 may be associated with decreased Treg activity.

In some embodiments, any of the methods of the invention may further comprise monitoring efficacy of treatment (e.g., treatment with an anti-human OX40 agonist antibody as described herein). In some embodiments, monitoring the efficacy of treatment in an individual may include measuring the expression level of one or more marker genes in a sample (e.g., a tumor sample) obtained from the individual after treatment. In some embodiments, the treatment may be classified as efficacious based on the expression level of one or more marker genes in a sample (e.g., a tumor sample) obtained from the individual, e.g., as compared with a reference. In some embodiments, the one or more marker genes may be selected from CCR5, CD274 (also known as PD-L1), IL-7, TNFRSF14, TGFB1, CD40, CD4, PRF1, TNFSF4, CD86, CXCL9, CD3E, LAG3, PDCD1, CCL28, GZMB, IFNg, and IL-2RA, and an increased expression level (e.g., as compared with a reference) may indicate treatment efficacy. In certain embodiments, increased expression of PD-L1 (e.g., as compared with a reference) may indicate treatment efficacy. In some embodiments, the one or more marker genes may be selected from CD8b, EOMES, GZMA, GZMB, IFNg, and PRF1, and an increased expression level (e.g., as compared with a reference) may indicate treatment efficacy. Without wishing to be bound to theory, it is thought that increased expression of CCR5, CD274 (also known as PD-L1), IL-7, TNFRSF14, TGFB1, CD40, CD4, PRF1, TNFSF4, CD86, CXCL9, CD3E, LAG3, PDCD1, CCL28, GZMB, IFNg, IL-2RA, GZMA, CD8b, and/or EOMES may be associated with increased Teff activity. In other embodiments, the one or more marker genes may be selected from CCL22, IL-2, RORC, IL-8, CTLA4, and FOXP3, and a decreased expression level (e.g., as compared with a reference) may indicate treatment efficacy. Without wishing to be bound to theory, it is thought that decreased expression of CCL22, IL-2, RORC, IL-8, CTLA4, and/or FOXP3 may be associated with decreased Treg activity.

In some embodiments, the expression level of one or more marker genes described herein is compared to a reference. In some embodiments, a reference may include a biopsy obtained from the individual before treatment, a biopsy obtained from an untreated individual, or a reference or baseline value. In some embodiments, the reference is the average, mean, or median level of expression of the corresponding marker gene(s) in samples obtained from individuals that have cancer (e.g., the same type of cancer as the individual receiving treatment). In some embodiments, the reference is the average, mean, or median level of expression of the corresponding marker gene in samples from other subjects having cancer who are not responsive to the OX40 agonist treatment after receiving treatment. For example, a set of samples obtained from cancers having a shared characteristic (e.g., the same cancer type and/or stage, or exposure to a common treatment such as an OX40 agonist) may be studied from a population, such as with a clinical outcome study. This set may be used to derive a reference, e.g., a reference number, to which a subject's sample may be compared.

In some embodiments, expression level of an mRNA or protein may be normalized to the expression level of a reference gene. Normalizing the expression level of a particular gene to a reference is thought to enhance reproducibility across samples by factoring differences in sample size and/or mRNA/protein extraction. In these examples, expression level relative to the reference is measured. In some embodiments, multiple reference genes may be used, either singly or in aggregate (e.g., by averaging). In other embodiments, expression level of an mRNA or protein may refer to absolute expression level.

In some embodiments, a reference gene may be a housekeeping gene. A housekeeping gene is thought to be constitutively expressed in a cell in normal and/or pathological states, such as a gene encoding a protein required for basic cellular function and/or maintenance. Housekeeping genes are typically used as a reference to ensure they will be expressed at a detectable and/or reproducible level across multiple samples. Exemplary housekeeping genes and further description of the use of such genes as a reference may be found, for example, in de Kok, J. B., et al. (2005) Lab Invest. 85(1):154-9.

Certain aspects of the present disclosure relate to measurement of the expression level of one or more genes in a sample. In some embodiments, a sample may include leukocytes. In some embodiments, the sample may be a tumor sample. A tumor sample may include cancer cells, lymphocytes, leukocytes, stroma, blood vessels, connective tissue, basal lamina, and any other cell type in association with the tumor. In some embodiments, the sample is a tumor tissue sample containing tumor-infiltrating leukocytes. As used herein, any leukocyte associated with a tumor may be considered a tumor-infiltrating leukocyte. Examples of tumor-infiltrating leukocytes include without limitation T lymphocytes (such as CD8+T lymphocytes and/or CD4+T lymphocytes), B lymphocytes, or other bone marrow-lineage cells including granulocytes (neutrophils, eosinophils, basophils), monocytes, macrophages, dendritic cells (i.e., interdigitating dendritic cells), histiocytes, and natural killer cells. In some embodiments, a tumor-infiltrating leukocyte may be associated with cancer cells of a tumor. In some embodiments, a tumor-infiltrating leukocyte may be associated with tumor stroma. In some embodiments, the tumor samples are enriched for tumor area by macrodissection.

In some embodiments, the sample may be processed to separate or isolate one or more cell types (e.g., leukocytes). In some embodiments, the sample may be used without separating or isolating cell types. A tumor sample may be obtained from a subject by any method known in the art, including without limitation a biopsy, endoscopy, or surgical procedure. In some embodiments, a tumor sample may be prepared by methods such as freezing, fixation (e.g., by using formalin or a similar fixative), and/or embedding in paraffin wax. In some embodiments, a tumor sample may be sectioned. In some embodiments, a fresh tumor sample (i.e., one that has not been prepared by the methods described above) may be used. In some embodiments, a sample may be prepared by incubation in a solution to preserve mRNA and/or protein integrity. A tumor sample containing leukocytes may be assayed by any technique described herein for measuring marker gene expression level.

Certain aspects of the present disclosure relate to measuring the expression level of one or more marker genes. Any suitable method for measuring gene expression known in the art may be used. In some embodiments, expression level may refer to mRNA expression level. mRNA expression level may be measured by many methods. Such methods may quantify the copies of a specific mRNA present in a sample by measuring the amount of hybridization to an mRNA-specific probe. Other methods may amplify mRNA, or cDNA generated from mRNA, and quantify the amount of amplicon generated to extrapolate how much mRNA was present in a sample. Yet other methods may involve next-generation sequencing of part or all of mRNA transcripts, or cDNA generated from mRNA, then quantifying the number of sequences detected that correspond to particular gene(s). In some embodiments, mRNA expression level is measured by quantitative PCR, semi-quantitative PCR, nucleotide microarray, RNA-seq, in situ hybridization, and/or Northern blotting.

In some embodiments, expression level may refer to protein expression level. Protein expression level may be measured by many methods. Such methods may quantify proteins present in a sample by using a probe that specifically binds to a particular protein, such as an antibody, then detecting the amount of specific binding in a sample. Other methods may fragment proteins into short peptides, then detect these peptides and quantify how many peptides correspond to particular protein(s). In some embodiments, protein expression level is measured by Western blotting, peptide microarray, immunohistochemistry, flow cytometry, and/or mass spectrometry.

An “individual” according to any of the above embodiments is preferably a human.

Antibodies of the invention can be used either alone or in combination with other agents in a therapy. For instance, an antibody of the invention may be co-administered with at least one additional therapeutic agent.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the anti-OX40 antibody and administration of an additional therapeutic agent occur within about one month, or within about one, two or three weeks, or within about one, two, three, four, five, or six days, of each other. Antibodies of the invention can also be used in combination with radiation therapy.

In some embodiments, an anti-human OX40 agonist antibody may be administered in conjunction with a chemotherapy or chemotherapeutic agent. In some embodiments, an anti-human OX40 agonist antibody may be administered in conjunction with a radiation therapy or radiotherapeutic agent. In some embodiments, an anti-human OX40 agonist antibody may be administered in conjunction with a targeted therapy or targeted therapeutic agent. In some embodiments, an anti-human OX40 agonist antibody may be administered in conjunction with an immunotherapy or immunotherapeutic agent, for example a monoclonal antibody.

In some embodiments, an anti-human OX40 agonist antibody may be administered in conjunction with a PARP inhibitor (e.g., Olaparanib, Rucaparib, Niraparib, Cediranib, BMN673, Veliparib), Trabectedin, nab-paclitaxel (albumen-bound paclitaxel, ABRAXANE), Trebananib, Pazopanib, Cediranib, Palbociclib, everolimus, fluoropyrimidine (e.g., FOLFOX, FOLFIRI), IFL, regorafenib, Reolysin, Alimta, Zykadia, Sutent, Torisel (temsirolimus), Inlyta (axitinib, Pfizer), Afinitor (everolimus, Novartis), Nexavar (sorafenib, Onyx/Bayer), Votrient, Pazopanib, axitinib, IMA-901, AGS-003, cabozantinib, Vinflunine, Hsp90 inhibitor (e.g., apatorsin), Ad-GM-CSF (CT-0070), Temazolomide, IL-2, IFNa, vinblastine, Thalomid, dacarbazine, cyclophosphamide, lenalidomide, azacytidine, lenalidomide, bortezomid (VELCADE), amrubicine, carfilzomib, pralatrexate, and/or enzastaurin.

In some embodiments, an anti-human OX40 agonist antibody may be administered in conjunction with a PD-1 axis binding antagonist. A PD-1 axis binding antagonist includes but is not limited to a PD-1 binding antagonist, a PD-L1 binding antagonist and a PD-L2 binding antagonist. Alternative names for “PD-1” include CD279 and SLEB2. Alternative names for “PD-L1” include B7-H1, B7-4, CD274, and B7-H. Alternative names for “PD-L2” include B7-DC, Btdc, and CD273. In some embodiments, PD-1, PD-L1, and PD-L2 are human PD-1, PD-L1 and PD-L2. In some embodiments, the PD-1 binding antagonist is a molecule that inhibits the binding of PD-1 to its ligand binding partners. In a specific aspect the PD-1 ligand binding partners are PD-L1 and/or PD-L2. In another embodiment, a PD-L1 binding antagonist is a molecule that inhibits the binding of PD-L1 to its binding partners. In a specific aspect, PD-L1 binding partners are PD-1 and/or B7-1. In another embodiment, the PD-L2 binding antagonist is a molecule that inhibits the binding of PD-L2 to its binding partners. In a specific aspect, a PD-L2 binding partner is PD-1. The antagonist may be an antibody, an antigen binding fragment thereof, an immunoadhesin, a fusion protein, or oligopeptide. In some embodiments, the PD-1 binding antagonist is an anti-PD-1 antibody (e.g., a human antibody, a humanized antibody, or a chimeric antibody). In some embodiments, the anti-PD-1 antibody is selected from the group consisting of MDX-1106 (nivolumab, OPDIVO), Merck 3475 (MK-3475, pembrolizumab, KEYTRUDA), CT-011 (Pidilizumab), MEDI-0680 (AMP-514), PDR001, REGN2810, and BGB-108. In some embodiments, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In some embodiments, the PD-1 binding antagonist is AMP-224. In some embodiments, the PD-L1 binding antagonist is anti-PD-L1 antibody. In some embodiments, the anti-PD-L1 binding antagonist is selected from the group consisting of YW243.55.S70, MPDL3280A, MEDI4736 (durvalumab), MDX-1105, and MSB0010718C (avelumab). MDX-1105, also known as BMS-936559, is an anti-PD-L1 antibody described in WO2007/005874. Antibody YW243.55.S70 (heavy and light chain variable region sequences shown in SEQ ID Nos. 20 and 21, respectively) is an anti-PD-L1 described in WO 2010/077634 A1. MDX-1106, also known as MDX-1106-04, ONO-4538, BMS-936558 or nivolumab, is an anti-PD-1 antibody described in WO2006/121168. Merck 3475, also known as MK-3475, SCH-900475 or pembrolizumab, is an anti-PD-1 antibody described in WO2009/114335. CT-011, also known as hBAT, hBAT-1 or pidilizumab, is an anti-PD-1 antibody described in WO2009/101611. AMP-224, also known as B7-DCIg, is a PD-L2-Fc fusion soluble receptor described in WO2010/027827 and WO2011/066342. In some embodiments, the anti-PD-1 antibody is MDX-1106. Alternative names for “MDX-1106” include MDX-1 106-04, ONO-4538, BMS-936558 or nivolumab. In some embodiments, the anti-PD-1 antibody is nivolumab (CAS Registry Number: 946414-94-4).

In some embodiments, an anti-human OX40 agonist antibody may be administered in conjunction with an agonist directed against an activating co-stimulatory molecule. In some embodiments, an activating co-stimulatory molecule may include CD40, CD226, CD28, GITR, CD137, CD27, HVEM, or CD127. In some embodiments, the agonist directed against an activating co-stimulatory molecule is an agonist antibody that binds to CD40, CD226, CD28, OX40, GITR, CD137, CD27, HVEM, or CD127. In some embodiments, an anti-human OX40 agonist antibody may be administered in conjunction with an antagonist directed against an inhibitory co-stimulatory molecule. In some embodiments, an inhibitory co-stimulatory molecule may include CTLA-4 (also known as CD152), PD-1, TIM-3, BTLA, VISTA, LAG-3, B7-H3, B7-H4, IDO, TIGIT, MICA/B, or arginase. In some embodiments, the antagonist directed against an inhibitory co-stimulatory molecule is an antagonist antibody that binds to CTLA-4, PD-1, TIM-3, BTLA, VISTA, LAG-3 (e.g., LAG-3-IgG fusion protein (IMP321)), B7-H3, B7-H4, IDO, TIGIT, MICA/B, or arginase.

In some embodiments, an anti-human OX40 agonist antibody may be administered in conjunction with an antagonist directed against CTLA-4 (also known as CD152), e.g., a blocking antibody. In some embodiments, an anti-human OX40 agonist antibody may be administered in conjunction with ipilimumab (also known as MDX-010, MDX-101, or Yervoy®). In some embodiments, an anti-human OX40 agonist antibody may be administered in conjunction with tremelimumab (also known as ticilimumab or CP-675,206). In some embodiments, an anti-human OX40 agonist antibody may be administered in conjunction with an antagonist directed against B7-H3 (also known as CD276), e.g., a blocking antibody. In some embodiments, an anti-human OX40 agonist antibody may be administered in conjunction with MGA271. In some embodiments, an anti-human OX40 agonist antibody may be administered in conjunction with an antagonist directed against a TGF beta, e.g., metelimumab (also known as CAT-192), fresolimumab (also known as GC1008), or LY2157299.

In some embodiments, an anti-human OX40 agonist antibody may be administered in conjunction with a treatment comprising adoptive transfer of a T cell (e.g., a cytotoxic T cell or CTL) expressing a chimeric antigen receptor (CAR). In some embodiments, an anti-human OX40 agonist antibody may be administered in conjunction with UCART19. In some embodiments, an anti-human OX40 agonist antibody may be administered in conjunction with WT128z. In some embodiments, an anti-human OX40 agonist antibody may be administered in conjunction with KTE-C19 (Kite). In some embodiments, an anti-human OX40 agonist antibody may be administered in conjunction with CTL019 (Novartis). In some embodiments, an anti-human OX40 agonist antibody may be administered in conjunction with a treatment comprising adoptive transfer of a T cell comprising a dominant-negative TGF beta receptor, e.g, a dominant-negative TGF beta type II receptor. In some embodiments, an anti-human OX40 agonist antibody may be administered in conjunction with a treatment comprising a HERCREEM protocol (see, e.g., ClinicalTrials.gov Identifier NCT00889954).

In some embodiments, an anti-human OX40 agonist antibody may be administered in conjunction with an antagonist directed against CD19. In some embodiments, an anti-human OX40 agonist antibody may be administered in conjunction with MOR00208. In some embodiments, an anti-human OX40 agonist antibody may be administered in conjunction with an antagonist directed against CD38. In some embodiments, an anti-human OX40 agonist antibody may be administered in conjunction with daratumumab.

In some embodiments, an anti-human OX40 agonist antibody may be administered in conjunction with an agonist directed against CD137 (also known as TNFRSF9, 4-1BB, or ILA), e.g., an activating antibody. In some embodiments, an anti-human OX40 agonist antibody may be administered in conjunction with urelumab (also known as BMS-663513). In some embodiments, an anti-human OX40 agonist antibody may be administered in conjunction with an agonist directed against CD40, e.g., an activating antibody. In some embodiments, an anti-human OX40 agonist antibody may be administered in conjunction with CP-870893. In some embodiments, an anti-human OX40 agonist antibody may be administered in conjunction with an agonist directed against OX40 (also known as CD134), e.g., an activating antibody. In some embodiments, an anti-human OX40 agonist antibody may be administered in conjunction with a different anti-OX40 antibody (e.g., AgonOX). In some embodiments, an anti-human OX40 agonist antibody may be administered in conjunction with an agonist directed against CD27, e.g., an activating antibody. In some embodiments, an anti-human OX40 agonist antibody may be administered in conjunction with CDX-1127. In some embodiments, an anti-human OX40 agonist antibody may be administered in conjunction with an antagonist directed against indoleamine-2,3-dioxygenase (IDO). In some embodiments, with the IDO antagonist is 1-methyl-D-tryptophan (also known as 1-D-MT).

In some embodiments, an anti-human OX40 agonist antibody may be administered in conjunction with an agonist directed against CD137 (also known as TNFRSF9, 4-1BB, or ILA), e.g., an activating antibody. In some embodiments, an anti-human OX40 agonist antibody may be administered in conjunction with urelumab (also known as BMS-663513). In some embodiments, an anti-human OX40 agonist antibody may be administered in conjunction with an agonist directed against CD40, e.g., an activating antibody. In some embodiments, an anti-human OX40 agonist antibody may be administered in conjunction with CP-870893 or R07009789. In some embodiments, an anti-human OX40 agonist antibody may be administered in conjunction with an agonist directed against OX40 (also known as CD134), e.g., an activating antibody.). In some embodiments, an anti-human OX40 agonist antibody may be administered in conjunction with an agonist directed against CD27, e.g., an activating antibody. In some embodiments, an anti-human OX40 agonist antibody may be administered in conjunction with CDX-1127 (also known as varlilumab). In some embodiments, an anti-human OX40 agonist antibody may be administered in conjunction with an antagonist directed against indoleamine-2,3-dioxygenase (IDO). In some embodiments, with the IDO antagonist is 1-methyl-D-tryptophan (also known as 1-D-MT). In some embodiments, the IDO antagonist is an IDO antagonist shown in WO2010/005958 (the contents of which are expressly incorporated by record herein). In some embodiments the IDO antagonist is 4-({2-[(Aminosulfonyl)amino]ethyl}amino)-N-(3-bromo-4-fluorophenyl)-N′-hydroxy-1,2,5-oxadiazole-3-carboximidamide (e.g., as described in Example 23 of WO2010/005958). In some embodiments the IDO antagonist is

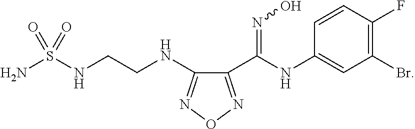 [SMILES: NS(=O)(=O)NCCNc1nonc1C(=NO)Nc1ccc(F)c(Br)c1]

In some embodiments, the IDO antagonist is INCB24360. In some embodiments, the IDO antagonist is Indoximod (the D isomer of 1-methyl-tryptophan). In some embodiments, an anti-human OX40 agonist antibody may be administered in conjunction with an antibody-drug conjugate. In some embodiments, the antibody-drug conjugate comprises mertansine or monomethyl auristatin E (MMAE). In some embodiments, an anti-human OX40 agonist antibody may be administered in conjunction with an anti-NaPi2b antibody-MMAE conjugate (also known as DNIB0600A, RG7599 or lifastuzumab vedotin). In some embodiments, an anti-human OX40 agonist antibody may be administered in conjunction with trastuzumab emtansine (also known as T-DM1, ado-trastuzumab emtansine, or KADCYLA®, Genentech). In some embodiments, an anti-human OX40 agonist antibody may be administered in conjunction with an anti-MUC16 antibody-MMAE conjugate, DMUC5754A. In some embodiments, an anti-human OX40 agonist antibody may be administered in conjunction with an anti-MUC16 antibody-MMAE conjugate, DMUC4064A. In some embodiments, an anti-human OX40 agonist antibody may be administered in conjunction with an antibody-drug conjugate targeting the endothelin B receptor (EDNBR), e.g., an antibody directed against EDNBR conjugated with MMAE. In some embodiments, an anti-human OX40 agonist antibody may be administered in conjunction with an antibody-drug conjugate targeting the lymphocyte antigen 6 complex, locus E (Ly6E), e.g., an antibody directed against Ly6E conjugated with MMAE, (also known as DLYE5953A). In some embodiments, an anti-human OX40 agonist antibody may be administered in conjunction with polatuzumab vedotin. In some embodiments, an anti-human OX40 agonist antibody may be administered in conjunction with an antibody-drug conjugate targeting CD30. In some embodiments, an anti-human OX40 agonist antibody may be administered in conjunction with ADCETRIS (also known as brentuximab vedotin). In some embodiments, an anti-human OX40 agonist antibody may be administered in conjunction with polatuzumab vedotin.

In some embodiments, an anti-human OX40 agonist antibody may be administered in conjunction with an angiogenesis inhibitor. In some embodiments, an anti-human OX40 agonist antibody may be administered in conjunction with an antibody directed against a VEGF, e.g., VEGF-A. In some embodiments, an anti-human OX40 agonist antibody may be administered in conjunction with bevacizumab (also known as AVASTIN®, Genentech). In some embodiments, an anti-human OX40 agonist antibody may be administered in conjunction with an antibody directed against angiopoietin 2 (also known as Ang2). In some embodiments, an anti-human OX40 agonist antibody may be administered in conjunction with MEDI3617. In some embodiments, an anti-human OX40 agonist antibody may be administered in conjunction with an antibody directed against VEGFR2. In some embodiments, an anti-human OX40 agonist antibody may be administered in conjunction with ramucirumab. In some embodiments, an anti-human OX40 agonist antibody may be administered in conjunction with a VEGF Receptor fusion protein. In some embodiments, an anti-human OX40 agonist antibody may be administered in conjunction with aflibercept. In some embodiments, an anti-human OX40 agonist antibody may be administered in conjunction with ziv-aflibercept (also known as VEGF Trap or Zaltrap®). In some embodiments, an anti-human OX40 agonist antibody may be administered in conjunction with a bispecific antibody directed against VEGF and Ang2. In some embodiments, an anti-human OX40 agonist antibody may be administered in conjunction with RG7221 (also known as vanucizumab). In some embodiments, an anti-human OX40 agonist antibody may be administered in conjunction with an angiogenesis inhibitor and in conjunction with a PD-1 axis binding antagonist (e.g., a PD-1 binding antagonist such as an anti-PD-1 antibody, a PD-L1 binding antagonist such as an anti-PD-L1 antibody, and a PD-L2 binding antagonist such as an anti-PD-L2 antibody). In some embodiments, an anti-human OX40 agonist antibody may be administered in conjunction with bevacizumab and a PD-1 axis binding antagonist (e.g., a PD-1 binding antagonist such as an anti-PD-1 antibody, a PD-L1 binding antagonist such as an anti-PD-L1 antibody, and a PD-L2 binding antagonist such as an anti-PD-L2 antibody). In some embodiments, an anti-human OX40 agonist antibody may be administered in conjunction with bevacizumab and MDX-1106 (nivolumab, OPDIVO). In some embodiments, an anti-human OX40 agonist antibody may be administered in conjunction with bevacizumab and Merck 3475 (MK-3475, pembrolizumab, KEYTRUDA). In some embodiments, an anti-human OX40 agonist antibody may be administered in conjunction with bevacizumab and CT-011 (Pidilizumab). In some embodiments, an anti-human OX40 agonist antibody may be administered in conjunction with bevacizumab and YW243.55.S70. In some embodiments, an anti-human OX40 agonist antibody may be administered in conjunction with bevacizumab and MPDL3280A. In some embodiments, an anti-human OX40 agonist antibody may be administered in conjunction with bevacizumab and MEDI4736. In some embodiments, an anti-human OX40 agonist antibody may be administered in conjunction with bevacizumab and MDX-1105.

In some embodiments, an anti-human OX40 agonist antibody may be administered in conjunction with an antineoplastic agent. In some embodiments, an anti-human OX40 agonist antibody may be administered in conjunction with an agent targeting CSF-1R (also known as M-CSFR or CD115). In some embodiments, an anti-human OX40 agonist antibody may be administered in conjunction with anti-CSF-1R antibody (also known as IMC-CS4 or LY3022855) In some embodiments, an anti-human OX40 agonist antibody may be administered in conjunction with anti-CSF-1R antibody, RG7155 (also known as R05509554 or emactuzumab). In some embodiments, an anti-human OX40 agonist antibody may be administered in conjunction with an interferon, for example interferon alpha or interferon gamma. In some embodiments, an anti-human OX40 agonist antibody may be administered in conjunction with Roferon-A (also known as recombinant Interferon alpha-2a). In some embodiments, an anti-human OX40 agonist antibody may be administered in conjunction with GM-CSF (also known as recombinant human granulocyte macrophage colony stimulating factor, rhu GM-CSF, sargramostim, or Leukine®). In some embodiments, an anti-human OX40 agonist antibody may be administered in conjunction with IL-2 (also known as aldesleukin or Proleukin®). In some embodiments, an anti-human OX40 agonist antibody may be administered in conjunction with IL-12. In some embodiments, an anti-human OX40 agonist antibody may be administered in conjunction with IL27. In some embodiments, an anti-human OX40 agonist antibody may be administered in conjunction with IL-15. In some embodiments, an anti-human OX40 agonist antibody may be administered in conjunction with ALT-803. In some embodiments, an anti-human OX40 agonist antibody may be administered in conjunction with an antibody targeting CD20. In some embodiments, the antibody targeting CD20 is obinutuzumab (also known as GA101 or Gazyva®) or rituximab. In some embodiments, an anti-human OX40 agonist antibody may be administered in conjunction with an antibody targeting GITR. In some embodiments, the antibody targeting GITR is TRX518. In some embodiments, the antibody targeting GITR is MK04166 (Merck).

In some embodiments, an anti-human OX40 agonist antibody may be administered in conjunction with an inhibitor of Bruton's tyrosine kinase (BTK). In some embodiments, an anti-human OX40 agonist antibody may be administered in conjunction with ibrutinib. In some embodiments, an anti-human OX40 agonist antibody may be administered in conjunction with an inhibitor of Isocitrate dehydrogenase 1 (IDH1) and/or Isocitrate dehydrogenase 2 (IDH2). In some embodiments, an anti-human OX40 agonist antibody may be administered in conjunction with AG-120 (Agios).

In some embodiments, an anti-human OX40 agonist antibody may be administered in conjunction with obinutuzumab and a PD-1 axis binding antagonist (e.g., a PD-1 binding antagonist such as an anti-PD-1 antibody, a PD-L1 binding antagonist such as an anti-PD-L1 antibody, and a PD-L2 binding antagonist such as an anti-PD-L2 antibody).

In some embodiments, an anti-human OX40 agonist antibody may be administered in conjunction with a cancer vaccine. In some embodiments, the cancer vaccine is a peptide cancer vaccine, which in some embodiments is a personalized peptide vaccine. In some embodiments the peptide cancer vaccine is a multivalent long peptide, a multi-peptide, a peptide cocktail, a hybrid peptide, or a peptide-pulsed dendritic cell vaccine (see, e.g., Yamada et al., Cancer Sci, 104:14-21, 2013). In some embodiments, an anti-human OX40 agonist antibody may be administered in conjunction with an adjuvant. In some embodiments, an anti-human OX40 agonist antibody may be administered in conjunction with a treatment comprising a TLR agonist, e.g., Poly-ICLC (also known as Hiltonol®), LPS, MPL, or CpG ODN. In some embodiments, an anti-human OX40 agonist antibody may be administered in conjunction with tumor necrosis factor (TNF) alpha. In some embodiments, an anti-human OX40 agonist antibody may be administered in conjunction with IL-1. In some embodiments, an anti-human OX40 agonist antibody may be administered in conjunction with HMGB1. In some embodiments, an anti-human OX40 agonist antibody may be administered in conjunction with an IL-10 antagonist. In some embodiments, an anti-human OX40 agonist antibody may be administered in conjunction with an IL-4 antagonist. In some embodiments, an anti-human OX40 agonist antibody may be administered in conjunction with an IL-13 antagonist. In some embodiments, an anti-human OX40 agonist antibody may be administered in conjunction with an IL-17 antagonist. In some embodiments, an anti-human OX40 agonist antibody may be administered in conjunction with an HVEM antagonist. In some embodiments, an anti-human OX40 agonist antibody may be administered in conjunction with an ICOS agonist, e.g., by administration of ICOS-L, or an agonistic antibody directed against ICOS. In some embodiments, an anti-human OX40 agonist antibody may be administered in conjunction with a treatment targeting CX3CL1. In some embodiments, an anti-human OX40 agonist antibody may be administered in conjunction with a treatment targeting CXCL9. In some embodiments, an anti-human OX40 agonist antibody may be administered in conjunction with a treatment targeting CXCL10. In some embodiments, an anti-human OX40 agonist antibody may be administered in conjunction with a treatment targeting CCL5. In some embodiments, an anti-human OX40 agonist antibody may be administered in conjunction with an LFA-1 or ICAM1 agonist. In some embodiments, an anti-human OX40 agonist antibody may be administered in conjunction with a Selectin agonist.

In some embodiments, an anti-human OX40 agonist antibody may be administered in conjunction with an inhibitor of B-Raf. In some embodiments, an anti-human OX40 agonist antibody may be administered in conjunction with vemurafenib (also known as Zelboraf®). In some embodiments, an anti-human OX40 agonist antibody may be administered in conjunction with dabrafenib (also known as Tafinlar®). In some embodiments, an anti-human OX40 agonist antibody may be administered in conjunction with encorafenib (LGX818).

In some embodiments, an anti-human OX40 agonist antibody may be administered in conjunction with an EGFR inhibitor. In some embodiments, an anti-human OX40 agonist antibody may be administered in conjunction with erlotinib (also known as Tarceva®). In some embodiments, an anti-human OX40 agonist antibody may be administered in conjunction with an inhibitor of EGFR-T790M. In some embodiments, an anti-human OX40 agonist antibody may be administered in conjunction with gefitinib. In some embodiments, an anti-human OX40 agonist antibody may be administered in conjunction with afatinib. In some embodiments, an anti-human OX40 agonist antibody may be administered in conjunction with cetuximab (also known as Erbitux®). In some embodiments, an anti-human OX40 agonist antibody may be administered in conjunction with panitumumab (also known as Vectibix®). In some embodiments, an anti-human OX40 agonist antibody may be administered in conjunction with rociletinib. In some embodiments, an anti-human OX40 agonist antibody may be administered in conjunction with AZD9291. In some embodiments, an anti-human OX40 agonist antibody may be administered in conjunction with an inhibitor of a MEK, such as MEK1 (also known as MAP2K1) and/or MEK2 (also known as MAP2K2). In some embodiments, an anti-human OX40 agonist antibody may be administered in conjunction with cobimetinib (also known as GDC-0973 or XL-518). In some embodiments, an anti-human OX40 agonist antibody may be administered in conjunction with trametinib (also known as Mekinist®). In some embodiments, an anti-human OX40 agonist antibody may be administered in conjunction with binimetinib.

In some embodiments, an anti-human OX40 agonist antibody may be administered in conjunction an inhibitor of B-Raf (e.g., vemurafenib or dabrafenib) and an inhibitor of MEK (e.g., MEK1 and/or MEK2 (e.g., cobimetinib or trametinib). In some embodiments, an anti-human OX40 agonist antibody may be administered in conjunction with an inhibitor of ERK (e.g., ERK1/2). In some embodiments, an anti-human OX40 agonist antibody may be administered in conjunction with GDC-0994). In some embodiments, an anti-human OX40 agonist antibody may be administered in conjunction with an inhibitor of B-Raf, an inhibitor of MEK, and an inhibitor of ERK1/2. In some embodiments, an anti-human OX40 agonist antibody may be administered in conjunction with an inhibitor of EGFR, an inhibitor of MEK, and an inhibitor of ERK1/2. In some embodiments, an anti-human OX40 agonist antibody may be administered in conjunction with one or more MAP kinase pathway inhibitor. In some embodiments, an anti-human OX40 agonist antibody may be administered in conjunction with CK127. In some embodiments, an anti-human OX40 agonist antibody may be administered in conjunction with an inhibitor of K-Ras.

In some embodiments, an anti-human OX40 agonist antibody may be administered in conjunction with an inhibitor of c-Met. In some embodiments, an anti-human OX40 agonist antibody may be administered in conjunction with onartuzumab (also known as MetMAb). In some embodiments, an anti-human OX40 agonist antibody may be administered in conjunction with an inhibitor of anaplatic lymphoma kinase (ALK). In some embodiments, an anti-human OX40 agonist antibody may be administered in conjunction with AF802 (also known as CH5424802 or alectinib). In some embodiments, an anti-human OX40 agonist antibody may be administered in conjunction with crizotinib. In some embodiments, an anti-human OX40 agonist antibody may be administered in conjunction with ceritinib. In some embodiments, an anti-human OX40 agonist antibody may be administered in conjunction with an inhibitor of a phosphatidylinositol 3-kinase (PI3K). In some embodiments, an anti-human OX40 agonist antibody may be administered in conjuction with buparlisib (BKM-120). In some embodiments, an anti-human OX40 agonist antibody may be administered in conjunction with pictilisib (also known as GDC-0941). In some embodiments, an anti-human OX40 agonist antibody may be administered in conjunction with buparlisib (also known as BKM-120). In some embodiments, an anti-human OX40 agonist antibody may be administered in conjunction with perifosine (also known as KRX-0401). In some embodiments, an anti-human OX40 agonist antibody may be administered in conjunction with a delta-selective inhibitor of a phosphatidylinositol 3-kinase (PI3K). In some embodiments, an anti-human OX40 agonist antibody may be administered in conjunction with idelalisib (also known as GS-1101 or CAL-101). In some embodiments, an anti-human OX40 agonist antibody may be administered in conjunction with taselisib (also known as GDC-0032). In some embodiments, an anti-human OX40 agonist antibody may be administered in conjunction with BYL-719. In some embodiments, an anti-human OX40 agonist antibody may be administered in conjunction with an inhibitor of an Akt. In some embodiments, an anti-human OX40 agonist antibody may be administered in conjunction with MK2206. In some embodiments, an anti-human OX40 agonist antibody may be administered in conjunction with GSK690693. In some embodiments, an anti-human OX40 agonist antibody may be administered in conjunction with ipatasertib (also known as GDC-0068). In some embodiments, an anti-human OX40 agonist antibody may be administered in conjunction with an inhibitor of mTOR. In some embodiments, an anti-human OX40 agonist antibody may be administered in conjunction with sirolimus (also known as rapamycin). In some embodiments, an anti-human OX40 agonist antibody may be administered in conjunction with temsirolimus (also known as CCI-779 or Torisel®). In some embodiments, an anti-human OX40 agonist antibody may be administered in conjunction with everolimus (also known as RAD001). In some embodiments, an anti-human OX40 agonist antibody may be administered in conjunction with ridaforolimus (also known as AP-23573, MK-8669, or deforolimus). In some embodiments, an anti-human OX40 agonist antibody may be administered in conjunction with OSI-027. In some embodiments, an anti-human OX40 agonist antibody may be administered in conjunction with AZD8055. In some embodiments, an anti-human OX40 agonist antibody may be administered in conjunction with INK128. In some embodiments, an anti-human OX40 agonist antibody may be administered in conjunction with a dual PI3K/mTOR inhibitor. In some embodiments, an anti-human OX40 agonist antibody may be administered in conjunction with XL765. In some embodiments, an anti-human OX40 agonist antibody may be administered in conjunction with GDC-0980. In some embodiments, an anti-human OX40 agonist antibody may be administered in conjunction with BEZ235 (also known as NVP-BEZ235). In some embodiments, an anti-human OX40 agonist antibody may be administered in conjunction with BGT226. In some embodiments, an anti-human OX40 agonist antibody may be administered in conjunction with GSK2126458. In some embodiments, an anti-human OX40 agonist antibody may be administered in conjunction with PF-04691502. In some embodiments, an anti-human OX40 agonist antibody may be administered in conjunction with PF-05212384 (also known as PKI-587).

In some embodiments, an anti-human OX40 agonist antibody may be administered in conjunction with an agent that selectively degrades the estrogen receptor. In some embodiments, an anti-human OX40 agonist antibody may be administered in conjunction with GDC-0927. In some embodiments, an anti-human OX40 agonist antibody may be administered in conjunction with an inhibitor of HER3. In some embodiments, an anti-human OX40 agonist antibody may be administered in conjunction with duligotuzumab. In some embodiments, an anti-human OX40 agonist antibody may be administered in conjunction with an inhibitor of LSD1. In some embodiments, an anti-human OX40 agonist antibody may be administered in conjunction with an inhibitor of MDM2. In some embodiments, an anti-human OX40 agonist antibody may be administered in conjunction with an inhibitor of BCL2. In some embodiments, an anti-human OX40 agonist antibody may be administered in conjunction with venetoclax. In some embodiments, an anti-human OX40 agonist antibody may be administered in conjunction with an inhibitor of CHK1. In some embodiments, an anti-human OX40 agonist antibody may be administered in conjunction with GDC-0575. In some embodiments, an anti-human OX40 agonist antibody may be administered in conjunction with an inhibitor of activated hedgehog signaling pathway. In some embodiments, an anti-human OX40 agonist antibody may be administered in conjunction with ERIVEDGE.

In some embodiments, an anti-human OX40 agonist antibody may be administered in conjunction with radiation therapy. In some embodiments, an anti-human OX40 agonist antibody may be administered in conjunction with gemcitabine. In some embodiments, an anti-human OX40 agonist antibody may be administered in conjunction with nab-paclitaxel (ABRAXANE). In some embodiments, an anti-human OX40 agonist antibody may be administered in conjunction with trastuzumab. In some embodiments, an anti-human OX40 agonist antibody may be administered in conjunction with TVEC. In some embodiments, an anti-human OX40 agonist antibody may be administered in conjunction with IL27. In some embodiments, an anti-human OX40 agonist antibody may be administered in conjunction with cyclophosphamide. In some embodiments, an anti-human OX40 agonist antibody may be administered in conjunction with an agent that recruits T cells to the tumor. In some embodiments, an anti-human OX40 agonist antibody may be administered in conjunction with lirilumab (IPH2102/BMS-986015). In some embodiments, an anti-human OX40 agonist antibody may be administered in conjunction with Idelalisib. In some embodiments, an anti-human OX40 agonist antibody may be administered in conjunction with an antibody that targets CD3 and CD20. In some embodiments, an anti-human OX40 agonist antibody may be administered in conjunction with REGN1979. In some embodiments, an anti-human OX40 agonist antibody may be administered in conjunction with an antibody that targets CD3 and CD19. In some embodiments, an anti-human OX40 agonist antibody may be administered in conjunction with blinatumomab.

In some embodiments, an anti-human OX40 agonist antibody may be administered in conjunction with an oncolytic virus. In some embodiments, an anti-human OX40 agonist antibody may be administered in conjunction with carboplatin and nab-paclitaxel. In some embodiments, an anti-human OX40 agonist antibody may be administered in conjunction with carboplatin and paclitaxel. In some embodiments, an anti-human OX40 agonist antibody may be administered in conjunction with cisplatin and pemetrexed. In some embodiments, an anti-human OX40 agonist antibody may be administered in conjunction with cisplatin and gemcitabine. In some embodiments, an anti-human OX40 agonist antibody may be administered in conjunction with FOLFOX. In some embodiments, an anti-human OX40 agonist antibody may be administered in conjunction with FOLFIRI.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent and/or adjuvant. Antibodies of the invention can also be used in combination with radiation therapy.

An antibody of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

In certain embodiments, the antibody is administered intravenously. In some embodiments, the antibody is administered by intravenous infusion. For example, the antibody may be delivered via intravenous infusion over approximately 90 minutes, approximately 60 minutes, or approximately 30 minutes. In some embodiments, if a patient tolerates an infusion over a particular duration (e.g., a 90 minute infusion), subsequent infusions may be administered over a shorter duration (e.g., 30 or 60 minutes). Infusions may be slowed or interrupted for infusion-associated symptoms.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 40 mg/kg of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. Such doses may be administered intermittently, e.g. every week or every three weeks (e.g. such that the patient receives from about two to about twenty, or e.g. about six doses of the antibody). An initial higher loading dose, followed by one or more lower doses may be administered.

However, other dosage regimens may be useful. The progress of this therapy is easily monitored by conventional techniques and assays.

It is understood that any of the above formulations or therapeutic methods may be carried out using an immunoconjugate of the invention in place of or in addition to an anti-OX40 antibody.

### III. Articles of Manufacture and Kits

In another aspect of the invention, an article of manufacture or kit containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

In some embodiments, the article of manufacture or kit contains a container including an anti-human OX40 agonist antibody of the present disclosure for administration of a dose described herein, e.g., a dose selected from about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg per administration. For example, the container may contain an amount of antibody higher than the intended dose, e.g., to account for incomplete transfer of the antibody during administration.

In some embodiments, provided herein is a kit comprising a medicament comprising an anti-human OX40 agonist antibody described herein and an optional pharmaceutically acceptable carrier. In some embodiments, the kit further comprises instructions for administration of the medicament for treatment of cancer.

It is understood that any of the above articles of manufacture may include an immunoconjugate of the invention in place of or in addition to an anti-OX40 antibody.

### Sequences

| Name | SEQUENCE | SEQ ID NO: |
|---|---|---|
| Human OX40 | LHCVGDTYPSNDRCCHECRPGNGMVSRCSRSQNTVCRPCGPG | 1 |
| (lacking the | FYNDVVSSKPCKPCTWCNLRSGSERKQLCTATQDTVCRCRAG | |
| signal peptide) | TQPLDSYKPGVDCAPCPPGHFSPGDNQACKPWTNCTLAGKHT | |
| | LQPASNSSDAICEDRDPPATQPQETQGPPARPITVQPTEAWPRT | |
| | SQGPSTRPVEVPGGRAVAAILGLGLVLGLLGPLAILLALYLLRR | |
| | DQRLPPDAHKPPGGGSFRTPIQEEQADAHSTLAKI | |
| HVR-H1- | DSYMS | 2 |
| 1A7.gr.1 | | |
| 1A7.gr.2 | | |
| 1A7.gr.3 | | |
| 1A7.gr.4 | | |
| 1A7.gr.5 | | |
| 1A7.gr.5′ | | |
| 1A7.gr.6 | | |
| 1A7.gr.7 | | |
| 1A7.gr.7′ | | |
| 1A7.gr.NADS | | |
| 1A7.gr.NADA | | |
| 1A7.gr.NGDA | | |
| 1A7.gr.SGDS | | |
| 1A7.gr.NGSS | | |
| 1A7.Ala.1 | | |
| 1A7.Ala.2 | | |
| 1A7.Ala.3 | | |
| 1A7.Ala.4 | | |
| 1A7.Ala.5 | | |
| 1A7.Ala.6 | | |
| 1A7.Ala.7 | | |
| 1A7.Ala.8 | | |
| 1A7.Ala.9 | | |
| 1A7.Ala.10 | | |
| 1A7.Ala.11 | | |
| 1A7.Ala.12 | | |
| 1A7.Ala.13 | | |
| 1A7.Ala.14 | | |
| 1A7.Ala.15 | | |
| 1A7.Ala.16 | | |
| HVR-H2- | DMYPDNGDSSYNQKFRE | 3 |
| 1A7.gr.1 | | |
| 1A7.gr.2 | | |
| 1A7.gr.3 | | |
| 1A7.gr.4 | | |
| 1A7.gr.5 | | |
| 1A7.gr.5′ | | |
| 1A7.gr.6 | | |
| 1A7.gr.7 | | |
| 1A7.gr.7′ | | |
| 1A7.gr.DA | | |
| 1A7.gr.ES | | |
| 1A7.Ala.1 | | |
| 1A7.Ala.2 | | |
| 1A7.Ala.3 | | |
| 1A7.Ala.4 | | |
| 1A7.Ala.5 | | |
| 1A7.Ala.6 | | |
| 1A7.Ala.7 | | |
| 1A7.Ala.8 | | |
| 1A7.Ala.9 | | |
| 1A7.Ala.10 | | |
| 1A7.Ala.11 | | |
| 1A7.Ala.12 | | |
| 1A7.Ala.13 | | |
| 1A7.Ala.14 | | |
| 1A7.Ala.15 | | |
| 1A7.Ala.16 | | |
| HVR-H3- | APRWYFSV | 4 |
| 1A7.gr.1 | | |
| 1A7.gr.2 | | |
| 1A7.gr.3 | | |
| 1A7.gr.4 | | |
| 1A7.gr.5 | | |
| 1A7.gr.5′ | | |
| 1A7.gr.6 | | |
| 1A7.gr.7 | | |
| 1A7.gr.7′ | | |
| 1A7.gr.DA | | |
| 1A7.gr.ES | | |
| 1A7.gr.NADS | | |
| 1A7.gr.NADA | | |
| 1A7.gr.NGDA | | |
| 1A7.gr.SGDS | | |
| 1A7.gr.NGSS | | |
| 1A7.gr.DANADA | | |
| 1A7.Ala.1 | | |
| 1A7.Ala.2 | | |
| 1A7.Ala.3 | | |
| 1A7.Ala.4 | | |
| 1A7.Ala.5 | | |
| 1A7.Ala.6 | | |
| 1A7.Ala.7 | | |
| 1A7-Ala.15 | | |
| 1A7.Ala.16 | | |
| HVR-L1- | RASQDISNYLN | 5 |
| 1A7.gr.1 | | |
| 1A7.gr.2 | | |
| 1A7.gr.3 | | |
| 1A7.gr.4 | | |
| 1A7.gr.5 | | |
| 1A7.gr.5′ | | |
| 1A7.gr.6 | | |
| 1A7.gr.7 | | |
| 1A7.gr.7′ | | |
| 1A7.gr.DA | | |
| 1A7.gr.ES | | |
| 1A7.gr.NADS | | |
| 1A7.gr.NADA | | |
| 1A7.gr.NGDA | | |
| 1A7.gr.SGDS | | |
| 1A7.gr.NGSS | | |
| 1A7.gr.DANADA | | |
| 1A7.Ala.1 | | |
| 1A7.Ala.2 | | |
| 1A7.Ala.3 | | |
| 1A7.Ala.4 | | |
| 1A7.Ala.5 | | |
| 1A7.Ala.6 | | |
| 1A7.Ala.7 | | |
| 1A7.Ala.8 | | |
| 1A7.Ala.9 | | |
| 1A7.Ala.10 | | |
| 1A7.Ala.11 | | |
| 1A7.Ala.12 | | |
| 1A7.Ala.13 | | |
| 1A7.Ala.14 | | |
| 1A7.Ala.15 | | |
| 1A7.Ala.16 | | |
| HVR-L2- | YTSRLRS | 6 |
| 1A7.gr.1 | | |
| 1A7.gr.2 | | |
| 1A7.gr.3 | | |
| 1A7.gr.4 | | |
| 1A7.gr.5 | | |
| 1A7.gr.5′ | | |
| 1A7.gr.6 | | |
| 1A7.gr.7 | | |
| 1A7.gr.7′ | | |
| 1A7.gr.DA | | |
| 1A7.gr.ES | | |
| 1A7.gr.NADS | | |
| 1A7.gr.NADA | | |
| 1A7.gr.NGDA | | |
| 1A7.gr.SGDS | | |
| 1A7.gr.NGSS | | |
| 1A7.gr.DANADA | | |
| 1A7.Ala.1 | | |
| 1A7.Ala.2 | | |
| 1A7.Ala.3 | | |
| 1A7.Ala.4 | | |
| 1A7.Ala.5 | | |
| 1A7.Ala.6 | | |
| 1A7.Ala.7 | | |
| 1A7.Ala.8 | | |
| 1A7.Ala.9 | | |
| 1A7.Ala.10 | | |
| 1A7.Ala.11 | | |
| 1A7.Ala.12 | | |
| 1A7.Ala.13 | | |
| 1A7.Ala.14 | | |
| 1A7.Ala.15 | | |
| 1A7.Ala.16 | | |
| HVR-L3- | QQGHTLPPT | 7 |
| 1A7.gr.1 | | |
| 1A7.gr.2 | | |
| 1A7.gr.3 | | |
| 1A7.gr.4 | | |
| 1A7.gr.5 | | |
| 1A7.gr.5′ | | |
| 1A7.gr.6 | | |
| 1A7.gr.7 | | |
| 1A7.gr.7′ | | |
| 1A7.gr.DA | | |
| 1A7.gr.ES | | |
| 1A7.gr.NADS | | |
| 1A7.gr.NADA | | |
| 1A7.gr.NGDA | | |
| 1A7.gr.SGDS | | |
| 1A7.gr.NGSS | | |
| 1A7.gr.DANADA | | |
| 1A7.Ala.8 | | |
| 1A7.Ala.9 | | |
| 1A7.Ala.10 | | |
| 1A7.Ala.11 | | |
| 1A7.Ala.12 | | |
| 1A7.Ala.13 | | |
| 1A7.Ala.14 | | |
| 1A7.Ala.15 | | |
| 1A7.Ala.16 | | |
| HVR-H1- | DAYMS | 8 |
| 1A7.gr.DA | | |
| HVR-H1- | ESYMS | 9 |
| 1A7.gr.ES | | |
| 1A7.gr.DANADA | DMYPDNADSSYNQKFRE | 10 |
| HVR-H2- | | |
| 1A7.gr.NADS | | |
| HVR-H2- | DMYPDNADASYNQKFRE | 11 |
| 1A7.gr.NADA | | |
| 1A7.gr.DANADA | | |
| HVR-H2- | DMYPDNGDASYNQKFRE | 12 |
| 1A7.gr.NGDA | | |
| HVR-H2- | DMYPDSGDSSYNQKFRE | 13 |
| 1A7.gr.SGDS | | |
| HVR-H2- | DMYPDNGSSSYNQKFRE | 14 |
| 1A7.gr.NGSS | | |
| HVR-H3- | APRWYFSA | 15 |
| 1A7.Ala.8 | | |
| HVR-H3- | APRWYASV | 16 |
| 1A7.Ala.9 | | |
| HVR-H3- | APRWAFSV | 17 |
| 1A7.Ala.10 | | |
| HVR-H3- | APAWYFSV | 18 |
| 1A7.Ala.11 | | |
| HVR-H3- | APRWYFAV | 19 |
| 1A7.Ala.12 | | |
| HVR-H3- | APRAYFSV | 20 |
| 1A7.Ala.13 | | |
| HVR-H3- | AARWYFSV | 21 |
| 1A7.Ala.14 | | |
| HVR-L3- | QQGHTLPAT | 22 |
| 1A7.Ala.1 | | |
| HVR-L3- | QQGHTAPPT | 23 |
| 1A7.Ala.2 | | |
| HVR-L3- | QQGATLPPT | 24 |
| 1A7.Ala.3 | | |
| HVR-L3- | QQGHALPPT | 25 |
| 1A7.Ala.4 | | |
| HVR-L3- | QQAHTLPPT | 26 |
| 1A7.Ala.5 | | |
| HVR-L3- | QQGHTLAPT | 27 |
| 1A7.Ala.6 | | |
| HVR-L3- | QAGHTLPPT | 28 |
| 1A7.Ala.7 | | |
| HVR-H1- | NYLIE | 29 |
| 3C8.gr.1 | | |
| 3C8.gr.2 | | |
| 3C8.gr.3 | | |
| 3C8.gr.4 | | |
| 3C8.gr.5 | | |
| 3C8.gr.5.SG | | |
| 3C8.gr.5.EG | | |
| 3C8.gr.5.QG | | |
| 3C9.gr.5.DQ | | |
| 3C8.gr.5.DA | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.1 | | |
| 3C8.A.2 | | |
| 3C8.A.3 | | |
| 3C8.A.4 | | |
| 3C8.A.5 | | |
| 3C8.A.6 | | |
| 3C8.A.7 | | |
| 3C8.A.8 | | |
| 3C8.A.9 | | |
| 3C8.A.10 | | |
| HVR-H2- | VINPGSGDTYYSEKFKG | 30 |
| 3C8.gr.1 | | |
| 3C8.gr.2 | | |
| 3C8.gr.3 | | |
| 3C8.gr.4 | | |
| 3C8.gr.5 | | |
| 3C8.gr.5.SG | | |
| 3C8.gr.5.EG | | |
| 3C8.gr.5.QG | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.1 | | |
| 3C8.A.2 | | |
| 3C8.A.3 | | |
| 3C8.A.4 | | |
| 3C8.A.5 | | |
| 3C8.A.6 | | |
| 3C8.A.7 | | |
| 3C8.A.8 | | |
| 3C8.A.9 | | |
| 3C8.A.10 | | |
| HVR-H2- | VINPGSGDAYYSEKFKG | 31 |
| 3C8.gr.5.DA | | |
| HVR-H2- | VINPGSGDQYYSEKFKG | 32 |
| 3C8.gr.5.DQ | | |
| HVR-H3- | DRLDY | 33 |
| 3C8.gr.1 | | |
| 3C8.gr.2 | | |
| 3C8.gr.3 | | |
| 3C8.gr.4 | | |
| 3C8.gr.5 | | |
| 3C8.gr.5.SG | | |
| 3C8.gr.5.EG | | |
| 3C8.gr.5.QG | | |
| 3C8.gr.5.DA | | |
| 3C8.gr.5.DQ | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.1 | | |
| 3C8.A.2 | | |
| 3C8.A.3 | | |
| 3C8.A.4 | | |
| 3C8.A.5 | | |
| 3C8.A.6 | | |
| 3C8.A.7 | | |
| HVR-H3- | ARLDY | 34 |
| 3C8.A.8 | | |
| HVR-H3- | DALDY | 35 |
| 3C8.A.9 | | |
| HVR-H3- | DRADY | 36 |
| 3C8.A.10 | | |
| HVR-L1- | HASQDISSYIV | 37 |
| 3C8.gr.1 | | |
| 3C8.gr.2 | | |
| 3C8.gr.3 | | |
| 3C8.gr.4 | | |
| 3C8.gr.5 | | |
| 3C8.gr.5.SG | | |
| 3C8.gr.5.EG | | |
| 3C8.gr.5.QG | | |
| 3C8.gr.5.DA | | |
| 3C8.gr.5.DQ | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.1 | | |
| 3C8.A.2 | | |
| 3C8.A.3 | | |
| 3C8.A.4 | | |
| 3C8.A.5 | | |
| 3C8.A.6 | | |
| 3C8.A.7 | | |
| 3C8.A.8 | | |
| 3C8.A.9 | | |
| 3C8.A.10 | | |
| HVR-L2- | HGTNLED | 38 |
| 3C8.gr.1 | | |
| 3C8.gr.2 | | |
| 3C8.gr.3 | | |
| 3C8.gr.4 | | |
| 3C8.gr.5 | | |
| 3C8.gr.5.DA | | |
| 3C8.gr.5.DQ | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.1 | | |
| 3C8.A.2 | | |
| 3C8.A.3 | | |
| 3C8.A.4 | | |
| 3C8.A.5 | | |
| 3C8.A.6 | | |
| 3C8.A.7 | | |
| 3C8.A.8 | | |
| 3C8.A.9 | | |
| 3C8.A.10 | | |
| HVR-L2- | HGTNLES | 39 |
| 3C8.gr5.SG | | |
| HVR-L2- | HGTNLEE | 40 |
| 3C8.gr.5.EG | | |
| HVR-L2- | HGTNLEQ | 41 |
| 3C8.gr.5.QG | | |
| HVR-L3 | VHYAQFPYT | 42 |
| 3C8.gr.1 | | |
| 3C8.gr.2 | | |
| 3C8.gr.3 | | |
| 3C8.gr.4 | | |
| 3C8.gr.5 | | |
| 3C8.gr.5.SG | | |
| 3C8.gr.5.EG | | |
| 3C8.gr.5.QG | | |
| 3C8.gr.5.DA | | |
| 3C8.gr.5.DQ | | |
| 3C8.gr.6 | | |
| 3C8.gr.7 | | |
| 3C8.gr.8 | | |
| 3C8.gr.9 | | |
| 3C8.gr.10 | | |
| 3C8.gr.11 | | |
| 3C8.A.8 | | |
| 3C8.A.9 | | |
| 3C8.A.10 | | |
| HVR-L3- | AHYAQFPYT | 43 |
| 3C8.A.1 | | |
| HVR-L3- | VAYAQFPYT | 44 |
| 3C8.A.2 | | |
| HVR-L3- | VHAAQFPYT | 45 |
| 3C8.A.3 | | |
| HVR-L3- | VHYAAFPYT | 46 |
| 3C8.A.4 | | |
| HVR-L3- | VHYAQAPYT | 47 |
| 3C8.A.5 | | |
| HVR-L3- | VHYAQFAYT | 48 |
| 3C8.A.6 | | |
| HVR-L3- | VHYAQFPAT | 49 |
| 3C8.A.7 | | |
| HVR-H1- | DYGVL | 50 |
| 1D2.gr.1 | | |
| 1D2.gr.2 | | |
| 1D2.gr.3 | | |
| HVR-H2- | MIWSGGTTDYNAAFIS | 51 |
| 1D2.gr.1 | | |
| 1D2.gr.2 | | |
| 1D2.gr.3 | | |
| HVR-H3- | EEMDY | 52 |
| 1D2.gr.1 | | |
| 1D2.gr.2 | | |
| 1D2.gr.3 | | |
| HVR-L1- | RASQDISNFLN | 53 |
| 1D2.gr.1 | | |
| 1D2.gr.2 | | |
| 1D2.gr.3 | | |
| HVR-L2- | YTSRLHS | 54 |
| 1D2.gr.1 | | |
| 1D2.gr.2 | | |
| 1D2.gr.3 | | |
| HVR-L3- | QQGNTLPWT | 55 |
| 1D2.gr.1 | | |
| 1D2.gr.2 | | |
| 1D2.gr.3 | | |
| 1A7.gr.1 | EVQLVQSGAEVKKPGASVKVSCKASGYTFTDSYMSWVRQAP | 56 |
| V_{H} | GQGLEWIGDMYPDNGDSSYNQKFRERVTITRDTSTSTAYLELS | |
| | SLRSEDTAVYYCVLAPRWYFSVWGQGTLVTVSS | |
| 1A7.gr.1 | DIQMTQSPSSLSASVGDRVTITCRASQDISNYLNWYQQKPGKA | 57 |
| V_{L} | PKLLIYYTSRLRSGVPSRFSGSGSGTDFTLTISSLQPEDFATYYC | |
| | QQGHTLPPTFGQGTKVEIK | |
| 1A7.gr.2 | EVQLVQSGAEVKKPGASVKVSCKASGYTFTDSYMSWVRQAP | 58 |
| V_{H} | GQGLEWIGDMYPDNGDSSYNQKFRERVTITVDTSTSTAYLELS | |
| | SLRSEDTAVYYCVLAPRWYFSVWGQGTLVTVSS | |
| 1A7.gr.2 | DIQMTQSPSSLSASVGDRVTITCRASQDISNYLNWYQQKPGKA | 59 |
| V_{L} | PKLLIYYTSRLRSGVPSRFSGSGSGTDFTLTISSLQPEDFATYYC | |
| | QQGHTLPPTFGQGTKVEIK | |
| 1A7.gr.3 | EVQLVQSGAEVKKPGASVKVSCKASGYTFTDSYMSWVRQAP | 60 |
| V_{H} | GQGLEWIGDMYPDNGDSSYNQKFRERVTLTVDTSTSTAYLEL | |
| | SSLRSEDTAVYYCVLAPRWYFSVWGQGTLVTVSS | |
| 1A7.gr.3 | DIQMTQSPSSLSASVGDRVTITCRASQDISNYLNWYQQKPGKA | 61 |
| V_{L} | PKLLIYYTSRLRSGVPSRFSGSGSGTDFTLTISSLQPEDFATYYC | |
| | QQGHTLPPTFGQGTKVEIK | |
| 1A7.gr.4 | EVQLVQSGAEVKKPGASVKVSCKASGYTFTDSYMSWVRQAP | 62 |
| V_{H} | GQGLEWIGDMYPDNGDSSYNQKFRERVTITVDTSTSTAYLELS | |
| | SLRSEDTAVYYCVLAPRWYFSVWGQGTLVTVSS | |
| 1A7.gr.4 | DIQMTQSPSSLSASVGDRVTITCRASQDISNYLNWYQQKPGKT | 63 |
| V_{L} | VKLLIYYTSRLRSGVPSRFSGSGSGTDFTLTISSLQPEDFATYYC | |
| | QQGHTLPPTFGQGTKVEIK | |
| 1A7.gr.5 | EVQLVQSGAEVKKPGASVKVSCKASGYTFTDSYMSWVRQAP | 64 |
| V_{H} | GQGLEWIGDMYPDNGDSSYNQKFRERVTITVDTSTSTAYLELS | |
| | SLRSEDTAVYYCVLAPRWYFSVWGQGTLVTVSS | |
| 1A7.gr.5 | DIQMTQSPSSLSASVGDRVTITCRASQDISNYLNWYQQKPGKT | 65 |
| V_{L} | VKLLIYYTSRLRSGVPSRFSGSGSGTDFTLTISSLQPEDFATYYC | |
| | QQGHTLPPTFGQGTKVEIK | |
| 1A7.gr.6 | EVQLVQSGAEVKKPGASVKVSCKASGYTFTDSYMSWVRQAP | 66 |
| V_{H} | GQGLEWIGDMYPDNGDSSYNQKFRERVTITVDTSTSTAYLELS | |
| | SLRSEDTAVYYCVLAPRWYFSVWGQGTLVTVSS | |
| 1A7.gr.6 | DIQMTQSPSSLSASVGDRVTITCRASQDISNYLNWYQQKPGKT | 67 |
| V_{L} | VKLLIYYTSRLRSGVPSRFSGSGSGKDYTLTISSLQPEDFATYFC | |
| | QQGHTLPPTFGQGTKVEIK | |
| 1A7.gr.7 | EVQLVQSGAEVKKPGASVKVSCKASGYTFTDSYMSWVRQAP | 68 |
| V_{H} | GQGLEWIGDMYPDNGDSSYNQKFRERVTITVDTSTSTAYLELS | |
| | SLRSEDTAVYYCVLAPRWYFSVWGQGTLVTVSS | |
| 1A7.gr.7 | DIQMTQSPSSLSASVGDRVTITCRASQDISNYLNWYQQKPGKT | 69 |
| V_{L} | VKLLIYYTSRLRSGVPSRFSGSGSGKDYTLTISSLQPEDFATYFC | |
| | QQGHTLPPTFGQGTKVEIK | |
| 1A7.gr.DA | EVQLVQSGAEVKKPGASVKVSCKASGYTFTDAYMSWVRQAP | 70 |
| V_{H} | GQGLEWIGDMYPDNGDSSYNQKFRERVTITRDTSTSTAYLELS | |
| | SLRSEDTAVYYCVLAPRWYFSVWGQGTLVTVSS | |
| 1A7.gr.DA | DIQMTQSPSSLSASVGDRVTITCRASQDISNYLNWYQQKPGKA | 71 |
| V_{L} | PKLLIYYTSRLRSGVPSRFSGSGSGTDFTLTISSLQPEDFATYYC | |
| | QQGHTLPPTFGQGTKVEIK | |
| 1A7.gr.ES | EVQLVQSGAEVKKPGASVKVSCKASGYTFTESYMSWVRQAP | 72 |
| V_{H} | GQGLEWIGDMYPDNGDSSYNQKFRERVTITRDTSTSTAYLELS | |
| | SLRSEDTAVYYCVLAPRWYFSVWGQGTLVTVSS | |
| 1A7.gr.ES | DIQMTQSPSSLSASVGDRVTITCRASQDISNYLNWYQQKPGKA | 73 |
| V_{L} | PKLLIYYTSRLRSGVPSRFSGSGSGTDFTLTISSLQPEDFATYYC | |
| | QQGHTLPPTFGQGTKVEIK | |
| 1A7.gr.NADS | EVQLVQSGAEVKKPGASVKVSCKASGYTFTDSYMSWVRQAP | 74 |
| V_{H} | GQGLEWIGDMYPDNADSSYNQKFRERVTITRDTSTSTAYLELS | |
| | SLRSEDTAVYYCVLAPRWYFSVWGQGTLVTVSS | |
| 1A7.gr.NADS | DIQMTQSPSSLSASVGDRVTITCRASQDISNYLNWYQQKPGKA | 75 |
| V_{L} | PKLLIYYTSRLRSGVPSRFSGSGSGTDFTLTISSLQPEDFATYYC | |
| | QQGHTLPPTFGQGTKVEIK | |
| 1A7.gr.NADA | EVQLVQSGAEVKKPGASVKVSCKASGYTFTDSYMSWVRQAP | 76 |
| V_{H} | GQGLEWIGDMYPDNADASYNQKFRERVTITRDTSTSTAYLELS | |
| | SLRSEDTAVYYCVLAPRWYFSVWGQGTLVTVSS | |
| 1A7.gr.NADA | DIQMTQSPSSLSASVGDRVTITCRASQDISNYLNWYQQKPGKA | 77 |
| V_{L} | PKLLIYYTSRLRSGVPSRFSGSGSGTDFTLTISSLQPEDFATYYC | |
| | QQGHTLPPTFGQGTKVEIK | |
| 1A7.gr.NGDA | EVQLVQSGAEVKKPGASVKVSCKASGYTFTDSYMSWVRQAP | 78 |
| V_{H} | GQGLEWIGDMYPDNGDASYNQKFRERVTITRDTSTSTAYLELS | |
| | SLRSEDTAVYYCVLAPRWYFSVWGQGTLVTVSS | |
| 1A7.gr.NGDA | DIQMTQSPSSLSASVGDRVTITCRASQDISNYLNWYQQKPGKA | 79 |
| V_{L} | PKLLIYYTSRLRSGVPSRFSGSGSGTDFTLTISSLQPEDFATYYC | |
| | QQGHTLPPTFGQGTKVEIK | |
| 1A7.gr.SGDS | EVQLVQSGAEVKKPGASVKVSCKASGYTFTDSYMSWVRQAP | 80 |
| V_{H} | GQGLEWIGDMYPDSGDSSYNQKFRERVTITRDTSTSTAYLELS | |
| | SLRSEDTAVYYCVLAPRWYFSVWGQGTLVTVSS | |
| 1A7.gr.SGDS | DIQMTQSPSSLSASVGDRVTITCRASQDISNYLNWYQQKPGKA | 81 |
| V_{L} | PKLLIYYTSRLRSGVPSRFSGSGSGTDFTLTISSLQPEDFATYYC | |
| | QQGHTLPPTFGQGTKVEIK | |
| 1A7.gr.NGSS | EVQLVQSGAEVKKPGASVKVSCKASGYTFTDSYMSWVRQAP | 82 |
| V_{H} | GQGLEWIGDMYPDNGSSSYNQKFRERVTITRDTSTSTAYLELS | |
| | SLRSEDTAVYYCVLAPRWYFSVWGQGTLVTVSS | |
| 1A7.gr.NGSS | DIQMTQSPSSLSASVGDRVTITCRASQDISNYLNWYQQKPGKA | 83 |
| V_{L} | PKLLIYYTSRLRSGVPSRFSGSGSGTDFTLTISSLQPEDFATYYC | |
| | QQGHTLPPTFGQGTKVEIK | |
| 1A7.gr.DANADA | EVQLVQSGAEVKKPGASVKVSCKASGYTFTDAYMSWVRQAP | 84 |
| V_{H} | GQGLEWIGDMYPDNADASYNQKFRERVTITRDTSTSTAYLELS | |
| | SLRSEDTAVYYCVLAPRWYFSVWGQGTLVTVSS | |
| 1A7.gr.DANADA | DIQMTQSPSSLSASVGDRVTITCRASQDISNYLNWYQQKPGKA | 85 |
| V_{L} | PKLLIYYTSRLRSGVPSRFSGSGSGTDFTLTISSLQPEDFATYYC | |
| | QQGHTLPPTFGQGTKVEIK | |
| 1A7.Ala.1 | EVQLVQSGAEVKKPGASVKVSCKASGYTFTDSYMSWVRQAP | 86 |
| V_{H} | GQGLEWIGDMYPDNGDSSYNQKFRERVTITRDTSTSTAYLELS | |
| | SLRSEDTAVYYCVLAPRWYFSVWGQGTLVTVSS | |
| 1A7.Ala.1 | DIQMTQSPSSLSASVGDRVTITCRASQDISNYLNWYQQKPGKA | 87 |
| V_{L} | PKLLIYYTSRLRSGVPSRFSGSGSGTDFTLTISSLQPEDFATYYC | |
| | QQGHTLPATFGQGTKVEIK | |
| 1A7.Ala.2 | EVQLVQSGAEVKKPGASVKVSCKASGYTFTDSYMSWVRQAP | 88 |
| V_{H} | GQGLEWIGDMYPDNGDSSYNQKFRERVTITRDTSTSTAYLELS | |
| | SLRSEDTAVYYCVLAPRWYFSVWGQGTLVTVSS | |
| 1A7.Ala.2 | DIQMTQSPSSLSASVGDRVTITCRASQDISNYLNWYQQKPGKA | 89 |
| V_{L} | PKLLIYYTSRLRSGVPSRFSGSGSGTDFTLTISSLQPEDFATYYC | |
| | QQGHTAPPTFGQGTKVEIK | |
| 1A7.Ala.3 | EVQLVQSGAEVKKPGASVKVSCKASGYTFTDSYMSWVRQAP | 90 |
| V_{H} | GQGLEWIGDMYPDNGDSSYNQKFRERVTITRDTSTSTAYLELS | |
| | SLRSEDTAVYYCVLAPRWYFSVWGQGTLVTVSS | |
| 1A7.Ala.3 | DIQMTQSPSSLSASVGDRVTITCRASQDISNYLNWYQQKPGKA | 91 |
| V_{L} | PKLLIYYTSRLRSGVPSRFSGSGSGTDFTLTISSLQPEDFATYYC | |
| | QQGATLPPTFGQGTKVEIK | |
| 1A7.Ala.4 | EVQLVQSGAEVKKPGASVKVSCKASGYTFTDSYMSWVRQAP | 92 |
| V_{H} | GQGLEWIGDMYPDNGDSSYNQKFRERVTITRDTSTSTAYLELS | |
| | SLRSEDTAVYYCVLAPRWYFSVWGQGTLVTVSS | |
| 1A7.Ala.4 | DIQMTQSPSSLSASVGDRVTITCRASQDISNYLNWYQQKPGKA | 93 |
| V_{L} | PKLLIYYTSRLRSGVPSRFSGSGSGTDFTLTISSLQPEDFATYYC | |
| | QQGHALPPTFGQGTKVEIK | |
| 1A7.Ala.5 | EVQLVQSGAEVKKPGASVKVSCKASGYTFTDSYMSWVRQAP | 94 |
| V_{H} | GQGLEWIGDMYPDNGDSSYNQKFRERVTITRDTSTSTAYLELS | |
| | SLRSEDTAVYYCVLAPRWYFSVWGQGTLVTVSS | |
| 1A7.Ala.5 | DIQMTQSPSSLSASVGDRVTITCRASQDISNYLNWYQQKPGKA | 95 |
| V_{L} | PKLLIYYTSRLRSGVPSRFSGSGSGTDFTLTISSLQPEDFATYYC | |
| | QQAHTLPPTFGQGTKVEIK | |
| 1A7.Ala.6 | EVQLVQSGAEVKKPGASVKVSCKASGYTFTDSYMSWVRQAP | 96 |
| V_{H} | GQGLEWIGDMYPDNGDSSYNQKFRERVTITRDTSTSTAYLELS | |
| | SLRSEDTAVYYCVLAPRWYFSVWGQGTLVTVSS | |
| 1A7.Ala.6 | DIQMTQSPSSLSASVGDRVTITCRASQDISNYLNWYQQKPGKA | 97 |
| V_{L} | PKLLIYYTSRLRSGVPSRFSGSGSGTDFTLTISSLQPEDFATYYC | |
| | QQGHTLAPTFGQGTKVEIK | |
| 1A7.Ala.7 | EVQLVQSGAEVKKPGASVKVSCKASGYTFTDSYMSWVRQAP | 98 |
| V_{H} | GQGLEWIGDMYPDNGDSSYNQKFRERVTITRDTSTSTAYLELS | |
| | SLRSEDTAVYYCVLAPRWYFSVWGQGTLVTVSS | |
| 1A7.Ala.7 | DIQMTQSPSSLSASVGDRVTITCRASQDISNYLNWYQQKPGKA | 99 |
| V_{L} | PKLLIYYTSRLRSGVPSRFSGSGSGTDFTLTISSLQPEDFATYYC | |
| | QAGHTLPPTFGQGTKVEIK | |
| 1A7.Ala.8 | EVQLVQSGAEVKKPGASVKVSCKASGYTFTDSYMSWVRQAP | 100 |
| V_{H} | GQGLEWIGDMYPDNGDSSYNQKFRERVTITRDTSTSTAYLELS | |
| | SLRSEDTAVYYCVLAPRWYFSAWGQGTLVTVSS | |
| 1A7.Ala.8 | DIQMTQSPSSLSASVGDRVTITCRASQDISNYLNWYQQKPGKA | 101 |
| V_{L} | PKLLIYYTSRLRSGVPSRFSGSGSGTDFTLTISSLQPEDFATYYC | |
| | QQGHTLPPTFGQGTKVEIK | |
| 1A7.Ala.9 | EVQLVQSGAEVKKPGASVKVSCKASGYTFTDSYMSWVRQAP | 102 |
| V_{H} | GQGLEWIGDMYPDNGDSSYNQKFRERVTITRDTSTSTAYLELS | |
| | SLRSEDTAVYYCVLAPRWYASVWGQGTLVTVSS | |
| 1A7.Ala.9 | DIQMTQSPSSLSASVGDRVTITCRASQDISNYLNWYQQKPGKA | 103 |
| V_{L} | PKLLIYYTSRLRSGVPSRFSGSGSGTDFTLTISSLQPEDFATYYC | |
| | QQGHTLPPTFGQGTKVEIK | |
| 1A7.Ala.10 | EVQLVQSGAEVKKPGASVKVSCKASGYTFTDSYMSWVRQAP | 104 |
| V_{H} | GQGLEWIGDMYPDNGDSSYNQKFRERVTITRDTSTSTAYLELS | |
| | SLRSEDTAVYYCVLAPRWAFSVWGQGTLVTVSS | |
| 1A7.Ala.10 | DIQMTQSPSSLSASVGDRVTITCRASQDISNYLNWYQQKPGKA | 105 |
| V_{L} | PKLLIYYTSRLRSGVPSRFSGSGSGTDFTLTISSLQPEDFATYYC | |
| | QQGHTLPPTFGQGTKVEIK | |
| 1A7.Ala.11 | EVQLVQSGAEVKKPGASVKVSCKASGYTFTDSYMSWVRQAP | 106 |
| V_{H} | GQGLEWIGDMYPDNGDSSYNQKFRERVTITRDTSTSTAYLELS | |
| | SLRSEDTAVYYCVLAPAWYFSVWGQGTLVTVSS | |
| 1A7.Ala.11 | DIQMTQSPSSLSASVGDRVTITCRASQDISNYLNWYQQKPGKA | 107 |
| V_{L} | PKLLIYYTSRLRSGVPSRFSGSGSGTDFTLTISSLQPEDFATYYC | |
| | QQGHTLPPTFGQGTKVEIK | |
| 1A7.Ala.12 | EVQLVQSGAEVKKPGASVKVSCKASGYTFTDSYMSWVRQAP | 108 |
| V_{H} | GQGLEWIGDMYPDNGDSSYNQKFRERVTITRDTSTSTAYLELS | |
| | SLRSEDTAVYYCVLAPRWYFAVWGQGTLVTVSS | |
| 1A7.Ala.12 | DIQMTQSPSSLSASVGDRVTITCRASQDISNYLNWYQQKPGKA | 109 |
| V_{L} | PKLLIYYTSRLRSGVPSRFSGSGSGTDFTLTISSLQPEDFATYYC | |
| | QQGHTLPPTFGQGTKVEIK | |
| 1A7.Ala.13 | EVQLVQSGAEVKKPGASVKVSCKASGYTFTDSYMSWVRQAP | 110 |
| V_{H} | GQGLEWIGDMYPDNGDSSYNQKFRERVTITRDTSTSTAYLELS | |
| | SLRSEDTAVYYCVLAPRAYFSVWGQGTLVTVSS | |
| 1A7.Ala.13 | DIQMTQSPSSLSASVGDRVTITCRASQDISNYLNWYQQKPGKA | 111 |
| V_{L} | PKLLIYYTSRLRSGVPSRFSGSGSGTDFTLTISSLQPEDFATYYC | |
| | QQGHTLPPTFGQGTKVEIK | |
| 1A7.Ala.14 | EVQLVQSGAEVKKPGASVKVSCKASGYTFTDSYMSWVRQAP | 112 |
| V_{H} | GQGLEWIGDMYPDNGDSSYNQKFRERVTITRDTSTSTAYLELS | |
| | SLRSEDTAVYYCVLAARWYFSVWGQGTLVTVSS | |
| 1A7.Ala.14 | DIQMTQSPSSLSASVGDRVTITCRASQDISNYLNWYQQKPGKA | 113 |
| V_{L} | PKLLIYYTSRLRSGVPSRFSGSGSGTDFTLTISSLQPEDFATYYC | |
| | QQGHTLPPTFGQGTKVEIK | |
| 1A7.Ala.15 | EVQLVQSGAEVKKPGASVKVSCKASGYTFTDSYMSWVRQAP | 114 |
| V_{H} | GQGLEWIGDMYPDNGDSSYNQKFRERVTITRDTSTSTAYLELS | |
| | SLRSEDTAVYYCALAPRWYFSVWGQGTLVTVSS | |
| 1A7.Ala.15 | DIQMTQSPSSLSASVGDRVTITCRASQDISNYLNWYQQKPGKA | 115 |
| V_{L} | PKLLIYYTSRLRSGVPSRFSGSGSGTDFTLTISSLQPEDFATYYC | |
| | QQGHTLPPTFGQGTKVEIK | |
| 1A7.Ala.16 | EVQLVQSGAEVKKPGASVKVSCKASGYTFTDSYMSWVRQAP | 116 |
| V_{H} | GQGLEWIGDMYPDNGDSSYNQKFRERVTITRDTSTSTAYLELS | |
| | SLRSEDTAVYYCVAAPRWYFSVWGQGTLVTVSS | |
| 1A7.Ala.16 | DIQMTQSPSSLSASVGDRVTITCRASQDISNYLNWYQQKPGKA | 117 |
| V_{L} | PKLLIYYTSRLRSGVPSRFSGSGSGTDFTLTISSLQPEDFATYYC | |
| | QQGHTLPPTFGQGTKVEIK | |
| 3C8.gr.1 | EVQLVQSGAEVKKPGASVKVSCKASGYAFTNYLIEWVRQAPG | 118 |
| V_{H} | QGLEWIGVINPGSGDTYYSEKFKGRVTITRDTSTSTAYLELSSL | |
| | RSEDTAVYYCARDRLDYWGQGTLVTVSS | |
| 3C8.gr.1 | DIQMTQSPSSLSASVGDRVTITCHASQDISSYIVWYQQKPGKAP | 119 |
| V_{L} | KLLIYHGTNLEDGVPSRFSGSGSGTDFTLTISSLQPEDFATYYC | |
| | VHYAQFPYTFGQGTKVEIK | |
| 3C8.gr.2 | EVQLVQSGAEVKKPGASVKVSCKASGYAFTNYLIEWVRQAPG | 120 |
| V_{H} | QGLEWIGVINPGSGDTYYSEKFKGRVTITADTSTSTAYLELSSL | |
| | RSEDTAVYYCARDRLDYWGQGTLVTVSS | |
| 3C8.gr.2 | DIQMTQSPSSLSASVGDRVTITCHASQDISSYIVWYQQKPGKAP | 121 |
| V_{L} | KLLIYHGTNLEDGVPSRFSGSGSGTDFTLTISSLQPEDFATYYC | |
| | VHYAQFPYTFGQGTKVEIK | |
| 3C8.gr.3 | EVQLVQSGAEVKKPGASVKVSCKASGYAFTNYLIEWVRQAPG | 122 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.gr.3 | DIQMTQSPSSLSASVGDRVTITCHASQDISSYIVWYQQKPGKAP | 123 |
| V_{L} | KLLIYHGTNLEDGVPSRFSGSGSGTDFTLTISSLQPEDFATYYC | |
| | VHYAQFPYTFGQGTKVEIK | |
| 3C8.gr.4 | EVQLVQSGAEVKKPGASVKVSCKASGYAFTNYLIEWVRQAPG | 124 |
| V_{H} | QGLEWIGVINPGSGDTYYSEKFKGRVTITADTSTSTAYLELSSL | |
| | RSEDTAVYYCARDRLDYWGQGTLVTVSS | |
| 3C8.gr.4 | DIQMTQSPSSLSASVGDRVTITCHASQDISSYIVWYQQKPGKSF | 125 |
| V_{L} | KGLIYHGTNLEDGVPSRFSGSGSGTDFTLTISSLQPEDFATYYC | |
| | VHYAQFPYTFGQGTKVEIK | |
| 3C8.gr.5 | EVQLVQSGAEVKKPGASVKVSCKASGYAFTNYLIEWVRQAPG | 126 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.gr.5 | DIQMTQSPSSLSASVGDRVTITCHASQDISSYIVWYQQKPGKSF | 127 |
| V_{L} | KGLIYHGTNLEDGVPSRFSGSGSGTDFTLTISSLQPEDFATYYC | |
| | VHYAQFPYTFGQGTKVEIK | |
| 3C8.gr.5.SG | EVQLVQSGAEVKKPGASVKVSCKASGYAFTNYLIEWVRQAPG | 128 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.gr.5.SG | DIQMTQSPSSLSASVGDRVTITCHASQDISSYIVWYQQKPGKSF | 129 |
| V_{L} | KGLIYHGTNLESGVPSRFSGSGSGTDFTLTISSLQPEDFATYYCV | |
| | HYAQFPYTFGQGTKVEIK | |
| 3C8.gr.5.EG | EVQLVQSGAEVKKPGASVKVSCKASGYAFTNYLIEWVRQAPG | 130 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.gr.5.EG | DIQMTQSPSSLSASVGDRVTITCHASQDISSYIVWYQQKPGKSF | 131 |
| V_{L} | KGLIYHGTNLEEGVPSRFSGSGSGTDFTLTISSLQPEDFATYYC | |
| | VHYAQFPYTFGQGTKVEIK | |
| 3C8.gr.5.QG | EVQLVQSGAEVKKPGASVKVSCKASGYAFTNYLIEWVRQAPG | 132 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.gr.5.QG | DIQMTQSPSSLSASVGDRVTITCHASQDISSYIVWYQQKPGKSF | 133 |
| V_{L} | KGLIYHGTNLEQGVPSRFSGSGSGTDFTLTISSLQPEDFATYYC | |
| | VHYAQFPYTFGQGTKVEIK | |
| 3C8.gr.6 | EVQLVQSGAEVKKPGASVKVSCKASGYAFTNYLIEWVRQAPG | 134 |
| V_{H} | QGLEWIGVINPGSGDTYYSEKFKGRVTITADTSTSTAYLELSSL | |
| | RSEDTAVYYCARDRLDYWGQGTLVTVSS | |
| 3C8.gr.6 | DIQMTQSPSSLSASVGDRVTITCHASQDISSYIVWYQQKPGKSF | 135 |
| V_{L} | KGLIYHGTNLEDGVPSRFSGSGSGADYTLTISSLQPEDFATYYC | |
| | VHYAQFPYTFGQGTKVEIK | |
| 3C8.gr.7 | EVQLVQSGAEVKKPGASVKVSCKASGYAFTNYLIEWVRQAPG | 136 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.gr.7 | DIQMTQSPSSLSASVGDRVTITCHASQDISSYIVWYQQKPGKSF | 137 |
| V_{L} | KGLIYHGTNLEDGVPSRFSGSGSGADYTLTISSLQPEDFATYYC | |
| | VHYAQFPYTFGQGTKVEIK | |
| 3C8.gr.8 | EVQLVQSGAEVKKPGASVKVSCKASGYAFTNYLIEWVRQAPG | 138 |
| V_{H} | QGLEWIGVINPGSGDTYYSEKFKGRVTLTRDTSTSTAYLELSSL | |
| | RSEDTAVYYCARDRLDYWGQGTLVTVSS | |
| 3C8.gr.8 | DIQMTQSPSSLSASVGDRVTITCHASQDISSYIVWYQQKPGKSF | 139 |
| V_{L} | KGLIYHGTNLEDGVPSRFSGSGSGTDFTLTISSLQPEDFATYYC | |
| | VHYAQFPYTFGQGTKVEIK | |
| 3C8.gr.9 | EVQLVQSGAEVKKPGASVKVSCKASGYAFTNYLIEWVRQAPG | 140 |
| V_{H} | QGLEWIGVINPGSGDTYYSEKFKGRVTLTRDTSTSTAYLELSSL | |
| | RSEDTAVYYCARDRLDYWGQGTLVTVSS | |
| 3C8.gr.9 | DIQMTQSPSSLSASVGDRVTITCHASQDISSYIVWYQQKPGKSP | 141 |
| V_{L} | KLLIYHGTNLEDGVPSRFSGSGSGTDFTLTISSLQPEDFATYYC | |
| | VHYAQFPYTFGQGTKVEIK | |
| 3C8.gr.10 | EVQLVQSGAEVKKPGASVKVSCKASGYAFTNYLIEWVRQAPG | 142 |
| V_{H} | QGLEWIGVINPGSGDTYYSEKFKGRVTLTRDTSTSTAYLELSSL | |
| | RSEDTAVYYCARDRLDYWGQGTLVTVSS | |
| 3C8.gr.10 | DIQMTQSPSSLSASVGDRVTITCHASQDISSYIVWYQQKPGKAF | 143 |
| V_{L} | KLLIYHGTNLEDGVPSRFSGSGSGTDFTLTISSLQPEDFATYYC | |
| | VHYAQFPYTFGQGTKVEIK | |
| 3C8.gr.11 | EVQLVQSGAEVKKPGASVKVSCKASGYAFTNYLIEWVRQAPG | 144 |
| V_{H} | QGLEWIGVINPGSGDTYYSEKFKGRVTLTRDTSTSTAYLELSSL | |
| | RSEDTAVYYCARDRLDYWGQGTLVTVSS | |
| 3C8.gr.11 | DIQMTQSPSSLSASVGDRVTITCHASQDISSYIVWYQQKPGKAP | 145 |
| V_{L} | KGLIYHGTNLEDGVPSRFSGSGSGTDFTLTISSLQPEDFATYYC | |
| | VHYAQFPYTFGQGTKVEIK | |
| 3C8.A.1 | EVQLVQSGAEVKKPGASVKVSCKASGYAFTNYLIEWVRQAPG | 146 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.A.1 | DIQMTQSPSSLSASVGDRVTITCHASQDISSYIVWYQQKPGKSF | 147 |
| V_{L} | KGLIYHGTNLEDGVPSRFSGSGSGTDFTLTISSLQPEDFATYYC | |
| | AHYAQFPYTFGQGTKVEIK | |
| 3C8.A.2 | EVQLVQSGAEVKKPGASVKVSCKASGYAFTNYLIEWVRQAPG | 148 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.A.2 | DIQMTQSPSSLSASVGDRVTITCHASQDISSYIVWYQQKPGKSF | 149 |
| V_{L} | KGLIYHGTNLEDGVPSRFSGSGSGTDFTLTISSLQPEDFATYYC | |
| | VAYAQFPYTFGQGTKVEIK | |
| 3C8.A.3 | EVQLVQSGAEVKKPGASVKVSCKASGYAFTNYLIEWVRQAPG | 150 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.A.3 | DIQMTQSPSSLSASVGDRVTITCHASQDISSYIVWYQQKPGKSF | 151 |
| V_{L} | KGLIYHGTNLEDGVPSRFSGSGSGTDFTLTISSLQPEDFATYYC | |
| | VHAAQFPYTFGQGTKVEIK | |
| 3C8.A.4 | EVQLVQSGAEVKKPGASVKVSCKASGYAFTNYLIEWVRQAPG | 152 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.A.4 | DIQMTQSPSSLSASVGDRVTITCHASQDISSYIVWYQQKPGKSF | 153 |
| V_{L} | KGLIYHGTNLEDGVPSRFSGSGSGTDFTLTISSLQPEDFATYYC | |
| | VHYAAFPYTFGQGTKVEIK | |
| 3C8.A.5 | EVQLVQSGAEVKKPGASVKVSCKASGYAFTNYLIEWVRQAPG | 154 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.A.5 | DIQMTQSPSSLSASVGDRVTITCHASQDISSYIVWYQQKPGKSF | 155 |
| V_{L} | KGLIYHGTNLEDGVPSRFSGSGSGTDFTLTISSLQPEDFATYYC | |
| | VHYAQAPYTFGQGTKVEIK | |
| 3C8.A.6 | EVQLVQSGAEVKKPGASVKVSCKASGYAFTNYLIEWVRQAPG | 156 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.A.6 | DIQMTQSPSSLSASVGDRVTITCHASQDISSYIVWYQQKPGKSF | 157 |
| V_{L} | KGLIYHGTNLEDGVPSRFSGSGSGTDFTLTISSLQPEDFATYYC | |
| | VHYAQFAYTFGQGTKVEIK | |
| 3C8.A.7 | EVQLVQSGAEVKKPGASVKVSCKASGYAFTNYLIEWVRQAPG | 158 |
| V_{H} | QGLEWIGVINPGSGDTYYSEKFKGRVTLTADTSTSTAYLELSSL | |
| | RSEDTAVYYCARDRLDYWGQGTLVTVSS | |
| 3C8.A.7 | DIQMTQSPSSLSASVGDRVTITCHASQDISSYIVWYQQKPGKSF | 159 |
| V_{L} | KGLIYHGTNLEDGVPSRFSGSGSGTDFTLTISSLQPEDFATYYC | |
| | VHYAQFPATFGQGTKVEIK | |
| 3C8.A.8 | EVQLVQSGAEVKKPGASVKVSCKASGYAFTNYLIEWVRQAPG | 160 |
| V_{H} | QGLEWIGVINPGSGDTYYSEKFKGRVTLTADTSTSTAYLELSSL | |
| | RSEDTAVYYCARARLDYWGQGTLVTVSS | |
| 3C8.A.8 | DIQMTQSPSSLSASVGDRVTITCHASQDISSYIVWYQQKPGKSF | 161 |
| V_{L} | KGLIYHGTNLEDGVPSRFSGSGSGTDFTLTISSLQPEDFATYYC | |
| | VHYAQFPYTFGQGTKVEIK | |
| 3C8.A.9 | EVQLVQSGAEVKKPGASVKVSCKASGYAFTNYLIEWVRQAPG | 162 |
| V_{H} | QGLEWIGVINPGSGDTYYSEKFKGRVTLTADTSTSTAYLELSSL | |
| | RSEDTAVYYCARDALDYWGQGTLVTVSS | |
| 3C8.A.9 | DIQMTQSPSSLSASVGDRVTITCHASQDISSYIVWYQQKPGKSF | 163 |
| V_{L} | KGLIYHGTNLEDGVPSRFSGSGSGTDFTLTISSLQPEDFATYYC | |
| | VHYAQFPYTFGQGTKVEIK | |
| 3C8.A.10 | EVQLVQSGAEVKKPGASVKVSCKASGYAFTNYLIEWVRQAPG | 164 |
| V_{H} | QGLEWIGVINPGSGDTYYSEKFKGRVTLTADTSTSTAYLELSSL | |
| | RSEDTAVYYCARDRADYWGQGTLVTVSS | |
| 3C8.A.10 | DIQMTQSPSSLSASVGDRVTITCHASQDISSYIVWYQQKPGKSF | 165 |
| V_{L} | KGLIYHGTNLEDGVPSRFSGSGSGTDFTLTISSLQPEDFATYYC | |
| | VHYAQFPYTFGQGTKVEIK | |
| 1D2.gr.1 | EVQLVESGPGLVKPSETLSLTCTVSGFSLTDYGVLWIRQPPGKG | 166 |
| V_{H} | LEWIGMIWSGGTTDYNAAFISRVTISVDTSKNQFSLKLSSVTAA | |
| | DTAVYYCVREEMDYWGQGTLVTVSS | |
| 1D2.gr.1 | DIQMTQSPSSLSASVGDRVTITCRASQDISNFLNWYQQKPGKA | 167 |
| V_{L} | PKLLIYYTSRLHSGVPSRFSGSGSGTDFTLTISSLQPEDFATYYC | |
| | QQGNTLPWTFGQGTKVEIK | |
| 1D2.gr.2 | EVQLVESGPGLVKPSETLSLTCTVSGFSLTDYGVLWIRQPPGKG | 168 |
| V_{H} | LEWIGMIWSGGTTDYNAAFISRVTISKDTSKNQVSLKLSSVTA | |
| | ADTAVYYCVREEMDYWGQGTLVTVSS | |
| 1D2.gr.2 | DIQMTQSPSSLSASVGDRVTITCRASQDISNFLNWYQQKPGKA | 169 |
| V_{L} | PKLLIYYTSRLHSGVPSRFSGSGSGTDFTLTISSLQPEDFATYYC | |
| | QQGNTLPWTFGQGTKVEIK | |
| 1D2.gr.3 | EVQLVESGPGLVKPSETLSLTCTVSGFSLTDYGVLWVRQPPGK | 170 |
| V_{H} | GLEWLGMIWSGGTTDYNAAFISRLTISKDTSKNQVSLKLSSVT | |
| | AADTAVYYCVREEMDYWGQGTLVTVSS | |
| 1D2.gr.3 | DIQMTQSPSSLSASVGDRVTITCRASQDISNFLNWYQQKPGKA | 171 |
| V_{L} | PKLLIYYTSRLHSGVPSRFSGSGSGTDFTLTISSLQPEDFATYYC | |
| | QQGNTLPWTFGQGTKVEIK | |
| CON1 | X₁X₂YMS, wherein X₁ is D or E, and X₂ is S or A | 172 |
| (1A7)HVR-H1 | | |
| CON1 (1A7) | DMYPDX₁X₂X₃X₄SYNQKFRE, wherein X₁ is N or S, X₂ is A or G, | 173 |
| HVR-H2 | X₃ is D or S, and X₄ is A or S | |
| CON1 (1A7) | APRWX₁X₂X₃X₄, wherein X₁ is Y or A, X₂ is A or F, X₃ is S or A, | 174 |
| HVR-H3 | and X₄ is A or V. | |
| CON1 (1A7) | QX₁X₂X₃X₄X₅X₆X₇T, wherein X₁ is A or Q, X₂ is A or G, X₃ is A or | 175 |
| HVR-L3 | H, X₄ is A or T, X₅ is A or L, X₆ is A or P, and X₇ is A or P. | |
| CON2 (3C8) | VINPGSGDX₁YYSEKFKG, wherein X₁ is T, A or Q. | 176 |
| HVR-H2 | | |
| CON2 (3C8) | HGTNLEX₁, wherein X₁ is S, E, or Q. | 177 |
| HVR-L2 | | |
| CON2 (3C8) | X₁X₂YAQFPYX₃, wherein X₁ is V or A, X₂ is H or A, and X₃ is Y or | 178 |
| HVR-L3 | A. | |
| 1A7 V_{L} | DIQMTQTTSSLSASLGDRVTISCRASQDISNYLNWYQQKPDGT | 179 |
| | VKLLIYYTSRLRSGVPSRFSGSGSGKDYFLTISNLEQEDVAAYF | |
| | CQQGHTLPPTFGGGTKLEIK | |
| 1A7 V_{H} | EVQLQQSGPELVKPGASVKISCKASGYTFTDSYMSWVKQSHG | 180 |
| | KTLEWIGDMYPDNGDSSYNQKFREKVTLTVDKSSTTAYMEFR | |
| | SLTSEDSAVYYCVLAPRWYFSVWGTGTTVTVSS | |
| 3C8 V_{L} | DILMTQSPSSMSVSLGDTVSITCHASQDISSYIVWLQQKPGKSF | 181 |
| | RGLIYHGTNLEDGIPSRFSGSGSGADYSLTISSLESEDFADYYCV | |
| | HYAQFPYTFGGGTKLEIK | |
| 3C8 V_{H} | QVQLQQSGAELVRPGTSVKVSCKASGYAFTNYLIEWVKQRPG | 182 |
| | QGLEWIGVINPGSGDTYYSEKFKGKVTLTADKSSSTAYMQLSS | |
| | LTSEDSAVYFCARDRLDYWGQGTTLTVSS | |
| 1A7.gr.5′ | EVQLVQSGAEVKKPGASVKVSCKASGYTFTDSYMSWVRQAP | 183 |
| V_{H} | GQGLEWIGDMYPDNGDSSYNQKFRERVTLTVDTSTSTAYLEL | |
| | SSLRSEDTAVYYCVLAPRWYFSVWGQGTLVTVSS | |
| 1A7.gr.7′ | EVQLVQSGAEVKKPGASVKVSCKASGYTFTDSYMSWVRQAP | 184 |
| V_{H} | GQGLEWIGDMYPDNGDSSYNQKFRERVTLTVDTSTSTAYLEL | |
| | SSLRSEDTAVYYCVLAPRWYFSVWGQGTLVTVSS | |

### EXAMPLES

### Example 1: A Phase I Dose Escalation Study of the Safety and Pharmacokinetics of MOXR0916 in Patients with Locally Advanced or Metastatic Solid Tumors

### Study Design

This is a first-in-human, Phase I, open-label, multicenter, dose-escalation study designed to evaluate the safety, tolerability, and pharmacokinetics of MOXR0916 (1A7.grl IgG1) in patients with locally advanced or metastatic solid tumors that have progressed after all available standard therapy or for which standard therapy has proven to be ineffective or intolerable, or is considered inappropriate. Approximately 200-400 patients may be enrolled in this study at multiple centers worldwide.

This study includes a screening period, an initial treatment period, a re-treatment period applicable to a subset of patients who discontinue MOXR0916 after demonstration of prolonged clinical benefit, and a post-treatment follow-up period. Patients are enrolled in two stages: a dose-escalation stage and an expansion stage.

As described in greater detail below, MOXR0916 is administered by IV infusion on Day 1 of 21-day cycles. In the absence of unacceptable toxicity or compelling evidence of disease progression, treatment may be continued beyond Cycle 1 based on a favorable assessment of benefit and risk by the investigator.

All adverse events (AEs) are monitored and recorded for at least 90 days after the last dose of study treatment or until initiation of another systemic anti-cancer therapy, whichever occurs first. After this period, the Sponsor should be notified if the investigator becomes aware of any post-study serious adverse events (SAEs), regardless of causality. Adverse events are graded according to the National Cancer Institute Common Terminology Criteria for Adverse Events Version 4.0 (NCI CTCAE v4.0).

To characterize the pharmacokinetic (PK) properties of MOXR0916 and pharmacodynamic (PD) responses to treatment, blood samples are taken at various timepoints before and after dosing. Patients undergo tumor assessments at screening and during the study. Patients may be permitted to continue study treatment even if standard RECIST v1.1 criteria for progressive disease are met, provided they meet the criteria for continued treatment. All patients who discontinue the initial study treatment for reasons other than disease progression (e.g., achievement of a confirmed complete response, adverse events) continue tumor assessments. Patients who discontinue study treatment after demonstration of prolonged clinical benefit may be eligible to reinitiate study treatment upon radiographic progression.

Patients who discontinue study treatment return to the clinic for a treatment discontinuation visit within 30 days after the last dose of MOXR0916 during the initial and re-treatment periods, respectively as applicable. All patients are followed for survival and subsequent anti-cancer therapy information approximately every 3 months until death, loss to follow-up, or study termination, unless the patient requests to be withdrawn from follow-up.

### Study Objectives

The primary objective for this study is to evaluate the safety and tolerability of MOXR0916 in patients with locally advanced or metastatic solid tumors.

The secondary objectives for this study are as follows:

(a) To estimate the maximum tolerated dose (MTD) and characterize the dose-limiting toxicities (DLTs) of MOXR0916;
(b) To identify a recommended Phase II dose for MOXR0916;
(c) To characterize the pharmacokinetics of MOXR0916;
(d) To characterize the immunogenic potential of MOXR0916 by measuring anti-MOXR0916 antibodies and assessing their relationship with other outcome measures; and
(e) To make a preliminary assessment of the anti-tumor activity of MOXR0916 in patients with locally advanced or metastatic solid tumors.

The exploratory objectives for this study are as follows:

(a) To make a preliminary assessment of biomarkers that might act as PD indicators of activity of MOXR0916 in patients with locally advanced or metastatic solid tumors; and
(b) To make a preliminary assessment of biomarkers that might act as predictors of anti-tumor activity of MOXR0916 in patients with locally advanced or metastatic solid tumors.

### Study Population

Patients must meet the following criteria for study entry, which include cancer-specific (both general and specific for the dose-expansion stage) and general inclusion criteria.

Cancer-specific inclusion criteria include the following:

(a) Histologic documentation of locally advanced, recurrent or metastatic incurable solid malignancy that has progressed after all available standard therapy or for which standard therapy has proven to be ineffective or intolerable, or is considered inappropriate;
(b) Confirmed availability of representative tumor specimens in paraffin blocks (preferred) or ≥15 unstained slides, with an associated pathology report. Only tissue from a surgical resection or a core needle, punch, or excisional/incisional biopsy sample collection may be accepted. Fine-needle aspiration, brushing, and lavage samples are not acceptable. If adequate tissue from distinct time points (such as time of initial diagnosis and time of disease recurrence) and/or multiple metastatic tumors is available, priority should be given to the tissue most recently collected (ideally subsequent to the most recent systemic therapy). Multiple samples may be collected for a given patient, on the basis of availability; however, the requirement for a block or ≥15 unstained slides should be satisfied by a single biopsy or resection specimen. A patient with insufficient or unavailable archival tissue may be eligible, upon discussion with the Medical Monitor, if the patient meets any of the following: can provide at least 10 unstained, serial slides; Is willing to consent to and undergo a pretreatment core, punch, or excisional/incisional biopsy sample collection of the tumor; or is enrolled in a dose-escalation cohort;
(c) Measurable disease per RECIST v1.1 (for RECIST v1.1 criteria and additional descriptions related to measurements of tumors and tumor response, see, e.g., Eisenhauer, E. A. et al. (2009) Eur. J. Cancer 45:228-247).

In some embodiments, modified RECIST criteria may be used to evaluate tumor response. Modified Response Evaluation Criteria in Solid Tumors (RECIST) is derived from RECIST, Version 1.1 (v1.1) conventions (see, e.g., Eisenhauer, E. A. et al. (2009) Eur. J. Cancer 45:228-247) and immune-related response criteria (irRC; see, e.g., Wolchok et al. (2009) Clin. Can. Res. 15:7412-7420; Nishino et al. (2014) J. Immunother. Can. 2:17; and Nishino et al. (2013) Clin. Can. Res. 19:3936-3943). Conventional response criteria may not be adequate to characterize the anti-tumor activity of immunotherapeutic agents like MOXR0916, which can produce delayed responses that may be preceded by initial apparent radiographic progression, including the appearance of new lesions. Therefore, modified response criteria have been developed that account for the possible appearance of new lesions and allow radiological progression to be confirmed at a subsequent assessment. For a summary of the changes between modified RECIST and RECIST v1.1, see Table B above.

When not otherwise specified, RECIST v1.1 conventions apply. Briefly, RECIST v1.1 criteria for determining objective tumor response for target lesions include:

(a) Complete response (CR): disappearance of all target lesions. Any pathological lymph nodes (whether target or non-target) must have reduction in short axis to <10 mm;
(b) Partial response (PR): at least a 30% decrease in the sum of diameters of target lesions, taking as reference the baseline sum of diameters;
(c) Progressive disease (PD): at least a 20% increase in the sum of diameters of target lesions, taking as reference the smallest sum on study (nadir), including baseline. In addition to the relative increase of 20%, the sum must also demonstrate an absolute increase of at least 5 mm. The appearance of one or more new lesions is also considered progression; and
(d) Stable disease (SD): neither sufficient shrinkage to qualify for PR nor sufficient increase to qualify for PD, taking as reference the smallest sum on study.

Cancer-specific inclusion criteria unique to patients in the dose-expansion stage include the following:

(a) Expansion Part I biopsy cohort: Accessible lesion(s) that permit a total of at least two biopsies (pretreatment and on-treatment) without unacceptable risk of a significant procedural complication. Acceptable samples include core needle biopsies for deep tumor tissue or lymph nodes or excisional, incisional, punch, or forceps biopsies for cutaneous, subcutaneous, or mucosal lesions. Fine needle aspirates are not permitted. Target lesions considered for core needle biopsies should be deemed suitable for retrieval of at least three cores;
(b) Expansion Part II biopsy cohort: Cutaneous or subcutaneous tumors ≥5 mm in diameter amenable to serial biopsy by excisional, incisional or punch biopsies without unacceptable risk of a major procedural complication. If more than one biopsy is planned to be taken from one lesion, the lesion must be large enough to permit successive biopsies ≥1 cm apart;
(c) Melanoma cohort: Histologically confirmed incurable, advanced metastatic melanoma (patients whose tumors have a known BRAF V600 mutation must also have experienced disease progression, during or after treatment, or intolerance to treatment with a BRAF and/or mitogen-activated protein kinase kinase (MEK) kinase inhibitor);
(d) RCC cohort: Histologically confirmed incurable, advanced RCC with component of clear cell histology and/or component of sarcomatoid histology;
(e) TNBC cohort: Histologically confirmed incurable, advanced estrogen receptor-, progesterone receptor-, and human epidermal growth factor receptor 2 (HER2)-negative (triple-negative) adenocarcinoma of the breast, as defined by the American Society of Clinical Oncology College of American Pathologists (ASCO-CAP) guidelines:
(i) <1% of tumor cell nuclei are immunoreactive for estrogen receptor and <1% of tumor cell nuclei are immunoreactive for progesterone receptor (Hammond, M. E. et al. (2010) J. Clin. Oncol. 28:2784-2795) AND
(ii) HER2 tests demonstrate either immunohistochemistry (IHC) 1+, IHC 0 or in situ hybridization (ISH) negative (Wolff, A. C. et al. (2013) J. Clin. Oncol. 31:3997:4013);
   (f) NSCLC cohort: Histologically confirmed incurable, advanced NSCLC:
(i) Patients whose tumors have a known sensitizing epidermal growth factor receptor (EGFR) mutation must also have experienced disease progression (during or after treatment) or intolerance to treatment with an EGFR tyrosine kinase inhibitor;
(ii) Patients whose tumors have a known anaplastic lymphoma kinase (ALK) rearrangement must also have experienced disease progression (during or after treatment) or intolerance to treatment with an ALK tyrosine kinase inhibitor;
   (g) UBC cohort: Histologically confirmed incurable, advanced transitional cell carcinoma of the urothelium (including renal pelvis, ureters, urinary bladder, urethra) (Patients with mixed histologies are required to have a dominant transitional cell pattern);
   (h) CRC cohort: Histologically confirmed incurable, advanced adenocarcinoma of the colon or rectum (Tumors of appendiceal origin are not eligible); and
   (i) OC cohort: Histologically confirmed incurable, advanced epithelial ovarian, fallopian tube, or primary peritoneal cancer.

General inclusion criteria include the following:

(a) Age ≥18 years;
(b) Eastern Cooperative Oncology Group (ECOG) performance status of 0 or 1 (see Table C below for a description of this 0-5 point scale);
(c) Life expectancy ≥12 weeks;
(d) Adequate hematologic and end organ function, defined by the following laboratory results obtained within 14 days prior to the first study treatment (Cycle 1, Day 1):
(i) Absolute neutrophil count (ANC) ≥1500 cells/μL;
(ii) White blood cell (WBC) counts ≥2,500/μL;
(iii) Lymphocyte count ≥500/μL;
(iv) Platelet count ≥100,000/μL (without transfusion within 14 days prior to Cycle 1, Day 1);
(v) Hemoglobin ≥9.0 g/dL (patients may be transfused or receive erythropoietic treatment to meet this criterion);
(vi) Total bilirubin ≤1.5× upper limit of normal (ULN);
(vii) Aspartate aminotransferase (AST) and alanine aminotransferase (ALT)≤3.0×ULN
(viii) Alkaline phosphatase ≤2.5×ULN with the following exception: Patients with documented liver or bone metastases: alkaline phosphatase ≤5×ULN;
(ix) Serum albumin ≥2.5 g/dL;
(x) Prothrombin time (PT) and activated partial thromboplastin time (aPTT)≤1.5×ULN (This applies only to patients who do not receive therapeutic anticoagulation; patients receiving therapeutic anticoagulation should be on a stable dose);
(xi) Measured or calculated creatinine clearance ≥50 mL/min on the basis of the Cockcroft-Gault glomerular filtration rate estimation:

$$\frac{( 140 - age ) \times ( weight in kg ) \times ( 0.85 if female )}{72 \times ( serum creatinine in mg \text{/} dL ) ;}$$

(e) For female patients of childbearing potential and male patients with partners of childbearing potential, agreement (by patient and/or partner) to use highly effective form(s) of contraception (i.e., one that results in a low failure rate [<1% per year] when used consistently and correctly) and to continue its use for 6 months after the last dose of MOXR0916.

TABLE C
Eastern Cooperative Oncology Group
(ECOG) performance status scale

| Grade | Description |
|---|---|
| 0 | Fully active, able to carry on all predisease performance without |
| | restriction |
| 1 | Restricted in physically strenuous activity but ambulatory and |
| | able to carry out work of a light or sedentary nature; e.g., light |
| | housework or office work |
| 2 | Ambulatory and capable of all self-care but unable to carry out |
| | any work activities; up and about >50% of waking hours |
| 3 | Capable of only limited self-care, confined to a bed or |
| | chair >50% of waking hours |
| 4 | Completely disabled; cannot carry on any self-care; totally |
| | confined to bed or chair |
| 5 | Dead |

In addition, patients who meet any of the following exclusion criteria are excluded from study entry. Types of exclusion criteria include cancer-specific, treatment-specific, and general exclusion criteria.

Cancer-specific exclusion criteria include the following:

(a) Any anti-cancer therapy, including chemotherapy, hormonal therapy, or radiotherapy, within 3 weeks prior to initiation of study treatment, with the following exceptions:
(i) Hormonal therapy with gonadotropin-releasing hormone (GnRH) agonists or antagonists for prostate cancer;
(ii) Hormone-replacement therapy or oral contraceptives;
(iii) Herbal therapy >1 week before Cycle 1, Day 1 (herbal therapy intended as anti-cancer therapy must be discontinued at least 1 week before Cycle 1, Day 1);
(iv) Palliative radiotherapy for painful metastases or metastases in potentially sensitive locations (e.g., epidural space) >2 weeks prior to Cycle 1, Day 1;
   (b) Eligibility based on prior treatment with immunomodulatory agents depends on the mechanistic class of the drug and the cohort for which the patient is being considered:
(i) Dose-escalation cohorts and immunotherapy-naïve expansion cohort: Prior treatment with costimulatory agonists (such as anti-OX40, anti-CD137, anti-CD27, anti-GITR, and anti-CD40) or immune checkpoint blockade therapies (including anti-CTLA4, anti-PD-1, and anti-PD-L1 therapeutic antibodies or pathway targeting agents) is not allowed;
(ii) Expansion cohorts other than immunotherapy-naive: Prior treatment with costimulatory agonists (such as anti-OX40, anti-CD137, anti-CD27, anti-GITR, and anti-CD40) or immune checkpoint blockade therapies (including anti-CTLA4, anti-PD-1, and anti-PD-L1 therapeutic antibodies or pathway targeting agents) is allowed provided that no treatment-related Grade ≥3 adverse events (other than endocrinopathy managed with replacement therapy) were observed and at least 6 weeks have elapsed between the last dose and the proposed Cycle 1, Day 1;
(iii) All cohorts: Treatment with systemic immunostimulatory agents not described above (including but not limited to IFNα, IL2) within 6 weeks or 5 half-lives of the drug, whichever is shorter, prior to Cycle 1, Day 1 is not allowed;
   (c) Adverse events from prior anti-cancer therapy that have not resolved to Grade ≤1 except for alopecia or endocrinopathy managed with replacement therapy;
   (d) Primary central nervous system (CNS) malignancy, or untreated/active CNS metastases (progressing or requiring anticonvulsants or corticosteroids for symptomatic control):
(i) Patients with a history of treated CNS metastases are eligible, provided they meet all of the following criteria: measurable disease outside the CNS; radiographic demonstration of improvement upon the completion of CNS-directed therapy and no evidence of interim progression between the completion of CNS-directed therapy and the screening radiographic study; the screening CNS radiographic study is ≥4 weeks since completion of radiotherapy; corticosteroids and anticonvulsants discontinued for ≥2 weeks prior to enrollment with no ongoing symptoms attributable to CNS metastases;
   (e) Leptomeningeal disease;
   (f) Uncontrolled tumor-related pain:
(i) Patients requiring pain medication must be on a stable regimen at study entry;
(ii) Symptomatic lesions amenable to palliative radiotherapy (e.g., bone metastases or metastases causing nerve impingement) should be treated prior to enrollment; and
(iii) Asymptomatic metastatic lesions whose further growth would likely cause functional deficits or intractable pain (e.g., epidural metastasis that is not currently associated with spinal cord compression) should be considered for loco-regional therapy if appropriate prior to enrollment;
   (g) Uncontrolled pleural effusion, pericardial effusion, or ascites requiring recurrent drainage procedures (once monthly or more frequently) (Patients with indwelling catheters, e.g., PleurX, are allowed);
   (h) Malignancies other than disease under study within 5 years prior to Cycle 1, Day 1, with the exception of those with a negligible risk of metastasis or death (such as adequately treated carcinoma in situ of the cervix, basal or squamous cell skin cancer, localized prostate cancer, or ductal carcinoma in situ).

Treatment-specific exclusion criteria include the following:

(a) History of autoimmune disease, including but not limited to systemic lupus erythematosus, rheumatoid arthritis, inflammatory bowel disease, vascular thrombosis associated with antiphospholipid syndrome, Wegener's granulomatosis, Sjögren's syndrome, Bell's palsy, Guillain-Barré syndrome, multiple sclerosis, vasculitis, or glomerulonephritis, with the following caveats:
(i) Patients with a history of autoimmune hypothyroidism on a stable dose of thyroid replacement hormone may be eligible;
(ii) Patients with a history of manageable, reversible immune-related adverse events (irAEs) on prior immunotherapy may be eligible after consultation with the Medical Monitor;
   (b) Treatment with systemic immunosuppressive medications (including but not limited to prednisone, cyclophosphamide, azathioprine, methotrexate, thalidomide, and TNFa antagonists) within 2 weeks prior to Cycle 1, Day 1;
   (c) Patients who have received acute, low-dose, systemic immunosuppressant medications (e.g., a one-time dose of dexamethasone for nausea) may be enrolled in the study after discussion with and approval by the Medical Monitor:
(i) The use of inhaled corticosteroids is allowed;
(ii) The use of mineralocorticoids (e.g., fludrocortisone) for patients with orthostatic hypotension is allowed; and
(iii) Physiologic doses of corticosteroids for adrenal insufficiency are allowed;
   (d) History of idiopathic pulmonary fibrosis, pneumonitis (including drug induced), organizing pneumonia (i.e., bronchiolitis obliterans, cryptogenic organizing pneumonia, etc.), or evidence of active pneumonitis on screening chest CT scan (History of radiation pneumonitis in the radiation field (fibrosis) is permitted);
   (e) Positive test for HIV infection;
   (f) Active hepatitis B (defined as having a positive hepatitis B surface antigen [HBsAg] test at screening). Patients with past or resolved hepatitis B infection (defined as having a negative HBsAg test and a positive IgG antibody to hepatitis B core antigen [anti-HBc]) are eligible;
   (g) Active hepatitis C (Patients positive for hepatitis C virus (HCV) antibody are eligible only if PCR is negative for HCV RNA);
   (h) Active tuberculosis;
   (i) Severe infections within 4 weeks prior to Cycle 1, Day 1, including but not limited to hospitalization for complications of infection, bacteremia, or severe pneumonia;
   (j) Signs or symptoms of infection within 2 weeks prior to Cycle 1, Day 1;
   (k) Received oral or IV antibiotics within 2 weeks prior to Cycle 1, Day 1. Patients receiving prophylactic antibiotics (e.g., for prevention of a urinary tract infection or chronic obstructive pulmonary disease) are eligible;
   (l) Prior allogeneic bone marrow transplantation or prior solid organ transplantation;
   (m) Administration of a live, attenuated vaccine within 4 weeks before Cycle 1, Day 1 or anticipation that such a live attenuated vaccine may be required during the study. Influenza vaccination should be given during influenza season only. Patients must not receive live, attenuated influenza vaccine (e.g., FluMist®) within 4 weeks prior to Cycle 1, Day 1 or at any time during the study;
   (n) History of severe allergic, anaphylactic, or other hypersensitivity reactions to chimeric or humanized antibodies or fusion proteins.

General exclusion criteria include the following:

(a) Inability to comply with study and follow-up procedures;
(b) Pregnancy, lactation, or breastfeeding. Serum pregnancy test (for women of childbearing potential, including women who have had a tubal ligation) must be performed and documented as negative within 14 days prior to Cycle 1,Day 1;
(c) Significant cardiovascular disease, such as New York Heart Association cardiac disease (Class II or greater), myocardial infarction within the previous 3 months, unstable arrhythmias, or unstable angina;
(d) Known clinically significant liver disease, including active viral, alcoholic, or other hepatitis, cirrhosis, and inherited liver disease;
(e) Major surgical procedure within 28 days prior to Cycle 1, Day 1 or anticipation of need for a major surgical procedure during the course of the study;
(f) Any other diseases, metabolic dysfunction, physical examination finding, or clinical laboratory finding giving reasonable suspicion of a disease or condition that contraindicates the use of an investigational drug or that may affect the interpretation of the results or render the patient at high risk from treatment complications.

### Dose Escalation Stage

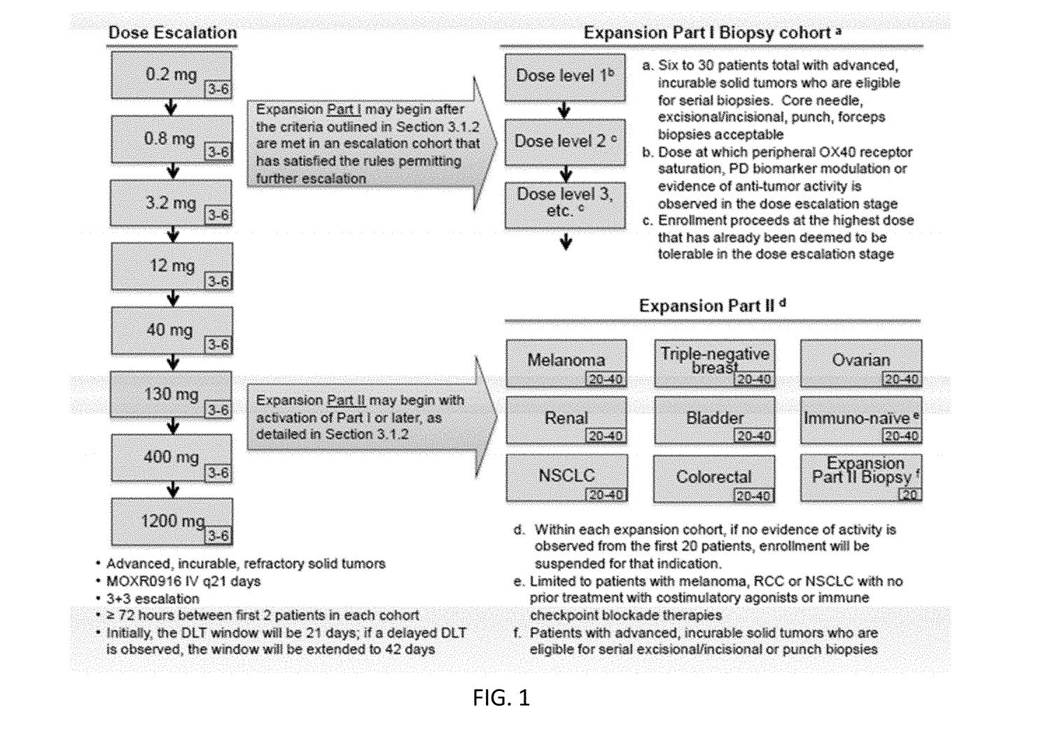

As set forth above and illustrated in FIG. 1, patients are enrolled in a dose-escalation stage and an expansion stage.

Approximately 21 to 36 patients are enrolled in the dose-escalation stage. Cohorts of at least 3 patients each are treated at escalating doses of MOXR0916 in accordance with the dose-escalation rules described below to determine the maximum tolerated dose (MTD) or maximum administered dose (MAD). Enrollment of the first two patients in each dose-escalation cohort is staggered such that their respective Cycle 1 Day 1 treatments are administered ≥72 hours apart.

Initially, the dose-limiting toxicity (DLT) assessment window is 21 days (Days 1-21 of Cycle 1). If a delayed DLT is observed (e.g., as described herein), the DLT assessment window is extended to 42 days after the first administration of MOXR0916 for all patients in that cohort and any subsequent dose-escalation cohorts. Adverse events identified as DLTs or delayed DLTs are reported to the Sponsor within 24 hours.

Any dose-escalation stage patient who does not complete the DLT assessment window (either 21 or 42 days, depending on the DLT assessment window in effect at the time) for a reason other than a DLT is considered non-evaluable for dose-escalation decisions and the MTD assessment and may be replaced by an additional patient at that same dose level. Patients who receive supportive care during the DLT assessment window that confounds the evaluation of DLTs (not including supportive care described below as part of the DLT definition) may be replaced at the discretion of the Medical Monitor. A patient who has MOXR0916 held during the DLT assessment window in order to manage a non-DLT toxicity such that administration of the next planned dose is delayed by more than 7 days, may be considered non-evaluable for dose-escalation decisions and the MTD assessment and may be replaced by an additional patient at that same dose level.

Any one of the following adverse events is considered a DLT if it occurs during the DLT assessment window in a patient enrolled in a dose-escalation cohort and is assessed by the investigator to be related to MOXR0916:

(a) Grade ≥3 non-hematologic, non-hepatic adverse event, with the following exceptions:
(i) Grade 3 nausea, vomiting, or diarrhea that resolves to Grade ≤2 with standard-of-care therapy in ≤3 days;
(ii) Grade 3 fatigue that resolves to Grade ≤2 in ≤3 days;
(iii) Grade 3 fever (>40 degrees C. for ≤24 hours);
(iv) Grade 3 adverse event of tumor flare (defined as local pain, irritation, or rash localized at sites of known or suspected tumor) that resolves to Grade ≤2 in ≤7 days;
(v) Grade 3 laboratory abnormalities that are asymptomatic and considered by the investigator not to be clinically significant that resolve to Grade ≤2 in ≤7 days;
(vi) Grade 3 rash that resolves to Grade ≤2 in ≤7 days with therapy equivalent to prednisone 10 mg/day or less;
   (b) Grade ≥4 neutropenia (absolute neutrophil count [ANC]<500/μL) lasting >7 days;
   (c) Grade ≥3 febrile neutropenia;
   (d) Grade ≥4 anemia;
   (e) Grade ≥4 thrombocytopenia, or Grade 3 thrombocytopenia associated with clinically significant bleeding;
   (f) Grade ≥3 elevation of serum hepatic transaminase (alanine aminotransferase [ALT] or aspartate aminotransferase [AST]) lasting >7 days;
   (g) Grade ≥3 elevation of serum bilirubin; and
   (h) ALT or AST >3× upper limit of normal (ULN) AND total bilirubin >2×ULN.

A delayed DLT is defined as an adverse event that meets one of the above DLT criteria but occurs between 3 and 6 weeks after the first administration of study treatment (Study Days 22-42).

The starting dose of MOXR0916 is 0.2 mg, administered by IV infusion every 21 days to patients in the first cohort. The escalation increment between successive dose levels is no greater than 400% and the proposed doses for evaluation are 0.2 mg, 0.8 mg, 3.2 mg, 12 mg, 40 mg, 130 mg, 400 mg, and 1200 mg.

In addition to any DLTs, other available relevant demographic, adverse event, laboratory, dose administration, and PK/PD data are reviewed prior to all dose-escalation decisions, which are made by the Medical Monitor in consultation with the Principal Investigators and a committee composed of the following Sponsor representatives: safety scientist, statistician, and PK scientist. Based on review of these emergent clinical data, intermediate dose levels may be evaluated.

Dose escalation occurs in accordance with the rules listed below irrespective of the duration of the DLT window:

(a) A minimum of 3 patients is initially enrolled in each cohort;
(b) If none of the first 3 DLT-evaluable patients experiences a DLT, enrollment of the next cohort at the next highest dose level may proceed;
(c) If 1 of the first 3 DLT-evaluable patients experiences a DLT, the cohort is expanded to 6 patients. If there are no further DLTs in the first 6 DLT-evaluable patients, enrollment of the next cohort at the next highest dose level may proceed;
(d) If 2 or more of the first 6 DLT-evaluable patients in a cohort experience a DLT, the MTD is exceeded and dose escalation stops. An additional 3 patients are then evaluated for DLTs at the preceding dose level, unless 6 patients have already been evaluated at that level. However, if the dose level at which the MTD is exceeded is >200% higher than the preceding dose level, 6 patients may be evaluated at an intermediate dose level;
(e) If the MTD is exceeded at any dose level, the highest dose at which fewer than 2 of 6 DLT-evaluable patients (i.e., <33%) experience a DLT is declared the MTD;
(f) If the MTD is not exceeded at any dose level, the highest dose administered in this study is declared the MAD;
(g) Any dose level may be expanded beyond 3 patients in the absence of a DLT if warranted based on Sponsor and investigator evaluation of non-DLT adverse events, including events occurring after Cycle 1 and events observed in the expansion cohorts; and
(h) If two or more patients in a single cohort experience Grade ≥2 adverse events attributed to MOXR0916 or one or more AEs meeting the criteria for DLT are observed at any time during study treatment, the maximum increment between dose levels for any subsequent dose escalation is 200%.

In addition, the following rules apply specifically to the first instance in which a delayed DLT is observed. The dose level at which the delayed DLT was observed is referred to as the “index” dose level or cohort:

(a) Enrollment at or above the index dose level is temporarily suspended, unless the index cohort has enrolled fewer than 3 patients, in which case a total of 3 patients may be initially enrolled in that cohort;
(b) The DLT assessment window is extended to 42 days after the first administration of MOXR0916. This extended window is effective immediately for patients already enrolled at or above the index dose level. Any subsequent enrollment and dose escalation may proceed according to the general rules above, with a 42-day assessment window; and
(c) Patients who have been enrolled at a dose level higher than the index dose level have the option to reduce their dose to a lower dose level, at the discretion of the investigator. A patient who undergoes dose reduction prior to completing the DLT assessment window and does not experience a DLT may be considered non-evaluable for dose-escalation decisions and the MTD assessment.

Based on available preliminary safety and PK data, dose escalation may be halted or modified by the Sponsor as deemed appropriate.

### Expansion Stage

Approximately 166-370 patients are enrolled in the expansion stage, which includes two parts (FIG. 1).

Part I includes a cohort of 6-30 patients. The objectives of Part I are to explore tumor biomarkers of PD activity and obtain additional safety, tolerability, and PK data at multiple dose levels that mirror the dose escalation scheme. Enrollment to this cohort may begin only after peripheral OX40 receptor saturation, PD biomarker modulation, or evidence of anti-tumor activity is observed in an escalation cohort that has satisfied the rules permitting further escalation. Thereafter, enrollment may proceed at the highest dose level that has already been deemed to be tolerable in the dose escalation stage. Patients who are eligible for serial biopsies (core needle, punch, forceps, or excisional/incisional) may be enrolled at each successive dose level. If a higher dose level has met escalation criteria in the dose escalation stage, newly enrolled patients in the Expansion Part I biopsy cohort may receive that dose.

Part II includes multiple cohorts to better characterize the safety, tolerability, PK variability, biomarkers of anti-tumor activity, and preliminary efficacy of MOXR0916 in different cancer types. Enrollment in expansion cohorts may begin with activation of Part I or later, at an initial dose to be determined by the Sponsor in consultation with study investigators, based on assessment of accumulating safety, tolerability, clinical PK, PD, and anti-tumor activity data. The planned expansion cohorts in Part II includes approximately:

(a) 20-40 patients with melanoma;
(b) 20-40 patients with renal cell cancer (RCC);
(c) 20-40 patients with triple negative breast cancer (TNBC);
(d) 20-40 patients with non-small cell lung cancer (NSCLC);
(e) 20-40 patients with urothelial bladder cancer (UBC);
(f) 20-40 patients with colorectal cancer (CRC);
(g) 20-40 patients with ovarian cancer (OC);
(h) 20-40 patients total with either melanoma, RCC, or NSCLC who have not received prior immune checkpoint blockade therapies or costimulatory agonists; and
(i) 20 patients with tumors amenable to serial excisional, incisional or punch biopsies.

In the event that a patient meets criteria for both the Expansion Part I and Expansion Part II biopsy cohorts while both parts are open to enrollment, the patient may be enrolled in Part II.

The Sponsor, in consultation with the investigators, evaluates all available safety data on an ongoing basis to assess the tolerability of the dose levels studied. If the frequency of Grade 3 or 4 toxicities observed in an expansion-stage cohort (including delayed adverse events and events that would otherwise meet the criteria for a DLT) or other unacceptable toxicities, suggest that the MTD has been exceeded at that dose level, accrual at that dose level is halted in the expansion and escalation cohorts and, if applicable, further dose escalation may be halted. Consideration is then given to resuming enrollment in the expansion stage at a lower dose level. In addition, if accumulating tolerability, PK, or PD data suggest that the dose level in an expansion stage cohort is suboptimal for evaluation of anti-tumor activity, consideration may be given to enrolling new patients in that cohort to a different dose level. At no time may a dose level studied in the expansion stage exceed the highest dose level that has met escalation criteria in the dose-escalation stage.

Patients enrolled in either of the dedicated expansion-stage biopsy cohorts may be required to undergo serial tumor biopsies: at baseline after eligibility criteria (other than the requirement for available archival tissue) have been fulfilled, and approximately 2 weeks after the first administration of MOXR0916 (on or between Days 15-21 of Cycle 1). Additional biopsies may be collected at the investigator's discretion, preferably at the time of radiographic response or progression. In the Expansion Part I biopsy cohort, tissue biopsy methods may include core needle, punch, forceps, or excisional/incisional biopsies. In the Expansion Part II biopsy cohort, punch or excisional/incisional biopsies are required. Patients whose baseline biopsy is found to be unevaluable (i.e., due to insufficient material or lack of tumor cells in the sample) may decline to undergo an on-treatment biopsy but may receive study treatment. Such patients may be replaced for the purpose of serial biopsy assessment.

Patients who are enrolled in cohorts other than the dedicated biopsy cohorts may be asked to undergo optional biopsies (core needle, punch, forceps, or excisional/incisional) to explore PD changes related to the activity of MOXR0916. Optional biopsies may be obtained from up to 6 patients in each malignancy-specific expansion cohort. The recommended biopsy timepoints are the same as described above. On-treatment biopsies may not be pursued if the baseline sample is unevaluable.

### Intrapatient Dose Escalation and Dose Reduction

Intrapatient dose escalation to a dose level that has already met criteria for further escalation may be allowed if all of the following conditions are met: the patient has completed at least 4 cycles at their originally assigned dose level or has demonstrated loss of MOXR0916 exposure associated with emergent ATA; the patient has not experienced a DLT or an AE occurring outside the DLT window that would otherwise meet the definition of a DLT; the patient is clinically stable with no decrement in performance status; and the medical monitor has approved the dose escalation.

Treatment after Disease Progression

Patients may continue study treatment after standard RECIST v1.1 criteria for progressive disease are met provided they meet all the following criteria: absence of symptoms and signs (including worsening of laboratory values, e.g., new or worsening hypercalcemia) indicating unequivocal progression of disease; no decline in ECOG performance status; and absence of tumor progression at critical anatomical sites that cannot be readily managed and stabilized by protocol-allowed medical interventions prior to repeat dosing. Critical anatomical sites include the CNS, central airway, the great vessels, and other organs or tissues where compromised function secondary to tumor progression would be expected to result acutely in severe and/or irreversible disability or death.

If radiographic disease progression is confirmed at a subsequent tumor assessment, patients may be considered for continued study treatment at the investigator's discretion after discussion with the Medical Monitor, if they continue to meet the criteria above and have evidence of clinical benefit, as evidenced by at least one of the following: tumor shrinkage (at least 30% decrease in diameter from baseline) of one or more evaluable lesions; or improvement in one or more symptoms or signs attributable to the underlying cancer (e.g., decreased requirement for narcotics for pain, decreased dyspnea associated with pleural effusion, weight gain) as assessed by the investigator.

### Dosage, Administration, and Compliance

The approximate dose levels of MOXR0916 proposed to be evaluated in this study include 0.2, 0.8, 3.2, 12, 40, 130, 400, and 1200 mg administered every 3 weeks by intravenous (IV) infusion. Additional intermediate dose levels of MOXR0916 may be evaluated based on new nonclinical efficacy, clinical safety, and clinical PK data after consultation with participating investigators. The doses are fixed and not dependent on body weight.

MOXR0916 is diluted in 0.9% sodium chloride and administered by intravenous (IV) infusion using syringe pumps or infusion bags, depending on the dose level. Compatibility testing has shown that MOXR0916 is stable when diluted to a concentration ≥0.06 mg/mL in 0.9% sodium chloride diluent in syringes or infusion bags. MOXR0916 may be delivered using syringe pumps and standard medical syringes for dose levels <10 mg and by infusion bags for dose levels ≥10 mg.

The initial dose of MOXR0916 may be delivered over 90±10 minutes (although the infusion may be slowed or interrupted for patients who experience infusion-associated symptoms), followed by a 90-minute observation period. If the 90-minute infusion is tolerated without infusion-associated adverse events, the second infusion may be delivered over 60±10 minutes, followed by a 60 minute observation period. If the 60-minute infusion is well tolerated, all subsequent infusions may be delivered over 30±10 minutes, followed by a 30-minute observation period.

In the event that a patient experiences a mild (NCI CTCAE Grade 1) infusion-related event, the infusion rate should be reduced to half the rate being given at the time of event onset. Approximately 30 minutes after the event has resolved, the infusion may be resumed at the original rate. In the event that a patient experiences a moderate infusion-related event (NCI CTCAE Grade 2) or flushing, fever, or throat pain, the infusion should be immediately interrupted and the patient should receive aggressive symptomatic treatment. The infusion should be restarted only after the symptoms have adequately resolved to baseline grade. The infusion rate at restart should be at most half of the infusion rate that was in progress at the time of the onset of the infusion-related event. For severe or life-threatening infusion-related events (NCI CTCAE Grade 3 or 4), the infusion should be stopped immediately, aggressive resuscitation and supportive measures should be initiated, and no further MOXR0916 for that cycle is administered. Patients who experience Grade 3 events can receive subsequent cycles with premedication following approval of the Medical Monitor, provided that the next dose is infused over 90 minutes. Patients experiencing Grade 4 events permanently discontinue MOXR0916.

No premedication may be allowed for the first dose of MOXR0916. Patients who experience an infusion-associated adverse event may be premedicated for Cycles ≥2 at the discretion of the treating physician after consultation with the Medical Monitor, but the infusion time may not be decreased for that infusion. If the next infusion is well tolerated with premedication, the subsequent infusion time may then be decreased by 30 minutes as long as the patient continues to be premedicated.

If a patient experiences an infusion-associated adverse event with the 60-minute infusion despite premedication, all subsequent doses should be delivered over 90±10 minutes. Similarly, if a patient experiences an infusion-associated adverse event with the 30-minute infusion despite premedication, all subsequent doses should be delivered over 60±10 minutes.

### Concomitant Therapy

Concomitant therapy includes any medication (e.g., prescription drugs, over-the-counter drugs, herbal or homeopathic remedies, nutritional supplements) used by a patient from 7 days prior to screening to the treatment discontinuation visit (and from 7 days prior to re-screening to the re-treatment discontinuation visit). All medications should be reported to the investigator and recorded.

Patients who experience infusion-associated symptoms may be treated symptomatically with acetaminophen, ibuprofen, diphenhydramine, and/or cimetidine or another H2 receptor antagonist, as per standard practice (for sites outside the U.S., equivalent medications may be substituted per local practice). Serious infusion-associated events manifested by dyspnea, hypotension, wheezing, bronchospasm, tachycardia, reduced oxygen saturation, or respiratory distress should be managed with supportive therapies as clinically indicated (e.g., supplemental oxygen and β2-adrenergic agonists). Premedication may be administered for Cycles ≥2 at the discretion of the treating physician after consultation with the Medical Monitor.

Systemic corticosteroids and TNFa antagonists may attenuate potential beneficial immunologic effects of treatment with MOXR0916 but may be administered at the discretion of the treating physician in an emergency or after consultation with the Medical Monitor. If feasible, alternatives to corticosteroids should be considered. The use of inhaled corticosteroids and mineralocorticoids (e.g., fludrocortisone for patients with orthostatic hypotension or adrenocortical insufficiency) is allowed. Physiologic doses of corticosteroids for adrenal insufficiency are allowed. Megestrol administered as an appetite stimulant is also permitted.

Patients who use oral contraceptives, hormone-replacement therapy, prophylactic or therapeutic anticoagulation therapy (such as low molecular weight heparin or warfarin at a stable dose level), or other maintenance therapy for non-malignant indications should continue their use. Males and females of reproductive potential should use highly effective means of contraception.

Use of the following therapies is prohibited during the study:

(a) Any concomitant therapy intended for the treatment of cancer, whether health authority-approved or experimental, including (but not limited to) the following: chemotherapy, hormonal therapy, immunotherapy, radiotherapy, investigational agents, or herbal therapy;
(i) Radiotherapy may be considered for pain palliation (e.g., treatment of known bony metastases) if patients are otherwise deriving benefit. For patients in a dose escalation cohort, palliative radiotherapy should be deferred until completion of the DLT assessment window. MOXR0916 administration may be suspended during radiotherapy with agreement from the Medical Monitor;
(ii) Patients experiencing a mixed response may undergo local therapy (e.g., surgery, stereotactic radiosurgery, radiotherapy, radiofrequency ablation) for control of three or fewer lesions upon approval by the Medical Monitor;
(iii) Patients who undergo radiotherapy to or resection of a target lesion may subsequently become unevaluable for response determination according to RECIST v1.1 or modified RECIST;
   (b) Immunostimulatory agents, including but not limited to IFNα, IFNγ, or IL2, during the entire study;
   (c) Immunosuppressive medications, including but not limited to cyclophosphamide, azathioprine, methotrexate, and thalidomide; and
   (d) Granulocyte colony-stimulating factors (e.g., granulocyte colony-stimulating factor, granulocyte macrophage colony-stimulating factor, and/or pegfilgrastim).

In addition, use of the following therapies is strongly discouraged during the study: traditional herbal medicines; and receptor activator of nuclear factor kappa B (RANK) inhibitor (i.e., denosumab).

### Outcome Measures

The safety and tolerability of MOXR0916 is assessed using the following primary safety outcome measures: incidence and nature of DLTs; and incidence, nature, and severity of adverse events graded according to NCI CTCAE v4.0.

In addition, safety may be assessed using the following secondary safety outcome measures: incidence of anti-MOXR0916 antibodies and the potential correlation with PK, PD, and safety parameters; change in vital signs; change in clinical laboratory results, including ECGs; and number of cycles received and dose intensity.

The following pharmacokinetic (PK) parameters may be derived from concentration-time profile of MOXR0916 following administration, when appropriate as data allow: total exposure (area under the concentration-time curve [AUC]); maximum serum concentration (Cmax); minimum concentration (Cmin); clearance (CL); and volume of distribution at steady state (Vss). Other parameters such as accumulation ratio, half-life, and dose proportionality may also be calculated.

The following activity outcome measures may be assessed:

(a) Objective response, defined as a complete response (CR) or partial response (PR) confirmed ≥4 weeks after initial documentation, determined using RECIST v.1.1;
(b) Duration of objective response, defined as the time from the first occurrence of a documented, objective response until the time of relapse or death from any cause, determined using RECIST v.1.1;
(c) Progression-free survival (PFS), defined as the time from the first study treatment (Day 1) to the first occurrence of progression or death from any cause, whichever occurs first, determined using RECIST v.1.1;
(d) Objective response, duration of objective response, and PFS determined using modified RECIST;
(e) Overall survival (OS), defined as the time from first study treatment to death from any cause.

The following exploratory PD outcome measures may be assessed: changes in TBNK numbers (TBNK assay) in blood; changes in prevalence of various immune cell subpopulations in blood (e.g., effector/memory T cells, regulatory T cells, and MDSCs); changes in activation, proliferation, and functional status of T-cell subsets in blood; identification and profiling of exploratory biomarkers in plasma (i.e., interleukin-2 [IL2], IFNy, and other markers); changes in tumor-infiltrating CD8+ T cells (and other exploratory markers) in freshly obtained tumor tissue prior to and during MOXR0916 treatment; and changes in tumor-infiltrating T-cell activity (measured by expression of granzyme B and other markers) in freshly obtained tumor tissue prior to and during MOXR0916 treatment.

The following additional exploratory biomarker outcome measures may be assessed when appropriate: status of OX40 (and other exploratory markers) in tumor tissue; status of immune infiltrate in tumor tissue, including enumeration and characterization of various immune cell subpopulations; and analysis of single nucleotide polymorphisms (SNPs) in genes including but not limited to those that encode Fc receptors.

### Study Assessments

A complete physical examination performed at screening should include an evaluation of the head, eyes, ears, nose, and throat, and the cardiovascular, dermatological, musculoskeletal, respiratory, gastrointestinal, genitourinary, and neurological systems. Any abnormality identified at baseline should be recorded.

At subsequent visits (or as clinically indicated), limited, symptom-directed physical examinations should be performed. Changes from baseline abnormalities should be recorded in the patient's medical record. New or worsened clinically significant abnormalities should be recorded as adverse events.

As part of tumor assessments, the physical exam should also include evaluation for lymphadenopathy, splenomegaly, hepatomegaly, and cutaneous neoplasms or metastases. All patients should be monitored for symptoms of CNS metastases and such reported symptoms should be followed by a full neurological examination. A brain MRI or contrast enhanced head CT should be done as clinically indicated to confirm or refute new or worsening brain involvement.

All known sites of disease must be documented at screening and re-assessed at each subsequent tumor evaluation. Screening and subsequent tumor assessments must include CT scans (with IV contrast unless contraindicated and oral contrast as appropriate per institutional standards) or MRI of the chest, abdomen, and pelvis. If a CT scan for tumor assessment is performed in a positron emission tomography (PET)/CT scanner, the CT acquisition must be consistent with the standards for a full-contrast CT scan. Brain imaging (either MRI or contrast-enhanced CT) is required at screening for patients with treated brain metastases and as clinically indicated based on symptoms or signs suggestive of new or worsening CNS metastases. In the event of an equivocal head CT, a brain MRI is required to clarify the presence or extent of suspected brain metastases. Further investigations such as bone scans and CT scans of the neck should also be performed if there is any clinical suspicion of disease at any site that may not be demonstrated by the minimum schedule of assessments listed above. At the investigator's discretion, other methods of assessment of measurable disease as per RECIST v1.1 may be used.

The same radiographic procedures used to assess disease sites at screening should be used throughout the study (e.g., the same contrast protocol for CT scans). Response will be assessed by the investigator on the basis of physical examinations and the imaging modalities detailed above, using both RECIST v1.1 and modified RECIST criteria. Assessments should be performed by the same evaluator if possible to ensure internal consistency across visits.

Patients who continue treatment beyond radiographic disease progression per RECIST v1.1 may be monitored with a follow-up scan in 6 (±2) weeks (i.e., at the next scheduled tumor assessment when the scan frequency is every 2 cycles or as an unscheduled tumor assessment when the scan frequency is every 4 cycles), or earlier if clinically indicated. Tumor assessments should be continued every 2 cycles thereafter until two consecutive scans demonstrate stability or improvement with respect to the first scan that showed radiographic disease progression, at which point the scan frequency should revert or transition to every 4 cycles if applicable.

After initial study treatment discontinuation, follow-up tumor assessments may be performed until death, disease progression, initiation of another systemic anti-cancer therapy, loss to follow-up, withdrawal of consent, or study termination, whichever occurs first. Follow-up tumor assessments are not required after discontinuation of MOXR0916 during the re-treatment period.

FDG-PET/CT imaging scans may be acquired at baseline and at the time of the first tumor assessment. In addition, an optional FDG-PET/CT scan may be performed at the first evidence of radiographic disease progression to assess whether apparent increases in tumor volume related to immunomodulatory activity of MOXR0916 (i.e., pseudoprogression) may be distinguished from neoplastic proliferation and disease progression. PET/CT scans at other timepoints are optional. All FDG-PET/CT scans are to be acquired in accordance with the specification provided in the imaging manual. A combination PET and CT scanner should be used for all acquisitions. The baseline FDG-PET/CT scan should be performed during the screening period only after all other inclusion and exclusion criteria have been satisfied, unless it was integrated with a diagnostic quality full-contrast CT scan that fulfills the screening tumor assessment requirement. All FDG-PET/CT scans should be acquired before any scheduled invasive procedure such as a tumor biopsy if at all possible (biopsy location may need to be noted to ensure accurate assessment during central PET imaging review).

The planned duration of the study is approximately 3 years. The end of this study is defined as the date of the initial treatment discontinuation visit for the last patient receiving MOXR0916 in the initial treatment period (LPLV). LPLV is expected to occur approximately 12 months after the last patient has been enrolled.

### Example 2: A First-in-Human Phase I Dose Escalation Study of the OX40 Agonist MOXR0916 in Patients with Refractory Solid Tumors

### Background

OX40 is a co-stimulatory receptor that is transiently expressed by T cells upon antigen recognition. In murine models, OX40 engagement by an agonist anti-OX40 antibody can promote durable tumor regression associated with co-stimulation of effector T cells and reduction of regulatory T cells. MOXR0916 is a humanized effector-competent agonist IgG1 monoclonal antibody that targets OX40. The purpose of this study is to examine the safety and pharmacokinetics (PK) of agonist anti-OX40 antibody treatment.

### Methods

A Phase I, open-label, multicenter study was conducted to evaluate the safety and pharmacokinetics (PK) of MOXR0916 in patients (pts) with locally advanced or metastatic refractory solid tumors that have progressed after available standard therapy. MOXR0916 was administered at fixed doses every 3 weeks (q3w), and treatment beyond RECIST progression was permitted in the absence of clinical deterioration. A 3+3 dose-escalation was conducted in immunotherapy-naïve pts with a 21-day window to evaluate dose-limiting toxicity (DLT). A dedicated expansion cohort enrolled pts who consented to serial tumor biopsies, enabling immune profiling by immunohistochemistry and gene expression methods. In the biopsy cohort, prior immunotherapy with adequate washout was permitted, provided there was no history of Grade (G) ≥3 immune-mediated adverse events (AEs).

### Results

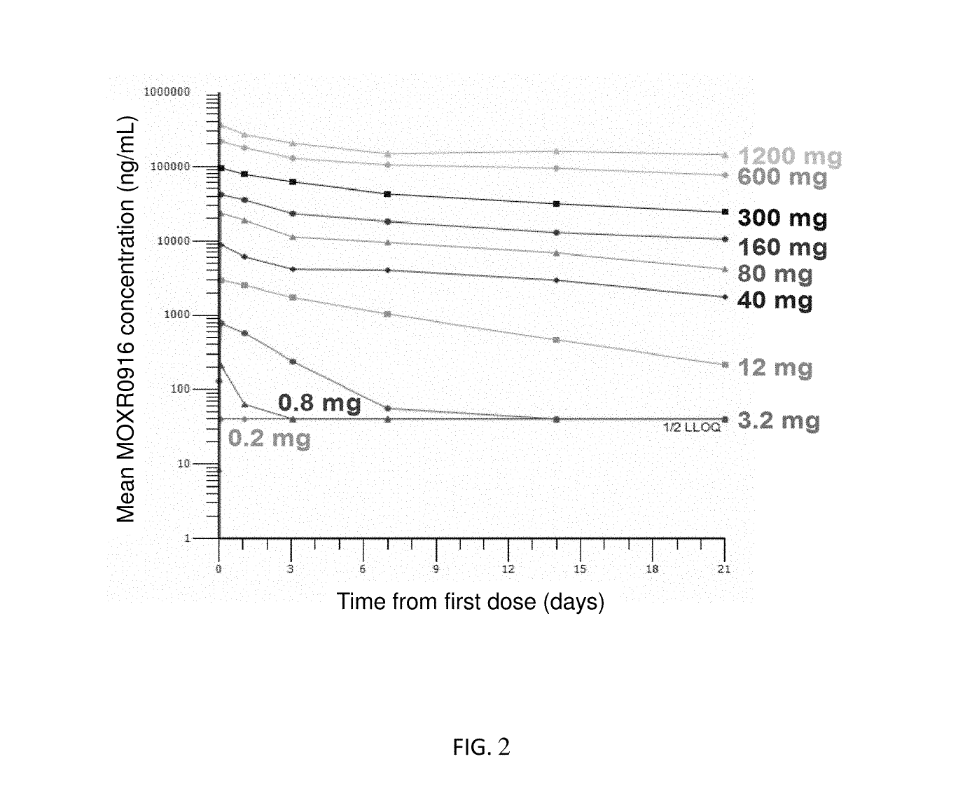

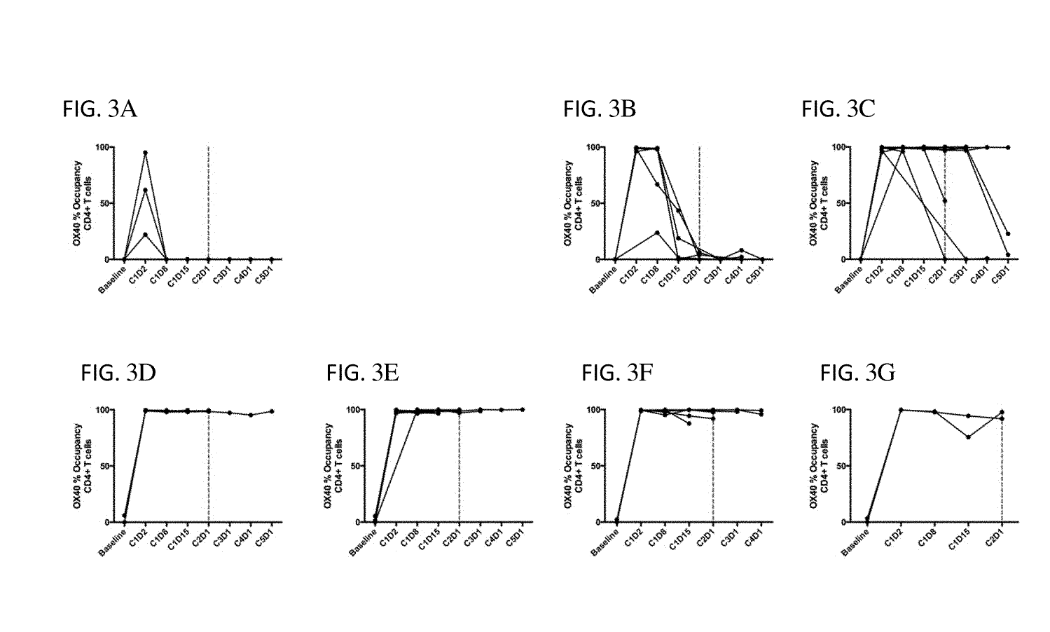

Enrollment in the dose-finding phase of the trial was completed, with 34 pts treated across 10 dose escalation cohorts (dose levels 0.2-1200 mg) and 36 pts treated in the serial biopsy cohort (dose levels 3.2-600 mg). While NSCLC (n=8), clear cell RCC (n=6), melanoma (n=2), and bladder (n=2) were represented, less immunogenic tumor types predominated. The median number of prior regimens for metastatic disease was 2 (range 0-9); 4 pts had received prior checkpoint inhibitors. No DLTs, G4/5 AEs attributed to study treatment, or AEs leading to treatment discontinuation were reported. The majority of treatment-related AEs were G1 in severity; 4 related G3 events (autoimmune hepatitis responsive to steroids, worsening dyspnea in a patient with malignant pleural effusions, hypertension, and fatigue) were reported. At doses ≥40 mg q3w, PK was linear and consistent with IgG1 mAb (FIG. 2) and sustained peripheral blood OX40 receptor saturation was achieved (FIGS. 3A-3G). Dose-dependent peripheral receptor occupancy was observed, with continuous peripheral OX40 saturation achieved at doses ≥40 mg. Doses ≥200 mg are projected to achieve continuous tumor OX40 saturation in cycle 1 (95% occupancy at trough assuming 20:1 blood:tumor partioning). Tumor pharmacodynamic (PD) biomarker modulation supportive of the mechanism of action was observed in a subset of pts.

A transient increase in plasma IP-10 and IFNy was observed as early as 3 hours post >0.2 mg dose and peaked at 24 hour post dose. This increase may be dose dependent above 0.8 mg, and may be mediated by FcR or MOXR0916 co-stimulatory activity. PD-L1 expression increased post MOXR0916 treatment in RCC, NSCLC, melanoma, and cervical tumors.

Significant ATA incidence was observed at low doses, with evidence of ATA impact on PK and receptor occupancy. ATA data suggest low/manageable ATA incidence at doses ≥40 mg. MOXR0916 was well tolerated across dose level with no clear immune-mediated toxicity signals. At the 300 mg dose, the ATA incidence was 1/18 pts.

Eleven of 70 pts (16%) were treated with MOXR0916 for >6 months (≥9 cycles) with a best response of stable disease per RECIST v1.1. The 70 patients were part of the dose escalation and Expansion Part I cohorts (see FIG. 1).

Two RCC patients with partial responses (PRs) were observed in the RCC expansion cohort of the Expansion Part II study population (FIG. 1), which received MOXR0916 with a dosing of 300 mg q3w. Patient 1 was an RCC patient that received 300 mg of MOXR0916 on cycle 1 day 1 (C1D1). Patient lwas a 52 year old male, ECOG 1, with clear cell RCC metastatic to the lungs, bone, and adrenal glands with no liver metastasis. Patient 1 had received prior therapies that included adjuvant axitinib vs placebo (ATLAS trial), first-line (1L) sunitinib (best response PR) and second-line (2L) everolimus (PD). The patient was immunotherapy naïve with a baseline PD-L1 IC 1%. An unconfirmed PR of −42% was observed at week 6 and at week 12, with the sum of the longest diameter (SLD) decreasing from 50 to 29 mm in lung and adrenal target lesions. Patient 2 had received prior therapies that included 1L sunitinib, 2L everolimus, 2L sorafenib, and interferon. A confirmed partial response of −48% at the 1st scan and −63% at the second scan was observed.

### Conclusions

In a heterogeneous, refractory population, MOXR0916 was well-tolerated at all doses evaluated. The recommended dose and schedule based on PK and OX40 receptor saturation was 300 mg q3w. Tumor PD modulation and evidence of prolonged stable disease support the ongoing expansion phase to evaluate anti-tumor activity in select indications.

No dose limiting toxicities were observed, and no deaths or Grade 4 AEs were attributed to MOXR0916. No treatment discontinuations were attributed to drug-related events. Of the 61 discontinuations, 59 were for disease progression, 1 was for physician decision, and 1 was a withdrawal by the subject.

### Example 3: Tumor Immune Modulation Observed in First-in-Human Phase I Dose Escalation Study of the OX40 Agonist MOXR0916 in Patients with Refractory Solid Tumors

Tumor biopsies representing a variety of cancer types were obtained from patients receiving MOXR0916 treatment as described in Examples 1 and 2. Cancer types included renal cell cancer (RCC), non-small cell lung cancer (NSCLC), melanoma, triple-negative breast cancer (TNBC), urothelial bladder cancer (UBC), ovarian cancer, and endometrial cancer. The biopsies also represented a range of MOXR0916 doses, from 3.2 mg to 300 mg.

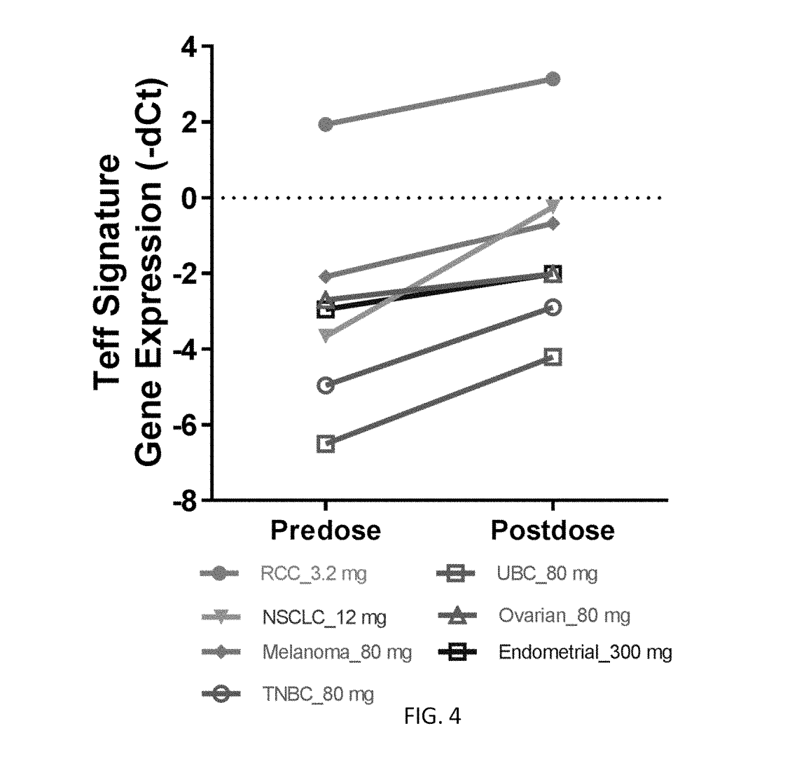

FIG. 4 shows the expression of the Teff gene signature in tumor biopsies measured before and after treatment with MOXR0916 at the indicated dose levels. These data demonstrate an on-treatment increase in Teff signature, which represents effector T cell activation, in a variety of cancer types and at a variety of dose levels. An increase in Teff gene expression was observed in 7 out of 23 tumor biopsies, with no significant change in Teff gene expression observed in 15/23 tumor biopsies, and a decrease in Teff signature observed in 1/23 tumor biopsies. These data indicate a tumoral Teff response in at least a portion of patients treated with MOXR0916.

Tumor biopsies were also analyzed for CD8-expressing cells using immunohistochemistry (IHC). An increase in CD8 infiltrate was observed in 9/23 tumor biopsies upon MOXR0916 treatment, including in biopsies representing TNBC and NSCLC.

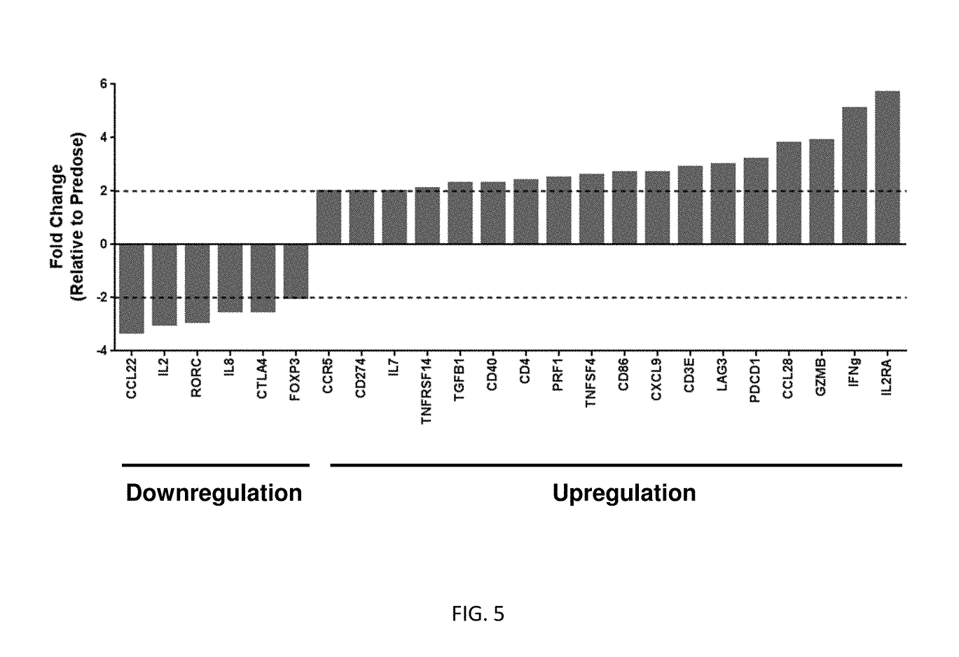

In an RCC tumor biopsy from one patient that received MOXR0916 at a dose of 3.2 mg administered as described above, tumor immune modulation was observed. FIG. 5 shows the postdose fold change in gene expression (as compared to predose levels) of various immune-related genes. Upregulated genes included CCR5, CD274, IL-7, TNFRSF14, TGFB1, CD40, CD4, PRF1, TNFSF4, CD86, CXCL9, CD3E, LAG3, PDCD1, CCL28, GZMB, IFNg, and IL-2RA. This gene expression pattern indicates an increase in Teff activation. Downregulated genes included CCL22, IL-2, RORC, IL-8, CTLA4, and FOXP3. Importantly, expression of these genes is thought to be associated with Treg cells, thus suggesting a decrease in Treg activity. PD-L1 expression was also assayed in the tumor biopsy using IHC, which demonstrated an increase in PD-L1-positive area (relative to overall tumor area) from a predose score of <1% to a postdose score of 5%. Treg cells were also enumerated in the tumor biopsy using immunofluorescence staining against CD3 and Foxp3 as markers. These data indicated a predose Treg frequency (i.e., CD3+FOXP3+ cells) of 2.15% of all cells, as compared to a postdose frequency of 0.58%. In summary, these data indicate a reduction in Tregs, an increase in Teff activation, and an increase in PD-L1 expression upon MOXR0916 treatment.

PD-L1 expression was assayed using IHC in 24 tumor biopsies representing a variety of cancer types. In total, an increase in PD-L1 expression was observed post-MOXR0916 treatment in 8/24 tumor biopsies, with increases observed in RCC, NSCLC, and melanoma samples. No significant change in PD-L1 expression was observed in 16/24 tumor biopsies. The observed increase in PD-L1 expression was enriched in tumors with a higher baseline CD8 prevalance.

In summary, these data show on-treatment immune activation in paired tumor biopsies, demonstrating T cell costimulation. MOXR0916-induced immune modulation was observed in both PD-L1-negative and -positive tumors. Overall, these data suggest that anti-OX40 agonist antibody treatment can increase Teff activation, CD8 infiltration, and PD-L1 expression and decrease tumoral Tregs.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating or delaying progression of cancer in an individual comprising administering to the individual an anti-human OX40 agonist antibody at a dose selected from the group consisting of about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg, wherein the antibody comprises (a) HVR-H1 comprising the amino acid sequence of SEQ ID NO:2; (b) HVR-H2 comprising the amino acid sequence of SEQ ID NO:3; (c) HVR-H3 comprising the amino acid sequence of SEQ ID NO:4; (d) HVR-L1 comprising the amino acid sequence of SEQ ID NO:5; (e) HVR-L2 comprising the amino acid sequence of SEQ ID NO:6; and (f) HVR-L3 comprising an amino acid sequence selected from SEQ ID NO:7, and wherein the individual is a human.

2. The method of claim 1, wherein the dose is about 300 mg.

3. The method of claim 1, wherein the dose is administered intravenously.

4. The method of claim 1, further comprising repeating the administration of the anti-human OX40 agonist antibody at one or more additional doses, wherein each dose of the one or more additional doses is selected from the group consisting of about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg per administration and is administered at an interval of about 2 weeks or about 14 days between each administration.

5. The method of claim 1, further comprising repeating the administration of the anti-human OX40 agonist antibody at one or more additional doses, wherein each dose of the one or more additional doses is selected from the group consisting of about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg per administration and is administered at an interval of about 3 weeks or about 21 days between each administration.

6. The method of claim 4, wherein 1-10 additional doses of the anti-human OX40 agonist antibody are administered.

7. The method of claim 4, wherein each dose of the anti-human OX40 agonist antibody administered to the individual is the same.

8. The method of claim 4, wherein each dose of the anti-human OX40 agonist antibody administered to the individual is not the same.

9. The method of claim 1, wherein each dose of the anti-human OX40 agonist antibody is administered intravenously.

10. The method of claim 9, wherein a first dose of the anti-human OX40 agonist antibody is administered to the individual at a first rate, wherein, after the administration of the first dose, one or more additional doses of the anti-human OX40 agonist antibody are administered to the individual at one or more subsequent rates, and wherein the first rate is slower than the one or more subsequent rates.

11. The method of claim 1, wherein the anti-human OX40 agonist antibody is a humanized antibody.

12. The method of claim 1, wherein the antibody comprises a heavy chain variable domain (VH) sequence having at least 90%, 91%, 92%, 93%, 94%, 95%, 96%, 97%, 98%, 99%, or 100% sequence identity to the amino acid sequence of SEQ ID NO: 56, 58, 60, 62, 64, 66, 68, 183, or 184.

13. The method of claim 12, wherein the VH sequence having at least 90%, 91%, 92%, 93%, 94%, 95%, 96%, 97%, 98%, or 99% identity contains substitutions, insertions, or deletions relative to the reference sequence, but an anti-human OX40 agonist antibody comprising that sequence retains the ability to bind to human OX40.

14. The method of claim 13, wherein a total of 1 to 10 amino acids have been substituted, inserted and/or deleted in SEQ ID NO:56.

15. The method of claim 1, wherein the antibody comprises a light chain variable domain (VL) having at least 90%, 91%, 92%, 93%, 94%, 95%, 96%, 97%, 98%, 99%, or 100% sequence identity to the amino acid sequence of SEQ ID NO: 57, 59, 61, 63, 65, 67, or 69.

16. The method of claim 15, wherein the VL sequence having at least 90%, 91%, 92%, 93%, 94%, 95%, 96%, 97%, 98%, or 99% identity contains substitutions, insertions, or deletions relative to the reference sequence, but an anti-human OX40 agonist antibody comprising that sequence retains the ability to bind to human OX40.

17. The method of claim 16, wherein a total of 1 to 10 amino acids have been substituted, inserted and/or deleted in SEQ ID NO: 57.

18. The method of claim 1, wherein the antibody comprises a VH sequence of SEQ ID NO: 56.

19. The method of claim 1, wherein the antibody comprises a VL sequence of SEQ ID NO: 57.

20. The method of claim 1, wherein the antibody comprises a VH sequence of SEQ ID NO:56 and a VL sequence of SEQ ID NO: 57.

21. The method of claim 1, wherein the antibody is a full length human IgG1 antibody.

22. The method of claim 1, wherein the antibody is MOXR0916.

23. The method of claim 1, wherein the antibody is formulated in a pharmaceutical formulation comprising (a) the antibody at a concentration between about 10 mg/mL and about 100 mg/mL, (b) a polysorbate, wherein the polysorbate concentration is about 0.02% to about 0.06%; (c) a histidine buffer at pH 5.0 to 6.0; and (d) a saccharide, wherein the saccharide concentration is about 120 mM to about 320 mM.

24. The method of claim 1, wherein the treatment results in a sustained response in the individual after cessation of the treatment.

25. The method of claim 1, wherein the treatment results in a complete response (CR) or partial response (PR) in the individual.

26. The method of claim 1, wherein the individual is immunotherapy-naïve.

27. The method of claim 1, wherein the individual has a cancer selected from the group consisting of melanoma, triple-negative breast cancer, ovarian cancer, renal cell cancer, bladder cancer, non-small cell lung cancer, gastric cancer, and colorectal cancer.

28. The method of claim 27, wherein the individual has melanoma, wherein the melanoma has a BRAF V600 mutation, and wherein, prior to the administration of the anti-human OX40 agonist antibody, the individual has been treated with a B-Raf and/or mitogen-activated protein kinase kinase (MEK) kinase inhibitor and exhibited disease progression or intolerance to the B-Raf and/or mitogen-activated protein kinase kinase (MEK) kinase inhibitor treatment.

29. The method of claim 27, wherein the individual has non-small cell lung cancer, wherein the non-small cell lung cancer has a sensitizing epidermal growth factor receptor (EGFR) mutation, and wherein, prior to the administration of the anti-human OX40 agonist antibody, the individual has been treated with an EGFR tyrosine kinase inhibitor and exhibited disease progression or intolerance to the EGFR tyrosine kinase inhibitor treatment.

30. The method of claim 27, wherein the individual has non-small cell lung cancer, wherein the non-small cell lung cancer has an anaplastic lymphoma kinase (ALK) rearrangement, and wherein, prior to the administration of the anti-human OX40 agonist antibody, the individual has been treated with an ALK tyrosine kinase inhibitor and exhibited disease progression or intolerance to the ALK tyrosine kinase inhibitor treatment.

31. The method of claim 27, wherein the individual has renal cell cancer, and wherein the renal cell cancer is refractory to a prior therapy.

32. The method of claim 31, wherein the prior therapy comprises treatment with a VEGF inhibitor, an mTOR inhibitor, or both.

33. The method of claim 1, wherein the anti-human OX40 agonist antibody is MOXR0916, wherein the dose of MOXR0916 is 300 mg, and wherein the cancer is selected from the group consisting of melanoma, triple-negative breast cancer, ovarian cancer, renal cell cancer, bladder cancer, non-small cell lung cancer, gastric cancer, and colorectal cancer.

34. The method of claim 33, further comprising repeating the administration of MOXR0916 at one or more additional doses of 300 mg per administration, administered at an interval of about 3 weeks or about 21 days between each administration.

35. The method of claim 33, wherein MOXR0916 is administered intravenously.

36. The method of claim 1, further comprising, after administering to the individual the anti-human OX40 agonist antibody, monitoring the responsiveness of the individual to said treatment by:
(a) measuring an expression level of one or more marker genes in a sample obtained from the cancer of the individual, wherein the one or more marker genes are selected from the group consisting of CCR5, CD274, IL-7, TNFRSF14, TGFB1, CD40, CD4, PRF1, TNFSF4, CD86, CXCL9, CD3E, LAG3, PDCD1, CCL28, GZMB, IFNg, and IL-2RA; and
(b) optionally, classifying the individual as responsive or non-responsive to treatment with the anti-human OX40 agonist antibody based on the expression level of the one or more marker genes in the sample, as compared with a reference, wherein an increased expression level of the one or more marker genes as compared with the reference indicates a responsive individual.

37. The method of claim 1, further comprising, after administering to the individual the anti-human OX40 agonist antibody, monitoring the responsiveness of the individual to said treatment by:
(a) measuring an expression level of one or more marker genes in a sample obtained from the cancer of the individual, wherein the one or more marker genes are selected from the group consisting of CD8b, EOMES, GZMA, GZMB, IFNg, and PRF1; and
(b) optionally, classifying the individual as responsive or non-responsive to treatment with the anti-human OX40 agonist antibody based on the expression level of the one or more marker genes in the sample, as compared with a reference, wherein an increased expression level of the one or more marker genes as compared with the reference indicates a responsive individual.

38. The method of claim 1, further comprising, after administering to the individual the anti-human OX40 agonist antibody, monitoring the responsiveness of the individual to said treatment by:
(a) measuring an expression level of one or more marker genes in a sample obtained from the cancer of the individual, wherein the one or more marker genes are selected from the group consisting of CCL22, IL-2, RORC, IL-8, CTLA4, and FOXP3; and
(b) optionally, classifying the individual as responsive or non-responsive to said treatment with the anti-human OX40 agonist antibody based on the expression level of the one or more marker genes in the sample, as compared with a reference, wherein a decreased expression level of the one or more marker genes as compared with the reference indicates a responsive individual.

39. A method for determining whether a cancer patient responds to a treatment with an anti-human OX40 agonist antibody, comprising measuring an expression level of one or more marker genes in a sample obtained from the cancer of the individual, wherein the one or more marker genes are selected from the group consisting of CCR5, CD274, IL-7, TNFRSF14, TGFB1, CD40, CD4, PRF1, TNFSF4, CD86, CXCL9, CD3E, LAG3, PDCD1, CCL28, GZMB, IFNg, and IL-2RA, wherein the expression level of the one or more marker genes is compared with a reference, and wherein an increased expression level of the one or more marker genes as compared with the reference indicates that the cancer patient responds to said treatment.

40. A method for determining whether a cancer patient responds to a treatment with an anti-human OX40 agonist antibody, comprising measuring an expression level of one or more marker genes in a sample obtained from the cancer of the individual, wherein the one or more marker genes are selected from the group consisting of CD8b, EOMES, GZMA, GZMB, IFNg, and PRF1, wherein the expression level of the one or more marker genes is compared with a reference, and wherein an increased expression level of the one or more marker genes as compared with the reference indicates that the cancer patient responds to said treatment.

41. A method for determining whether a cancer patient responds to a treatment with an anti-human OX40 agonist antibody, comprising measuring an expression level of one or more marker genes in a sample obtained from the cancer of the individual, wherein the one or more marker genes are selected from the group consisting of CCL22, IL-2, RORC, IL-8, CTLA4, and FOXP3, wherein the expression level of the one or more marker genes is compared with a reference, and wherein a decreased expression level of the one or more marker genes as compared with the reference indicates that the cancer patient responds to said treatment.

42. A kit for treating or delaying progression of cancer in an individual, comprising:
(a) a container comprising anti-human OX40 agonist antibody for administration at a dose selected from the group consisting of about 0.2 mg, about 0.8 mg, about 3.2 mg, about 12 mg, about 40 mg, about 80 mg, about 130 mg, about 160 mg, about 300 mg, about 320 mg, about 400 mg, about 600 mg, and about 1200 mg, wherein the antibody comprises: an HVR-H1 comprising the amino acid sequence of SEQ ID NO:2; an HVR-H2 comprising the amino acid sequence of SEQ ID NO:3; an HVR-H3 comprising the amino acid sequence of SEQ ID NO:4; an HVR-L1 comprising the amino acid sequence of SEQ ID NO:5; an HVR-L2 comprising the amino acid sequence of SEQ ID NO:6; and an HVR-L3 comprising an amino acid sequence selected from SEQ ID NO:7; and
(b) a package insert with instructions for treating or delaying progression of cancer in an individual, wherein the individual is a human.
